(19) United States
(12) Patent Application Publication
(10) Pub. No.: US 2025/0170182 A1
(43) Pub. Date: May 29, 2025

(54) EPITOPE ENGINEERING OF CELL-SURFACE RECEPTORS

(71) Applicants: Dana-Farber Cancer Institute, Inc., Boston, MA (US); The Children's Medical Center Corporation, Boston, MA (US)

(72) Inventors: Pietro Genovese, Boston, MA (US); Gabriele Casirati, Boston, MA (US)

(21) Appl. No.: 18/838,922

(22) PCT Filed: Feb. 16, 2023
(86) PCT No.: PCT/US2023/062757

§ 371 Date: Aug. 15, 2024

Related U.S. Application Data
(60) Provisional application No. 63/311,707, filed on Feb. 18, 2022.
(60) Provisional application No. 63/426,138, filed on Nov. 17, 2022.

(51) Int. Cl. A61K 35/28; A61K 35/17; A61K 40/11; A61K 40/31; A61K 40/42; A61P 35/02; C07K 14/715; C07K 14/725; C07K 16/28; C07K 16/40; C12N 5/0783; C12N 5/0789; C12N 9/12; C12N 9/22; C12N 15/11

(52) U.S. Cl. CPC A61K 35/28; A61K 35/17; A61K 40/11; A61K 40/31; A61K 40/4217; A61K 40/4251; A61P 35/02; C07K 14/7051; C07K 14/7155; C07K 16/2866; C07K 16/40; C12N 5/0636; C12N 5/0647; C12N 9/12; C12N 9/22; C12N 15/111; C07K 2317/31; C07K 2317/34; C07K 2317/53; C07K 2317/92; C07K 2319/02; C07K 2319/03; C12N 2310/20; C12N 2510/00; C12Y 207/10001

(57) ABSTRACT

Genetically engineered hematopoietic cells such as hematopoietic stem cells having one or more genetically edited genes of cell-surface proteins and therapeutic uses thereof, either alone or in combination with immune therapy that targets the cell-surface protein(s).

### CROSS-REFERENCE TO RELATED APPLICATIONS

This application is the § 371 U.S. National Stage of International Application No. PCT/US2023/062757, filed Feb. 16, 2023, which claims the priority benefit of U.S. Provisional Application No. 63/311,707, filed Feb. 18, 2022, and to U.S. Provisional Application No. 63/426,138, filed Nov. 17, 2022, the contents of which are incorporated by reference in their entireties herein.

### TECHNICAL FIELD

Provided herein are genetically engineered hematopoietic cells such as hematopoietic stem cells having one or more genetically edited genes of cell-surface proteins, that can be used in combination with immunotherapies (i.e., cytotoxic agents, such as chimeric antigen receptor T cells), and therapeutic uses thereof.

### BACKGROUND

Innovations in gene transfer have made it possible to reprogram immune cells to target molecules expressed on cancer cells. Exceptionally promising results led the FDA to approve the first adoptive cellular immunotherapy, known as CD19 CAR-T cells, for the treatment of B lymphoblastic leukemia. Although these successes are expected to revolutionize the oncology field, their application has been hampered because most suitable candidates are often shared with healthy bone marrow cells, leading to immunosuppression and severe hematopoietic toxicity. Anti-myeloid/stem cell CAR-T-induced toxicity restricts their applicability to a salvage therapy in a limited time window before HSCT, which may be insufficient for disease eradication. Thus, there remains an unmet need to effectively target cells of interest, e.g., cancer cells, without targeting or harming normal cell populations.

### SUMMARY

The present disclosure generally relates to genetically engineered hematopoietic cells such as hematopoietic stem cells having one or more genetically edited genes of cell-surface proteins, and chimeric antigen receptors that are capable of targeting the same cell-surface proteins.

In one embodiment, provided is a genetically engineered hematopoietic stem cell (HSPC), comprising a genetically engineered FLT3 gene, wherein the genetically engineered FLT3 gene is engineered such that its encoded protein has reduced binding to a therapeutic anti-FLT3 antibody. In an embodiment, the genetically engineered FLT3 gene comprises at least one mutation in exon 9 of the FLT3 gene. In an embodiment, at least one mutation in exon 9 of the genetically engineered FLT3 gene results in a polypeptide bearing a mutation at position N399. In an embodiment, the mutation at position N399 is N399D or N399G. In an embodiment, the therapeutic anti-FLT3 antibody is anti-FLT3 clone 4G8 antibody. In an embodiment, the therapeutic anti-FLT3 antibody is an antibody that has the same six CDRs as, or competes with, 4G8 antibody. In an embodiment, the genetically engineered HSPCs are genetically engineered using a CRISPR system comprising a guide nucleic acid and a nuclease. In an embodiment, the nuclease is either Streptococcus pyogenes Cas9 (SpCas9), Staphylococcus aureus (SaCas9), Lachnospiraceae bacterium Cas12a (LbCas12a), or Acidaminococcus sp. BV3L6 (AsCas12a). In one embodiment, the CRISPR system comprises SpCas9. In an embodiment, the guide nucleic acid is selected from the group consisting of SEQ ID NO: 13, SEQ ID NO: 14, SEQ ID NO: 15, and SEQ ID NO: 16. In an embodiment, CRISPR system further comprises a template DNA. In an embodiment, the template DNA is selected from the group consisting of SEQ ID NO: 40, SEQ ID NO: 41, SEQ ID NO: 42, and SEQ ID NO: 43. In an embodiment, the nuclease is a catalytically impaired SpCas9 linked to a base editor enzyme. In an embodiment, the base editor enzyme is a nucleotide deaminase. In an embodiment, the nucleotide deaminase is either a cytosine deaminase or an adenosine deaminase. In another embodiment, the catalytically impaired SpCas9 is NG-SpCas9 or SpRY-SpCas9. In an embodiment, the catalytically impaired SpCas9 comprises a mutation at position D10A. In an embodiment, the catalytically impaired SpCas9 further comprises a mutation at position K918N. In an embodiment, the guide RNA is selected from the group consisting of SEQ ID NO: 17, SEQ ID NO: 18, SEQ ID NO: 19, SEQ ID NO: 20, and SEQ ID NO: 23. In an embodiment, the genetically engineered FLT3 gene encodes a polypeptide which comprises the amino acid sequence of SEQ ID NO: 51 or SEQ ID NO: 52. Also provided is a population of genetically engineered hematopoietic stem cells (HSPCs), comprising the genetically engineered HSPCs as described above. In some embodiments, provided is a method of treating a hematopoietic malignancy, the method comprising administering to a human subject: (a) the population of genetically engineered hematopoietic stem cells as described above, and (b) a therapeutically effective amount of at least one agent comprising an anti-FLT3 antibody binding domain or an antibody or antibody fragment comprising the anti-FLT3 antibody binding domain. In an embodiment, the at least one agent comprises a Chimeric Antigen Receptor-T (CAR-T) cell comprising the anti-FLT3 antibody binding domain. In an embodiment, the hematopoietic malignancy is B-lymphoblastic leukemia (BLL), acute myeloid leukemia (AML), or T-cell acute lymphoblastic leukemia (T-ALL). In an embodiment, the method further comprises obtaining HSPCs from a biological sample from the human subject and genetically engineering the HSPCs from the biological sample from the human subject, thereby forming the population of genetically engineered HSPCs. In an embodiment, the biological sample is bone marrow cells, blood, cord blood cells, or mobilized peripheral blood-derived CD34+ hematopoietic stem and progenitor cells.

Also provided in an embodiment, is a genetically engineered hematopoietic stem cell (HSPC), comprising a genetically engineered CD123 gene, wherein the genetically engineered CD123 gene is engineered such that its encoded protein has reduced binding to a therapeutic anti-CD123 antibody. In an embodiment, the therapeutic anti-CD123 antibody is clone 7G3 antibody or its humanized counterpart CSL362. In an embodiment, the genetically engineered CD123 gene comprises at least one mutation in exon 2 of the CD123 gene. In an embodiment, at least one mutation in exon 2 of the genetically engineered CD123 gene results in a polypeptide bearing a mutation at position S59. In an embodiment, the mutation at S59 is S59P or S59F. In an embodiment, the therapeutic anti-CD123 antibody is anti-CD123 clone 6H6 antibody or anti-CD123 clone S18016F antibody. In an embodiment, the genetically engineered CD123 gene comprises at least one mutation in exon 3 of the CD123 gene. In an embodiment, at least one mutation in exon 3 of the genetically engineered CD123 gene results in a polypeptide bearing a mutation at position P88. In an embodiment, the mutation at P88 is P88L or P88S. In an embodiment, the genetically engineered HSPCs are genetically engineered using a CRISPR system comprising a guide nucleic acid and a nuclease. In an embodiment, the guide nucleic acid is selected from the group consisting of SEQ ID NO: 24, SEQ ID NO: 27, SEQ ID NO: 30, SEQ ID NO: 32, SEQ ID NO: 33, and SEQ ID NO: 34. In an embodiment, the nuclease is a catalytically impaired SpCas9 linked to a base editor enzyme. In an embodiment, the base editor enzyme is a nucleotide deaminase. In an embodiment, the base editor enzyme is either a cytosine deaminase or an adenosine deaminase. In an embodiment, the catalytically impaired SpCas9 is NG-SpCas9 or SpRY-SpCas9. In an embodiment, the catalytically impaired SpCas9 comprises a mutation at position D10A. In an embodiment, the catalytically impaired SpCas9 further comprises a mutation at position K918N. In an embodiment, the genetically engineered CD123 gene encodes a polypeptide which comprises the amino acid sequence of SEQ ID NO: 54, SEQ ID NO: 55, SEQ ID NO: 56, SEQ ID NO: 57, or SEQ ID NO: 58. Also provided herein is a population of genetically engineered hematopoietic stem cells (HSPCs), comprising the genetically engineered HSPCs as described above. Also provided is a method of treating a hematopoietic malignancy, the method comprising administering to a human subject: (a) the population of genetically engineered hematopoietic stem cells as described above, and (b) a therapeutically effective amount of at least one agent comprising an anti-CD123 antibody binding domain or an antibody or antibody fragment comprising the anti-CD123 binding domain. In an embodiment, the at least one agent comprises a Chimeric Antigen Receptor-T (CAR-T) cell comprising the anti-CD123 antibody binding domain. In an embodiment, the hematopoietic malignancy is B-lymphoblastic leukemia (BLL), acute myeloid leukemia (AML), T-cell acute lymphoblastic leukemia (T-ALL), or Blastic Plasmacytoid Dendritic Cell Leukemia (BPCDN). In an embodiment, the method further comprises obtaining HSPCs from a biological sample from the human subject and genetically engineering the HSPCs from the biological sample from the human subject, thereby forming the population of genetically engineered HSPCs. In an embodiment, the biological sample is bone marrow cells, blood, cord blood cells, or mobilized peripheral blood-derived CD34+ hematopoietic stem and progenitor cells.

In some embodiments provided is a population of genetically engineered hematopoietic stem cells (HSPCs) comprising: (i) a genetically engineered FLT3 gene, wherein the genetically engineered FLT3 gene encodes a protein that has reduced binding to a therapeutic anti-FLT3 antibody, and (ii) a genetically engineered CD123 gene, wherein the genetically engineered CD123 gene encodes a protein that has reduced binding to a therapeutic anti-CD123 antibody. In an embodiment, the genetically engineered FLT3 gene comprises at least one mutation in exon 9 of the FLT3 gene. In an embodiment, at least one mutation in exon 9 of the genetically engineered FLT3 gene results in a polypeptide bearing a mutation at position N399. In an embodiment, the genetically engineered CD123 gene comprises at least one mutation in exon 2 of the CD123 gene. In an embodiment, at least one mutation in exon 2 of the genetically engineered CD123 gene results in a polypeptide bearing a mutation at position S59. In an embodiment, the therapeutic anti-FLT3 antibody is anti-FLT3 clone 4G8 antibody. In an embodiment, the therapeutic anti-CD123 antibody is anti-CD123 clone 7G3 antibody or CSL362 antibody. In an embodiment, the population of HSPCs are genetically engineered using a CRISPR system comprising at least two guide nucleic acids and a nuclease. In an embodiment, the at least two guide nucleic acids are 1) SEQ ID NO: 18 or SEQ ID NO: 20 and 2) SEQ ID NO: SEQ ID NO: 24 or SEQ ID NO: 27. In an embodiment, the at least two guide nucleic acids are SEQ ID NO: 20 and SEQ ID NO: 27. In an embodiment, the nuclease is a catalytically impaired SpCas9 linked to a base editor enzyme. In an embodiment, the base editor enzyme is a nucleotide deaminase. In an embodiment, the base editor enzyme is either a cytosine deaminase or an adenosine deaminase. In an embodiment, the catalytically impaired SpCas9 is NG-SpCas9 or SpRY-SpCas9. In an embodiment, the catalytically impaired SpCas9 comprises a mutation at position D10A. In an embodiment, the SpCas9 further comprises a mutation at position K918N. Also provided is a method of treating a hematopoietic malignancy, the method comprising administering to a human subject: (a) the population of HSPCs as described above, and (b) a therapeutically effective amount of at least one agent comprising one or both of: (1) an anti-FLT3 antibody binding domain or an antibody or antibody fragment comprising the anti-FLT3 antibody binding domain, and/or (2) an anti-CD123 antibody binding domain or an antibody or antibody fragment comprising the anti-CD123 binding domain. In an embodiment, the at least one agent comprises a Chimeric Antigen Receptor-T (CAR-T) cell comprising the anti-FLT3 antibody binding domain and/or the anti-CD123 antibody binding domain. In an embodiment, the hematopoietic malignancy is B-lymphoblastic leukemia (BLL), acute myeloid leukemia (AML), T-cell acute lymphoblastic leukemia (T-ALL), or Blastic Plasmacytoid Dendritic Cell Leukemia (BPCDN). In an embodiment, the method further comprises obtaining HSPCs from a biological sample from the human subject and genetically engineering the HSPCs from the biological sample from the human subject, thereby forming the population of genetically engineered HSPCs. In an embodiment, the biological sample is bone marrow cells, blood, cord blood cells, or mobilized peripheral blood-derived CD34+ hematopoietic stem and progenitor cells.

Also provided in an embodiment is a genetically engineered hematopoietic stem cell (HSPC), comprising a genetically engineered KIT gene, wherein the genetically engineered KIT gene is engineered such that its encoded protein has reduced binding to a therapeutic anti-KIT antibody. In an embodiment, the genetically engineered KIT gene comprises at least one mutation in exon 7 of the KIT gene. In an embodiment, at least one mutation in exon 7 of the genetically engineered KIT gene results in a polypeptide bearing a mutation at position H378. In an embodiment, the mutation at position H378 is H378R. In an embodiment, the therapeutic anti-KIT antibody is anti-KIT clone Fab79D antibody. In an embodiment, the genetically engineered HSPCs are genetically engineered using a CRISPR system comprising a guide nucleic acid and a nuclease. In an embodiment, the guide nucleic acid is selected from the group consisting of SEQ ID NO: 36, SEQ ID NO: 37, SEQ ID NO: 38, and SEQ ID NO: 39. In an embodiment, the nuclease is a catalytically impaired SpCas9 linked to a base editor enzyme. In an embodiment, wherein the base editor enzyme is a nucleotide deaminase. In an embodiment, the base editor enzyme is either a cytosine deaminase or an adenosine deaminase. In an embodiment, the catalytically impaired SpCas9 is NG-SpCas9 or SpRY-SpCas9. In an embodiment, the catalytically impaired SpCas9 comprises a mutation at position D10A. In an embodiment, SpCas9 further comprises a mutation at position K918N. Also provided is a population of genetically engineered hematopoietic stem cells (HSPCs), comprising the genetically engineered HSPCs as described above. Also provided is a method of treating a hematopoietic malignancy, the method comprising administering to a human subject: (a) the population of genetically engineered hematopoietic stem cells as described above, and (b) a therapeutically effective amount of at least one agent comprising the anti-KIT antibody binding domain or an antibody or antibody fragment comprising the anti-KIT binding domain. In an embodiment, the at least one agent comprises a Chimeric Antigen Receptor-T (CAR-T) cell comprising the anti-KIT antibody binding domain. In an embodiment, the hematopoietic malignancy is B-lymphoblastic leukemia (BLL), acute myeloid leukemia (AML), or T-cell acute lymphoblastic leukemia (T-ALL). In an embodiment, the method further comprising obtaining HSPCs from a biological sample from the human subject and genetically engineering the HSPCs from the biological sample from the human subject, thereby forming the population of genetically engineered HSPCs. In an embodiment, the biological sample is bone marrow cells, blood, cord blood cells, or mobilized peripheral blood-derived CD34+ hematopoietic stem and progenitor cells.

Also provided in some embodiments is a population of genetically engineered hematopoietic stem cells (HSPCs) comprising: (i) a genetically engineered KIT gene, wherein the genetically engineered KIT gene encodes a protein that has reduced binding to a therapeutic anti-KIT antibody, and (ii) a genetically engineered CD123 gene, wherein the genetically engineered CD123 gene encodes a protein that has reduced binding to a therapeutic anti-CD123 antibody. In an embodiment, the genetically engineered KIT gene comprises at least one mutation in exon 7 of the KIT gene. In an embodiment, at least one mutation in exon 7 of the genetically engineered KIT gene results in a polypeptide bearing a mutation at position H378. In an embodiment, the genetically engineered CD123 gene comprises at least one mutation in exon 2 of the CD123 gene. In an embodiment, at least one mutation in exon 2 of the genetically engineered CD123 gene results in a polypeptide bearing a mutation at position S59. In an embodiment, the therapeutic anti-KIT antibody is anti-KIT clone Fab79D antibody. In an embodiment, the therapeutic anti-CD123 antibody is anti-CD123 clone 7G3 antibody or CSL362 antibody. In an embodiment, the population of HSPCs are genetically engineered using a CRISPR system comprising at least two guide nucleic acids and a nuclease. In an embodiment, the at least two guide nucleic acids are 1) SEQ ID NO: 36, SEQ ID NO: 37, SEQ ID NO: 38, or SEQ ID NO: 39 and 2) SEQ ID NO: 24 or SEQ ID NO: 27. In an embodiment, the at least two guide nucleic acids are SEQ ID NO: 37 and SEQ ID NO: 27. In an embodiment, the nuclease is a catalytically impaired SpCas9 linked to a base editor enzyme. In an embodiment, the base editor enzyme is a nucleotide deaminase. In an embodiment, the base editor enzyme is either a cytosine deaminase or an adenosine deaminase. In an embodiment, the catalytically impaired SpCas9 is NG-SpCas9 or SpRY-SpCas9. In an embodiment, the catalytically impaired SpCas9 comprises a mutation at position D10A. In an embodiment, the SpCas9 further comprises a mutation at position K918N. Also provided is a method of treating a hematopoietic malignancy, the method comprising administering to a human subject: (a) the population of HSPCs as described above, and (b) a therapeutically effective amount of at least one agent comprising one or both of: (1) an anti-KIT antibody binding domain or an antibody or antibody fragment comprising the anti-KIT binding domain, and/or (2) an anti-CD123 antibody binding domain or an antibody or antibody fragment comprising the anti-CD123 binding domain. In an embodiment, the at least one agent comprises a Chimeric Antigen Receptor-T (CAR-T) cell comprising the anti-KIT antibody binding domain and/or the anti-CD123 antibody binding domain. In an embodiment, the hematopoietic malignancy is B-lymphoblastic leukemia (BLL), acute myeloid leukemia (AML), T-cell acute lymphoblastic leukemia (T-ALL), or Blastic Plasmacytoid Dendritic Cell Leukemia (BPCDN). In an embodiment, the method further comprises obtaining HSPCs from a biological sample from the human subject and genetically engineering the HSPCs from the biological sample from the human subject, thereby forming the population of genetically engineered HSPCs. In an embodiment, the biological sample is bone marrow cells, blood, cord blood cells, or mobilized peripheral blood-derived CD34+ hematopoietic stem and progenitor cells.

Also provided herein is a population of genetically engineered hematopoietic stem cells (HSPCs) comprising: (i) a genetically engineered FLT3 gene, wherein the genetically engineered FLT3 gene encodes a protein that has reduced binding to a therapeutic anti-FLT3 antibody, and (ii) a genetically engineered KIT gene, wherein the genetically engineered KIT gene encodes a protein that has reduced binding to a therapeutic anti-KIT antibody. In an embodiment, the genetically engineered FLT3 gene comprises at least one mutation in exon 9 of the FLT3 gene. In an embodiment, at least one mutation in exon 9 of the genetically engineered FLT3 gene results in a polypeptide bearing a mutation at position N399. In an embodiment, the genetically engineered KIT gene comprises at least one mutation in exon 7 of the KIT gene. In an embodiment, at least one mutation in exon 7 of the genetically engineered KIT gene results in a polypeptide bearing a mutation at position H378. In an embodiment, the therapeutic anti-FLT3 antibody is anti-FLT3 clone 4G8 antibody. In an embodiment, the therapeutic anti-KIT antibody is anti-KIT clone Fab79D antibody. In an embodiment, the population of HSPCs are genetically engineered using a CRISPR system comprising at least two guide nucleic acids and a nuclease. In an embodiment, the at least two guide nucleic acids are 1) SEQ ID NO: 18 or SEQ ID NO: 20 and 2) SEQ ID NO: 36, SEQ ID NO: 37, SEQ ID NO: 38, or SEQ ID NO: 39. In an embodiment, the at least two guide nucleic acids are SEQ ID NO: 20 and SEQ ID NO: 37. In an embodiment, the nuclease is a catalytically impaired SpCas9 linked to a base editor enzyme. In an embodiment, the base editor enzyme is a nucleotide deaminase. In an embodiment, the base editor enzyme is either a cytosine deaminase or an adenosine deaminase. In an embodiment, the catalytically impaired SpCas9 is NG-SpCas9 or SpRY-SpCas9. In an embodiment, the catalytically impaired SpCas9 comprises a mutation at position D10A. In an embodiment, the SpCas9 further comprises a mutation at position K918N. Also provided is a method of treating a hematopoietic malignancy, the method comprising administering to a human subject: (a) the population of HSPCs as described above, and (b) a therapeutically effective amount of at least one agent comprising one or both of: (1) an anti-FLT3 antibody binding domain or an antibody or antibody fragment comprising the anti-FLT3 antibody binding domain, and/or (2) an anti-KIT antibody binding domain or an antibody or antibody fragment comprising the anti-KIT binding domain. In an embodiment, the at least one agent comprises a Chimeric Antigen Receptor-T (CAR-T) cell comprising the anti-FLT3 antibody binding domain and/or the anti-KIT antibody binding domain. In an embodiment, the hematopoietic malignancy is B-lymphoblastic leukemia (BLL), acute myeloid leukemia (AML), T-cell acute lymphoblastic leukemia (T-ALL, or Blastic Plasmacytoid Dendritic Cell Leukemia (BPCDN). In an embodiment, the method further comprises obtaining HSPCs from a biological sample from the human subject and genetically engineering the HSPCs from the biological sample from the human subject, thereby forming the population of genetically engineered HSPCs. In an embodiment, the biological sample is bone marrow cells, blood, cord blood cells, or mobilized peripheral blood-derived CD34+ hematopoietic stem and progenitor cells.

Also provided in some embodiments is a chimeric antigen receptor (CAR) comprising a polypeptide comprising: (a) one or more epitope binding fragments that binds to an epitope of one or more cell-surface lineage-specific proteins, (b) a hinge domain, (c) a transmembrane domain, (d) a co-stimulatory domain, and (e) a cytoplasmic signaling domain, wherein the one or more cell-surface lineage-specific proteins are selected from FLT3, CD123, and/or KIT.

In some embodiments, the cell-surface lineage-specific protein is FLT3 and the CAR comprises the amino acid sequence of SEQ ID NO: 73. In some embodiments, the cell-surface lineage-specific protein is FLT3 and the one or more epitope binding fragments comprises the one or more epitope binding fragments from SEQ ID NO: 73. In some embodiments, the cell-surface lineage-specific protein is FLT3 and the one or more epitope binding fragments comprise the following CDR sequences: GYTFTSYWMH (SEQ ID NO: 96), EIDPSDSYKDYNQKFK (SEQ ID NO: 97), RAITTTPFDF (SEQ ID NO: 98), RASQSISNNLH (SEQ ID NO: 99), YASQSIS (SEQ ID NO: 100), and QQSNTWPYT (SEQ ID NO: 101). In some embodiments, the cell-surface lineage-specific protein is CD123 and the CAR comprises the amino acid sequence of SEQ ID NO: 75, SEQ ID NO: 86, or SEQ ID NO: 87. In some embodiments, the cell-surface lineage-specific protein is CD123 and the one or more epitope binding fragments comprises the one or more epitope binding fragments from SEQ ID NO: 75, SEQ ID NO: 86, or SEQ ID NO: 87. In some embodiments, the cell-surface lineage-specific protein is CD123 and the one or more epitope binding fragments comprise the following CDR sequences: GYSFTDYYMK (SEQ ID NO: 104), DIIPSNGATFYNQKFKG (SEQ ID NO: 105), ARSHLLRASWFAY (SEQ ID NO: 106), SQSLLNSGNQKNYLT (SEQ ID NO: 107), WASTRES (SEQ ID NO: 108), and QNDYSYPYT (SEQ ID NO: 109). In some embodiments, the cell-surface lineage-specific protein is CD123 and the one or more epitope binding fragments comprise the following CDR sequences: DIIPSNGATFYNQKFKG (SEQ ID NO: 105), SQSLLNSGNQKNYLT (SEQ ID NO: 107), WASTRES (SEQ ID NO: 108), and QNDYSYPYT (SEQ ID NO: 109). In some embodiments, the cell-surface lineage-specific protein is KIT and the CAR comprises the amino acid sequence of SEQ ID NO: 69 or SEQ ID NO: 71. In some embodiments, the cell-surface lineage-specific protein is KIT and the one or more epitope binding fragments comprises the one or more epitope binding fragments from SEQ ID NO: 69 or SEQ ID NO: 71. In some embodiments, the cell-surface lineage-specific protein is KIT and the one or more epitope binding fragments comprise the following CDR sequences: GFNISVYMMH (SEQ ID NO: 88), SIYPYSGYTYYADSVKG (SEQ ID NO: 89), ARYVYHALDY (SEQ ID NO: 90), RASQRGLRNVAVA (SEQ ID NO: 91), SASSLYS (SEQ ID NO: 92), and QQWAVHSLIT (SEQ ID NO: 93). In some embodiments, the one or more cell-surface lineage-specific proteins are FLT3 and CD123 and the CAR comprises the amino acid sequence of SEQ ID NO: 77 or SEQ ID NO: 79. In some embodiments, the one or more cell-surface lineage-specific proteins are FLT3 and CD123 and the one or more epitope binding fragments comprises the one or more epitope binding fragments from SEQ ID NO: 77 or SEQ ID NO: 79. In some embodiments, the one or more cell-surface lineage-specific proteins are FLT3 and CD123 and the one or more epitope binding fragments comprise the following CDR sequences: GYTFTSYWMH (SEQ ID NO: 96), EIDPSDSYKDYNQKFK (SEQ ID NO: 97), RAITTTPFDF (SEQ ID NO: 98), RASQSISNNLH (SEQ ID NO: 99), YASQSIS (SEQ ID NO: 100), QQSNTWPYT (SEQ ID NO: 101), GYSFTDYYMK (SEQ ID NO: 104), DIIPSNGATFYNQKFKG (SEQ ID NO: 105), ARSHLLRASWFAY (SEQ ID NO: 106), SQSLLNSGNQKNYLT (SEQ ID NO: 107), WASTRES (SEQ ID NO: 108), and QNDYSYPYT (SEQ ID NO: 109). In some embodiments of any of the CARs described above, the hinge domain is a CD28 hinge, an IgG4 hinge, or a CD8α hinge. In some embodiments of any of the CARs described above, the transmembrane domain is a CD28 TM, a CD8α TM, or a 4-1BB™. In some embodiments of any of the CARs described above, the co-stimulatory domain is CD28z, 4-1BB, ICOS, or OX40. In some embodiments of any of the CARs described above, wherein the cytoplasmic signaling domain is CD3z.

Also provided herein are cells expressing any of the above-described CARs. In some embodiments, the cell is an immune cell. In some embodiments, the immune cell is a T-cell.

Also provided herein are methods of treating a hematopoietic malignancy, the method comprising administering to a human subject: (a) a population of genetically engineered hematopoietic stem cells (such as any of those described above, and (b) cells expressing any of the above-described CARs (such as an immune cell).

Also provided herein is a polypeptide sequence comprising a polypeptide sequence that is at least 80% identical to the sequence set forth in SEQ ID NO: 51, wherein the polypeptide sequence comprises a mutation at N399D and wherein the polypeptide sequence has reduced binding to a therapeutic anti-FLT3 antibody.

Also provided herein is a polypeptide sequence comprising a polypeptide sequence that is at least 80% identical to the sequence set forth in SEQ ID NO: 52, wherein the polypeptide sequence comprises a mutation at N399G and wherein the polypeptide sequence has reduced binding to a therapeutic anti-FLT3 antibody.

Also provided herein is a polypeptide sequence comprising a polypeptide sequence that is at least 80% identical to the sequence set forth in SEQ ID NO: 54, wherein the polypeptide sequence comprises a mutation at S59P and wherein the polypeptide sequence has reduced binding to a therapeutic anti-CD123 antibody.

Also provided herein is a polypeptide sequence comprising a polypeptide sequence that is at least 80% identical to the sequence set forth in SEQ ID NO: 55, wherein the polypeptide sequence comprises mutations at Y58H and S59P and wherein the polypeptide sequence has reduced binding to a therapeutic anti-CD123 antibody.

Also provided herein is a polypeptide sequence comprising a polypeptide sequence that is at least 80% identical to the sequence set forth in SEQ ID NO: 56, wherein the polypeptide sequence comprises a mutation at S59F and wherein the polypeptide sequence has reduced binding to a therapeutic anti-CD123 antibody.

Also provided herein is a polypeptide sequence comprising a polypeptide sequence that is at least 80% identical to the sequence set forth in SEQ ID NO: 57, wherein the polypeptide sequence comprises a mutation at P88S and wherein the polypeptide sequence has reduced binding to a therapeutic anti-CD123 antibody.

Also provided herein is a polypeptide sequence comprising a polypeptide sequence that is at least 80% identical to the sequence set forth in SEQ ID NO: 58, wherein the polypeptide sequence comprises a mutation at P88L and wherein the polypeptide sequence has reduced binding to a therapeutic anti-CD123 antibody.

Also provided herein is a polypeptide sequence comprising a polypeptide sequence that is at least 80% identical to the sequence set forth in SEQ ID NO: 67, wherein the polypeptide sequence comprises mutations at F316S, M318V, I319K, V323I, I334V, E360K, P363V, E366D, E376Q, and H378R and wherein the polypeptide sequence has reduced binding to a therapeutic anti-KIT antibody.

Also provided herein is a polypeptide sequence comprising a polypeptide sequence that is at least 80% identical to the sequence set forth in SEQ ID NO: 68, wherein the polypeptide sequence comprises a mutation at H378R and wherein the polypeptide sequence has reduced binding to a therapeutic anti-KIT antibody.

Also provided herein are nucleic encoding any of the above described polypeptides.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a depiction of the structure of cytokine receptors encoded by genes such as FLT3 or KIT. FIG. 1 (Left) exemplifies the epitope engineering approach to abrogate the binding of a therapeutic antibody by introducing mutations on the extracellular domain. FIG. 1 (Right) shows the main features of epitope engineered surface proteins: loss of antibody recognition with preservation of ligand affinity, protein function and intracellular signal transduction.

FIG. 2, on the left, is a schematic of a sleeping beauty transposase experiment, including transfer vector design, to introduce the cDNA sequence of the desired receptor variant in human or murine cell lines, in order to evaluate the recognition of such variant by different antibody clones or measure ligand affinity by flow cytometry. K562 cells were electroporated with 100 ng transfer plasmid and 500 ng pSB100x transposase using a Lonza 4D-Nucleofector. FIG. 2, on the right, shows flow cytometry plots of K562 cells transduced by sleeping beauty transposase with the human wild-type FLT3 (hFLT3), murine FLT3 (mFLT3) and an epitope modified variant, eFLT3-01, which bears amino-acid substitution within the extracellular domain 4. Transduced cells are identified by mCherry fluorescence (y axis). Cells were stained with either FLT3 BV10A4 clone (control antibody, binding ECD2), FLT3 4G8 therapeutic clone (binding ECD4), anti-murine FLT3 antibody and AF488-conjugate human FLT3L to evaluate binding affinity.

FIG. 3, on the top, is a sequence alignment of the human FLT3 extracellular domain 4 with several animal species ortholog genes. Black rectangles highlight less conserved residues which have been mutated in eFLT3-01 and included in the combinatorial library shown in FIG. 5. FIG. 3, on the bottom, is a sequence alignment of the human KIT extracellular domain 4 with several animal species ortholog genes. Black rectangles highlight less conserved residues which have been mutated in eKIT-01 and are predicted to contribute to the epitope bound by clone Fab79D.

FIG. 4A is a flow cytometry plot showing co-staining of sleeping-beauty transduced K562 cells with a control antibody (clone BV10A4) and a therapeutic antibody (clone 4G8). eFLT3-01 variant overexpression shows lack of recognition by 4G8 clone, while wild type FLT3 is bound by both therapeutic and control Abs.

FIG. 4B is a flow cytometry plot showing fluorescent FLT3-ligand binding by sleeping-beauty transduced K562 cells. Cells were incubated with AF488-conjugated human FLT3L and control antibody BV10A4, which recognizes ECD2. The MFI ratio between the control antibody and AF488-conjugated human FLT3L is reported in the plot. eFLT3-01 variant demonstrates comparable ligand binding affinity as wild type FLT3.

FIG. 4C is a western blot of sleeping-beauty transduced K562 cells protein extracts. Cells were serum starved for 16 h, then stimulated with FLT3L 100 ng/ml or non-stimulated and lysed to obtain protein extracts. K562 (untransduced), K562 overexpressing wild type FLT3 and K562 overexpressing eFLT3-01 were probed with antibodies recognizing FLT3 extracellular domain, phospho-FLT3 Y589-591 or actin (control). eFLT3-01 variant shows kinase domain phosphorylation in response to FLT3L stimulation similar to wild type FLT3.

FIG. 5 (top left) shows the design of the FLT3 combinatorial library cloned in a sleeping beauty plasmid. FIG. 5 (bottom left) shows flow cytometry plots of K562 cells transduced by wild-type FLT3 or the FLT3 combinatorial library, stained with a control antibody (clone BV10A4) and a therapeutic antibody (clone 4G8). NGS sequencing of sorted single positive and double positive cells highlighted the presence of N399D mutations only in the sorted single positive sample. FIG. 5 (right) is the validation of the FLT3 N399D mutation overexpressed in K562 cells through sleeping beauty transduction. Co-staining with the BV10A4 and 4G8 clones shows lack of binding by the latter, while ligand affinity is preserved as assessed with AF488-conjugated FLT3L.

FIG. 6A is a flow cytometry plot showing co-staining of sleeping-beauty transduced NIH3T3 cells with a control antibody (clone 104D2) and a therapeutic antibody (clone Fab79D). eKIT-01 variant overexpression shows lack of recognition by Fab79D clone, while wild type KIT is bound by both therapeutic and control Abs.

FIG. 6B is a flow cytometry plot showing fluorescent SCF binding by sleeping-beauty transduced NIH3T3 cells. Cells were incubated with AF488-conjugated human SCF and control antibody 104D2. The MFI ratio between the control antibody and AF488-conjugated human SCF is reported in the plot. eKIT-01 variant demonstrates comparable ligand binding affinity as wild type KIT.

FIG. 6C is a western blot of sleeping-beauty transduced NIH3T3 cells protein extracts. Cells were serum starved for 16 h, then stimulated with SCF 100 ng/ml or non-stimulated and lysed to obtain protein extracts. NIH3T3 (untransduced), NIH3T3 overexpressing wild type KIT and NIH3T3 overexpressing eKIT-01 were probed with antibodies recognizing KIT extracellular domain, phospho-KIT Y719 or actin (control). eKIT-01 variant shows kinase domain phosphorylation in response to SCF stimulation similar to wild type KIT.

FIG. 6D reports flow cytometry plots of HEK-293T cells transduced with a sleeping-beauty system with several mutated KIT variants, in addition to wild type KIT. Cells were stained with a control antibody (clone 104D2) and a therapeutic antibody (Fab79D) to identify the best candidate mutations to abrogate Fab79D binding while preserving surface expression.

FIG. 7, top left, is the experimental design for KIT extracellular domain 4 degenerated library screening, which was performed to map the epitope of clone Fab79D. Each amino-acid residue within ECD 4 was substituted with a fully degenerated codon (NNN) to allow for any amino-acid substitution and cloned in a sleeping beauty plasmid with mTagBFP2 as co-expressed marker for transduced cells. The library was electroporated in HEK-293T cells. FIG. 7, bottom left, is the gating strategy for FACS sorting of the KIT extracellular domain 4 degenerated library to isolate single positive cells (for KIT control antibody clone 104D2) to be NGS sequenced. FIG. 7, on the right, is a heatmap matrix with each amino acid position within the extracellular domain 4 library region as columns and the amino acid substitutions enriched in single positive vs double positive FACS-sorted and sequenced cells. The most frequently mutated amino acid positions represent the residues which take part in Fab79D epitope recognition (reported on the top part of the plot).

FIG. 8 is the experimental layout for targeted EF1-alpha promoter insertion upstream of the FLT3 and IL3RA (CD123) reading frames, mediated by CRISPR-Cas9 or Cas12a homology directed repair. This experiment was performed to generate reporter cell lines to allow for enhanced and faster evaluation of editing outcomes by flow cytometry, as unmodified K562 cells do not express either gene. The same strategy has been applied to the KIT gene, to overexpress it from its endogenous locus. K562 were electroporated with RNP complexes together with dsDNA donor template with homology arms for the FLT3 or IL3RA promoter region. Cells expressing the desired gene were then FACS sorted, single cell-cloned and screened to identify the clones with highest expression.

FIG. 9 is the mapping of anti-CD123 clone 7G3 epitope on CD123 N-terminal domain. Each amino acid position has been substituted either with Alanine or evolutionary conserved amino acids and screened for 7G3 binding. White squares identify residues with no impact on 7G3 binding, while progressively darker grey-colored squares are associate with 25-50-75% loss of clone 7G3 binding. Similarly, the bottom part of FIG. 9 shows the binding site for IL3 on CD123.

FIG. 10A shows the position of several sgRNAs in relation to IL3RA exon 2 and 3 used in a base editing screening experiment to identify gRNAs and mutations capable of abrogating clone 7G3 binding while preserving CD123 surface expression. The type of Cas protein associated with each sgRNA is reported in the legend.

FIG. 10B shows representative flow cytometry plots of K562 reporter cells expressing CD123 from its endogenous locus edited with different sgRNA and base editor pairs (indicate above and at the left of each plot). Cells are stained with a control antibody, CD123 clone 9F5, and 3 different therapeutic antibodies, CD123 clones 7G3, 6H6 and S18016F. sgRNA and plasmid dose electroporated into the cells is reported in the figure.

FIG. 11 Base editing screening experiment on CD123 reporter cells edited with several sgRNA-base editor pairs (reported on the left or at the top of the plots, respectively). Cells were electroporated with 360 pmol of sgRNA and 500 ng of base editor plasmid with a Lonza 4D-Nucleofector and evaluated by flow cytometry after 72h. Cells are stained with a control antibody, CD123 clone 9F5, and 3 different therapeutic antibody, CD123 clones 7G3, 6H6 and S18016F.

FIG. 12A reports the design of the lentiviral vector encoding for the FLT3 4G8 CAR and a truncated EGFR transduction (LV) marker/safety switch. A 3^{rd} generation LV vector was produced, and fresh PBMC-derived T cells were transduced at day 2-3 of stimulation with CD3-CD28 Dynabeads and cultured for 14 days in medium containing IL7 and IL15. For co-culture killing experiments, K562 cells expressing either no FLT3, wild type FLT3 or epitope engineered variants of FLT3 were plated in a 96-well plate with 4G8-CAR T cells or untransduced (UT) T cells at different effector: target ratios (ET).

FIG. 12B (top row) are flow cytometry plots of the plated K562 cells showing the expression of FLT3 by co-staining with a control antibody (clone BV10A4) and a therapeutic antibody (clone 4G8). FIG. 12B (middle and bottom row) are flow cytometry plots of live cells (LiveDead yellow-AnnexinV-) from the co-culture killing experiment at 4 and 48 hours after plating. Effector cells are identified by CellTrace fluorescence or CD4/CD8 expression, while target cells are CellTrace and CD4/CD8-negative and FSC-A high.

FIG. 12C is a plot showing the percentage of viable target cells (LiveDead-AnnexinV-) at 4h in all tested conditions.

FIG. 12D is a plot showing CD107a surface staining on T lymphocytes at 4h as marker of degranulation during co-culture with target cells.

FIG. 12E is a plot reporting CellTrace yellow MFI at 48h after co-culture with target cells to evaluate T cell proliferation by dye-dilution.

FIG. 13A is a schematic showing the position of 3 gRNAs relative to FLT3 exon 9 for a CRISPR-Cas homology directed repair (HDR) editing strategy. 2 gRNAs are designed for use in combination with AsCas12a and one with SpCas9 nucleases. Single strand oligo-deoxynucleotides are (ssODN) are used as template donor and the design of 4 different variants are reported. Black squares within the ssODN sequence identify the mutated codons differing from wild type sequence (additional mutations are included to reduce the rate of Cas nuclease re-cutting after HDR mediated repair). The reverse complement of each ssODN was also tested.

FIG. 13B are flow cytometry plots reporting the outcome of a CRISPR-Cas HDR editing experiment performed on reporter K562 cells expressing FLT3 with the gRNAs and ssODN reported in FIG. 13A. Cells were co-stained with a control antibody (clone BV10A4) and a therapeutic antibody (clone 4G8). The black square highlight edited cells.

FIG. 14A is a schematic showing the position of 2 sgRNAs relative to FLT3 exon 9 and the N399 codon to be used in combination with adenine base editors (ABE).

FIG. 14B reports the sequence of each gRNA in relationship with the N399 codon and the PAM sequence.

FIG. 14C is a schematic drawing of 3 adenine base editors variants with mutated Cas9 to allow the use with alternative PAM sequences.

FIG. 14D are flow cytometry plots showing the outcome of a base editing experiment on FLT3-expressing K562 reporter cells with the sgRNAs depicted in FIG. 14A and FIG. 14B and the ABE reported in FIG. 14C. Cells were co-stained with a control antibody (clone BV10A4) and a therapeutic antibody (clone 4G8). Each plot reports the gating for edited cells and the percentage of edited cells.

FIG. 15A is a schematic showing the position of 5 sgRNAs relative to FLT3 exon 9 and the N399 codon to be used in combination with adenine base editors (ABE) to introduce the N399D mutation. The legend shows the different PAM and SpCas9 requirements for each sgRNA.

FIG. 15B reports the sequence of each gRNA in relationship with the N399 codon and the PAM sequence.

FIG. 15C are flow cytometry plots showing the outcome of a base editing experiment on FLT3-expressing K562 reporter cells with the sgRNAs depicted in FIG. 15A and FIG. 15B and two adenine base editors, NG-ABE8e and SpRY-ABE8e-V106W 3xNLS. Cells were co-stained with a control antibody (clone BV10A4) and a therapeutic antibody (clone 4G8). Each plot reports the gating for edited cells and the percentage of edited cells.

FIG. 16A are flow cytometry plots of FLT3 reporter K562 cells overexpressing different FLT3 variants by sleeping beauty transduction and co-stained with anti-FLT3 control antibody (clone BV10A4) and AF488-conjugated human FLT3L. The slope of the double positive population is proportional to FLT3L affinity (MFI ratios between FLT3 control antibody and FLT3L staining are reported in each plot).

FIG. 16B are histograms showing the distribution of the MFI ratio between FLT3 control antibody and AF488-conjugated FLT3L staining in comparison with wild type FLT3 (grey overlay).

FIG. 17A shows the design of the custom cloned pmRNA plasmid to produce base editor mRNA for editing of human CD34+ cells.

FIG. 17B describes the workflow for the in vitro transcription protocol used to produce BE mRNA.

FIG. 17C is an Agilent Fragment Analyzer profile of an in vitro transcribed SpRY-ABE8e-V106W mRNA. x axis is nucleotide size, y axis is relative fluorescence units (RFU) signal.

FIG. 18, panel A are flow cytometry plots showing the outcome of a base editing experiment on FLT3-expressing K562 reporter cells with the FLT3-18-NRN sgRNA depicted in FIG. 15 (panel B) and IVT adenine base editors mRNA (SpRY-ABE8e-V106W 3xNLS). Cells were co-stained with a control antibody (clone BV10A4) and a therapeutic antibody (clone 4G8). Each plot reports the gating for edited cells and the percentage of edited cells. The experiment was performed with different mRNA and sgRNA doses (reported at the top and on the left of the plots, respectively). FIG. 18B are dot plots illustrating the dose-effect correlation between mRNA, gRNA and editing efficiency by flow cytometry. FIG. 18 (panel C) are dot plots illustrating the dose-effect correlation between mRNA, gRNA and cell viability after editing.

FIG. 19A is a heatmap reporting editing efficiencies at each adenine base position of the FLT3-18-NRN sgRNA with different mRNA and sgRNA doses (reported on the left), performed on FLT3-reporter K562 cells (same as FIG. 18). The gRNA sequence and the expected editing window are reported at the top of the heatmap.

FIG. 19B is a dot plot illustrating the dose-effect correlation between mRNA, gRNA and editing efficiency by gDNA sequencing (only editing on A6 and A7 base positions is reported).

FIG. 20A is the experimental design of a FLT3 base editing experiment on human mobilized peripheral blood-derived CD34+ HSPCs using IVT mRNA and FLT3-18-NRN sgRNA. The composition of the culture medium is reported on the right, while the timeline reports the timepoints for flow cytometry and gDNA collection.

FIG. 20B reports the gating strategy for flow cytometry evaluation of the stem cell phenotype of cultured CD34+ HSPCs.

FIG. 20C is a bar plot reporting the fold expansion of cultured CD34+ HSPCs at day 0, day 3 and day 6 post editing.

FIG. 20D is a bar plot showing the composition of cultured CD34+ HSPCs at day 3 and 6 after editing by flow cytometry (gating is reported in FIG. 20B).

FIG. 21A is a heatmap reporting editing efficiencies at each adenine base position of the FLT3-18-NRN sgRNA with different mRNA and sgRNA doses (reported on the left), performed on mobilized peripheral blood-derived CD34+ HSPCs (same as FIG. 20).

FIG. 21B is a dot plot illustrating the dose-effect correlation between mRNA, gRNA and editing efficiency by gDNA sequencing on CD34+ HSPCs.

FIG. 22A are flow cytometry plots showing a CAR-T co-culture killing assay in which the target cells were either unmodified or base edited FLT3 expressing K562 cells. Target cells were plated with 4G8-CAR T cells or untransduced T cells at different effector: target ratios (reported on top) and evaluated at 6h by flow cytometry. Live cells (Annexin V-LiveDead yellow-) are plotted and the relative % is reported.

FIG. 22B reports the viability (AnnexinV-LiveDead yellow-) of the target cells at each E:T ratio at 6h after co-culture.

FIG. 22C reports the degranulation of the T cells by CD107a surface staining at each E:T ratio at 6h after co-culture.

FIG. 23A are flow cytometry plots showing a CAR-T co-culture killing assay in which the target cells were either unmodified or base edited human CD34+ HSPCs (editing efficiency 46%). Target cells were plated with 4G8-CAR T cells or untransduced T cells at different effector: target ratios (reported on top) and evaluated at 48h by flow cytometry.

FIG. 23B reports the specific killing of CD34+ cells by 4G8 CAR-T cells at 48h.

FIG. 23C reports the specific killing of CD34+CD90+ stem-cell enriched subset by 4G8 CAR-T cells at 48h.

FIG. 24A reports the experimental design of a pilot in vivo experiment to evaluate the resistance of FLT3-epitope engineered CD34+ HSPCs to 4G8 CAR-T cells. Experimental timeline and procedures are reported, as well as treatment group numerosity.

FIG. 24B are bar plots reporting the relative abundance and absolute counts of human CD45+ engraftment in the bone marrow (BM) at sacrifice for each treatment group.

FIG. 24C is a bar plot describing the lineage composition of the human engraftment derived from xenotransplanted CD34+ HSPCs, wither unmodified or base edited in the FLT3 gene.

FIG. 25A are bar plots reporting the relative abundance and absolute counts of human CD34+CD38− progenitors within human CD45+ engraftment in the bone marrow at sacrifice (same experiment as FIG. 24). Mice who were xenotransplanted with unmodified CD34+ HSPCs show significant reduction of CD34+CD38− progenitors upon 4G8 CAR-T administration.

FIG. 25B is a bar plot reporting the percentage of CD69+ cells (T cell activation marker) on CAR-T cells in the spleen of treated mice (same experiment as FIG. 24).

FIG. 25C is a bar plot reporting the T cell phenotype (evaluated by surface expression of CD62L and CD45RA) divided in central memory (CD45RA−CD62L+, CM), effector memory (CD45RA−CD62L−, EM), Naïve (CD45RA+CD62L+) or effector memory cells re-expressing CD45RA (CD45RA+CD62L−, TEMRA).

FIG. 25D depicts the gating strategy for flow cytometry of bone marrow samples from an in vivo xenotransplantation experiment (same as FIG. 24) to identify lineage negative human progenitors (live/hCD45+/CD3−/CD19−/CD33−/CD34+/CD38−).

FIG. 25E reports cumulative flow cytometry plots obtained from pooled events of mice from the same condition (same experiment as FIG. 24) showing the percentage of human CD34+CD38− progenitors in the bone marrow at sacrifice.

FIG. 26A is the experimental design of a FLT3 and CD123 dual base editing experiment on human mobilized peripheral blood-derived CD34+ HSPCs using IVT mRNA and FLT3-18-NRN and CD123-N sgRNAs. The composition of the culture medium is reported on the right, while the timeline reports the timepoints for flow cytometry and gDNA collection.

FIG. 26B is a bar plot reporting the fold expansion of cultured CD34+ HSPCs at day 0, day 3 and day 7 post editing.

FIG. 26C are flow cytometry plots of edited CD34+ HSPCs to highlight the loss of CD123 clone 7G3 recognition in CD123 base edited conditions. Cells were co-stained with a control CD123 antibody (clone 9F5) and the therapeutic antibody (7G3). Cells were pre-gated on CD34+CD90+CD45RA−.

FIG. 27 is a heatmap reporting editing efficiencies at each adenine base position of the FLT3-18-NRN and CD123-N sgRNAs with different mRNA and sgRNA doses (reported on the left), performed on mobilized peripheral blood-derived CD34+ HSPCs (same as FIG. 26).

FIG. 28A are flow cytometry plots of K562 cells base edited with FLT3-18-NRN sgRNA or CD123-N sgRNA in combination with either SpRY-ABE8e-V106W or SpRY-K918-ABE8e-V106W to show the improvement of CD123 base editing efficiency.

FIG. 28B reports the sequences for 3 different gRNAs targeting CD123 S59 residue.

FIG. 28C depicts several different adenine base editor designs with modified SpCas9, including higher fidelity variants (HF1, Sniper), K918N variants, BlackJack variants (more tolerant to longer gRNAs) and combinations of these mutations.

FIG. 28D is a heatmap reporting the editing efficiencies using the 3 gRNAs from FIG. 28B (plus FLT3-18-NRN as control) and the base editor variants from FIG. 28C.

FIG. 29A is the experimental design of a FLT3 and CD123 dual base editing experiment on human mobilized peripheral blood-derived CD34+ HSPCs using IVT mRNA and FLT3-18-NRN and CD123-R sgRNAs. The composition of the culture medium is reported on the right, while the timeline reports the timepoints for flow cytometry and gDNA collection.

FIG. 29B are flow cytometry plots demonstrating the gating strategy to obtain CD123 base editing efficiencies in cultured CD34+ HSPCs.

FIG. 29C is a bar plot reporting the fold expansion of cultured CD34+ HSPCs at day 0, day 3 and day 6 post editing.

FIG. 30 are heatmaps reporting the base editing efficiencies at each adenine position for FLT3-18-NRN and CD123-R sgRNAs (same experiment as FIG. 29). The target bases are reported on the bottom of the heatmap. On the right, a heatmap reports the % of CD123 base edited by flow cytometry.

FIG. 31 is a heatmap reporting the base editing efficiencies on CD34+ HSPCs at each adenine position for KIT-gRNA-Y targeting residue H378 from the same experiment as FIG. 29.

FIG. 32A reports the experimental design for an in vivo xenotransplantation experiment to confirm the resistance of FLT3-epitope engineered human CD34+ HSPCs to 4G8 CAR-T cells. Experimental timeline and treatment groups and numerosity are reported.

FIG. 32B reports the FLT3 base editing efficiencies on 8 week post-transplant peripheral blood samples for all mice.

FIG. 33A report the percentage of polymorphonucleate granulocytes (CD3-CD19-CD33+SSChi, PMN) and granulo-mono progenitors (lineage-CD34+CD38+CD45RA+FLT3+, GMP) on the human CD45+ engraftment in the bone marrow at sacrifice (same experiment as FIG. 32).

FIG. 33B shows the FLT3 editing efficiency on genomic DNA at several time-points for 4G8 CAR-T cell treated and untreated mice. LC, liquid culture (pre transplantation sample); W8, W12, week 8 and 12 bleeding; BM, bone marrow; CFU, colony forming unit-derived cells (plated from bone marrow samples at sacrifice).

FIG. 34 are representative flow cytometry plots showing the relative abundance of polymorphonucleate granulocytes (CD3−CD19−CD33+SSChi, PMN), granulo-mono progenitors (lineage-CD34+CD38+CD45RA+FLT3+, GMP) and hematopoietic stem cells (lineage-CD34+CD38−CD90+CD45RA−, HSC) in the bone marrow of mice xenotransplanted with FLT3 or AAVS1 edited CD34+ HSPCs.

FIG. 35 depicts several alternative designs for single or dual specificity chimeric antigen receptors (CAR), either expressed as two separate constructs or as tandem-CAR (two scFv on the same molecule, separated by a linker).

FIG. 36A is a schematic of type-III receptor tyrosine kinase (FLT3, KIT) with the extracellular domains (ECD) recognized by control or therapeutic (magenta) monoclonal antibodies. AF488-conjugated FLT3L or SCF ligands were used to assess binding affinity of the mutated receptors.

FIG. 36B are flow cytometry plots showing loss of therapeutic mAb (4G8 and Fab79D for FLT3 and KIT, respectively) to chimeric receptors with 16 or 10 amino acid substitutions, respectively (top); and a fluorescent ligand binding assay for wild-type (WT) and mutated receptor variants (bottom).

FIG. 36C are western blots of pFLT3 Y589-591 and pKIT Y719 of cell lines expressing FLT3 and KIT variants either unstimulated or incubated with 100 ng/ml FLT3L or SCF.

FIG. 36D is a schematic of the Sleeping Beauty plasmid containing FLT3 cDNA with either human or murine codons in 16 amino acid positions of the ECD4 (top left); K562 cells transduced with the FLT3 library and FACS-sorted (4G8− and 4G8+) for NGS sequencing of the library region (top right); and sequence logo showing the relative amino acid frequency at positions 384-413 (bottom).

FIG. 36E is a schematic of the Sleeping Beauty plasmid containing degenerated codon (NNN) at each position of ECD4 (top left); K562 cells transduced with the KIT library and FACS-sorted (Fab79D− and Fab79D+) for NGS sequencing of the library region (top right); and sequence logo showing the log-fold change of amino-acid substitutions enriched in the Fab-79D^{low} vs double positive cells (aa. 314 to 381) (bottom; positions with multiple enriched amino-acid substitutions are consistent with previously predicted contact-points).

FIG. 36F is a schematic of the tested gRNAs targeting FLT3 codon N399 (left); and a representative plot of K562 FLT3 reporter cells electroporated with base editor expression plasmid (NG-ABE8e or SpRY-ABE8e) and sgRNAs, evaluated by flow cytometry 72h after editing (right; the % of cells positive for control mAb BV10A4 and negative for clone 4G8 is reported in each plot; the unedited condition shows the gating strategy).

FIG. 36G shows CD123 epitope screening by base editing. Top: sgRNAs for targeted base editing of 7G3 contact residues used for the screening. Dark blue, NGG PAM; grey, NGN PAM; light blue, NRN PAM. The PAM position is indicated by the arrowhead. Bottom: FACS plots of CD123-reporter K562 cells treated with SpRY-cytidine (top row) or adenine (bottom row) base editors. The % of cells positive for control mAb 9F5 and negative for therapeutic clone 7G3 is reported in the lower-right of each plot.

FIG. 36H shows affinity of therapeutic antibodies to stealth receptor variants measured on K562 (for FLT3 and CD123) or NIH-3T3 (for KIT) cells expressing the receptor variants. Mean±SD (N=3) of the MFI of the therapeutic mAb normalized on the control mAb, after background subtraction. Comparison by two-way ANOVA.

FIG. 36I are FLT3, SCF and IL3 affinity assay. Cell lines expressing receptor variants through Sleeping Beauty transposase were incubated with fluorescent ligands and evaluated by flow cytometry. Mean±SD (N=3 FLT3/CD123; N=4 KIT) of the MFI of the ligand. Comparison by two-way ANOVA.

FIG. 37A are full length sequence logo of the FLT3 EC4 combinatorial library showing the amino-acid frequency at each position of ECD4 (357 to 421) in FACS-sorted 4G8− and 4G8+ cells.

FIG. 37B shows the design of Sleeping Beauty transposon encoding for FLT3 variants with a mCherry and puromycin N-acetyltransferase (PAC) reporter/resistance cassette (top) and flow cytometry plots showing loss of 4G8 recognition for N399D and N399G variants expressed in K562 cells (bottom).

FIG. 37C shows generation of FLT3, KIT and CD123 reporter K562 cells through targeted homology-directed repair integration of a EF1α promoter upstream of the gene transcriptional start site (TSS). A dsDNA donor with 50-bp long homology arms was generated by PCR on a plasmid template encoding for a full EF1α promoter. K562 cells were electroporated with SpCas9 (FLT3, KIT) or AsCas12a nuclease (CD123) and gRNAs recognizing a region upstream of the coding sequence of each gene. 0.5 to 10 μg of dsDNA donor template was co-electroporated with Cas RNPs in 20 μL electroporation volume. Representative flow cytometry plots show the population of cells positive for the over-expressed gene, which were FACS-sorted and expanded. For FLT3 and CD123, single cell cloning of sorted cells was performed to isolate clones with the highest surface expression. All epitope-editing tests and optimization were performed on K562 reporter cells, unless otherwise specified. Dual FLT3/CD123 reporters were obtained through a second round of CD123-targeted RNP+donor electroporation on FLT3-expressing K562 cells (data shown in FIG. 49B).

FIG. 37D shows introduction of the FLT3 N399D mutation through CRISPR-Cas mediated homology directed repair. K562 reporter cells were electroporated with SpCas9 or AsCas12a nuclease, gRNAs and 200-bp ssODN template donor (or their reverse complement, rev.comp.) encoding for the N399D mutation. Additional silent mutations were included to reduce the risk of nuclease re-cutting after HDR repair. Cells were evaluated by flow cytometry 72h after editing. The percentage of FLT3+ cells (by control mAb BV10A4) but 4G8− is reported in the right bottom corner.

FIG. 37E shows characterization of KIT mutations derived from epitope mapping. For amino-acid positions deriving from the KIT epitope mapping, substitutions that could be obtained with adenine BE (ABE, red) or cytidine BE (CBE, blue) were individually cloned in a Sleeping Beauty transposon and electroporated into HEK-293T cells. After puromycin selection, cells were stained with both Fab-79D and control Ab 104D2. The ratio between Fab-79D MFI and 104D2 MFI is reported for each mutation. To exclude variants affecting SCF binding to KIT, the same variants were incubated with AF488-conjugated SCF and control mAb 104D2 (which does not impair SCF binding). The ratio of SCF to 104D2 median fluorescence is reported in the bar plots. Horizontal lines show the reference mutation H378R.

FIG. 37F shows KIT H378R adenine base editing optimization. sgRNAs targeting codon H378 within exon 7 were co-electroporated with SpRY-ABE8e in K562 cells. Editing efficiency on gDNA is reported for each adenine within the protospacer (with position numbers relative to KIT-Y sgRNA).

FIG. 37G shows, top, design of Sleeping Beauty transposon encoding for KIT variants with a mTagBFP2 and puromycin N-acetyltransferase (PAC) reporter/resistance cassette; and, bottom, flow cytometry plots showing loss of Fab79D recognition for KIT H378R expressed in HEK-293T cells.

FIG. 37H shows CD123 epitope screening by base editing. sgRNAs for targeted base editing of 7G3 contact residues reported in FIG. 1G were co-electroporated with adenine (ABE) or cytidine base editor (CBE) expression plasmids in CD123-reporter K562 cells. The percentage of cells positive for control mAb 9F5 and negative for therapeutic clone 7G3 is reported in each plot. The unedited condition shows the gating strategy. BE4, evoApobec-1 BE4max.

FIG. 37I shows CD123 CBE with sgRNA-F results in loss of clone 6H6 and S18016F binding. The same conditions from the BE screening reported in FIG. 37H were stained with CD123-targeting clones 6H6 and S18016F which have a different epitope than clone 7G3.

FIG. 37J shows design of Sleeping Beauty transposon encoding for CD123 variants with co-expression of the common β-chain CSFR2B to allow intracellular signal transduction.

FIG. 38A shows FLT3 epitope engineered variants preserve kinase activation. Western blot of protein extracts from K562 cells expressing FLT3 variants by Sleeping Beauty transposase. Cells were serum-starved overnight and stimulated with different concentrations of human FLT3L for 10 minutes at 37° C. pFLT3 Y589-591, total FLT3 and actin were probed on the same lysates. Total FLT3 was probed after stripping of the pFLT3 membrane. Normalized pFLT3 signal intensity (on actin) is reported on the right. Comparison by 2-way ANOVA.

FIG. 38B shows KIT epitope engineered variant preserves kinase activation. Western blot of protein extracts from NIH-3T3 cells expressing KIT variants by Sleeping Beauty transposase. Cells were serum-starved overnight and stimulated with different concentrations of human SCF for 10 minutes at 37° C. pFLT3 Y719, total KIT and actin were probed on the same lysates. Normalized pKIT signal intensity (on total KIT) is reported in the right plot. Comparison by 2-way ANOVA.

FIG. 38C shows CD123 epitope engineered variants preserve STAT5 activation. BaF3 cells expressing CD123 variants by Sleeping Beauty transposase were starved for murine IL-3 and stimulated with different concentrations of human IL-3. Cells were evaluated for STAT5 phosphorylation by intracellular flow cytometry after 48h (left, representative FACS plots show the CD123 S59P condition at different hIL-3 doses; right, pSTAT5 PE MFI).

FIG. 38D shows FLT3, KIT, CD123 epitope engineered variants induce proliferative responses similar to WT receptors. BaF3 cells expressing FLT3, KIT and CD123 variants by Sleeping Beauty transposase were starved for murine IL-3 overnight and stimulated with different concentrations of human FLT3, SCF and IL-3, respectively. Cells were cultured for 5 days and analyzed by flow cytometry to obtain absolute counts (CountBeads). Plots report absolute counts normalized by the unstimulated condition.

FIG. 39A shows, top, schematic of the bidirectional lentiviral vector (LV) co-expressing a 2^{nd} generation CAR and a truncated human epidermal growth factor receptor (EGFRt); and, bottom, schematic of T cell culture, transduction and analysis for the generation of CAR-T cells.

FIG. 39B shows percentage of EGFRt surface expression (left) and fold expansion (right) on T cells at the indicated days (D) after transduction with LV 4G8-CAR at different multiplicity of infection (MOI).

FIG. 39C shows FLT3 N399D or N399G avoid 4G8 CAR killing. K562 cells expressing FLT3 variants by Sleeping Beauty transposase were cultured with 4G8 CAR-T cells at different effector:target ratios (E:T). Left: representative flow cytometry plots of K562 cells expressing either FLT3 WT or N399D, after 48h of co-culture with 4G8 CAR. T cells and target cells are identified by CD3 and FLT3 staining, respectively. From Left to Right, i) plots reporting the fraction of persisting live target cells (absolute counts of AnnexinV-7AAD-cells) relative to E:T=0; ii) T cell activation by CD69 staining (%) and iii) surface expression of FLT3 by BV10A4 staining on residual live target cells, normalized on E:T=0. Mean±SD (N=4). Comparison by two-way ANOVA.

FIG. 39D shows KIT H378R avoid 79D CAR killing. Left: representative flow cytometry plots of K562 cells expressing either KIT WT or H378R by Sleeping Beauty transposase after 48h of co-culture with 79D CAR. T cells and target cells are identified by CD3 and KIT staining, respectively. From Left to Right, plots reporting i) the fraction of persisting live target cells (absolute counts of AnnexinV-7AAD-cells) relative to E:T=0; ii) T cell activation by CD69 staining (%) and iii) surface expression of KIT by 104D2 staining on residual live target cells, normalized on E:T=0. K562: untransduced. Mean±SD (N=4). Comparison by two-way ANOVA.

FIG. 39E shows CD123^{S59} BE cells are resistant to CSL362 CAR. Left: representative flow cytometry plots of CD123-reporter K562 base-edited for CD123 or unmodified after 48h of co-culture with CSL362 CAR. T cells are identified by CD3/4/8 staining. From Left to Right-plots reporting i) fraction of persisting live target cells (absolute counts of AnnexinV-7AAD-cells) relative to E:T=0; ii) T cell activation by CD69 staining (%) and iii) surface expression of CD123 by 9F5 staining on residual live target cells. Mean±SD (N=4). Comparison by two-way ANOVA.

FIG. 40A shows CAR-T cell CD4/CD8 composition during in vitro culture. Fresh healthy donor-derived PBMC were cultured with CD3/CD28 Dynabeads (bead: cell ratio=3:1), IL-7 5 ng/ml and IL-15 5 ng/mL and transduced at day (D) 2 with a lentiviral vector (LV) encoding for the 4G8 CAR. The culture composition was evaluated by flow cytometry at days 2, 4, 6, 12. The plot reports N=5 conditions LV-transduced with different multiplicity of infection (MOI). Mean±SD.

FIG. 40B shows CAR-T cell phenotype by flow cytometry. T cell subsets were evaluated by CD62L and CD45RA staining (CD45RA+62L+, Naïve/T stem memory cells; CD45RA−62L+, central memory, CM; CD45RA−62L−, effector memory, EM; CD45RA+62L−, terminally differentiated EM cells re-expressing CD45RA, EMRA). Representative FACS plots (left) and the culture composition by CD4+ and CD8+ subsets (right) are reported. DO refers to uncultured peripheral blood T cells after Ficoll separation. Mean±SD (N=5).

FIG. 40C shows FLT3^{WT} cells are eliminated by 4G8 CAR-T cell while FLT3^{N399} BE cells are protected. 4G8 CAR-T cells co-culture assay with FLT3 reporter K562 cells either unmodified or FLT3^{N399} base edited. (Left) Representative flow cytometry plots at early timepoint (6h) gated on live cells (AnnexinV-7AAD-). T cells are identified by CellTrace marking, while K562 target are FLT3+. (Left to Right) Target cell viability at 6 h (%), T cell degranulation by CD107a surface staining at 6 h (%) and FLT3 expression on surviving target cells at 48h (MFI, normalized on E:T=0). N=2. Comparison by 2-way ANOVA.

FIG. 40D shows epitope engineered receptors provide protection from CAR-T cells. Each row reports additional plots from co-cultures with FLT3, CD123 and KIT expressing K562 cells (same experiments reported in FIG. 39 C, D, E). By column from left to right: CellTrace MFI of CAR-T cells at 48h of co-culture, Target cell viability after 48 h of co-culture with CAR-T cells (%), Target cell viability after 48 h of co-culture with untransduced T cells (%), absolute counts of live cells (AnnexinV-7AAD-, normalized on E:T=0) after 48 h of co-culture with untransduced T cells, CD69+ untransduced T cells after 48 h of co-culture (%), FLT3, CD123 and KIT MFI (non-normalized) on target cells after 48 h of co-culture with untransduced T cells. Mean±SD, N=4.

FIG. 40E shows experimental layout for co-culture assays with two populations of target cells, one expressing FLT3 and the other expressing CD123. Unmodified or epitope edited FLT3 and CD123 K562 reporter cells were mixed at ˜1:1 ratio and co-cultured with either expressing 4G8 CAR, CSL362 CAR or untransduced T cells. The FLT3+/CD123+% composition of live target cells (pre-gated on FLT3+ or CD123+) is reported as bar plots for each combination at different effector: target (E:T) ratios. Mean±SD, N=4.

FIG. 41A is a schematic of CD34+ HSPCs culture, base editing and analysis. mPB, mobilized peripheral blood; HSPC, hematopoietic stem and progenitor cells; FLT3L, FLT3 ligand; SCF, Stem Cell Factor; TPO, Thrombopoietin; SR-1, StemRegenin.

FIG. 41B are representative plots reporting the editing windows and editing efficiencies of CD34+ HSPCs at each adenine within the gRNA sequence for FLT3, KIT and CD123 measured after electroporation with different doses of adenine BE mRNA. Mean±SD.

FIG. 41C shows editing efficiencies in bulk CD34+ cells or FACS-sorted stem-enriched CD90+ or stem-depleted CD90-subsets. Mean±SD (N=2 donors).

FIG. 41D shows immunophenotype of epitope edited CD34+ HSPCs. Left: representative flow cytometry plots displaying CD90/CD45RA subsets within CD34+133+ HSPCs during in vitro culture at day 5 after base editing. Right, bar plot showing the CD90/CD45RA subset composition of FLT3^{N399}, KIT^{H378R}, CD123^{S59} epitope edited CD34+ cells. Mean±SD. Sample size is reported within the bars.

FIG. 41E is an In vitro 4G8 CAR killing assay on FLT3^{N399} epitope edited HSPCs. Fraction of persisting live cells (Left) and viability by AnnexinV-7AAD staining (Right) of CD34+45RA+ cells, either FLT3- or AAVS1-base edited, 48h after co-culture with 4G8 CAR or untransduced T cells. Mean±SD (N=4). Comparison by 2-way ANOVA.

FIG. 41F is an In vitro CSL362 CAR killing assay on CD123^{S59} epitope edited HSPCs. Fraction of persisting live CD90+ cells (Left) and viability of CD34+ cells by Annexin V-7AAD staining (Right), of CD123^{S59} or AAVS1-BE HSPCs, 48h after co-culture with CSL362 CAR or untransduced T cells. N=8 on 2 different HSPCs donors. Comparison by 2-way ANOVA

FIG. 41G shows fraction of absolute live CD34+ cells relative to no mAb control of KIT^{H378} BE or AAVS1 BE HSPCs plated with increasing concentrations of Fab-79D mAb and cultured for 48h. Mean±SD. N=6 on 2 different HSPCs donors. Comparison by 2-way ANOVA

FIG. 41H shows absolute counts of total CD34+ and CD90+45RA− cells (top left) and of myeloid and erythroid colonies (N=2, bottom left) of base edited HSPCs. Right: representative images of the CFU plates (one of 2 replicates) 14 days after plating.

FIG. 41I is a schematic representation of primary and secondary xeno-transplantation and analyses of FLT3^{N399} or AAVS1 BE HSPCs.

FIG. 41J shows, left, human engraftment (hCD45+ cells) by flow cytometry at different time-points in primary recipients; and, right, BM lineage composition as percentage of total human (hCD45+) cells by flow cytometry. PMN, polymorphonucleate granulocytes; mono, monocytes; lin-, lineage negative. Mean±SD. FLT3^{N399} N=7, AAVS1 BE N=4. Comparisons by 2-way ANOVA.

FIG. 41K shows FLT3 editing levels measured on total circulating cells, hematopoietic organs or CFU at different timepoints on primary and secondary transplanted mice. Mean±SD. LC, liquid culture; W8, week 8; W12 week 12; SP, spleen; BM, bone marrow; CFU, colony forming unit.

FIG. 42A is a schematic representation of the plasmid template used for in vitro transcription (IVT) of base editor mRNAs. Type-IIS restriction enzyme BbsI was used to linearize the template. T7, T7 RNA polymerase promoter; UTR, untranslated region; HBB, hemoglobin β gene; polyA, poly-adenine sequence (˜110-120 nt).

FIG. 42B is a representative plot of purified IVT SpRY-ABE8e mRNA analyzed with Agilent Fragment Analyzer RNA for quality control. >90% of IVT mRNA corresponds to the predicted size.

FIG. 42C is a SpRY-ABE8e V106W mRNA dose finding test on FLT3-reporter K562 cells base edited for FLT3^{N399} with sgRNA-18. Right, correlation of FLT3 editing efficiency by flow cytometry with mRNA×sgRNA dose and correlation of FLT3 editing efficiency by flow cytometry and by gDNA analysis. Spearman r² and p values are reported.

FIG. 42D shows CD34+ HSPC base editing mRNA optimization. Several SpRY-ABE8e mRNA variants were tested in a dual FLT3/CD123 editing experiment. Tested variables include: mRNA purification method (beads, sparQ PureMag magnetic beads; column, NEB Monarch RNA columns), dephosphorylation, substitution of UTP with N1-methyl-pseudo-uridine (N1m-U) or 5-methoxy-uridine (5me-U), capping technology (CleanCap, Trilink CleanCap AG; ARCA, NEB 3′-O-Me-m7G(5′)ppp(5′)G RNA Cap Structure Analog) and the addition of the K918N SpCas9 mutation (which was reported to improve nuclease efficiency)⁶⁴. Bar plots showing FLT3 and CD123 editing efficiencies by genomic DNA (gDNA) analysis (%) and absolute counts of bulk (CD34+) and stem-enriched (CD90+45RA−) cells at the end of in vitro culture.

FIG. 42E shows optimization of culture conditions for base editing. CD34+ HSPCs were base edited with SpRY-ABE8e mRNA and FLT3^{N399} sgRNA with addition of supplements during electroporation (RNAsin, Promega RNAsin RNAse-inhibitor; glycerol) or with different culture conditions, including modulation of cytokine concentrations (standard: 100 ng/mL FLT3L, SCF and 50 ng/ml TPO; 1.5×: 150 ng/ml FLT3L, SCF and 75 ng/ml TPO; 3×: 300 ng/mL FLT3L, SCF and 150 ng/ml TPO; +IL-3: standard with addition of hIL-3 20 ng/ml), different stem-cell preserving compounds (standard: SR-1 0.75 μM, UM171 35 nM; SR-1 only 0.75 μM; UM171 only 35 nM; no SR-1/UM171), addition of anti-inflammatory compounds (PGE2, Prostaglandin-E2 10 μM; DEX, dexamethasone 1 μM). Bar plots showing FLT3 editing efficiencies by gDNA analysis (%) and absolute counts of bulk (CD34+) and stem-enriched (CD90+45RA−) cells at the end of in vitro culture.

FIG. 42F are bar plots. CD34+ HSPCs were electroporated at different timepoints (24 h, 48 h, 72 h) after thawing. Each condition was edited for all combinations of two of our selected targets (FLT3, CD123, KIT). Bar plots showing editing efficiencies by gDNA analysis (%) and absolute counts of bulk (CD34+) and stem-enriched (CD90+45RA−) cells at the end of in vitro culture.

FIG. 42G are representative flow cytometry plots showing the gating strategy used for analysis of edited CD34+ HSPCs. From left to right, cells were gated for singlets (FSC-H/FSC-A plot), Live (PI/FSC-A plot; PI, propidium iodide), physical parameters (SSC-A/FSC-A plot), CD34+ (CD34/CD90 plot), CD133+ (CD34/CD133 plot), and CD45RA-90+, CD45RA−90−, CD45RA+90− (CD45RA/90 plot). After pre-gating on singlets(S), a bead (B) gate identifies CountBeads (FSC-A^{low}PI^{high}, which are further gated on two additional fluorescent parameters to exclude debris (not shown).

FIG. 42H shows epitope-edited HSPCs retain proliferative response to cytokine stimulation. FLT3, KIT and CD123 base edited CD34+ HSPCs were plated with different concentration of the respective ligand and cultured for 4 days. Absolute counts were obtained by flow cytometry using CountBeads.

FIG. 42I shows editing efficiencies at experiment endpoint (technical triplicate were pooled together for gDNA analysis) (see FIG. 42H). Mean±SD. N=4 on 2 healthy donors. 2-way ANOVA multiple comparisons.

FIG. 43A shows human engraftment by flow cytometry (% hCD45+) in the peripheral blood at 12 weeks (W12) and in the bone marrow (BM) at endpoint of secondary recipients NBSGW mice xenotransplanted with BM cells from the experiment depicted in FIG. 3I (each primary transplanted in one secondary recipient). Mean±SD. Comparison by 2-way ANOVA.

FIG. 43B shows absolute counts of myeloid (left) and lymphoid (right) lineages in the BM of secondary xenotransplanted mice. AAVS1^{BE} N=4, FLT3^{N399} N=7. Mean±SD. Comparison by 2-way ANOVA. HSC, hematopoietic stem cells; MPP, multipotent progenitors; LMPP, lymphoid-primed multipotent progenitors; CMP, common myeloid progenitors; GMP, granulo-mono progenitors; myeloblasts, defined as CD33/66b+19−14−11c−34−SSC^{low}; mono, monocytes; iPMN, immature polymorphonucleate granulocytes; mature PMN, mature granulocytes.

FIG. 43C shows human engraftment by flow cytometry (% hCD45+) in the peripheral blood at 9, 11 weeks (W9, W11) and in the BM of NBSGW xenotransplanted with 1 M CD34+ HSPCs, either AAVS1 or KIT^{H378} edited. Mean±SD. Comparison by 2-way ANOVA.

FIG. 43D shows absolute counts of myeloid (left) and lymphoid (right) lineages in the BM of mice from C. Comparison by 2-way ANOVA. AAVS1 N=5, KIT^{H378} N=4.

FIG. 43E shows KIT editing efficiencies measured on liquid culture (LC), total blood cells at week 9 after transplant (W9) and on FACS-sorted B (CD19) and myeloid (CD33) BM cells at the end of the experiment. Mean±SD.

FIG. 43F is a schematic representation of a lentiviral vector encoding for the mNeonGreen fluorescent protein under a hPGK promoter used to transduce human PDXs (left); and representative flow cytometry plots (right) showing the transduction efficiency of patient-derived AML xenografts on bone marrow (PDX-1) or spleen (PDX-2) samples. PDX cells were transduced ex vivo overnight, transplanted into NBSGW mice for expansion, FACS-sorted for mNeonGreen+ cells and injected into secondary recipients.

FIG. 43G shows genetic features (left) and surface immunophenotype (right) at thawing of AML PDX used for in vivo experiments. Genetic mutations and the % of positive cells for each marker is reported in the heatmap. ITD, internal tandem duplication; TKD, tyrosine kinase domain mutation.

FIG. 44A is a schematic representation of xeno-transplantation and analysis of FLT3^{N399} or AAVS1 BE HSPCs co-engrafted with AML PDX-1 and treated with 4G8 CAR-T cells.

FIG. 44B shows FLT3 base editing measured on liquid culture (LC), total blood cells (weeks 8, 9) and on sorted CD33+ and CD19+ bone marrow (BM) cells at endpoint of mice either treated or untreated with 4G8 CAR-T cells. Multiple unpaired t tests. Mean±SD.

FIG. 44C are representative flow cytometry plots of BM samples from mice engrafted with CD34+ HSPCs, CD34+ HSPCs+AML PDX-1, or CD34+ HSPCs+AML PDX-1 and treated with 4G8 CAR-T. Plots are pre-gated on human CD45+; CAR T cells are identified by CD3 staining, AML PDX cells are mNeonGreen+. Mean±SD. Comparisons by 1-way ANOVA.

FIG. 44D is a bar plot showing the % of CD3+ cells within hCD45+mNeonGreen-BM cells. Mean±SD. Comparisons by 1-way ANOVA.

FIG. 44E is a bar plot showing percentage of AML cells within hCD45+CD3− BM cells. Mean±SD.

FIG. 44F is a bar plot showing percent of AML (mNeonGreen+) cells measured by flow cytometry on total BM-derived CFU. Mean±SD. Statistical comparison of FLT3^{N399} vs AAVS1 BE conditions by one-way ANOVA.

FIG. 44G are representative FACS plots showing depletion of BM B cells (CD19+) by 4G8 CAR-T in mice transplanted with FLT3^{N399} or AAVS1 BE HSPCs. Plots are pre-gated on hCD45+CD3−mNeonGreen-.

FIGS. 44H, 44I, and 44J are bar plots showing the % of pre-B (CD19+10+34−) (FIG. 44H), pro-B (CD19+10+34+) cells within hCD45+CD3−mNeonGreen− (FIG. 44I), and immature granulocytes (CD33/66b+14−10−11c−SSC^{high}) within myeloid cells (CD33/66b+) (FIG. 44I). Mean±SD. Statistical comparison of FLT3^{N399} vs AAVS1 BE conditions by one-way ANOVA.

FIG. 44K are representative FACS plots showing the composition of lineage-CD34+38+10− progenitors. Granulo-mono progenitors (GMP) are defined as CD45RA+FLT3+, Common myeloid progenitors (CMP) as CD45RA−FLT3+ and Mega-Erythroid progenitors (MEP) as CD45RA−FLT3−.

FIG. 44L are bar blots showing GMP % within lin-CD34+38+. Mean±SD.

FIG. 44M shows absolute counts of myeloid lineage subsets in the BM, from HSC to differentiated leukocytes. Untreated mice are pooled together (grey bars), 4G8-treated FLT3^{N399} and AAVS1 BE mice are reported in pink and yellow, respectively. Mean±SD. The fold change in absolute counts (FLT3^{N399}/AAVS1) for CAR treated groups is reported above each population bar plot. One-way ANOVA with multiple comparisons. FDR-adjusted p values of the comparison between FLT3^{N399} vs AAVS1 BE conditions treated with 4G8 CAR are reported (p<0.05 are in bold).

FIG. 44N are representative FACS plots showing the composition of lineage-CD34+38−10− progenitors. Stem cells (HSC) are defined as CD45RA−90+, multipotent progenitors (MPP) as CD45RA−90− and lymphoid-primed MPP (LMPP) as CD45RA+90−.

FIG. 44O are bar plots showing LMPP % within lin-CD34+38−. Mean±SD.

FIG. 44P shows absolute counts of lymphoid lineage subsets in the BM, from HSC to differentiated leukocytes. preB/NK are defined as CD33/66b−19−56−34+38+10+, B-prolymphocytes as CD33/66b−19−56−34−10+, pro-B cells as CD33/66b−19+10+34+, pre-B cells as CD33/66b−19+10+34−, mature B cells as CD33/66b−19+10−34−. Untreated mice are pooled together (grey bars), 4G8-treated FLT3^{N399} and AAVS1 BE mice are reported in pink and yellow, respectively. The fold change in absolute counts (FLT3^{N399} AAVS1) for CAR treated groups is reported above each population bar plot. Mean±SD. One-way ANOVA with multiple comparisons. FDR-adjusted p values of the comparison between FLT3^{N399} vs AAVS1 BE conditions treated with 4G8 CAR are reported (p<0.05 are in bold).

FIG. 45A shows peripheral blood lineage composition of NBSGW mice xeno-transplanted with AAVS1^{BE} and FLT3^{N399} HSPCs at 8 weeks. Mean±SD. AAVS1^{BE} N=13, FLT3^{N399} N=18. Statistical comparisons by unpaired t test.

FIG. 45B shows lineage-negative CD34+ progenitors are depleted by 4G8 CAR-T in vivo and protected by FLT3^{N399} editing (experiment from FIG. 44). Representative flow cytometry plots of lineage-neg cells (mNeonGreen-CD3−19−14−11c−56−) with gating of CD34+ progenitors (% reported within the gate).

FIG. 45C shows % of lin-CD34+ cells within hCD45+3-mNeonGreen-BM cells. Mean±SD. Comparison by 1-way ANOVA.

FIG. 45D shows relative composition of BM lin-CD34+ of mice from FIG. 44. Mean±SD. 2-way ANOVA with multiple comparisons (only AAVS1 vs FLT3^{N399} comparisons are reported). HSC, hematopoietic stem cells; MPP, multipotent progenitors; LMPP, lymphoid-primed multipotent progenitors; CMP, common myeloid progenitors; GMP, granulo-mono progenitors; MEP, mega-erythroid progenitors.

FIG. 45E shows FLT3 expression (MFI) on myeloid (left) and lymphoid (right) BM subsets at the endpoint. LMPP, MPP and HSC from 4G8 CAR treated AAVS1^{BE} conditions are not evaluable (NE) due to low cell number. Mean±SD. Statistical differences by multiple unpaired t test are reported.

FIG. 45F shows CAR cell phenotype by flow cytometry in the BM of mice from FIG. 44. CD45RA+62L+, Naïve; CD45RA−62L+, central memory, CM; CD45RA−62L−, effector memory, EM; CD45RA+62L, terminally differentiated EM cells re-expressing CD45RA, EMRA.

FIG. 45G are bar plots reporting (from left to right) % of EGFRt+ within BM CD3+ cells, PD1 (CD279) MFI on BM CD8+ CAR T cells and PD1 (CD279) MFI on BM CD4+ CAR T cells. Mean±SD. Comparison between AAVS1^{BE} and FLT3^{N399} conditions by 1-way ANOVA is reported.

FIG. 45H shows peripheral blood lineage composition of NBSGW mice xeno-transplanted with AAVS1^{BE} and CD123^{S59} HSPCs at 10 weeks (experiment from FIG. 46). Mean±SD (N=11). Statistical comparisons by unpaired t test are reported.

FIG. 45I shows lineage-negative CD34+ progenitors are depleted by CSL362 CAR-T in vivo (experiment from FIG. 46). Left, representative flow cytometry plots of lineage-negative cells (mNeonGreen-CD3−19−14−11c−56−) with gating of CD34+progenitors. Right, representative flow cytometry plots of lin-CD34+38−10− cells with gating of HSC (CD45RA−90+), MPP (CD45RA−90−), LMPP (CD45RA+90−) subsets.

FIG. 45J shows relative composition of BM lin-CD34+ of mice from FIG. 46. Mean±SD. 2-way ANOVA with multiple comparisons (only AAVS1 vs CD123^{S59} comparisons are reported). HSC, hematopoietic stem cells; MPP, multipotent progenitors; LMPP, lymphoid-primed multipotent progenitors; CMP, common myeloid progenitors; GMP, granulo-mono progenitors; MEP, meka-erythroid progenitors.

FIG. 46A is a schematic representation of xeno-transplantation and analysis of CD123^{S59} or AAVS1 BE HSPCs co-engrafted with AML PDX-1 and treated with 5M CSL362 CAR-T cells.

FIG. 46B shows CD123 base editing measured on total blood cells (week 8, 10) and on sorted CD33+ and CD19+ bone marrow (BM) cells at endpoint of mice either treated or untreated with CSL362 CAR-T cells. CD123^{S59} N=6, AAVS1 BE N=5. Mean±SD. Statistical comparison by multiple unpaired t test.

FIG. 46C shows representative flow cytometry plots of BM samples from mice engrafted with CD34+ HSPCs+AML PDX-1 either treated or untreated with CSL362 CAR-T and engrafted with AML PDX-1 only. Plots are pre-gated on total human CD45+; CAR-T cells are identified by CD3 staining, while AML PDX cells are mNeonGreen+.

FIG. 46D are bar plots showing the % of AML PDX cells within hCD45+CD3− BM cells. Mean±SD.

FIG. 46E are bar plots showing % of CD3+ cells within hCD45+mNeonGreen-BM cells. Mean±SD.

FIG. 46F are bar plots showing absolute counts of total hCD45+3−mNeonGreen-cells in the BM. Mean±SD.

FIG. 46G are bar plots showing % of pro-B cells (CD19+10−34−) within human CD45+3−mNeonGreen− BM cells. Mean±SD. Statistical comparison of CD123^{S59} vs AAVS1 BE conditions by one-way ANOVA.

FIG. 46H shows, left, representative FACS plots showing depletion of BM myeloid cells (CD33/66b+19−, highlighted by the orange gate) by CSL362 CAR-T in mice transplanted with CD123^{S59} or AAVS1 BE HSPCs; and right, representative FACS plots showing depletion of granulocytes (PMN, CD33/66b+19−14−SSC^{high}, orange gate)

FIGS. 46I, 46J, and 46K are bar plots showing the % of total myeloid cells (CD33/66b+19− within hCD45+ cells) (46I), PMN (CD33/66b+19− within CD33/66b+19− cells) (46J), immature PMN (CD33/66b+19−14−10−11c−SSC^{high} within CD33/66b+19− cells) (46K). Mean±SD. Statistical comparison of CD123^{S59} vs AAVS1 BE conditions by one-way ANOVA.

FIG. 46L are representative flow cytometry plots showing loss of dendritic cells (DC) subsets by CSL362 CAR-T in mice transplanted with CD123^{S59} or AAVS1 BE HSPCs. Left: conventional DC (cDC, CD33/66b+14−11c+FLT3+SSC^{low}), plots are gated on CD33/66b+14−SSC^{low} cells. Right: plasmacytoid DC (pDC, CD33/66b+14−11c−FLT3+CD123^{high}SSC^{low}), plots are gated on CD33/66b+14−11c−SSC^{low} cells.

FIGS. 46M and 46N are bar blots showing percentage of cDC and pDC within hCD45+3−mNeonGreen-cells, respectively.

FIG. 46O shows percentage of lineage-CD34+ progenitors within hCD45+3−mNeonGreen-cells. Mean±SD. Statistical comparison of CD123^{S59} vs AAVS1 BE conditions by one-way ANOVA.

FIGS. 46P and 46Q shows absolute counts of myeloid (P) and lymphoid (Q) lineage subsets in the BM, from HSC to differentiated leukocytes. Untreated mice are pooled together (grey bars), while CSL362-treated CD123^{S59} and AAVS1 BE mice are reported in pink and blue, respectively. The fold change in absolute counts (CD123^{S59} AAVS1) for CAR treated groups is reported above each population bar plot. Mean±SD. One-way ANOVA with multiple comparisons. FDR-adjusted p values of the comparison between CD123^{S59} vs AAVS1 BE conditions treated with 4G8 CAR are reported (p<0.05 are in bold).

FIG. 47A shows CD123 expression (MFI) on myeloid (left) and lymphoid (right) BM subsets at the endpoint. Mean±SD. Statistical differences by multiple unpaired t test are reported.

FIG. 47B shows 4G8 CAR T cells deplete PDX-1 in vivo but fail to eradicate PDX-2. NSBGW female mice were xeno-transplanted with AML PDX cells and, after 10 days, treated with 4G8 CAR-T cells. Experimental endpoint was 14 days after CAR-T administration. From left to right, bar plots showing the % of AML PDX cells within total CD45+ cells in the bone marrow (BM), % of AML PDX cells within total CD45+ cells in the spleen (SP), absolute counts of BM T cells, FLT3 MFI on surviving AML cells in the BM and FLT3 MFI on surviving AML cells in the SP. Mean±SD. Comparison by 1-way ANOVA.

FIG. 47C shows 4G8 CAR T cells deplete PDX-1 in vivo but fail to eradicate PDX-2 in mice pre-engrafted with AAVS1^{BE} and FLT3^{N399} HSPCs. NBSGW mice were xeno-transplanted with edited HSPCs and, after 11 weeks, injected with PDX-1 or PDX-2 cells. After 10 days, mice were treated with 2.5 M 4G8 CAR-T cells and the outcome evaluated after 2 weeks. The % of AML cells within total BM hCD45+ cells in reported in the bar plot. Mean±SD. Comparison by 1-way ANOVA.

FIG. 47D shows combinations of CAR T cells have improved efficacy on PDX-2 in vivo. NBSGW mice were xeno-transplanted with 0.75 M PDX-2 cells and, after 10 days, treated with 2.5 M 4G8 CAR or combinations of 4G8+CSL362, 4G8+Fab79D or CSL362+Fab79D CAR T cells (2.5 M each). The outcome was evaluated after 2 weeks. The bar plot shows % of AML cells within total BM hCD45+ cells. Mean±SD. Multiple comparisons vs untreated condition by 1-way ANOVA.

FIG. 47E shows peripheral blood lineage composition of NBSGW mice xeno-transplanted with AAVS1^{BE} and dual-edited FLT3^{N399}/CD123559 HSPCs at 9 weeks (experiment from FIG. 49D). Statistical comparisons by unpaired t test. Mean±SD.

FIG. 48A are representative flow cytometry gating strategy for bone marrow analysis of mice xeno-transplanted with edited HSPCs and/or AML PDX (experiment from FIG. 46). Cells are pre-gated on singles, live and physical parameter gates. Populations: 1. human CD45+; 2.T cells (CAR); 3. mNeonGreen+ (AML PDX); 4. human CD45+w/o CAR or AML; 5. total CD19+; 6. total myeloid cells (CD33/66b+); 7. granulocytes+mast cells; 8. Monocytes; 9. mast cells; 10. total granulocytes; 11. mature granulocytes (CD10+/11c+); 12. immature granulocytes (CD10−/11c−); 13. classical dendritic cells, cDC; 14. pro-B cells; 15. pre-B cells; 16. mature B cells; 17. natural killer cells, NK; 18. Prolymphocytes; 19. Myeloblasts; 20. lin-CD34+ (CD33/66+lin-CD34+ and CD33/66-lin-CD34+ are pooled for downstream gating); 21. Lin-CD34+38+; 22. Lin-CD34+38−; 23. Pre-B/NK; 24. granulo-mono progenitors, GMP; 25. common myeloid progenitors, CMP; 26. meka-erythroid progenitors, MEP; 27. hematopoietic stem cells, HSC; 28. multipotent progenitors, MPP; 29. lymphoid-primed multipotent progenitors, LMPP; 30. Beads gate; 31. murine CD45+; 32. plasmacytoid DC; 33. multipotent lymphoid progenitors MLP. CountBeads are first gated on singlets as FSC-A^{low}PI^{high} and then with two additional fluorescent parameters (plot in the bottom left corner).

FIG. 48B are representative flow cytometry gating strategy for T cell panels on the bone marrow and spleen. Cells are pre-gated on singles, live and physical parameter gates. Human CD45+ cells are separated in CD3+ (T cells) and CD3−, within which the residual AML is gated as mNeonGreen+. CAR+ cells are identified by EGFR staining. T cell phenotype is evaluated on gated CD8+ and CD4+ cells by CD45RA and CD62L.

FIG. 49A shows efficiency of CD34+ HSPCs base editing for FLT3^{N399} alone or in combination with KIT^{H378}R, measured on bulk cells or FACS-sorted CD90+ primitive progenitors. KIT^{H378}R single editing are reported from FIG. 41C for comparison. Mean±SD.

FIG. 49B are representative flow cytometry plots of dual FLT3/CD123 reporter K562 cells showing loss of recognition by 4G8 and 7G3 mAbs after multiplex epitope editing.

FIG. 49C shows edited cells in each quadrant of B were FACS-sorted and then co-cultured with dual-specific FLT3/CD123 CAR-T cells. Plots showing the fraction of live target cells (left), % of T cell activation (CD69 expression, middle), and degranulation (CD107a surface expression, right), the median fluorescent intensity (MFI) of CellTrace marking on T cells and FLT3 and CD123 surface expression (MFI) on target cells are reported at different E:T ratio. Mean±SD (N=4). Statistical comparisons by two-way ANOVA.

FIG. 49D is a schematic representation of xeno-transplantation and analysis of dual FLT3^{N399}/CD123^{S59} epitope-edited or AAVS1 BE HSPCs co-engrafted with AML PDX-2 and treated with a 1:1 pool of 4G8 and CSL362 CAR-T cells.

FIG. 49E shows representative flow cytometry plots of BM samples from mice engrafted with CD34+ HSPC, CD34+ HSPCs+AML PDX-2, or CD34+ HSPCs+AML PDX-2 treated with 4G8/CSL362 CARs. Plots are pre-gated on total human CD45+; CAR T cells are identified by CD3 staining, AML PDX cells are mNeonGreen+.

FIG. 49F are bar plots showing the % of AML PDX cells within hCD45+CD3− BM cells. Mean±SD. Statistical comparisons by one-way ANOVA.

FIG. 49G shows FLT3 (left) and CD123 (right) base editing measured on total blood cells (week 9, 10) and on sorted CD33+ and CD19+ BM cells at endpoint of mice from D. Mean±SD. Statistical comparison by multiple unpaired t test.

FIG. 49H shows absolute counts of myeloid (Left) and lymphoid (Right) lineage subsets in the BM, from HSC to differentiated leukocytes. Untreated mice are pooled (grey bars), CAR-treated FLT3^{N399}+CD123^{S59} mice are reported in pink and CAR-treated AAVS1^{BE} mice in blue. The fold change in absolute counts (FLT3^{N399}+CD123^{S59}/AAVS1) for CAR treated groups is reported above each population bar plot. Mean±SD. One-way ANOVA with multiple comparisons. FDR-adjusted p values of the comparison between FLT3^{N399}/CD123^{S59} vs AAVS1 BE conditions treated with 4G8 CAR are reported (p<0.05 are in bold).

### DETAILED DESCRIPTION

Identifying suitable proteins for targeted cancer therapies presents a significant challenge. Many potential target proteins are present on both the cell surface of a cancer cell and on the cell surface of normal, non-cancer cells, which can be required or critically involved in the development and/or survival of the subject. Many of the target proteins contribute to the functionality of such essential cells. Thus, therapies targeting these proteins can lead to deleterious effects in the subject, such as significant toxicity and/or other side effects. Further, resistance to CAR-T therapy remains a challenge in treatment of hematopoietic malignancies, such as acute myeloid leukemia (AML) due to switch of cancer antigens on cancer cells, thereby escaping CAR-T therapy.

Accordingly, the present disclosure provides methods, cells, compositions, and kits aimed at addressing at least the above-stated problems. The methods, cells, compositions, and kits described herein provide a safe and effective treatment for hematological malignancies, allowing for targeting of one or more cell surface proteins that are present not only on cancer cells but also on cells critical for the development and/or survival of the subject. In some instances, described herein are genetically engineered hematopoietic cells such as hematopoietic stem cells (HSPCs) having genetic editing in one or more genes coding for cell-surface proteins, for example, FLT3, CD123, and/or KIT; methods of producing such, for examples, via the CRISPR approach using specific guide RNAs; methods of treating a hematopoietic malignancy using the engineered hematopoietic cells, either taken alone, or in combination with one or more cytotoxic agents (e.g., CAR-T cells) that can target the wild-type cell-surface antigens but not those encoded by the edited genes in the engineered hematopoietic cells; and kits comprising the engineered hematopoietic cells.

### I. Genetically Engineered Hematopoietic Cells

In some embodiments, the genetically engineered hematopoietic cells have an edited FLT3 gene, CD123 gene, or KIT gene. In some in, one or more of these genes are mutated. In some instances, the mutated FLT3 gene, CD123 gene, or KIT gene include mutations or deletions in one or more non-essential epitopes so as to retain (in whole or in part) the bioactivity of FLT3 gene, CD123 gene, or KIT gene.

i. Hematopoietic Stem Cells

In some embodiments, the hematopoietic cells described herein are hematopoietic stem cells. Hematopoietic stem/progenitor cells (HSPCs) are capable of giving rise to both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSPCs are characterized by the expression of the cell surface marker CD34 (e.g., CD34+), which can be used for the identification and/or isolation of HSPCs.

In some embodiments, the HSPCs are obtained from a subject, such as a mammalian subject. In some embodiments, the mammalian subject is a non-human primate, a rodent (e.g., mouse or rat), a bovine, a porcine, an equine, or a domestic animal. In some embodiments, the HSPCs are obtained from a human patient, such as a human patient suffering from a hematopoietic malignancy. In some embodiments, the HSPCs are obtained from a healthy donor. In some embodiments, the HSPCs are obtained from the subject to whom the genetically engineered HSPCs will be subsequently administered. HSPCs that are administered to the same subject from which the cells were obtained are referred to as autologous cells, whereas HSPCs that are obtained from a subject who is not the subject to whom the cells will be administered are referred to as allogeneic cells (methods to reduce incidence of rejection are standard and well known in the art).

HSPCs can be obtained from any suitable source using convention means known in the art. In some embodiments, HSPCs are obtained from a sample from a subject (or donor), such as bone marrow sample or from a blood sample. Alternatively or in addition, HSPCs can be obtained from an umbilical cord (i.e. cord blood cells). In some embodiments, the HSPCs are from bone marrow, cord blood cells, or peripheral blood mononuclear cells (PBMCs). In general, bone marrow cells can be obtained from iliac crest, femora, tibiae, spine, rib or other medullary spaces of a subject (or donor). Bone marrow can be taken out of the patient and isolated through various separations and washing procedures known in the art. An exemplary procedure for isolation of bone marrow cells comprises the following steps: a) extraction of a bone marrow sample; b) centrifugal separation of bone marrow suspension in three fractions and collecting the intermediate fraction, or buffy coat; c) the buffy coat fraction from step (b) is centrifuged one more time in a separation fluid, commonly Ficoll™, and an intermediate fraction which contains the bone marrow cells is collected; and d) washing of the collected fraction from step (c) for recovery of re-transfusable bone marrow cells.

HSPCs typically reside in the bone marrow but can be mobilized into the circulating blood by administering a mobilizing agent in order to harvest HSPCs from the peripheral blood. In some embodiments, the subject (or donor) from which the HSPCs are obtained is administered a mobilizing agent, such as granulocyte colony-stimulating factor (G-CSF). The number of the HSPCs collected following mobilization using a mobilizing agent is typically greater than the number of cells obtained without use of a mobilizing agent.

In some embodiments, a sample is obtained from a subject (or donor) and is then enriched for a desired cell type (e.g. CD34+, CD34+CD38−, CD133+, CD90+, CD49f+). For example, PBMCs and/or CD34+ hematopoietic cells can be isolated from blood as described herein. Cells can also be isolated from other cells, for example by isolation and/or activation with an antibody binding to an epitope on the cell surface of the desired cell type. Another method that can be used includes negative selection using antibodies to cell surface markers to selectively enrich for a specific cell type without activating the cell by receptor engagement.

ii. Mutated Cell-Surface Antigens

In some embodiments, the hematopoietic stem cells (HSPCs) described herein can contain an edited gene encoding one or more cell-surface proteins of interest (e.g. FLT3, CD123, KIT) in mutated form (mutants or variants, which are used herein interchangeably), which has reduced binding or no binding to a cytotoxic agent as described herein (e.g. anti-FLT3 antibody, anti-CD123 antibody, anti-KIT antibody). The mutants can carry one or more mutations of the epitope to which the cytotoxic agent binds, such that binding to the cytotoxic agent is reduced or abolished as compared to the natural or wild-type cell-surface protein counterpart. Such a mutant is preferred to maintain substantially similar biological activity as the wild-type counterpart.

As used herein, the term “reduced binding” refers to binding that is reduced by at least 25%. The level of binding can refer to the amount of binding of the cytotoxic agent to a hematopoietic stem cell or the amount of binding of the cytotoxic agent to the cell-surface protein as compared to a wild-type (i.e., non-engineered, non-mutated) protein. In some embodiments, the binding is reduced by at least 25%, 30%, 40%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100%. In some embodiments, the binding is reduced such that there is substantially no detectable binding in a conventional assay. As used herein, “no binding” refers to substantially no binding, e.g., no detectable binding or only baseline binding as determined in a conventional binding assay.

In some instances, the variant contains one or more amino acid residue substitutions (e.g., 2, 3, 4, 5, or more) within the epitope of interest such that the cytotoxic agent does not bind or has reduced binding to the mutated epitope. Such a mutant can have substantially reduced binding affinity to the cytotoxic agent (e.g., having a binding affinity that is at least 40%, 50%, 60%, 70%, 80% or 90% lower than its wild-type counterpart). In some examples, such a variant can have abolished binding activity to the cytotoxic agent. In other instances, the mutant contains a deletion of a region that comprises the epitope of interest. Such a region can be encoded by an exon. In some embodiments, the region is a domain of the cell-surface protein of interest that encodes the epitope. In one example, the variant has just the epitope deleted. The length of the deleted region can range from 3-60 amino acids, e.g., 5-50, 5-40, 10-30, 10-20, etc.

The mutation(s) or deletions in a mutant of a cell-surface antigen can be within or surround a non-essential epitope such that the mutation(s) or deletion(s) do not substantially affect the bioactivity of the protein.

As used herein, the term “epitope” refers to an amino acid sequence (linear or conformational) of a protein, such as a cell-surface antigen, that is bound by the CDRs of an antibody. In some embodiments, the cytotoxic agent binds to one or more (e.g., at least 2, 3, 4, 5 or more) epitopes of a cell-surface antigen. In some embodiments, the cytotoxic agent binds to more than one epitope of the cell-surface antigen and the hematopoietic cells are manipulated such that each of the epitopes is absent and/or unavailable for binding by the cytotoxic agent.

In some embodiments, the genetically engineered HSPCs described herein have one or more edited genes of cell-surface antigens such that the edited genes express mutated cell-surface antigens with mutations in one or more non-essential epitopes. A “non-essential epitope” (or a fragment comprising such) refers to a domain within the cell surface protein/antigen, the mutation in which is less likely to substantially affect the bioactivity of the cell surface protein. For example, hematopoietic cells comprising a deletion or mutation of a non-essential epitope of a cell-surface antigen, such hematopoietic cells are able to proliferate and/or undergo erythropoietic differentiation to a similar level as hematopoietic cells that express a wild-type cell-surface antigen.

Methods for identifying and/or verifying non-essential epitopes in cell-surface antigens would be known and recognized by one of ordinary skill in the art and is also within the scope of the present disclosure. Further, methods for assessing the functionality of the cell-surface antigen and the hematopoietic cells are known in the art and include, for example, proliferation assays, differentiation assays, colony formation, expression analysis (e.g., gene and/or protein), protein localization, intracellular signaling, functional assays, and in vivo humanized mouse models.

iii. Preparation of Genetically Engineered Hematopoietic Cells

Any of the genetically engineering hematopoietic cells, such as HSPCs, that carry edited genes of one or more cell-surface antigens can be prepared by a routine method or by a method described herein. In some embodiments, the genetic engineering is performed using genome editing. As used herein, “genome editing” refers to a method of modifying the genome, including any protein-coding or non-coding nucleotide sequence, of an organism to alter the expression of a target gene. In general, genome editing methods involve use of an endonuclease that is capable of cleaving the nucleic acid of the genome, for example at a targeted nucleotide sequence. In some instances, genome editing methods involve use of a dead nuclease or a nuclease that is a nickase. Repair of the double-stranded breaks in the genome can be repaired introducing mutations and/or exogenous nucleic acid can be inserted into the targeted site. In some cases, genome editing methods involve use of a catalytically inactive or partially inactive endonuclease fused to a functional domain, e.g. an adenine or cytidine deaminase domain in the case of base editors. Other functional domains include prime editors, CRISPR-Cas activators or repressors, etc.

Genome editing methods are generally classified based on the type of endonuclease that is involved in generating double stranded breaks in the target nucleic acid. These methods include use of zinc finger nucleases (ZFN), transcription activator-like effector-based nuclease (TALEN), meganucleases, and CRISPR/Cas systems.

In one aspect of the present disclosure, the replacement of cancer cells by a modified population of normal cells is performed using normal cells that have been manipulated such that the cells do not bind the cytotoxic agent. Such modification can include the deletion or mutation of an epitope of the specific cell-surface protein using a CRISPR-Cas system, where the Clustered Regularly Interspaced Short Palindromic Repeats (CRISPR)-Cas system is an engineered, non-naturally occurring CRISPR-Cas system.

CRISPR systems encode RNA-guided endonucleases that are essential for bacterial adaptive immunity. CRISPR-associated (Cas) nucleases can be readily programmed to cleave target DNA sequences for genome editing in various. One class of these nucleases, referred to as Cas9 proteins, complex with two short RNAs: a crRNA and a trans-activating crRNA (tracrRNA). The most commonly used Cas9 ortholog, SpCas9, uses a crRNA that has 20 nucleotides (nt) at its 5′ end that are complementary to the “protospacer” region of the target DNA site. Efficient cleavage also requires that SpCas9 recognizes a protospacer adjacent motif (PAM). The crRNA and tracrRNA are usually combined into a single ˜100-nt guide RNA (gRNA) that directs the DNA cleavage activity of SpCas9. A Cas protein named Cpf1 has been identified that can also be programmed to cleave target DNA sequences. Unlike SpCas9, Cpf1 requires only a single 42-nt crRNA, which has 23 nt at its 3′ end that are complementary to the protospacer of the target DNA sequence.

In some embodiments, the Cas endonuclease is a Cas9 nuclease or variant thereof. Cas9 endonucleases cleave double stranded DNA of a target nucleic acid resulting in blunt ends. In some embodiments, the Cas endonuclease is a Cpf1 nuclease or variant thereof. Cleavage with Cpf1 nucleases results in staggered ends of the nucleic acid.

### Cas9

In some embodiments, the Cas endonuclease is a Cas9 enzyme or variant thereof. In some embodiments, the Cas9 endonuclease is derived from Streptococcus pyogenes (SpCas9) or Staphylococcus aureus (SaCas9).

The SpCas9 wild type sequence is as follows (SEQ ID NO: 1):

MDKKYSIGLD IGTNSVGWAV ITDEYKVPSK KFKVLGNTDR HSIKKNLIGA
LLFDSGETAE ATRLKRTARR RYTRRKNRIC YLQEIFSNEM AKVDDSFFHR
LEESELVEED KKHERHPIFG NIVDEVAYHE KYPTIYHLRK KLVDSTDKAD
LRLIYLALAH MIKFRGHFLI EGDLNPDNSD VDKLFIQLVQ TYNQLFEENP
INASGVDAKA ILSARLSKSR RLENLIAQLP GEKKNGLFGN LIALSLGLTP
NFKSNFDLAE DAKLQLSKDT YDDDLDNLLA QIGDQYADLF LAAKNLSDAI
LLSDILRVNT EITKAPLSAS MIKRYDEHHQ DLTLLKALVR QQLPEKYKEI
FFDQSKNGYA GYIDGGASQE EFYKFIKPIL EKMDGTEELL VKLNREDLLR
KQRTFDNGSI PHQIHLGELH AILRRQEDFY PFLKDNREKI EKILTFRIPY
YVGPLARGNS RFAWMTRKSE ETITPWNFEE VVDKGASAQS FIERMINQDK
NLPNEKVLPK HSLLYEYFTV YNELTKVKYV TEGMRKPAFL SGEQKKAIVD
LLFKTNRKVT VKQLKEDYFK KIECFDSVEI SGVEDRFNAS LGTYHDLLKI
IKDKDFLDNE ENEDILEDIV LTLTLFEDRE MIEERLKTYA HLFDDKVMKQ
LKRRRYTGWG RLSRKLINGI RDKFSGKTIL DFLKSDGFAN RNFMQLIHDD
SLTFKEDIQK AQVSGQGDSL HEHIANLAGS PAIKKGILQT VKVVDELVKV
MGRHKPENIV IEMARENQTT QKGQKNSRER MKRIEEGIKE LGSQILKEHP
VENTQLQNEK LYLYYLQNGR DMYVDQELDI NRLSDYDVDH IVPQSFLKDD
SIDNKVLTRS DKNRGKSDNV PSEEVVKKMK NYWRQLLNAK LITQRKFDNL
TKAERGGLSE LDKAGFIKRQ LVETRQITKH VAQILDSRMN TKYDENDKLI
REVKVITLKS KLVSDFRKDF QFYKVREINN YHHAHDAYLN AVVGTALIKK
YPKLESEFVY GDYKVYDVRK MIAKSEQEIG KATAKYFFYS NIMNFFKTEI
TLANGEIRKR PLIETNGETG EIVWDKGRDF ATVRKVLSMP QVNIVKKTEV
QTGGFSKESI LPKRNSDKLI ARKKDWDPKK YGGFDSPTVA YSVLVVAKVE
KGKSKKLKSV KELLGITIME RSSFEKNPID FLEAKGYKEV KKDLIIKLPK
YSLFELENGR KRMLASAGEL QKGNELALPS KYVNFLYLAS HYEKLKGSPE
DNEQKQLFVE QHKHYLDEII EQISEFSKRV ILADANLDKV LSAYNKHRDK
PIREQAENII HLFTLTNLGA PAAFKYEDTT IDRKRYTSTK EVLDATLIHQ
SITGLYETRI DLSQLGGD
The SaCas9 wild type sequence is as follows (SEQ ID NO: 2):
MKRNYILGLD IGITSVGYGI IDYETRDVID AGVRLFKEAN VENNEGRRSK
RGARRLKRRR RHRIQRVKKL LFDYNLLTDH SELSGINPYE ARVKGLSQKL
SEEEFSAALL HLAKRRGVHN VNEVEEDTGN ELSTKEQISR NSKALEEKYV
AELQLERLKK DGEVRGSINR FKTSDYVKEA KQLLKVQKAY HQLDQSFIDT
YIDLLETRRT YYEGPGEGSP FGWKDIKEWY EMLMGHCTYF PEELRSVKYA
YNADLYNALN DLNNLVITRD ENEKLEYYEK FQIIENVFKQ KKKPTLKQIA
KEILVNEEDI KGYRVTSTGK PEFTNLKVYH DIKDITARKE IIENAELLDQ
IAKILTIYQS SEDIQEELTN LNSELTQEEI EQISNLKGYT GTHNLSLKAI
NLILDELWHT NDNQIAIFNR LKLVPKKVDL SQQKEIPTTL VDDFILSPVV
KRSFIQSIKV INAIIKKYGL PNDIIIELAR EKNSKDAQKM INEMQKRNRQ
TNERIEEIIR TTGKENAKYL IEKIKLHDMQ EGKCLYSLEA IPLEDLLNNP
FNYEVDHIIP RSVSFDNSFN NKVLVKQEEN SKKGNRTPFQ YLSSSDSKIS
YETFKKHILN LAKGKGRISK TKKEYLLEER DINRFSVQKD FINRNLVDTR
YATRGLMNLL RSYFRVNNLD VKVKSINGGF TSFLRRKWKF KKERNKGYKH
HAEDALIIAN ADFIFKEWKK LDKAKKVMEN QMFEEKQAES MPEIETEQEY
KEIFITPHQI KHIKDFKDYK YSHRVDKKPN RELINDTLYS TRKDDKGNTL
IVNNLNGLYD KDNDKLKKLI NKSPEKLLMY HHDPQTYQKL KLIMEQYGDE
KNPLYKYYEE TGNYLTKYSK KDNGPVIKKI KYYGNKLNAH LDITDDYPNS
RNKVVKLSLK PYRFDVYLDN GVYKFVTVKN LDVIKKENYY EVNSKCYEEA
KKLKKISNQA EFIASFYNND LIKINGELYR VIGVNNDLLN RIEVNMIDIT
YREYLENMND KRPPRIIKTI ASKTQSIKKY STDILGNLYE VKSKKHPQII KKG

In general, the target nucleic acid is flanked on the 3′ side or 5′ side by a protospacer adjacent motif (PAM) that can interact with the endonuclease and be further involved in targeting the endonuclease activity to the target nucleic acid. It is generally thought that the PAM sequence flanking the target nucleic acid depends on the endonuclease and the source from which the endonuclease is derived. For example, for Cas9 endonucleases that are derived from Streptococcus pyogenes, the PAM sequence is NGG, although the PAM sequences NAG and NGA can be recognized with lower efficiency. For Cas9 endonucleases derived from Staphylococcus aureus, the PAM sequence is NNGRRT (SEQ ID NO: 3).

Accordingly, in some instances, the endonuclease is engineered/modified such that it can recognize one or more PAM sequence. In some embodiments, the endonuclease has been engineered/modified to recognize one or more PAM sequence that is different than the PAM sequence the endonuclease recognizes without engineering/modification. In some embodiments, the endonuclease has been engineered/modified to reduce off-target activity of the enzyme. In some embodiments, the nucleotide sequence encoding the endonuclease is modified to alter the PAM recognition of the endonuclease. For example, the Cas endonuclease (e.g., SpCas9) has mutations at one or more of the following positions: A61, L1111, D1135, S1136, G1218, E1219, N1317, A1322, R1333, R1335, T1337. See, for example, International Patent Application Publication Nos. WO 2016/141224 and WO 2017/040348, US Patent Application Publication No. 2021/0284978A1, all of which are all incorporated herein by reference.

In some embodiments, the Cas9 endonuclease is the wild-type version of the nuclease. For example, the Cas9 endonuclease is an SpCas9 endonuclease having the sequence shown above in SEQ ID NO: 1. In some embodiments, the SpCas9 endonuclease is at least 80%, e.g., at least 85%, 90%, or 95% identical to the amino acid sequence of SEQ ID NO: 1, e.g., have differences at up to 5%, 10%, 15%, or 20% of the residues of SEQ ID NO: 1 replaced, e.g., with conservative mutations. In preferred embodiments, the endonuclease retains desired activity of the parent, e.g., the nuclease activity (except where the parent is a nickase or a dead Cas9), and/or the ability to interact with a guide RNA and target DNA). In other instances, the Cas9 endonuclease is an SaCas9 endonuclease having the sequence shown above in SEQ ID NO: 2. In some embodiments, the SaCas9 endonuclease is at least 80%, e.g., at least 85%, 90%, or 95% identical to the amino acid sequence of SEQ ID NO: 2, e.g., have differences at up to 5%, 10%, 15%, or 20% of the residues of SEQ ID NO: 2 replaced, e.g., with conservative mutations. In preferred embodiments, the endonuclease retains desired activity of the parent, e.g., the nuclease activity (except where the parent is a nickase or a dead Cas9), and/or the ability to interact with a guide RNA and target DNA).

In some embodiments, the Cas9 endonuclease is a catalytically inactive Cas9. For example, dCas9 contains mutations at catalytically active residues (D10, E762, D839, H983, or D986; and/or at H840 or N863) and does not have nuclease activity. For example, the mutations are: (i) D10A or D10N, and/or (ii) H840A, H840N, or H840Y.

In some embodiments, the Cas9 endonuclease includes a mutation at K918. For instance the mutation is K918N.

In some embodiments, the nucleotide sequence encoding the Cas9 endonuclease is further modified to alter the activity of the protein. In some embodiments, the Cas9 endonuclease has been modified to inactivate one or more catalytic residues of the endonuclease. In some embodiments, the Cas9 endonuclease has been modified to inactivate one of the catalytic residues of the endonuclease, referred to as a “nickase” or “Cas9n.” Cas9 nickase endonucleases cleave one DNA strand of the target nucleic acid.

In some instances, the endonuclease is a NG-SpCas9 nickase and has the following mutations: D10A, L1111R, D1135V, G1218R, E1219F, A1322R, R1335V, T1337R (relative to wild-type SpCas9). In some instances, the endonuclease is SpRY-Cas9 nickase and has the following mutations: D10A, A61R, L1111R, D1135L, S1136W, G1218K, E1219Q, N1317R, A1322R, R1333P, R1335Q, T1337R (relative to wild-type SpCas9).

### Cpf1 (Cas12a)

In some embodiments, the Cas endonuclease is a Cpf1 nuclease or variant thereof. Cpf1 endonuclease generally recognizes a PAM sequence located at the 5′ end of the target nucleic acid. For a Cpf1 nuclease, the PAM sequence is TTTN. As will be appreciated by one of skill in the art, the Cas endonuclease Cpf1 nuclease can also be referred to as Cas12a. In some embodiments, the host cell expresses a Cpf1 nuclease derived from Lachnospiraceae bacterium (LbCpf1), Acidaminococcus sp. (AsCpf1), or Francisella tularensis (FnCpf1). Wild-type sequences for each are shown below.

Type V CRISPR-associated protein Cpf1 [Lachnospiraceae bacterium ND2006],
GenBank Acc No. WP_051666128.1
(SEQ ID NO: 4)
MLKNVGIDRL DVEKGRKNMS KLEKFTNCYS LSKTLREKAI PVGKTQENID
NKRLLVEDEK RAEDYKGVKK LLDRYYLSFI NDVLHSIKLK NLNNYISLFR
KKTRTEKENK ELENLEINLR KEIAKAFKGN EGYKSLFKKD IIETILPEFL
DDKDEIALVN SFNGFTTAFT GFFDNRENMF SEEAKSTSIA FRCINENLTR
YISNMDIFEK VDAIFDKHEV QEIKEKILNS DYDVEDFFEG EFFNFVLTQE
GIDVYNAIIG GFVTESGEKI KGLNEYINLY NQKTKQKLPK FKPLYKQVLS
DRESLSFYGE GYTSDEEVLE VFRNTLNKNS EIFSSIKKLE KLFKNFDEYS
SAGIFVKNGP AISTISKDIF GEWNVIRDKW NAEYDDIHLK KKAVVTEKYE
DDRRKSFKKI GSFSLEQLQE YADADLSVVE KLKEIIIQKV DEIYKVYGSS
EKLFDADFVL EKSLKKNDAV VAIMKDLLDS VKSFENYIKA FFGEGKETNR
DESFYGDFVL AYDILLKVDH IYDAIRNYVT QKPYSKDKFK LYFQNPQFMG
GWDKDKETDY RATILRYGSK YYLAIMDKKY AKCLQKIDKD DVNGNYEKIN
YKLLPGPNKM LPKVFFSKKW MAYYNPSEDI QKIYKNGTFK KGDMFNLNDC
HKLIDFFKDS ISRYPKWSNA YDFNFSETEK YKDIAGFYRE VEEQGYKVSF
ESASKKEVDK LVEEGKLYMF QIYNKDFSDK SHGTPNLHTM YFKLLFDENN
HGQIRLSGGA ELFMRRASLK KEELVVHPAN SPIANKNPDN PKKTTTLSYD
VYKDKRFSED QYELHIPIAI NKCPKNIFKI NTEVRVLLKH DDNPYVIGID
RGERNLLYIV VVDGKGNIVE QYSLNEIINN FNGIRIKTDY HSLLDKKEKE
RFEARQNWTS IENIKELKAG YISQVVHKIC ELVEKYDAVI ALEDLNSGFK
NSRVKVEKQV YQKFEKMLID KLNYMVDKKS NPCATGGALK GYQITNKFES
FKSMSTQNGF IFYIPAWLTS KIDPSTGFVN LLKTKYTSIA DSKKFISSFD
RIMYVPEEDL FEFALDYKNF SRTDADYIKK WKLYSYGNRI RIFRNPKKNN
VFDWEEVCLT SAYKELENKY GINYQQGDIR ALLCEQSDKA FYSSEMALMS
LMLQMRNSIT GRIDVDFLIS PVKNSDGIFY DSRNYEAQEN AILPKNADAN
GAYNIARKVL WAIGQFKKAE DEKLDKVKIA ISNKEWLEYA QTSVKH
Type V CRISPR-associated protein Cpf1 [Acidaminococcus sp. BV3L6], NCBI
Reference Sequence: WP_021736722.1
(SEQ ID NO: 5)
MTQFEGFTNL YQVSKTLRFE LIPQGKTLKH IQEQGFIEED KARNDHYKEL
KPIIDRIYKT YADQCLQLVQ LDWENLSAAI DSYRKEKTEE TRNALIEEQA
TYRNAIHDYF IGRTDNLTDA INKRHAEIYK GLFKAELFNG KVLKQLGTVT
TTEHENALLR SFDKFTTYFS GFYENRKNVF SAEDISTAIP HRIVQDNFPK
FKENCHIFTR LITAVPSLRE HFENVKKAIG IFVSTSIEEV FSFPFYNQLL
TQTQIDLYNQ LLGGISREAG TEKIKGLNEV LNLAIQKNDE TAHIIASLPH
RFIPLFKQIL SDRNTLSFIL EEFKSDEEVI QSFCKYKTLL RNENVLETAE
ALFNELNSID LTHIFISHKK LETISSALCD HWDTLRNALY ERRISELTGK
ITKSAKEKVQ RSLKHEDINL QEIISAAGKE LSEAFKQKTS EILSHAHAAL
DQPLPTTLKK QEEKEILKSQ LDSLLGLYHL LDWFAVDESN EVDPEFSARL
TGIKLEMEPS LSFYNKARNY ATKKPYSVEK FKLNFQMPTL ASGWDVNKEK
NNGAILFVKN GLYYLGIMPK QKGRYKALSF EPTEKTSEGF DKMYYDYFPD
AAKMIPKCST QLKAVTAHFQ THTTPILLSN NFIEPLEITK EIYDLNNPEK
EPKKFQTAYA KKTGDQKGYR EALCKWIDFT RDFLSKYTKT TSIDLSSLRP
SSQYKDLGEY YAELNPLLYH ISFQRIAEKE IMDAVETGKL YLFQIYNKDF
AKGHHGKPNL HTLYWTGLFS PENLAKTSIK LNGQAELFYR PKSRMKRMAH
RLGEKMLNKK LKDQKTPIPD TLYQELYDYV NHRLSHDLSD EARALLPNVI
TKEVSHEIIK DRRFTSDKFF FHVPITLNYQ AANSPSKFNQ RVNAYLKEHP
ETPIIGIDRG ERNLIYITVI DSTGKILEQR SLNTIQQFDY QKKLDNREKE
RVAARQAWSV VGTIKDLKQG YLSQVIHEIV DLMIHYQAVV VLENLNFGFK
SKRTGIAEKA VYQQFEKMLI DKLNCLVLKD YPAEKVGGVL NPYQLTDQFT
SFAKMGTQSG FLFYVPAPYT SKIDPLTGFV DPFVWKTIKN HESRKHFLEG
FDFLHYDVKT GDFILHFKMN RNLSFQRGLP GFMPAWDIVF EKNETQFDAK
GTPFIAGKRI VPVIENHRFT GRYRDLYPAN ELIALLEEKG IVFRDGSNIL
PKLLENDDSH AIDTMVALIR SVLQMRNSNA ATGEDYINSP VRDLNGVCFD
SRFQNPEWPM DADANGAYHI ALKGQLLLNH LKESKDLKLQ NGISNQDWLA
YIQELRN
Type V CRISPR-associated protein Cpf1 [Francisella tularensis], GenBank
Acc No. WP_003040289.1
(SEQ ID NO: 6)
MSIYQEFVNK YSLSKTLRFE LIPQGKTLEN IKARGLILDD EKRAKDYKKA
KQIIDKYHQF FIEEILSSVC ISEDLLQNYS DVYFKLKKSD DDNLQKDFKS
AKDTIKKQIS EYIKDSEKFK NLFNQNLIDA KKGQESDLIL WLKQSKDNGI
ELFKANSDIT DIDEALEIIK SFKGWTTYFK GFHENRKNVY SSNDIPTSII
YRIVDDNLPK FLENKAKYES LKDKAPEAIN YEQIKKDLAE ELTFDIDYKT
SEVNQRVFSL DEVFEIANFN NYLNQSGITK FNTIIGGKFV NGENTKRKGI
NEYINLYSQQ INDKTLKKYK MSVLFKQILS DTESKSFVID KLEDDSDVVT
TMQSFYEQIA AFKTVEEKSI KETLSLLFDD LKAQKLDLSK IYFKNDKSLT
DLSQQVEDDY SVIGTAVLEY ITQQIAPKNL DNPSKKEQEL IAKKTEKAKY
LSLETIKLAL EEFNKHRDID KQCRFEEILA NFAAIPMIFD EIAQNKDNLA
QISIKYQNQG KKDLLQASAE DDVKAIKDLL DQTNNLLHKL KIFHISQSED
KANILDKDEH FYLVFEECYF ELANIVPLYN KIRNYITQKP YSDEKFKLNF
ENSTLANGWD KNKEPDNTAI LFIKDDKYYL GVMNKKNNKI FDDKAIKENK
GEGYKKIVYK LLPGANKMLP KVFFSAKSIK FYNPSEDILR IRNHSTHTKN
GSPQKGYEKF EFNIEDCRKF IDFYKQSISK HPEWKDFGER FSDTQRYNSI
DEFYREVENQ GYKLTFENIS ESYIDSVVNQ GKLYLFQIYN KDFSAYSKGR
PNLHTLYWKA LFDERNLQDV VYKLNGEAEL FYRKQSIPKK ITHPAKEAIA
NKNKDNPKKE SVFEYDLIKD KRFTEDKFFF HCPITINFKS SGANKFNDEI
NLLLKEKAND VHILSIDRGE RHLAYYTLVD GKGNIIKQDT FNIIGNDRMK
TNYHDKLAAI EKDRDSARKD WKKINNIKEM KEGYLSQVVH EIAKLVIEYN
AIVVFEDLNF GFKRGRFKVE KQVYQKLEKM LIEKLNYLVF KDNEEDKTGG
VLRAYQLTAP FETFKKMGKQ TGIIYYVPAG FTSKICPVTG FVNQLYPKYE
SVSKSQEFFS KFDKICYNLD KGYFEFSFDY KNFGDKAAKG KWTIASFGSR
LINFRNSDKN HNWDTREVYP TKELEKLLKD YSIEYGHGEC IKAAICGESD
KKFFAKLTSV LNTILQMRNS KIGTELDYLI SPVADVNGNF FDSRQAPKNM
PQDADANGAY HIGLKGLMLL GRIKNNQEGK KLNLVIKNEE YFEFVQNRNN

In some embodiments, the Cpf1 endonuclease is the wild-type version of the nuclease. For example, the Cpf1 endonuclease is a Cpf1 endonuclease having the sequence shown above in SEQ ID NO: 4, SEQ ID NO: 5, or SEQ ID NO: 6. In some embodiments, the Cpf1 endonuclease is at least 80%, e.g., at least 85%, 90%, or 95% identical to the amino acid sequence of SEQ ID NO: 4, SEQ ID NO: 5, or SEQ ID NO: 6, e.g., have differences at up to 5%, 10%, 15%, or 20% of the residues of SEQ ID NO: 4, SEQ ID NO: 5, or SEQ ID NO: 6 replaced, e.g., with conservative mutations. In preferred embodiments, the endonuclease retains desired activity of the parent, e.g., the nuclease activity (except where the parent is a nickase or a dead Cas9), and/or the ability to interact with a guide RNA and target DNA).

A catalytically inactive variant of Cpf1 (Cas12a) can be referred to dCas12a. Thus, in some embodiments, for AsCpf1, catalytic activity-destroying mutations are made at D908 and E993, e.g., D908A and E993A; and for LbCpf1 catalytic activity-destroying mutations at D832 and E925, e.g., D832A and E925A; and for FnCpf1 catalytic activity-destroying mutations at D917A and E1006A.

### Functional Domains

Alternatively or in addition, the Cas endonuclease (i.e., Cas9 or Cas12a) can be fused to another protein or portion thereof, e.g., a heterologous functional domain. In some embodiments, the heterologous functional domain is a transcriptional activation domain (e.g., VP64 or NF-KB p65). In some embodiments, the heterologous functional domain is a transcriptional silencer or transcriptional repression domain (e.g., wherein the transcriptional repression domain is Krueppel-associated box (KRAB) domain, ERF repressor domain (ERD), or mSin3A interaction domain (SID); wherein the transcriptional silencer is Heterochromatin Protein 1 (HP1)). In some embodiments, the heterologous functional domain is an enzyme that modifies the methylation state of DNA (e.g., a DNA methyltransferase (DNMT) or a TET protein (such as, TET1)). In some embodiments, the heterologous functional domain is an enzyme that modifies a histone subunit (e.g., a histone acetyltransferase (HAT), histone deacetylase (HDAC), histone methyltransferase (HMT), or histone demethylase). In some embodiments, the heterologous functional domain is a biological tether (e.g., MS2, Csy4 or lambda N). In some embodiments, the heterologous functional domain is FokI.

In some embodiments, the heterologous functional domain is a base editor, such as a deaminase that modifies cytosine DNA bases, e.g., a cytidine deaminase from the apolipoprotein B mRNA-editing enzyme, catalytic polypeptide-like (APOBEC) family of deaminases, including APOBEC1, APOBEC2, APOBEC3A, APOBEC3B, APOBEC3C, APOBEC3D/E, APOBEC3F, APOBEC3G, APOBEC3H, APOBEC4, activation-induced cytidine deaminase (AID), cytosine deaminase 1 (CDA1), and CDA2, and cytosine deaminase acting on tRNA (CDAT). Specific examples include, evoAPOBEC1-BE4max, eA3A-BE5, and EA-BE4max.

In some embodiments, the heterologous functional domain is a deaminase that modifies adenosine DNA bases, e.g., the deaminase is an adenosine deaminase 1 (ADA1), ADA2; adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA). For example, ABE8e-TadA-8e. In some embodiments, the TadA adenosine deaminase domain includes a V106W substitution.

In some embodiments, the heterologous functional domain is an enzyme, domain, or peptide that inhibits or enhances endogenous DNA repair or base excision repair (BER) pathways, e.g., uracil DNA glycosylase inhibitor (UGI) that inhibits uracil DNA glycosylase (UDG, also known as uracil N-glycosylase, or UNG) mediated excision of uracil to initiate BER; or DNA end-binding proteins such as Gam from the bacteriophage Mu.

In some embodiments, the endonuclease is a base editor. Base editor endonuclease generally comprises a catalytically inactive Cas endonuclease fused to a base editor. For example, the endonuclease is SpCas9 with a mutation at D10, E762, D839, H983, or D986; and/or at H840 or N863 and fused to a base editor, such as those mentioned above.

In some instances, the endonuclease (Cas9 or Cas12a) is fused to one or more of a nuclear localization sequence, cell penetrating peptide sequence, affinity tag, and/or a fluorescent protein. For example, the nuclear localization sequence is the SV40 large T-antigen nuclear localization sequence (PKKKRKV; SEQ ID NO: 82), the nucleoplasmin nuclear localization sequence (KRPAATKKAGQAKKKK; SEQ ID NO: 83) or the c-Myc nuclear localization sequence (PAAKRVKLD; SEQ ID NO: 84). For example, the nuclear localization sequence(s) is fused to the N-terminus and/or to the C-terminus of the Cas9 or Cas12a protein. In some embodiments, when a heterologous functional domain is fused to the N-terminus and/or to the C-terminus of the Cas9 or Cas12a protein, the nuclear localization sequence(s) is fused to the N-terminus and/or to the C-terminus of the heterologous functional domain-Cas protein complex or interposed between the heterologous functional domain and the Cas protein.

Sequences of exemplary Cas endonucleases are provided below:

SEQ ID NO: 7 (Amino acid sequence of the SpRY-ABE8e-V106W 3xNLS adenine
base editor):
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIG
RVVFGWRNSKRGAAGSLMNVLNYPGMNHRVEITEGILADECAALLCDFYRMPRQVENAQ
KKAQSSINSGGSSGGSSGSETPGTSESATPESSGGSSGGSDKKYSIGLAIGTNSVGWAV
ITDEYKVPSKKFKVLGNTDRHSIKKNLIGALLFDSGETAERTRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDELDNE
ENEDILEDIVLTLTLFEDREMIEERLKTYAHLFDDKVMKQLKRRRYTGWGRLSRKLING
IRDKQSGKTILDFLKSDGFANRNEMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESIRPKRNSDKLIARKKDWDPKKYGGFLWPTVAY
SVLVVAKVEKGKSKKLKSVKELLGITIMERSSFEKNPIDFLEAKGYKEVKKDLIIKLPK
YSLFELENGRKRMLASAKQLQKGNELALPSKYVNFLYLASHYEKLKGSPEDNEQKQLFV
EQHKHYLDEIIEQISEFSKRVILADANLDKVLSAYNKHRDKPIREQAENIIHLFTLTRL
GAPRAFKYFDTTIDPKQYRSTKEVLDATLIHQSITGLYETRIDLSQLGGDSGGSKRTAD
GSEFEPKKKRKVGSGSKRPAATKKAGQAKKKKLE
SEQ ID NO: 85 (Amino acid sequence of the SpRY-ABE8e 3xNLS adenine base
editor):
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCWMCAGAMIHSRIG
RVVFGWRNSKRGAAGSLMNVLNYPGMNHRVEITEGILADECAALLCDFYRMPRQVENAQ
KKAQSSINSGGSSGGSSGSETPGTSESATPESSGGSSGGSDKKYSIGLAIGTNSVGWAV
ITDEYKVPSKKFKVLGNTDRHSIKKNLIGALLFDSGETAERTRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNE
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDELDNE
ENEDILEDIVLTLTLFEDREMIEERLKTYAHLEDDKVMKQLKRRRYTGWGRLSRKLING
IRDKQSGKTILDFLKSDGFANRNEMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFIKRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESIRPKRNSDKLIARKKDWDPKKYGGFLWPTVAY
SVLVVAKVEKGKSKKLKSVKELLGITIMERSSFEKNPIDFLEAKGYKEVKKDLIIKLPK
YSLFELENGRKRMLASAKQLQKGNELALPSKYVNFLYLASHYEKLKGSPEDNEQKQLFV
EQHKHYLDEIIEQISEFSKRVILADANLDKVLSAYNKHRDKPIREQAENIIHLFTLTRL
GAPRAFKYFDTTIDPKQYRSTKEVLDATLIHQSITGLYETRIDLSQLGGDSGGSKRTAD
GSEFEPKKKRKVGSGSKRPAATKKAGQAKKKKLE
SEQ ID NO: 8-Amino acid sequence of the SpRY-evoAPOBEC1-BE4 3xNLS adenine
base editor
MKRTADGSEFESPKKKRKVSSKTGPVAVDPTLRRRIEPHEFEVFFDPRELRKETCLLYE
INWGGRHSIWRHTSQNTNKHVEVNFIEKFTTERYFCPNTRCSITWFLSWSPCGECSRAI
TEFLSRYPNVTLFIYIARLYHLANPRNRQGLRDLISSGVTIQIMTEQESGYCWHNFVNY
SPSNESHWPRYPHLWVRLYVLELYCIILGLPPCLNILRRKQSQLTSFTIALQSCHYQRL
PPHILWATGLKSGGSSGGSSGSETPGTSESATPESSGGSSGGSDKKYSIGLAIGTNSVG
WAVITDEYKVPSKKFKVLGNTDRHSIKKNLIGALLFDSGETAERTRLKRTARRRYTRRK
NRICYLQEIFSNEMAKVDDSFFHRLEESFLVEEDKKHERHPIFGNIVDEVAYHEKYPTI
YHLRKKLVDSTDKADLRLIYLALAHMIKFRGHFLIEGDLNPDNSDVDKLFIQLVQTYNQ
LFEENPINASGVDAKAILSARLSKSRRLENLIAQLPGEKKNGLFGNLIALSLGLTPNEK
SNFDLAEDAKLQLSKDTYDDDLDNLLAQIGDQYADLFLAAKNLSDAILLSDILRVNTEI
TKAPLSASMIKRYDEHHQDLTLLKALVRQQLPEKYKEIFFDQSKNGYAGYIDGGASQEE
FYKFIKPILEKMDGTEELLVKLNREDLLRKQRTEDNGSIPHQIHLGELHAILRRQEDFY
PFLKDNREKIEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRENASLGTYHDLLKIIKDKDEL
DNEENEDILEDIVLTLTLFEDREMIEERLKTYAHLEDDKVMKQLKRRRYTGWGRLSRKL
INGIRDKQSGKTILDFLKSDGFANRNFMQLIHDDSLTFKEDIQKAQVSGQGDSLHEHIA
NLAGSPAIKKGILQTVKVVDELVKVMGRHKPENIVIEMARENQTTQKGQKNSRERMKRI
EEGIKELGSQILKEHPVENTQLQNEKLYLYYLQNGRDMYVDQELDINRLSDYDVDHIVP
QSFLKDDSIDNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIREVKVITLKSK
LVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRD
FATVRKVLSMPQVNIVKKTEVQTGGFSKESIRPKRNSDKLIARKKDWDPKKYGGFLWPT
VAYSVLVVAKVEKGKSKKLKSVKELLGITIMERSSFEKNPIDFLEAKGYKEVKKDLIIK
LPKYSLFELENGRKRMLASAKQLQKGNELALPSKYVNFLYLASHYEKLKGSPEDNEQKQ
LEVEQHKHYLDEIIEQISEFSKRVILADANLDKVLSAYNKHRDKPIREQAENIIHLETL
TRLGAPRAFKYFDTTIDPKQYRSTKEVLDATLIHQSITGLYETRIDLSQLGGDSGGSGG
SGGSTNLSDIIEKETGKQLVIQESILMLPEEVEEVIGNKPESDILVHTAYDESTDENVM
LLTSDAPEYKPWALVIQDSNGENKIKMLSGGSGGSGGSTNLSDIIEKETGKQLVIQESI
LMLPEEVEEVIGNKPESDILVHTAYDESTDENVMLLTSDAPEYKPWALVIQDSNGENKI
KMLSGGSKRTADGSEFEPKKKRKVGSGSKRPAATKKAGQAKKKKLE
SEQ ID NO: 9-Amino acid sequence of the SpRY-K918N-ABE8e-V106W 3xNLS
adenine base editor
MKRTADGSEFESPKKKRKVSEVEFSHEYWMRHALTLAKRARDEREVPVGAVLVLNNRVI
GEGWNRAIGLHDPTAHAEIMALRQGGLVMQNYRLIDATLYVTFEPCVMCAGAMIHSRIG
RVVFGWRNSKRGAAGSLMNVLNYPGMNHRVEITEGILADECAALLCDFYRMPRQVENAQ
KKAQSSINSGGSSGGSSGSETPGTSESATPESSGGSSGGSDKKYSIGLAIGTNSVGWAV
ITDEYKVPSKKFKVLGNTDRHSIKKNLIGALLFDSGETAERTRLKRTARRRYTRRKNRI
CYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSLGLTPNFKSNF
DLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSFI
ERMTNFDKNLPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKIIKDKDFLDNE
ENEDILEDIVLTLTLFEDREMIEERLKTYAHLFDDKVMKQLKRRRYTGWGRLSRKLING
IRDKQSGKTILDFLKSDGFANRNEMQLIHDDSLTFKEDIQKAQVSGQGDSLHEHIANLA
GSPAIKKGILQTVKVVDELVKVMGRHKPENIVIEMARENQTTQKGQKNSRERMKRIEEG
IKELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKAER
GGLSELDKAGFINRQLVETRQITKHVAQILDSRMNTKYDENDKLIREVKVITLKSKLVS
DFRKDFQFYKVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIVWDKGRDFAT
VRKVLSMPQVNIVKKTEVQTGGFSKESIRPKRNSDKLIARKKDWDPKKYGGFLWPTVAY
SVLVVAKVEKGKSKKLKSVKELLGITIMERSSFEKNPIDFLEAKGYKEVKKDLIIKLPK
YSLFELENGRKRMLASAKQLQKGNELALPSKYVNFLYLASHYEKLKGSPEDNEQKQLFV
EQHKHYLDEIIEQISEFSKRVILADANLDKVLSAYNKHRDKPIREQAENIIHLETLTRL
GAPRAFKYFDTTIDPKQYRSTKEVLDATLIHQSITGLYETRIDLSQLGGDSGGSKRTAD
GSEFEPKKKRKVGSGSKRPAATKKAGQAKKKKLE
SEQ ID NO: 10-Nucleotide sequence of the SpRY-ABE8e-V106W 3xNLS adenine
base editor:
atgaaacggacagccgacggaagcgagttcgagtcaccaaagaagaagcggaaagtctc
tgaggtggagttttcccacgagtactggatgagacatgccctgaccctggccaagaggg
cacgggatgagagggaggtgcctgtgggagccgtgctggtgctgaacaatagagtgatc
ggcgagggctggaacagagccatcggcctgcacgacccaacagcccatgccgaaattat
ggccctgagacagggcggcctggtcatgcagaactacagactgattgacgccaccctgt
acgtgacattcgagccttgcgtgatgtgcgccggcgccatgatccactctaggatcggc
cgcgtggtgtttggatggagaaattctaaaagaggcgccgcaggctccctgatgaacgt
gctgaactaccccggcatgaatcaccgcgtcgaaattaccgagggaatcctggcagatg
aatgtgccgccctgctgtgcgatttctatcggatgcctagacaggtgttcaatgctcag
aagaaggcccagagctccatcaactccggaggatctagcggaggctcctctggctctga
gacacctggcacaagcgagagcgcaacacctgaaagcagcgggggcagcagcggggggt
cagacaagaagtacagcatcggcctggccatcggcaccaactctgtgggctgggccgtg
atcaccgacgagtacaaggtgcccagcaagaaattcaaggtgctgggcaacaccgaccg
gcacagcatcaagaagaacctgatcggagccctgctgttcgacagcggcgaaacagccg
agAGAacccggctgaagagaaccgccagaagaagatacaccagacggaagaaccggatc
tgctatctgcaagagatcttcagcaacgagatggccaaggtggacgacagcttcttcca
cagactggaagagtccttcctggtggaagaggataagaagcacgagcggcaccccatct
tcggcaacatcgtggacgaggtggcctaccacgagaagtaccccaccatctaccacctg
agaaagaaactggtggacagcaccgacaaggccgacctgcggctgatctatctggccct
ggcccacatgatcaagttccggggccacttcctgatcgagggcgacctgaaccccgaca
acagcgacgtggacaagctgttcatccagctggtgcagacctacaaccagctgttcgag
gaaaaccccatcaacgccagcggcgtggacgccaaggccatcctgtctgccagactgag
caagagcagacggctggaaaatctgatcgcccagctgcccggcgagaagaagaatggcc
tgttcggaaacctgattgccctgagcctgggcctgacccccaacttcaagagcaacttc
gacctggccgaggatgccaaactgcagctgagcaaggacacctacgacgacgacctgga
caacctgctggcccagatcggcgaccagtacgccgacctgtttctggccgccaagaacc
tgtccgacgccatcctgctgagcgacatcctgagagtgaacaccgagatcaccaaggcc
cccctgagcgcctctatgatcaagagatacgacgagcaccaccaggacctgaccctgct
gaaagctctcgtgcggcagcagctgcctgagaagtacaaagagattttcttcgaccaga
gcaagaacggctacgccggctacattgacggcggagccagccaggaagagttctacaag
ttcatcaagcccatcctggaaaagatggacggcaccgaggaactgctcgtgaagctgaa
cagagaggacctgctgcggaagcagcggaccttcgacaacggcagcatcccccaccaga
tccacctgggagagctgcacgccattctgcggcggcaggaagatttttacccattcctg
aaggacaaccgggaaaagatcgagaagatcctgaccttccgcatcccctactacgtggg
ccctctggccaggggaaacagcagattcgcctggatgaccagaaagagcgaggaaacca
tcaccccctggaacttcgaggaagtggtggacaagggcgcttccgcccagagcttcatc
gagcggatgaccaacttcgataagaacctgcccaacgagaaggtgctgcccaagcacag
cctgctgtacgagtacttcaccgtgtataacgagctgaccaaagtgaaatacgtgaccg
agggaatgagaaagcccgccttcctgagcggcgagcagaaaaaggccatcgtggacctg
ctgttcaagaccaaccggaaagtgaccgtgaagcagctgaaagaggactacttcaagaa
aatcgagtgcttcgactccgtggaaatctccggcgtggaagatcggttcaacgcctccc
tgggcacataccacgatctgctgaaaattatcaaggacaaggacttcctggacaatgag
gaaaacgaggacattctggaagatatcgtgctgaccctgacactgtttgaggacagaga
gatgatcgaggaacggctgaaaacctatgcccacctgttcgacgacaaagtgatgaagc
agctgaagcggcggagatacaccggctggggcaggctgagccggaagctgatcaacggc
atccgggacaagcagtccggcaagacaatcctggatttcctgaagtccgacggcttcgc
caacagaaacttcatgcagctgatccacgacgacagcctgacctttaaagaggacatcc
agaaagcccaggtgtccggccagggcgatagcctgcacgagcacattgccaatctggcc
ggcagccccgccattaagaagggcatcctgcagacagtgaaggtggtggacgagctcgt
gaaagtgatgggccggcacaagcccgagaacatcgtgatcgaaatggccagagagaacc
agaccacccagaagggacagaagaacagccgcgagagaatgaagcggatcgaagagggc
atcaaagagctgggcagccagatcctgaaagaacaccccgtggaaaacacccagctgca
gaacgagaagctgtacctgtactacctgcagaatgggcgggatatgtacgtggaccagg
aactggacatcaaccggctgtccgactacgatgtggaccatatcgtgcctcagagcttt
ctgaaggacgactccatcgacaacaaggtgctgaccagaagcgacaagaaccggggcaa
gagcgacaacgtgccctccgaagaggtcgtgaagaagatgaagaactactggcggcagc
tgctgaacgccaagctgattacccagagaaagttcgacaatctgaccaaggccgagaga
ggcggcctgagcgaactggataaggccggcttcatcaagagacagctggtggaaacccg
gcagatcacaaagcacgtggcacagatcctggactcccggatgaacactaagtacgacg
agaatgacaagctgatccgggaagtgaaagtgatcaccctgaagtccaagctggtgtcc
gatttccggaaggatttccagttttacaaagtgcgcgagatcaacaactaccaccacgc
ccacgacgcctacctgaacgccgtcgtgggaaccgccctgatcaaaaagtaccctaagc
tggaaagcgagttcgtgtacggcgactacaaggtgtacgacgtgcggaagatgatcgcc
aagagcgagcaggaaatcggcaaggctaccgccaagtacttcttctacagcaacatcat
gaactttttcaagaccgagattaccctggccaacggcgagatccggaagcggcctctga
tcgagacaaacggcgaaaccggggagatcgtgtgggataagggccgggattttgccacc
gtgcggaaagtgctgagcatgccccaagtgaatatcgtgaaaaagaccgaggtgcagac
aggcggcttcagcaaagagtctatcAGAcccaagaggaacagcgataagctgatcgcca
gaaagaaggactgggaccctaagaagtacggcggcttcCTTTGGcccaccgtggcctat
tctgtgctggtggtggccaaagtggaaaagggcaagtccaagaaactgaagagtgtgaa
agagctgctggggatcaccatcatggaaagaagcagcttcgagaagaatcccatcgact
ttctggaagccaagggctacaaagaagtgaaaaaggacctgatcatcaagctgcctaag
tactccctgttcgagctggaaaacggccggaagagaatgctggcctctgccAAGCaact
gcagaagggaaacgaactggccctgccctccaaatatgtgaacttcctgtacctggcca
gccactatgagaagctgaagggctcccccgaggataatgagcagaaacagctgtttgtg
gaacagcacaagcactacctggacgagatcatcgagcagatcagcgagttctccaagag
agtgatcctggccgacgctaatctggacaaagtgctgtccgcctacaacaagcaccggg
ataagcccatcagagagcaggccgagaatatcatccacctgtttaccctgaccaGActg
ggagcccctAGAgccttcaagtactttgacaccaccatcgaccCTaagCAAtacaGAag
caccaaagaggtgctggacgccaccctgatccaccagagcatcaccggcctgtacgaga
cacggatcgacctgtctcagctgggaggtgactctggcggctcaaaaagaaccgccgac
ggcagcgaattcgagcccaagaagaagaggaaagtcggcagcggaagcaaaaggccggc
ggccacgaaaaaggccggccaggcaaaaaagaaaaagctcgagtaa
SEQ ID NO: 11-Nucleotide sequence of the SpRY-evoAPOBEC1-BE4 3xNLS
adenine base editor:
atgaaacggacagccgacggaagcgagttcgagtcaccaaagaagaagcggaaagtcag
ttcaaagactgggcctgtcgccgtcgatccaaccctgcgccgccggattgaacctcacg
agtttgaagtgttctttgacccccgggagctgagaaaggagacatgcctgctgtacgag
atcaactggggaggcaggcactccatctggaggcacacctctcagaacacaaataagca
cgtggaggtgaacttcatcgagaagtttaccacagagcggtacttctgccccaatacca
gatgtagcatcacatggtttctgagctggtccccttgcggagagtgtagcagggccatc
accgagttcctgtccagatatccaaatgtgacactgtttatctacatcgccaggctgta
tcacctggcaaacccaaggaataggcagggcctgcgcgatctgatcagctccggcgtga
ccatccagatcatgacagagcaggagtccggctactgctggcacaacttcgtgaattat
tctcctagcaacgagtcccactggcctaggtacccacacctgtgggtgcgcctgtacgt
gctggagctgtattgcatcatcctgggcctgcccccttgtctgaatatcctgcggagaa
agcagagccagctgacctcctttacaatcgccctgcagtcttgtcactatcagaggctg
ccaccccacatcctgtgggccacaggcctgaagtctggcggatctagcggaggatcctc
tggcagcgagacaccaggaacaagcgagtcagcaacaccagagagcagtggcggcagca
gcggcggcagcgacaagaagtacagcatcggcctggccatcggcaccaactctgtgggc
tgggccgtgatcaccgacgagtacaaggtgcccagcaagaaattcaaggtgctgggcaa
caccgaccggcacagcatcaagaagaacctgatcggagccctgctgttcgacagcggcg
aaacagccgagAGAacccggctgaagagaaccgccagaagaagatacaccagacggaag
aaccggatctgctatctgcaagagatcttcagcaacgagatggccaaggtggacgacag
cttcttccacagactggaagagtccttcctggtggaagaggataagaagcacgagcggc
accccatcttcggcaacatcgtggacgaggtggcctaccacgagaagtaccccaccatc
taccacctgagaaagaaactggtggacagcaccgacaaggccgacctgcggctgatcta
tctggccctggcccacatgatcaagttccggggccacttcctgatcgagggcgacctga
accccgacaacagcgacgtggacaagctgttcatccagctggtgcagacctacaaccag
ctgttcgaggaaaaccccatcaacgccagcggcgtggacgccaaggccatcctgtctgc
cagactgagcaagagcagacggctggaaaatctgatcgcccagctgcccggcgagaaga
agaatggcctgttcggaaacctgattgccctgagcctgggcctgacccccaacttcaag
agcaacttcgacctggccgaggatgccaaactgcagctgagcaaggacacctacgacga
cgacctggacaacctgctggcccagatcggcgaccagtacgccgacctgtttctggccg
ccaagaacctgtccgacgccatcctgctgagcgacatcctgagagtgaacaccgagatc
accaaggcccccctgagcgcctctatgatcaagagatacgacgagcaccaccaggacct
gaccctgctgaaagctctcgtgcggcagcagctgcctgagaagtacaaagagattttct
tcgaccagagcaagaacggctacgccggctacattgacggcggagccagccaggaagag
ttctacaagttcatcaagcccatcctggaaaagatggacggcaccgaggaactgctcgt
gaagctgaacagagaggacctgctgcggaagcagcggaccttcgacaacggcagcatcc
cccaccagatccacctgggagagctgcacgccattctgcggcggcaggaagatttttac
ccattcctgaaggacaaccgggaaaagatcgagaagatcctgaccttccgcatccccta
ctacgtgggccctctggccaggggaaacagcagattcgcctggatgaccagaaagagcg
aggaaaccatcaccccctggaacttcgaggaagtggtggacaagggcgcttccgcccag
agcttcatcgagcggatgaccaacttcgataagaacctgcccaacgagaaggtgctgcc
caagcacagcctgctgtacgagtacttcaccgtgtataacgagctgaccaaagtgaaat
acgtgaccgagggaatgagaaagcccgccttcctgagcggcgagcagaaaaaggccatc
gtggacctgctgttcaagaccaaccggaaagtgaccgtgaagcagctgaaagaggacta
cttcaagaaaatcgagtgcttcgactccgtggaaatctccggcgtggaagatcggttca
acgcctccctgggcacataccacgatctgctgaaaattatcaaggacaaggacttcctg
gacaatgaggaaaacgaggacattctggaagatatcgtgctgaccctgacactgtttga
ggacagagagatgatcgaggaacggctgaaaacctatgcccacctgttcgacgacaaag
tgatgaagcagctgaagcggcggagatacaccggctggggcaggctgagccggaagctg
atcaacggcatccgggacaagcagtccggcaagacaatcctggatttcctgaagtccga
cggcttcgccaacagaaacttcatgcagctgatccacgacgacagcctgacctttaaag
aggacatccagaaagcccaggtgtccggccagggcgatagcctgcacgagcacattgcc
aatctggccggcagccccgccattaagaagggcatcctgcagacagtgaaggtggtgga
cgagctcgtgaaagtgatgggccggcacaagcccgagaacatcgtgatcgaaatggcca
gagagaaccagaccacccagaagggacagaagaacagccgcgagagaatgaagcggatc
gaagagggcatcaaagagctgggcagccagatcctgaaagaacaccccgtggaaaacac
ccagctgcagaacgagaagctgtacctgtactacctgcagaatggggggatatgtacg
tggaccaggaactggacatcaaccggctgtccgactacgatgtggaccatatcgtgcct
cagagctttctgaaggacgactccatcgacaacaaggtgctgaccagaagcgacaagaa
ccggggcaagagcgacaacgtgccctccgaagaggtcgtgaagaagatgaagaactact
ggcggcagctgctgaacgccaagctgattacccagagaaagttcgacaatctgaccaag
gccgagagaggcggcctgagcgaactggataaggccggcttcatcaagagacagctggt
ggaaacccggcagatcacaaagcacgtggcacagatcctggactcccggatgaacacta
agtacgacgagaatgacaagctgatccgggaagtgaaagtgatcaccctgaagtccaag
ctggtgtccgatttccggaaggatttccagttttacaaagtgcgcgagatcaacaacta
ccaccacgcccacgacgcctacctgaacgccgtcgtgggaaccgccctgatcaaaaagt
accctaagctggaaagcgagttcgtgtacggcgactacaaggtgtacgacgtgcggaag
atgatcgccaagagcgagcaggaaatcggcaaggctaccgccaagtacttcttctacag
caacatcatgaactttttcaagaccgagattaccctggccaacggcgagatccggaagc
ggcctctgatcgagacaaacggcgaaaccggggagatcgtgtgggataagggccgggat
tttgccaccgtgcggaaagtgctgagcatgccccaagtgaatatcgtgaaaaagaccga
ggtgcagacaggcggcttcagcaaagagtctatcAGgcccaagaggaacagcgataagc
tgatcgccagaaagaaggactgggaccctaagaagtacggcggcttcCTGTGGcccacc
gtggcctattctgtgctggtggtggccaaagtggaaaagggcaagtccaagaaactgaa
gagtgtgaaagagctgctggggatcaccatcatggaaagaagcagcttcgagaagaatc
ccatcgactttctggaagccaagggctacaaagaagtgaaaaaggacctgatcatcaag
ctgcctaagtactccctgttcgagctggaaaacggccggaagagaatgctggcctctgc
CAAGCAGctgcagaagggaaacgaactggccctgccctccaaatatgtgaacttcctgt
acctggccagccactatgagaagctgaagggctcccccgaggataatgagcagaaacag
ctgtttgtggaacagcacaagcactacctggacgagatcatcgagcagatcagcgagtt
ctccaagagagtgatcctggccgacgctaatctggacaaagtgctgtccgcctacaaca
agcaccgggataagcccatcagagagcaggccgagaatatcatccacctgtttaccctg
accaGGctgggagcccctAGAgccttcaagtactttgacaccaccatcgaccCCaagCA
gtacaGGagcaccaaagaggtgctggacgccaccctgatccaccagagcatcaccggcc
tgtacgagacacggatcgacctgtctcagctgggaggtgacagcggcgggagcggcggg
agcggggggagcactaatctgagcgacatcattgagaaggagactgggaaacagctggt
cattcaggagtccatcctgatgctgcctgaggaggtggaggaagtgatcggcaacaagc
cagagtctgacatcctggtgcacaccgcctacgacgagtccacagatgagaatgtgatg
ctgctgacctctgacgcccccgagtataagccttgggccctggtcatccaggattctaa
cggcgagaataagatcaagatgctgagcggaggatccggaggatctggaggcagcacca
acctgtctgacatcatcgagaaggagacaggcaagcagctggtcatccaggagagcatc
ctgatgctgcccgaagaagtcgaagaagtgatcggaaacaagcctgagagcgatatcct
ggtccataccgcctacgacgagagtaccgacgaaaatgtgatgctgctgacatccgacg
ccccagagtataagccctgggctctggtcatccaggattccaacggagagaacaaaatc
aaaatgctgtctggcggctcaaaaagaaccgccgacggcagcgaattcgagcccaagaa
gaagaggaaagtcggcagcggaagcaaaaggccggcggccacgaaaaaggccggccagg
caaaaaagaaaaagctcgagtaa
SEQ ID NO: 12-Nucleotide sequence of the SpRY-K918N-ABE8e-V106W 3xNLS
adenine base editor
atgaaacggacagccgacggaagcgagttcgagtcaccaaagaagaagcggaaagtctc
tgaggtggagttttcccacgagtactggatgagacatgccctgaccctggccaagaggg
cacgggatgagagggaggtgcctgtgggagccgtgctggtgctgaacaatagagtgatc
ggcgagggctggaacagagccatcggcctgcacgacccaacagcccatgccgaaattat
ggccctgagacagggcggcctggtcatgcagaactacagactgattgacgccaccctgt
acgtgacattcgagccttgcgtgatgtgcgccggcgccatgatccactctaggatcggc
cgcgtggtgtttggatggagaaattctaaaagaggcgccgcaggctccctgatgaacgt
gctgaactaccccggcatgaatcaccgcgtcgaaattaccgagggaatcctggcagatg
aatgtgccgccctgctgtgcgatttctatcggatgcctagacaggtgttcaatgctcag
aagaaggcccagagctccatcaactccggaggatctagcggaggctcctctggctctga
gacacctggcacaagcgagagcgcaacacctgaaagcagcgggggcagcagcggggggt
cagacaagaagtacagcatcggcctggccatcggcaccaactctgtgggctgggccgtg
atcaccgacgagtacaaggtgcccagcaagaaattcaaggtgctgggcaacaccgaccg
gcacagcatcaagaagaacctgatcggagccctgctgttcgacagcggcgaaacagccg
agAGAacccggctgaagagaaccgccagaagaagatacaccagacggaagaaccggatc
tgctatctgcaagagatcttcagcaacgagatggccaaggtggacgacagcttcttcca
cagactggaagagtccttcctggtggaagaggataagaagcacgagcggcaccccatct
tcggcaacatcgtggacgaggtggcctaccacgagaagtaccccaccatctaccacctg
agaaagaaactggtggacagcaccgacaaggccgacctgcggctgatctatctggccct
ggcccacatgatcaagttccggggccacttcctgatcgagggcgacctgaaccccgaca
acagcgacgtggacaagctgttcatccagctggtgcagacctacaaccagctgttcgag
gaaaaccccatcaacgccagcggcgtggacgccaaggccatcctgtctgccagactgag
caagagcagacggctggaaaatctgatcgcccagctgcccggcgagaagaagaatggcc
tgttcggaaacctgattgccctgagcctgggcctgacccccaacttcaagagcaacttc
gacctggccgaggatgccaaactgcagctgagcaaggacacctacgacgacgacctgga
caacctgctggcccagatcggcgaccagtacgccgacctgtttctggccgccaagaacc
tgtccgacgccatcctgctgagcgacatcctgagagtgaacaccgagatcaccaaggcc
cccctgagcgcctctatgatcaagagatacgacgagcaccaccaggacctgaccctgct
gaaagctctcgtgcggcagcagctgcctgagaagtacaaagagattttcttcgaccaga
gcaagaacggctacgccggctacattgacggcggagccagccaggaagagttctacaag
ttcatcaagcccatcctggaaaagatggacggcaccgaggaactgctcgtgaagctgaa
cagagaggacctgctgcggaagcagcggaccttcgacaacggcagcatcccccaccaga
tccacctgggagagctgcacgccattctgcggcggcaggaagatttttacccattcctg
aaggacaaccgggaaaagatcgagaagatcctgaccttccgcatcccctactacgtggg
ccctctggccaggggaaacagcagattcgcctggatgaccagaaagagcgaggaaacca
tcaccccctggaacttcgaggaagtggtggacaagggcgcttccgcccagagcttcatc
gagcggatgaccaacttcgataagaacctgcccaacgagaaggtgctgcccaagcacag
cctgctgtacgagtacttcaccgtgtataacgagctgaccaaagtgaaatacgtgaccg
agggaatgagaaagcccgccttcctgagcggcgagcagaaaaaggccatcgtggacctg
ctgttcaagaccaaccggaaagtgaccgtgaagcagctgaaagaggactacttcaagaa
aatcgagtgcttcgactccgtggaaatctccggcgtggaagatcggttcaacgcctccc
tgggcacataccacgatctgctgaaaattatcaaggacaaggacttcctggacaatgag
gaaaacgaggacattctggaagatatcgtgctgaccctgacactgtttgaggacagaga
gatgatcgaggaacggctgaaaacctatgcccacctgttcgacgacaaagtgatgaagc
agctgaagcggcggagatacaccggctggggcaggctgagccggaagctgatcaacggc
atccgggacaagcagtccggcaagacaatcctggatttcctgaagtccgacggcttcgc
caacagaaacttcatgcagctgatccacgacgacagcctgacctttaaagaggacatcc
agaaagcccaggtgtccggccagggcgatagcctgcacgagcacattgccaatctggcc
ggcagccccgccattaagaagggcatcctgcagacagtgaaggtggtggacgagctcgt
gaaagtgatgggccggcacaagcccgagaacatcgtgatcgaaatggccagagagaacc
agaccacccagaagggacagaagaacagccgcgagagaatgaagcggatcgaagagggc
atcaaagagctgggcagccagatcctgaaagaacaccccgtggaaaacacccagctgca
gaacgagaagctgtacctgtactacctgcagaatgggcgggatatgtacgtggaccagg
aactggacatcaaccggctgtccgactacgatgtggaccatatcgtgcctcagagcttt
ctgaaggacgactccatcgacaacaaggtgctgaccagaagcgacaagaaccggggcaa
gagcgacaacgtgccctccgaagaggtcgtgaagaagatgaagaactactggcggcagc
tgctgaacgccaagctgattacccagagaaagttcgacaatctgaccaaggccgagaga
ggcggcctgagcgaactggataaggccggcttcatcaaCagacagctggtggaaacccg
gcagatcacaaagcacgtggcacagatcctggactcccggatgaacactaagtacgacg
agaatgacaagctgatccgggaagtgaaagtgatcaccctgaagtccaagctggtgtcc
gatttccggaaggatttccagttttacaaagtgcgcgagatcaacaactaccaccacgc
ccacgacgcctacctgaacgccgtcgtgggaaccgccctgatcaaaaagtaccctaagc
tggaaagcgagttcgtgtacggcgactacaaggtgtacgacgtgcggaagatgatcgcc
aagagcgagcaggaaatcggcaaggctaccgccaagtacttcttctacagcaacatcat
gaactttttcaagaccgagattaccctggccaacggcgagatccggaagcggcctctga
tcgagacaaacggcgaaaccggggagatcgtgtgggataagggccgggattttgccacc
gtgcggaaagtgctgagcatgccccaagtgaatatcgtgaaaaagaccgaggtgcagac
aggcggcttcagcaaagagtctatcAGAcccaagaggaacagcgataagctgatcgcca
gaaagaaggactgggaccctaagaagtacggcggcttcCTTTGGcccaccgtggcctat
tctgtgctggtggtggccaaagtggaaaagggcaagtccaagaaactgaagagtgtgaa
agagctgctggggatcaccatcatggaaagaagcagcttcgagaagaatcccatcgact
ttctggaagccaagggctacaaagaagtgaaaaaggacctgatcatcaagctgcctaag
tactccctgttcgagctggaaaacggccggaagagaatgctggcctctgccAAGCaact
gcagaagggaaacgaactggccctgccctccaaatatgtgaacttcctgtacctggcca
gccactatgagaagctgaagggctcccccgaggataatgagcagaaacagctgtttgtg
gaacagcacaagcactacctggacgagatcatcgagcagatcagcgagttctccaagag
agtgatcctggccgacgctaatctggacaaagtgctgtccgcctacaacaagcaccggg
ataagcccatcagagagcaggccgagaatatcatccacctgtttaccctgaccaGActg
ggagcccctAGAgccttcaagtactttgacaccaccatcgaccCTaagCAAtacaGAag
caccaaagaggtgctggacgccaccctgatccaccagagcatcaccggcctgtacgaga
cacggatcgacctgtctcagctgggaggtgactctggcggctcaaaaagaaccgccgac
ggcagcgaattcgagcccaagaagaagaggaaagtcggcagcggaagcaaaaggccggc
ggccacgaaaaaggccggccaggcaaaaaagaaaaagctcgag

gRNA

The terms “gRNA,” “guide RNA” and “CRISPR guide sequence” can be used interchangeably throughout and refer to a nucleic acid comprising a sequence that determines the specificity of a Cas DNA binding protein of a CRISPR/Cas system. A gRNA hybridizes to (complementary to, partially or completely) a target nucleic acid sequence in the genome of a host cell. In some instances, the gRNA refers collectively to the crRNA and the tracrRNA (for instance, when a Cas9 nuclease is being used—in those instances, the guide RNA may be referred to as a single guide RNA, i.e., sgRNA). In other instances, the gRNA refers only to the crRNA (for instance, when a Cpf1 endonuclease is being used). The gRNA or portion thereof that hybridizes to the target nucleic acid can be between 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In some embodiments, the gRNA sequence that hybridizes to the target nucleic acid is between 10-30, or between 15-25, nucleotides in length. In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid.

Exemplary guide RNAs for editing FLT3, CD123, and KIT are provided in the Table 1 below. As will be evident to one of ordinary skill in the art, selection of gRNA sequences can depend on factors such as the number of predicted on-target and/or off-target binding sites. In some embodiments, the gRNA sequence is selected to maximize potential on-target and minimize potential off-target sites.

TABLE 1

| Target | | SEQ | | | base | | Cas |
|---|---|---|---|---|---|---|---|
| gene | | ID | | mutation | editor | PAM | require- |
| | gRNA name | NO: | sequence | (s) | type | sequence | ment |
| FLT3 | FLT3_e9 Cas9-4 | 13 | GAGCAAAAGGGTCTTGATAA | N399 and | Cas9 | | Cas9 |
| (CD135) | | | | nearby | nuclease | | |
| | | | | region | | | |
| FLT3 | FLT3_e9_Cas9-13 | 14 | AGGGTCTTGATAACGGATAC | N399 and | Cas9 | | Cas9 |
| (CD135) | | | | nearby | nuclease | | |
| | | | | region | | | |
| FLT3 | FLT3 e9 Cas12a- | 15 | TGGTCTCACCTGTATCCGTTATC | N399 and | Cas12a | | Cas12a |
| (CD135) | 15 | | | nearby | nuclease | | |
| | | | | region | | | |
| FLT3 | FLT3_e9_Cas12a- | 16 | CTTGTGAGCAAAAGGGTCTTGAT | N399 and | Cas12a | | Cas12a |
| (CD135) | 15 | | | nearby | nuclease | | |
| | | | | region | | | |
| FLT3 | FLT3_e9_15 | 17 | GTCTTGATAACGGATACAGG | N399D, | Adenine | TGA | NG-Cas9 |
| (CD135) | | | | N399G | base | | |
| | | | | | editor | | |
| FLT3 | FLT3_e9_16 | 18 | CTTGATAACGGATACAGGTG | N399D, | Adenine | AGA | NG-Cas9 |
| (CD135) | | | | N399G | base | | |
| | | | | | editor | | |
| FLT3 | FLT3_e9_17 | 19 | TCTTGATAACGGATACAGGT | N399D, | Adenine | GAG | SpRY-Cas9 |
| (CD135) | | | | N399G | base | | |
| | | | | | editor | | |
| FLT3 | FLT3_e9_18 | 20 | TTGATAACGGATACAGGTGA | N399D, | Adenine | GAC | SpRY-Cas9 |
| (CD135) | | | | N399G | base | | |
| | | | | | editor | | |
| FLT3 | FLT3_e9_1821 | 21 | CTTGATAACGGATACAGGTGA | N399D, | Adenine | GAC | SpRY-Cas9 |
| (CD135) | | | | N399G | base | | |
| | | | | | editor | | |
| FLT3 | FLT3_e9_1819 | 22 | TGATAACGGATACAGGTGA | N399D, | Adenine | GAC | SpRY-Cas9 |
| (CD135) | | | | N399G | base | | |
| | | | | | editor | | |
| FLT3 | FLT3_e9_19 | 23 | ATAACGGATACAGGTGAGAC | N399D, | Adenine | CAC | SpRY-Cas9 |
| (CD135) | | | | N399G | base | | |
| | | | | | editor | | |
| IL3RA | IL3RA_gRNA_N | 24 | CATAGAATAGTCGGCGTCTT | S59P, | Adenine | TAA | SpRY-Cas9 |
| (CD123) | | | | Y58H | base | | |
| | | | | | editor | | |
| IL3RA | IL3RA_gRNA_N21 | 25 | GCATAGAATAGTCGGCGTCTT | S59P, | Adenine | TAA | SpRY-Cas9 |
| (CD123) | | | | Y58H | base | | |
| | | | | | editor | | |
| IL3RA | IL3RA_gRNA_N19 | 26 | ATAGAATAGTCGGCGTCTT | S59P, | Adenine | TAA | SpRY-Cas9 |
| (CD123) | | | | Y58H | base | | |
| | | | | | editor | | |
| IL3RA | IL3RA_gRNA_R | 27 | ATAGAATAGTCGGCGTCTTT | S59P, | Adenine | AAC | SpRY-Cas9 |
| (CD123) | | | | Y58H | base | | |
| | | | | | editor | | |
| IL3RA | IL3RA_gRNA_R21 | 28 | CATAGAATAGTCGGCGTCTTT | S59P, | Adenine | AAC | SpRY-Cas9 |
| (CD123) | | | | Y58H | base | | |
| | | | | | editor | | |
| IL3RA | IL3RA_gRNA_R19 | 29 | TAGAATAGTCGGCGTCTTT | S59P, | Adenine | AAC | SpRY-Cas9 |
| (CD123) | | | | Y58H | base | | |
| | | | | | editor | | |
| IL3RA | IL3RA_gRNA_H | 30 | GGCCACTCGGACGGTGTAGT | R84Q, | Cytidine | TGG | SpCas9 |
| (CD123) | | | | V85M/I, | base | | |
| | | | | A86T | editor | | |
| IL3RA | IL3RA_gRNA_Q | 31 | GCCACTCGGACGGTGTAGTT | R84Q, | Cytidine | GGT | SpCas9 |
| (CD123) | | | | V85M/I, | base | | |
| | | | | A86T | editor | | |
| IL3RA | IL3RA_gRNA_L | 32 | TATTCTATGCCGGTAAATCA | S59F | Cytidine | TAC | SpRY-Cas9 |
| (CD123) | | | | | base | | |
| | | | | | editor | | |
| IL3RA | IL3RA_gRNA_M | 33 | ACTATTCTATGCCGGTAAAT | S59F | Cytidine | CAT | SpRY-Cas9 |
| (CD123) | | | | | base | | |
| | | | | | editor | | |
| IL3RA | IL3RA_gRNA_F | 34 | CCAACCCACCATTCTCCACG | P88L/S, | Cytidine | TGG | SpCas9 |
| (CD123) | | | | P89L/S | base | | |
| | | | | | editor | | |
| IL3RA | IL3RA_gRNA_Z | 35 | CAACCCACCATTCTCCACGT | P88L/S, | Cytidine | GGA | NG-Cas9 |
| (CD123) | | | | P89L/S | base | | |
| | | | | | editor | | |
| KIT | KIT-e7-X | 36 | AACTTCATCTAACGAGATTA | H378R | Adenine | AAA | SpRY-Cas9 |
| (CD117) | | | | | base | | |
| | | | | | editor | | |
| KIT | KIT-e7-Y | 37 | ACTTCATCTAACGAGATTAA | H378R | Adenine | AAG | SpRY-Cas9 |
| (CD117) | | | | | base | | |
| | | | | | editor | | |
| KIT | KIT-e7-3 | 38 | CTTCATCTAACGAGATTAAA | H378R | Adenine | AGG | SpCas9 |
| (CD117) | | | | | base | | |
| | | | | | editor | | |
| KIT | KIT-e7-Z | 39 | TTCATCTAACGAGATTAAAA | H378R | Adenine | GGC | NG-Cas9 |
| (CD117) | | | | | base | | |
| | | | | | editor | | |

In some embodiments, multiple gRNAs are introduced into the cell (e.g., one for FLT3 and one for CD123). In some embodiments, the two or more guide RNAs are transfected into cells in equimolar amounts. In some embodiments, the two or more guide RNAs are provided in amounts that are not equimolar. In some embodiments, the two or more guide RNAs are provided in amounts that are optimized so that editing of each target occurs at equal frequency. In some embodiments, the two or more guide RNAs are provided in amounts that are optimized so that editing of each target occurs at optimal frequency.

### Template Donor Sequence

In some embodiments, provided herein is a “template” donor sequence. The template donor sequence includes a 100-500 nucleotide (e.g., 200 nucleotides) long single strand oligo-deoxynucleotide (ssODN), which functions as a donor template for homology directed repair (HDR), such that the desired mutation is introduced into the specific gene. Each donor template additionally includes selected silent mutations in bystander amino-acids to reduce the risk of re-cutting by the CRISPR-Cas ribonucleoprotein nuclease complex after successful DNA repair.

Exemplary template donor sequences for the introduction of the mutation N399D in FLT are provided in Table 2 below.

TABLE 2
ssODN 200-bp long oligos donors to serve as template for FLT3 N399D
introduction through CRISPR-Cas nuclease homology directed repair

| Donor | | |
|---|---|---|
| Template | SEQ ID | |
| Name | NO: | Sequence |
| FLT3- | 40 | aagagttttgtttttctgtcaggtttaaagcctacccacaaatcagatgtacgtggac |
| N399D-A | | cttctctcgaaaatcatttccttgtgagcaaaagggtcttgatGacggatacaggt |
| | | gagaccacaaaggactctaagataccatttttagcataattctccttgcttgtcgcc |
| | | cttgagaaggacagttttatacttatgga |
| FLT3- | 41 | aagagttttgtttttctgtcaggtttaaagcctacccacaaatcagatgtacgtggac |
| N399D-C | | cttctctcgaaaatcatttccttgtgagcaaaagggtTtGgaCGacggatacag |
| | | gtgagaccacaaaggactctaagataccatttttagcataattctccttgcttgtcg |
| | | cccttgagaaggacagttttatacttatgga |
| FLT3- | 42 | tgaagagttttgtttttctgtcaggtttaaagcctacccacaaatcagatgtacgtgg |
| N399D-F | | accttctctcgaaaatcattCccAtgCgaAcaaaagggATtGgaCGaTg |
| | | gatacaggtgagaccacaaaggactctaagataccatttttagcataattctccttg |
| | | cttgtcgcccttgagaaggacagttttatacttatg |
| FLT3- | 43 | agagttttgtttttctgtcaggtttaaagcctacccacaaatcagatgtacgtggacc |
| N399D-H | | ttctctcgaaaatcatttccttgtgagcaaaagggtcttgaCGaTggTtaTagg |
| | | tgagaccacaCaggactctaagataccatttttagcataattctccttgcttgtcgc |
| | | ccttgagaaggacagttttatacttatggaa |
| FLT3- | 44 | tccataagtataaaactgtccttctcaagggcgacaagcaaggagaattatgcta |
| N399D-A | | aaaatggtatcttagagtcctttgtggtctcacctgtatccgtCatcaagaccctttt |
| RC | | gctcacaaggaaatgattttcgagagaaggtccacgtacatctgatttgtgggtag |
| | | gctttaaacctgacagaaaaacaaaactctt |
| FLT3- | 45 | tccataagtataaaactgtccttctcaagggcgacaagcaaggagaattatgcta |
| N399D-C | | aaaatggtatcttagagtcctttgtggtctcacctgtatccgtCGtcCaAaccctt |
| RC | | ttgctcacaaggaaatgattttcgagagaaggtccacgtacatctgatttgtgggt |
| | | aggctttaaacctgacagaaaaacaaaactctt |
| FLT3- | 46 | cataagtataaaactgtccttctcaagggcgacaagcaaggagaattatgctaaa |
| N399D-F | | aatggtatcttagagtcctttgtggtctcacctgtatccAtCGtcCaATccctttt |
| RC | | gTtcGcaTggGaatgattttcgagagaaggtccacgtacatctgatttgtgggt |
| | | aggctttaaacctgacagaaaaacaaaactcttca |
| FLT3- | 47 | ttccataagtataaaactgtccttctcaagggcgacaagcaaggagaattatgcta |
| N399D-H | | aaaatggtatcttagagtcctGtgtggtctcacctAtaAccAtCGtcaagacc |
| RC | | cttttgctcacaaggaaatgattttcgagagaaggtccacgtacatctgatttgtgg |
| | | gtaggctttaaacctgacagaaaaacaaaactct |

iv. Genetically Engineered Hematopoietic Cells

Provided herein are methods of producing the genetically engineered hematopoietic cells as described herein, which carry edited genes for expressing one or more cell-surface antigens in mutated form. Such methods can involve providing a cell and introducing into the cell components of a CRISPR Cas system for genome editing. In some embodiments, a nucleic acid that comprises a CRISPR-Cas guide RNA (gRNA) that hybridizes or is predicted to hybridize to a portion of the nucleotide sequence that encodes the cell-surface antigen is introduced into the cell. In some embodiments, the gRNA is introduced into the cell on a vector. In some embodiments, a Cas endonuclease is introduced into the cell. In some embodiments, the Cas endonuclease is introduced into the cell as a nucleic acid encoding a Cas endonuclease. In some embodiments, the gRNA and a nucleotide sequence encoding a Cas endonuclease are introduced into the cell on the same nucleic acid (e.g., the same vector). In some embodiments, the Cas endonuclease is introduced into the cell in the form of a protein. In some embodiments, the Cas endonuclease and the gRNA are pre-formed in vitro and are introduced to the cell in as a ribonucleoprotein complex.

v. Mutant FLT3

In some embodiments, the cell-surface protein is FLT3. The amino acid sequence of wild-type FLT3 is shown below:

(FLT3 wild type amino acid sequence)
SEQ ID NO: 48
MPALARDGGQLPLLVVFSAMIFGTITNQDLPVIKCVLINHKNNDSSVGKSSSYPMVSES
PEDLGCALRPQSSGTVYEAAAVEVDVSASITLQVLVDAPGNISCLWVFKHSSLNCQPHF
DLQNRGVVSMVILKMTETQAGEYLLFIQSEATNYTILFTVSIRNTLLYTLRRPYFRKME
NQDALVCISESVPEPIVEWVLCDSQGESCKEESPAVVKKEEKVLHELFGTDIRCCARNE
LGRECTRLFTIDLNQTPQTTLPQLFLKVGEPLWIRCKAVHVNHGFGLTWELENKALEEG
NYFEMSTYSTNRTMIRILFAFVSSVARNDTGYYTCSSSKHPSQSALVTIVEKGFINATN
SSEDYEIDQYEEFCFSVRFKAYPQIRCTWTFSRKSFPCEQKGLDNGYSISKFCNHKHQP
GEYIFHAENDDAQFTKMFTLNIRRKPQVLAEASASQASCFSDGYPLPSWTWKKCSDKSP
NCTEEITEGVWNRKANRKVFGQWVSSSTLNMSEAIKGFLVKCCAYNSLGTSCETILLNS
PGPFPFIQDNISFYATIGVCLLFIVVLTLLICHKYKKQFRYESQLQMVQVTGSSDNEYF
YVDFREYEYDLKWEFPRENLEFGKVLGSGAFGKVMNATAYGISKTGVSIQVAVKMLKEK
ADSSEREALMSELKMMTQLGSHENIVNLLGACTLSGPIYLIFEYCCYGDLLNYLRSKRE
KFHRTWTEIFKEHNFSFYPTFQSHPNSSMPGSREVQIHPDSDQISGLHGNSFHSEDEIE
YENQKRLEEEEDLNVLTFEDLLCFAYQVAKGMEFLEFKSCVHRDLAARNVLVTHGKVVK
ICDFGLARDIMSDSNYVVRGNARLPVKWMAPESLFEGIYTIKSDVWSYGILLWEIFSLG
VNPYPGIPVDANFYKLIQNGFKMDQPFYATEEIYIIMQSCWAFDSRKRPSFPNLTSFLG
CQLADAEEAMYQNVDGRVSECPHTYQNRRPFSREMDLGLLSPQAQVEDS

In some embodiments, the methods described herein involve genetically engineering a population of hematopoietic cells using a Cas nuclease (or variant thereof). In some embodiments, the methods described herein involve genetically engineering a gene encoding a cell-surface antigen in a population of hematopoietic cells using a Cas nuclease or variant thereof (e.g., SpCas9 or AsCpf1). In some embodiments, the methods described herein involve genetically modifying or editing a FLT3 gene, or genetically modifying or editing a CD123 gene, or genetically modifying or editing a KIT gene, or genetically modifying or editing a FLT3 gene and a CD123 gene in the population of hematopoietic cells using the Cas nuclease.

In some embodiments, the methods described herein involve genetically engineering a mutant FLT3 gene in a population of hematopoietic cells using a Cas nuclease or variant thereof. In some embodiments, the methods described herein involve genetically engineering a mutation in exon 9 of FLT3 (e.g., thereby resulting in the mutation of position N399 in the encoded polypeptide) in a population of hematopoietic cells using a Cas nuclease or variant thereof. In some embodiments, the methods described herein involve genetically engineering a mutant FLT3 gene in a population of hematopoietic cells using a Cas nuclease or variant thereof and a guide sequence provided by any one of SEQ ID NOs: 13-23. In some embodiments, a template donor DNA sequence is also provided. For instance, a template donor DNA sequence provided by any one of SEQ ID NOs: 40-43.

In some embodiments, the genetically engineered FLT3 gene encodes a protein that has reduced binding to a therapeutic anti-FLT3 antibody. In some instances, the genetically engineered FLT3 gene includes at least one mutation in exon 9 of the FLT3 gene. In some instances, at least one mutation in exon 9 of the genetically engineered FLT3 gene results in a polypeptide bearing a mutation at position N399. In some instances, the mutation at position N399 is N399D or N399G. Exemplary amino acid sequences of the genetically engineered FLT3 are provided below:

FLT3 N354S, S356Q, D358E, Q363P, E366K, Q378R, T384I, R387Q, K389A,
K395R, D398E, N399D, N408D, H411N, Q412K, H419Y; FLT3-ECD4 variant
(bearing 16 amino-acid substitutions within the extracellular domain 4, which
decrease the binding of clone 4G8, including N399D)
SEQ ID NO: 49
MPALARDGGQLPLLVVFSAMIFGTITNQDLPVIKCVLINHKNNDSSVGKSSSYPMVSES
PEDLGCALRPQSSGTVYEAAAVEVDVSASITLQVLVDAPGNISCLWVFKHSSLNCQPHF
DLQNRGVVSMVILKMTETQAGEYLLFIQSEATNYTILFTVSIRNTLLYTLRRPYFRKME
NQDALVCISESVPEPIVEWVLCDSQGESCKEESPAVVKKEEKVLHELFGTDIRCCARNE
LGRECTRLFTIDLNQTPQTTLPQLFLKVGEPLWIRCKAVHVNHGFGLTWELENKALEEG
NYFEMSTYSTNRTMIRILFAFVSSVARNDTGYYTCSSSKHPSQSALVTIVEKGFINATS
SQEEYEIDPYEKFCFSVRFKAYPRIRCTWIFSQASFPCEQRGLEDGYSISKFCDHKNKP
GEYIFYAENDDAQFTKMFTLNIRRKPQVLAEASASQASCESDGYPLPSWTWKKCSDKSP
NCTEEITEGVWNRKANRKVFGQWVSSSTLNMSEAIKGFLVKCCAYNSLGTSCETILLNS
PGPFPFIQDNISFYATIGVCLLFIVVLTLLICHKYKKQFRYESQLQMVQVTGSSDNEYF
YVDFREYEYDLKWEFPRENLEFGKVLGSGAFGKVMNATAYGISKTGVSIQVAVKMLKEK
ADSSEREALMSELKMMTQLGSHENIVNLLGACTLSGPIYLIFEYCCYGDLLNYLRSKRE
KEHRTWTEIFKEHNFSFYPTFQSHPNSSMPGSREVQIHPDSDQISGLHGNSFHSEDEIE
YENQKRLEEEEDLNVLTFEDLLCFAYQVAKGMEFLEFKSCVHRDLAARNVLVTHGKVVK
ICDFGLARDIMSDSNYVVRGNARLPVKWMAPESLFEGIYTIKSDVWSYGILLWEIFSLG
VNPYPGIPVDANFYKLIQNGFKMDQPFYATEEIYIIMQSCWAFDSRKRPSFPNLTSFLG
CQLADAEEAMYQNVDGRVSECPHTYQNRRPFSREMDLGLLSPQAQVEDS
FLT3 N354S, S356Q, D358E, Q363P, E366K, Q378R, T384I, R387Q, K389A, K395R,
D398E, N399D (FLT3 exon 9 mutations)
SEQ ID NO: 50
MPALARDGGQLPLLVVFSAMIFGTITNQDLPVIKCVLINHKNNDSSVGKSSSYPMVSES
PEDLGCALRPQSSGTVYEAAAVEVDVSASITLQVLVDAPGNISCLWVFKHSSLNCQPHF
DLQNRGVVSMVILKMTETQAGEYLLFIQSEATNYTILFTVSIRNTLLYTLRRPYFRKME
NQDALVCISESVPEPIVEWVLCDSQGESCKEESPAVVKKEEKVLHELFGTDIRCCARNE
LGRECTRLFTIDLNQTPQTTLPQLFLKVGEPLWIRCKAVHVNHGFGLTWELENKALEEG
NYFEMSTYSTNRTMIRILFAFVSSVARNDTGYYTCSSSKHPSQSALVTIVEKGFINATS
SQEEYEIDPYEKFCFSVRFKAYPRIRCTWIFSQASFPCEQRGLEDGYSISKFCNHKHQP
GEYIFHAENDDAQFTKMFTLNIRRKPQVLAEASASQASCFSDGYPLPSWTWKKCSDKSP
NCTEEITEGVWNRKANRKVFGQWVSSSTLNMSEAIKGELVKCCAYNSLGTSCETILLNS
PGPFPFIQDNISFYATIGVCLLFIVVLTLLICHKYKKQFRYESQLQMVQVTGSSDNEYF
YVDFREYEYDLKWEFPRENLEFGKVLGSGAFGKVMNATAYGISKTGVSIQVAVKMLKEK
ADSSEREALMSELKMMTQLGSHENIVNLLGACTLSGPIYLIFEYCCYGDLLNYLRSKRE
KFHRTWTEIFKEHNFSFYPTFQSHPNSSMPGSREVQIHPDSDQISGLHGNSFHSEDEIE
YENQKRLEEEEDLNVLTFEDLLCFAYQVAKGMEFLEFKSCVHRDLAARNVLVTHGKVVK
ICDFGLARDIMSDSNYVVRGNARLPVKWMAPESLFEGIYTIKSDVWSYGILLWEIFSLG
VNPYPGIPVDANFYKLIQNGFKMDQPFYATEEIYIIMQSCWAFDSRKRPSFPNLTSFLG
CQLADAEEAMYQNVDGRVSECPHTYQNRRPFSREMDLGLLSPQAQVEDS
(FLT3 N399D variant amino acid sequence)
SEQ ID NO: 51
MPALARDGGQLPLLVVFSAMIFGTITNQDLPVIKCVLINHKNNDSSVGKSSSYPMVSES
PEDLGCALRPQSSGTVYEAAAVEVDVSASITLQVLVDAPGNISCLWVFKHSSLNCQPHF
DLQNRGVVSMVILKMTETQAGEYLLFIQSEATNYTILFTVSIRNTLLYTLRRPYFRKME
NQDALVCISESVPEPIVEWVLCDSQGESCKEESPAVVKKEEKVLHELFGTDIRCCARNE
LGRECTRLFTIDLNQTPQTTLPQLFLKVGEPLWIRCKAVHVNHGFGLTWELENKALEEG
NYFEMSTYSTNRTMIRILFAFVSSVARNDTGYYTCSSSKHPSQSALVTIVEKGFINATN
SSEDYEIDQYEEFCFSVRFKAYPQIRCTWTFSRKSFPCEQKGLDDGYSISKFCNHKHQP
GEYIFHAENDDAQFTKMFTLNIRRKPQVLAEASASQASCFSDGYPLPSWTWKKCSDKSP
NCTEEITEGVWNRKANRKVFGQWVSSSTLNMSEAIKGFLVKCCAYNSLGTSCETILLNS
PGPFPFIQDNISFYATIGVCLLFIVVLTLLICHKYKKQFRYESQLQMVQVTGSSDNEYF
YVDFREYEYDLKWEFPRENLEFGKVLGSGAFGKVMNATAYGISKTGVSIQVAVKMLKEK
ADSSEREALMSELKMMTQLGSHENIVNLLGACTLSGPIYLIFEYCCYGDLLNYLRSKRE
KEHRTWTEIFKEHNFSFYPTFQSHPNSSMPGSREVQIHPDSDQISGLHGNSFHSEDEIE
YENQKRLEEEEDLNVLTFEDLLCFAYQVAKGMEFLEFKSCVHRDLAARNVLVTHGKVVK
ICDFGLARDIMSDSNYVVRGNARLPVKWMAPESLFEGIYTIKSDVWSYGILLWEIFSLG
VNPYPGIPVDANFYKLIQNGFKMDQPFYATEEIYIIMQSCWAFDSRKRPSFPNLTSFLG
CQLADAEEAMYQNVDGRVSECPHTYQNRRPFSREMDLGLLSPQAQVEDS
(FLT3 N399G variant amino acid sequence)
SEQ ID NO: 52
MPALARDGGQLPLLVVFSAMIFGTITNQDLPVIKCVLINHKNNDSSVGKSSSYPMVSES
PEDLGCALRPQSSGTVYEAAAVEVDVSASITLQVLVDAPGNISCLWVFKHSSLNCQPHF
DLQNRGVVSMVILKMTETQAGEYLLFIQSEATNYTILFTVSIRNTLLYTLRRPYFRKME
NQDALVCISESVPEPIVEWVLCDSQGESCKEESPAVVKKEEKVLHELFGTDIRCCARNE
LGRECTRLFTIDLNQTPQTTLPQLFLKVGEPLWIRCKAVHVNHGFGLTWELENKALEEG
NYFEMSTYSTNRTMIRILFAFVSSVARNDTGYYTCSSSKHPSQSALVTIVEKGFINATN
SSEDYEIDQYEEFCFSVRFKAYPQIRCTWTFSRKSFPCEQKGLDGGYSISKFCNHKHQP
GEYIFHAENDDAQFTKMETLNIRRKPQVLAEASASQASCESDGYPLPSWTWKKCSDKSP
NCTEEITEGVWNRKANRKVFGQWVSSSTLNMSEAIKGFLVKCCAYNSLGTSCETILLNS
PGPFPFIQDNISFYATIGVCLLFIVVLTLLICHKYKKQFRYESQLQMVQVTGSSDNEYF
YVDFREYEYDLKWEFPRENLEFGKVLGSGAFGKVMNATAYGISKTGVSIQVAVKMLKEK
ADSSEREALMSELKMMTQLGSHENIVNLLGACTLSGPIYLIFEYCCYGDLLNYLRSKRE
KFHRTWTEIFKEHNFSFYPTFQSHPNSSMPGSREVQIHPDSDQISGLHGNSFHSEDEIE
YENQKRLEEEEDLNVLTFEDLLCFAYQVAKGMEFLEFKSCVHRDLAARNVLVTHGKVVK
ICDFGLARDIMSDSNYVVRGNARLPVKWMAPESLFEGIYTIKSDVWSYGILLWEIFSLG
VNPYPGIPVDANFYKLIQNGFKMDQPFYATEEIYIIMQSCWAFDSRKRPSFPNLTSFLG
CQLADAEEAMYQNVDGRVSECPHTYQNRRPFSREMDLGLLSPQAQVEDS

In some embodiments, provided herein is a polypeptide sequence that is at least 80%, 85%, 90%, 91%, 92%, 93%, 94%, 95%, 96%, 97%, 98%, 99%, or 100% identical to the sequence set forth in SEQ ID NO: 51, wherein the polypeptide sequence comprises a mutation at N399D and wherein the polypeptide sequence has reduced binding to a therapeutic anti-FLT3 antibody. Also provided herein are nucleic acids encoding the polypeptide sequence.

In some embodiments, provided herein is a polypeptide sequence that is at least 80%, 85%, 90%, 91%, 92%, 93%, 94%, 95%, 96%, 97%, 98%, 99%, or 100% identical to the sequence set forth in SEQ ID NO: 52, wherein the polypeptide sequence comprises a mutation at N399G and wherein the polypeptide sequence has reduced binding to a therapeutic anti-FLT3 antibody. Also provided herein are nucleic acids encoding the polypeptide sequence.

vi. Mutant CD123

In some embodiments, the cell-surface protein is CD123. The amino acid sequence of wild-type CD123 is shown below:

(CD123 wild type amino acid sequence)
SEQ.ID NO: 53
MVLLWLTLLLIALPCLLQTKEDPNPPITNLRMKAKAQQLTWDLNRNVTDIECVKDADYS
MPAVNNSYCQFGAISLCEVTNYTVRVANPPFSTWILFPENSGKPWAGAENLTCWIHDVD
FLSCSWAVGPGAPADVQYDLYLNVANRRQQYECLHYKTDAQGTRIGCREDDISRLSSGS
QSSHILVRGRSAAFGIPCTDKFVVFSQIEILTPPNMTAKCNKTHSFMHWKMRSHFNRKF
RYELQIQKRMQPVITEQVRDRTSFQLLNPGTYTVQIRARERVYEFLSAWSTPQRFECDQ
EEGANTRAWRTSLLIALGTLLALVCVFVICRRYLVMQRLFPRIPHMKDPIGDSFQNDKL
VVWEAGKAGLEECLVTEVQVVQKT

In some embodiments, the methods described herein involve genetically engineering a mutant CD123 gene in a population of hematopoietic cells using a Cas nuclease or variant thereof. In some embodiments, the methods described herein involve genetically engineering a mutation in exon 2 of CD123 (e.g., thereby resulting in the mutation of position S59 in the encoded polypeptide) or in exon 3 of CD123 (e.g., thereby resulting in the mutation of position P88 in the encoded polypeptide) in a population of hematopoietic cells using a Cas nuclease or variant thereof. In some embodiments, the methods described herein involve genetically engineering a mutant CD123 gene in a population of hematopoietic cells using a Cas nuclease or variant thereof and a guide sequence provided by any one of SEQ ID NOs: 24-35.

In some embodiments, the genetically engineered HSPC includes a genetically engineered CD123 gene, wherein the genetically engineered CD123 gene encodes a protein that has reduced binding to a therapeutic anti-CD123 antibody. In some instances, the genetically engineered FLT3 gene includes at least one mutation in exon 2 or exon 3 of the CD123 gene. In some instances, at least one mutation in exon 2 of the genetically engineered CD123 gene results in a polypeptide bearing a mutation at position S59. In some instances, the mutation at position S59 is S59P or S59F. In some instances, at least one mutation in exon 3 of the genetically engineered CD123 gene results in a polypeptide bearing a mutation at position P88. In some instances, the mutation at position P88 is P88L or P88S. Exemplary amino acid sequences of the genetically engineered CD123 are provided below:

(CD123 S59P variant amino acid sequence)
SEQ ID NO: 54
MVLLWLTLLLIALPCLLQTKEDPNPPITNLRMKAKAQQLTWDLNRNVTDIECVKDADYP
MPAVNNSYCQFGAISLCEVINYTVRVANPPFSTWILFPENSGKPWAGAENLTCWIHDVD
FLSCSWAVGPGAPADVQYDLYLNVANRRQQYECLHYKTDAQGTRIGCREDDISRLSSGS
QSSHILVRGRSAAFGIPCTDKFVVESQIEILTPPNMTAKCNKTHSFMHWKMRSHENRKF
RYELQIQKRMQPVITEQVRDRTSFQLLNPGTYTVQIRARERVYEFLSAWSTPQRFECDQ
EEGANTRAWRTSLLIALGTLLALVCVFVICRRYLVMQRLFPRIPHMKDPIGDSFQNDKL
VVWEAGKAGLEECLVTEVQVVQKT
(CD123 Y58H S59P variant amino acid sequence)
SEQ ID NO: 55
MVLLWLTLLLIALPCLLQTKEDPNPPITNLRMKAKAQQLTWDLNRNVTDIECVKDADHP
MPAVNNSYCQFGAISLCEVTNYTVRVANPPFSTWILFPENSGKPWAGAENLTCWIHDVD
FLSCSWAVGPGAPADVQYDLYLNVANRRQQYECLHYKTDAQGTRIGCREDDISRLSSGS
QSSHILVRGRSAAFGIPCTDKFVVESQIEILTPPNMTAKCNKTHSFMHWKMRSHENRKF
RYELQIQKRMQPVITEQVRDRTSFQLLNPGTYTVQIRARERVYEFLSAWSTPQRFECDQ
EEGANTRAWRTSLLIALGTLLALVCVFVICRRYLVMQRLFPRIPHMKDPIGDSFQNDKL
VVWEAGKAGLEECLVTEVQVVQKT
(CD123 S59F variant amino acid sequence)
SEQ ID NO: 56
MVLLWLTLLLIALPCLLQTKEDPNPPITNLRMKAKAQQLTWDLNRNVTDIECVKDADYF
MPAVNNSYCQFGAISLCEVTNYTVRVANPPESTWILFPENSGKPWAGAENLTCWIHDVD
FLSCSWAVGPGAPADVQYDLYLNVANRRQQYECLHYKTDAQGTRIGCREDDISRLSSGS
QSSHILVRGRSAAFGIPCTDKFVVFSQIEILTPPNMTAKCNKTHSFMHWKMRSHENRKF
RYELQIQKRMQPVITEQVRDRTSFQLLNPGTYTVQIRARERVYEFLSAWSTPQRFECDQ
EEGANTRAWRTSLLIALGTLLALVCVFVICRRYLVMQRLFPRIPHMKDPIGDSFQNDKL
VVWEAGKAGLEECLVTEVQVVQKT
(CD123 P88S variant amino acid sequence)
SEQ ID NO: 57
MVLLWLTLLLIALPCLLQTKEDPNPPITNLRMKAKAQQLTWDLNRNVTDIECVKDADYS
MPAVNNSYCQFGAISLCEVTNYTVRVANSPESTWILFPENSGKPWAGAENLTCWIHDVD
FLSCSWAVGPGAPADVQYDLYLNVANRRQQYECLHYKTDAQGTRIGCREDDISRLSSGS
QSSHILVRGRSAAFGIPCTDKFVVFSQIEILTPPNMTAKCNKTHSFMHWKMRSHENRKF
RYELQIQKRMQPVITEQVRDRTSFQLLNPGTYTVQIRARERVYEFLSAWSTPQRFECDQ
EEGANTRAWRTSLLIALGTLLALVCVFVICRRYLVMQRLFPRIPHMKDPIGDSFQNDKL
VVWEAGKAGLEECLVTEVQVVQKT
(CD123 P88L variant amino acid sequence)
SEQ ID NO: 58
MVLLWLTLLLIALPCLLQTKEDPNPPITNLRMKAKAQQLTWDLNRNVTDIECVKDADYS
MPAVNNSYCQFGAISLCEVTNYTVRVANLPESTWILFPENSGKPWAGAENLTCWIHDVD
FLSCSWAVGPGAPADVQYDLYLNVANRRQQYECLHYKTDAQGTRIGCREDDISRLSSGS
QSSHILVRGRSAAFGIPCTDKFVVFSQIEILTPPNMTAKCNKTHSEMHWKMRSHFNRKF
RYELQIQKRMQPVITEQVRDRTSFQLLNPGTYTVQIRARERVYEFLSAWSTPQRFECDQ
EEGANTRAWRTSLLIALGTLLALVCVFVICRRYLVMQRLFPRIPHMKDPIGDSFQNDKL
VVWEAGKAGLEECLVTEVQVVQKT

Nucleotide sequences are as follows:

CD123 exon2 wild type sequence
(SEQ.ID NO: 59)
Atccaaacccaccaatcacgaacctaaggatgaaagcaaaggctcagcagttgacctgg
gaccttaacagaaatgtgaccgatatcgagtgtgttaaagacgccgactattctatgcc
g
CD123 exon2 sequence with base editing induced by IL3RA_gRNA N or
IL3RA_gRNA_R and SpRY_ABE8e_V106W (I variant)
(SEQ.ID NO: 60)
atccaaacccaccaatcacgaacctaaggatgaaagcaaaggctcagcagttgacctgg
gaccttaacagaaatgtgaccgatatcgagtgtgttaaagacgccgactaCtctatgcc
g
CD123 exon2 sequence with base editing induced by IL3RA_gRNA_N or
IL3RA_gRNA_R and SpRY_ABE8e_V106W (II variant)
(SEQ.ID NO: 61)
atccaaacccaccaatcacgaacctaaggatgaaagcaaaggctcagcagttgacctgg
gaccttaacagaaatgtgaccgatatcgagtgtgttaaagacgccgactatCctatgcc
g
CD123 exon2 sequence with base editing induced by IL3RA_gRNA N or
IL3RA_gRNA_R and SpRY_ABE8e_V106W (III variant)
(SEQ.ID NO: 62)
atccaaacccaccaatcacgaacctaaggatgaaagcaaaggctcagcagttgacctgg
gaccttaacagaaatgtgaccgatatcgagtgtgttaaagacgccgactaCCctatgcc
g
CD123 exon2 sequence with base editing induced by IL3RA_gRNA_N or
IL3RA_gRNA_R and SpRY_ABE8e_V106W (IV variant)
(SEQ.ID NO: 63)
atccaaacccaccaatcacgaacctaaggatgaaagcaaaggctcagcagttgacctgg
gaccttaacagaaatgtgaccgatatcgagtgtgttaaagacgccgactaCCcCatgcc
g
CD123 exon2 sequence with base editing induced by IL3RA_gRNA_N or
IL3RA_gRNA_R and SpRY_ABE8e_V106W (V variant)
(SEQ.ID NO: 64)
atccaaacccaccaatcacgaacctaaggatgaaagcaaaggctcagcagttgacctgg
gaccttaacagaaatgtgaccgatatcgagtgtgttaaagacgccgactatCcCatgcc
g
CD123 exon2 sequence with base editing induced by IL3RA_gRNA_N or
IL3RA_gRNA_R and SpRY_ABE8e_V106W (VI variant)
(SEQ.ID NO: 65)
atccaaacccaccaatcacgaacctaaggatgaaagcaaaggctcagcagttgacctgg
gaccttaacagaaatgtgaccgatatcgagtgtgttaaagacgccgactattcCatgcc
g

In some embodiments, provided herein is a polypeptide sequence that is at least 80%, 85%, 90%, 91%, 92%, 93%, 94%, 95%, 96%, 97%, 98%, 99%, or 100% identical to the sequence set forth in SEQ ID NO: 54, wherein the polypeptide sequence comprises a mutation at S59P and wherein the polypeptide sequence has reduced binding to a therapeutic anti-CD123 antibody. Also provided herein are nucleic acids encoding the polypeptide sequence.

In some embodiments, provided herein is a polypeptide sequence that is at least 80%, 85%, 90%, 91%, 92%, 93%, 94%, 95%, 96%, 97%, 98%, 99%, or 100% identical to the sequence set forth in SEQ ID NO: 55, wherein the polypeptide sequence comprises mutations at Y58H and S59P and wherein the polypeptide sequence has reduced binding to a therapeutic anti-CD123 antibody. Also provided herein are nucleic acids encoding the polypeptide sequence. Also provided herein are nucleic acids encoding the polypeptide sequence.

In some embodiments, provided herein is a polypeptide sequence that is at least 80%, 85%, 90%, 91%, 92%, 93%, 94%, 95%, 96%, 97%, 98%, 99%, or 100% identical to the sequence set forth in SEQ ID NO: 56, wherein the polypeptide sequence comprises a mutation at S59F and wherein the polypeptide sequence has reduced binding to a therapeutic anti-CD123 antibody. Also provided herein are nucleic acids encoding the polypeptide sequence.

In some embodiments, provided herein is a polypeptide sequence that is at least 80%, 85%, 90%, 91%, 92%, 93%, 94%, 95%, 96%, 97%, 98%, 99%, or 100% identical to the sequence set forth in SEQ ID NO: 57, wherein the polypeptide sequence comprises a mutation at P88S and wherein the polypeptide sequence has reduced binding to a therapeutic anti-CD123 antibody. Also provided herein are nucleic acids encoding the polypeptide sequence.

In some embodiments, provided herein is a polypeptide sequence that is at least 80%, 85%, 90%, 91%, 92%, 93%, 94%, 95%, 96%, 97%, 98%, 99%, or 100% identical to the sequence set forth in SEQ ID NO: 58, wherein the polypeptide sequence comprises a mutation at P88L and wherein the polypeptide sequence has reduced binding to a therapeutic anti-CD123 antibody. Also provided herein are nucleic acids encoding the polypeptide sequence.

vii. Mutant KIT

In some embodiments, the cell-surface protein is KIT. The amino acid sequence of wild-type KIT is shown below:

(KIT wild type amino acid sequence)
SEQ ID NO: 66
MRGARGAWDFLCVLLLLLRVQTGSSQPSVSPGEPSPPSIHPGKSDLIVRVGDEIRLLCT
DPGFVKWTFEILDETNENKQNEWITEKAEATNTGKYTCTNKHGLSNSIYVFVRDPAKLF
LVDRSLYGKEDNDTLVRCPLTDPEVTNYSLKGCQGKPLPKDLRFIPDPKAGIMIKSVKR
AYHRLCLHCSVDQEGKSVLSEKFILKVRPAFKAVPVVSVSKASYLLREGEEFTVTCTIK
DVSSSVYSTWKRENSQTKLQEKYNSWHHGDFNYERQATLTISSARVNDSGVFMCYANNT
FGSANVTTTLEVVDKGFINIFPMINTTVFVNDGENVDLIVEYEAFPKPEHQQWIYMNRT
FTDKWEDYPKSENESNIRYVSELHLTRLKGTEGGTYTFLVSNSDVNAAIAFNVYVNTKP
EILTYDRLVNGMLQCVAAGFPEPTIDWYFCPGTEQRCSASVLPVDVQTLNSSGPPFGKL
VVQSSIDSSAFKHNGTVECKAYNDVGKTSAYFNFAFKGNNKEQIHPHTLFTPLLIGFVI
VAGMMCIIVMILTYKYLQKPMYEVQWKVVEEINGNNYVYIDPTQLPYDHKWEFPRNRLS
FGKTLGAGAFGKVVEATAYGLIKSDAAMTVAVKMLKPSAHLTEREALMSELKVLSYLGN
HMNIVNLLGACTIGGPTLVITEYCCYGDLLNFLRRKRDSFICSKQEDHAEAALYKNLLH
SKESSCSDSTNEYMDMKPGVSYVVPTKADKRRSVRIGSYIERDVTPAIMEDDELALDLE
DLLSFSYQVAKGMAFLASKNCIHRDLAARNILLTHGRITKICDFGLARDIKNDSNYVVK
GNARLPVKWMAPESIFNCVYTFESDVWSYGIFLWELFSLGSSPYPGMPVDSKFYKMIKE
GFRMLSPEHAPAEMYDIMKTCWDADPLKRPTFKQIVQLIEKQISESTNHIYSNLANCSP
NRQKPVVDHSVRINSVGSTASSSQPLLVHDDV

In some embodiments, the methods described herein involve genetically engineering a mutant KIT gene in a population of hematopoietic cells using a Cas nuclease or variant thereof. In some embodiments, the methods described herein involve genetically engineering a mutation in exon 7 of KIT (e.g., mutating position H378) in a population of hematopoietic cells using a Cas nuclease or variant thereof. In some embodiments, the methods described herein involve genetically engineering a mutant KIT gene in a population of hematopoietic cells using a Cas nuclease or variant thereof and a guide sequence provided by any one of SEQ ID NOs: 36-47.

In some embodiments, the genetically engineered HSPC includes a genetically engineered KIT gene, wherein the genetically engineered KIT gene encodes a protein that has reduced binding to a therapeutic anti-KIT antibody. In some instances, the genetically engineered KIT gene includes at least one mutation in exon 6 and/or exon 7 of the KIT gene. In some instances, at least one mutation in exon 7 of the genetically engineered KIT gene results in a polypeptide bearing a mutation at position H378. In some instances, the mutation at position H378 is H378R or H378S or H378P or H378A or H378F or H378K or H378G or H378L or H378M. In some instances, mutation in exon 6 of KIT results in the encoded polypeptide having one or more of the following mutations F316S, M318V, I319K, V323I, 1334V, E360K, P363V, E366D. In some instances, the KIT exon 7 mutation results in a polypeptide having mutations at E376Q and/or H378R.

Exemplary amino acid sequences of the genetically engineered KIT are provided below:

KIT F316S, M318V, I319K, V323I, I334V, E360K, P363V, E366D, E376Q,
H378R (KIT-ECD4 variant amino acid sequence)
SEQ ID NO: 67
MRGARGAWDFLCVLLLLLRVQTGSSQPSVSPGEPSPPSIHPGKSDLIVRVGDEIRLLCT
DPGFVKWTFEILDETNENKQNEWITEKAEATNTGKYTCTNKHGLSNSIYVFVRDPAKLF
LVDRSLYGKEDNDTLVRCPLTDPEVTNYSLKGCQGKPLPKDLRFIPDPKAGIMIKSVKR
AYHRLCLHCSVDQEGKSVLSEKFILKVRPAFKAVPVVSVSKASYLLREGEEFTVTCTIK
DVSSSVYSTWKRENSQTKLQEKYNSWHHGDFNYERQATLTISSARVNDSGVFMCYANNT
FGSANVTTTLEVVDKGFINISPVKNTTIFVNDGENVDLVVEYEAFPKPEHQQWIYMNRT
FTDKWKDYVKSDNESNIRYVSQLRLTRLKGTEGGTYTFLVSNSDVNAAIAFNVYVNTKP
EILTYDRLVNGMLQCVAAGFPEPTIDWYFCPGTEQRCSASVLPVDVQTLNSSGPPFGKL
VVQSSIDSSAFKHNGTVECKAYNDVGKTSAYFNFAFKGNNKEQIHPHTLFTPLLIGFVI
VAGMMCIIVMILTYKYLQKPMYEVQWKVVEEINGNNYVYIDPTQLPYDHKWEFPRNRLS
FGKTLGAGAFGKVVEATAYGLIKSDAAMTVAVKMLKPSAHLTEREALMSELKVLSYLGN
HMNIVNLLGACTIGGPTLVITEYCCYGDLLNFLRRKRDSFICSKQEDHAEAALYKNLLH
SKESSCSDSTNEYMDMKPGVSYVVPTKADKRRSVRIGSYIERDVTPAIMEDDELALDLE
DLLSFSYQVAKGMAFLASKNCIHRDLAARNILLTHGRITKICDFGLARDIKNDSNYVVK
GNARLPVKWMAPESIFNCVYTFESDVWSYGIFLWELFSLGSSPYPGMPVDSKFYKMIKE
GFRMLSPEHAPAEMYDIMKTCWDADPLKRPTFKQIVQLIEKQISESTNHIYSNLANCSP
NRQKPVVDHSVRINSVGSTASSSQPLLVHDDV
(KIT H378R variant amino acid sequence)
SEQ ID NO: 68
MRGARGAWDFLCVLLLLLRVQTGSSQPSVSPGEPSPPSIHPGKSDLIVRVGDEIRLLCT
DPGFVKWTFEILDETNENKQNEWITEKAEATNTGKYTCTNKHGLSNSIYVFVRDPAKLF
LVDRSLYGKEDNDTLVRCPLTDPEVTNYSLKGCQGKPLPKDLRFIPDPKAGIMIKSVKR
AYHRLCLHCSVDQEGKSVLSEKFILKVRPAFKAVPVVSVSKASYLLREGEEFTVTCTIK
DVSSSVYSTWKRENSQTKLQEKYNSWHHGDFNYERQATLTISSARVNDSGVFMCYANNT
FGSANVTTTLEVVDKGFINIFPMINTTVFVNDGENVDLIVEYEAFPKPEHQQWIYMNRT
FTDKWEDYPKSENESNIRYVSELRLTRLKGTEGGTYTFLVSNSDVNAAIAFNVYVNTKP
EILTYDRLVNGMLQCVAAGFPEPTIDWYFCPGTEQRCSASVLPVDVQTLNSSGPPFGKL
VVQSSIDSSAFKHNGTVECKAYNDVGKTSAYFNFAFKGNNKEQIHPHTLFTPLLIGFVI
VAGMMCIIVMILTYKYLQKPMYEVQWKVVEEINGNNYVYIDPTQLPYDHKWEFPRNRLS
FGKTLGAGAFGKVVEATAYGLIKSDAAMTVAVKMLKPSAHLTEREALMSELKVLSYLGN
HMNIVNLLGACTIGGPTLVITEYCCYGDLLNELRRKRDSFICSKQEDHAEAALYKNLLH
SKESSCSDSTNEYMDMKPGVSYVVPTKADKRRSVRIGSYIERDVTPAIMEDDELALDLE
DLLSFSYQVAKGMAFLASKNCIHRDLAARNILLTHGRITKICDFGLARDIKNDSNYVVK
GNARLPVKWMAPESIFNCVYTFESDVWSYGIFLWELFSLGSSPYPGMPVDSKFYKMIKE
GFRMLSPEHAPAEMYDIMKTCWDADPLKRPTFKQIVQLIEKQISESTNHIYSNLANCSP
NRQKPVVDHSVRINSVGSTASSSQPLLVHDDV

In some embodiments, provided herein is a polypeptide sequence that is at least 80%, 85%, 90%, 91%, 92%, 93%, 94%, 95%, 96%, 97%, 98%, 99%, or 100% identical to the sequence set forth in SEQ ID NO: 67, wherein the polypeptide sequence comprises mutations at F316S, M318V, I319K, V323I, 1334V, E360K, P363V, E366D, E376Q, and H378R and wherein the polypeptide sequence has reduced binding to a therapeutic anti-KIT antibody. Also provided herein are nucleic acids encoding the polypeptide sequence.

In some embodiments, provided herein is a polypeptide sequence that is at least 80%, 85%, 90%, 91%, 92%, 93%, 94%, 95%, 96%, 97%, 98%, 99%, or 100% identical to the sequence set forth in SEQ ID NO: 68, wherein the polypeptide sequence comprises a mutation at H378R and wherein the polypeptide sequence has reduced binding to a therapeutic anti-KIT antibody. Also provided herein are nucleic acids encoding the polypeptide sequence.

viii. Genetically Engineered Hematopoietic Cells Expressing Mutant FLT3, CD123, and or KIT Genes

In some embodiments, the cell-surface proteins are FLT3 and CD123. In some embodiments, the cell-surface proteins are FLT3 and KIT. In some embodiments, the cell-surface proteins are KIT and CD123. In some embodiments, the two or more guides are transfected concurrently with each other. In some embodiments, the two or more guides are provided sequentially or consecutively, i.e., in two or more separate transfections. For example, a FLT3 guide RNA (any one of SEQ ID NOs: 13-23), a CD123 guide RNA (any one of SEQ ID NOs: 24-35), a KIT guide RNA (any one of SEQ ID NOs: 36-47).

In some embodiments, the genetically engineered HSPC includes a genetically engineered FLT3 gene, wherein the genetically engineered FLT3 gene encodes a protein that has reduced binding to a therapeutic anti-FLT3 antibody. In some instances, the genetically engineered FLT3 gene includes at least one mutation in exon 9 of the FLT3 gene. In some instances, at least one mutation in exon 9 of the genetically engineered FLT3 gene results in a polypeptide bearing a mutation at position N399. In some instances, the mutation at position N399 is N399D or N399G. Exemplary amino acid sequences of the genetically engineered FLT3 are shown above (see SEQ ID NOs: 49-52).

In some embodiments, the genetically engineered HSPC includes a genetically engineered CD123 gene, wherein the genetically engineered CD123 gene encodes a protein that has reduced binding to a therapeutic anti-CD123 antibody. In some instances, the genetically engineered CD123 gene includes at least one mutation in exon 2 or exon 3 of the CD123 gene. In some instances, a mutation in exon 2 of the genetically engineered CD123 gene results in a polypeptide bearing a mutation at position S59. In some instances, the mutation at position S59 is S59P or S59F. In some instances, the mutations are S59P and Y58H. In some instances, at least one mutation in exon 3 of the genetically engineered CD123 gene results in a polypeptide bearing a mutation at position P88. In some instances, the mutation at position P88 is P88L or P88S. Exemplary amino acid sequences of the genetically engineered CD123 are shown above (see SEQ ID NOs: 54-58).

In some embodiments, the genetically engineered HSPC includes a genetically engineered KIT gene, wherein the genetically engineered KIT gene encodes a protein that has reduced binding to a therapeutic anti-KIT antibody. In some instances, the genetically engineered KIT gene includes at least one mutation in exon 6 and/or exon 7 of the KIT gene. In some instances, at least one mutation in exon 7 of the genetically engineered KIT gene results in a polypeptide bearing a mutation at position H378. In some instances, the mutation at position H378 is H378R or H378S or H378P or H378A or H378F or H378K or H378G or H378L or H378M. In some instances, the mutations in exon 6 of KIT results in the encoded polypeptide having one or more of the following mutations: F316S, M318V, I319K, V323I, 1334V, E360K, P363V, E366D. In some instances, the mutations of exon 7 in KIT result in a polypeptide having mutations at one or more of E376Q and H378R. Exemplary amino acid sequences of the genetically engineered KIT are provided are shown above (see SEQ ID NOs: 67-68).

### II. Immunotherapy Agents Specific to Cell-Surface Antigens

Cytotoxic agents targeting cells (e.g., cancer cells) expressing a cell-surface antigen can be co-used with the genetically engineered hematopoietic cells as described herein. As used herein, the term “cytotoxic agent” refers to any agent that can directly or indirectly induce cytotoxicity of a target cell, which expresses the specific cell-surface antigen (e.g., a target cancer cell). Such a cytotoxic agent can comprise a protein-binding fragment that binds and targets an epitope of the specific cell-surface antigen.

i. Therapeutic Antibodies Antibody-Drug Conjugates

In some instances, the cytotoxic agent includes a therapeutic antibody, which can be conjugated to a drug (e.g., an anti-cancer drug) to form an antibody-drug conjugate (ADC). In some embodiments, the agent is an antibody-drug conjugate. In some embodiments, the antibody-drug conjugate comprises an epitope binding fragment and a toxin or drug that induces cytotoxicity in a target cell.

In some embodiments, the therapeutic anti-FLT3 antibody is anti-FLT3 clone 4G8 antibody. In some embodiments, the therapeutic anti-CD123 antibody is clone 7G3 antibody or its humanized counterpart CSL362 (“talacotuzumab”). In some embodiments, the therapeutic anti-CD123 antibody is anti-CD123 clone 6H6 antibody or anti-CD123 clone S18016F antibody. In some embodiments, the therapeutic anti-KIT antibody is anti-KIT clone Fab79D antibody.

Toxins or drugs compatible for use in antibody-drug conjugates are well known in the art and will be evident to one of ordinary skill in the art. See, e.g., Peters et al. Biosci. Rep. (2015) 35 (4): e00225, Beck et al. Nature Reviews Drug Discovery (2017) 16:315-337; Marin-Acevedo et al. J. Hematol. Oncol. (2018) 11:8; Elgundi et al. Advanced Drug Delivery Reviews (2017) 122:2-19. In some embodiments, the antibody-drug conjugate can further comprise a linker (e.g., a peptide linker, such as a cleavable linker or a non-cleavable linker) attaching the antibody and drug molecule. Examples of antibody-drug conjugates include, without limitation, brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDS0780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab veodtin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MEDI3726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861. In one example, the antibody-drug conjugate is gemtuzumab ozogamicin.

In some embodiments, binding of the antibody-drug conjugate to the epitope of the cell-surface protein induces internalization of the antibody-drug conjugate, and the drug (or toxin) can be released intracellularly. In some embodiments, binding of the antibody-drug conjugate to the epitope of a cell-surface protein induces internalization of the toxin or drug, which allows the toxin or drug to kill the cells expressing the cell surface protein (target cells). In some embodiments, binding of the antibody-drug conjugate to the epitope of a cell-surface protein induces internalization of the toxin or drug, which can regulate the activity of the cell expressing the cell surface protein (target cells). The type of toxin or drug used in the antibody-drug conjugates described herein is not limited to any specific type.

In some embodiments, two or more (e.g., 2, 3, 4, 5 or more) epitopes of a cell-surface antigen have been modified, enabling two or more (e.g., 2, 3, 4, 5 or more) different cytotoxic agents (e.g., two ADCs) to be targeted to the two or more epitopes. In some embodiments, the toxins carried by the ADCs could work synergistically to enhance efficacy (e.g., death of the target cells). In some embodiments, epitopes of two or more (e.g., 2, 3, 4, 5 or more) cell-surface proteins have been modified, enabling two or more (e.g., 2, 3, 4, 5 or more) different cytotoxic agents (e.g., two ADCs) to be targeted to epitopes of the two or more cell-surface antigens. In some embodiments, one or more (e.g., 1, 2, 3, 4, 5 or more) epitopes of a cell-surface antigen have been modified and one or more (e.g., 1, 2, 3, 4, 5 or more) epitopes of an additional cell-surface protein have been modified, enabling two or more (e.g., 2, 3, 4, 5 or more) different cytotoxic agents (e.g., two ADCs) to be targeted to epitopes of the cell-surface antigen and epitopes of additional cell-surface antigen. In some embodiments, targeting of more than one cell-surface antigen or a cell-surface antigen and one or more additional cell-surface protein/antigen can reduce relapse of a hematopoietic malignancy.

In some embodiments, the methods described herein involve administering ADCs that target an epitope of a cell-surface antigen that is mutated in the population of genetically engineered hematopoietic cells. In some embodiments, the methods described herein involve administering ADCs that target an epitope of a cell-surface antigen that is mutated in the population of genetically engineered hematopoietic cells and one or more additional cytotoxic agents that can target one or more additional cell-surface proteins. In some embodiments, the agents could work synergistically to enhance efficacy by targeting more than one cell-surface protein.

An ADC described herein can be used as a follow-on treatment to subjects who have been undergone the combined therapy as described herein.

In some embodiments, the methods described herein involve administering to the subject a population of genetically engineered cells lacking a non-essential epitope in a cell-surface antigen and one or more immunotherapeutic agents (e.g., ADCs) that target cells expressing the cell-surface antigen. In some embodiments, the methods described herein involve administering to the subject a population of genetically engineered cells lacking a non-essential epitope in a type 1 cell-surface antigen and one or more immunotherapeutic agents (e.g., ADCs) that target cells expressing the cell-surface antigen. In some embodiments, the methods described herein involve administering to the subject a population of genetically engineered cells lacking a non-essential epitope in a type 2 cell-surface antigen and one or more immunotherapeutic agents (e.g., ADCs) that target cells expressing the cell-surface antigen. In any of the embodiments described herein, one or more additional immunotherapeutic agents can be further administered to the subject (e.g., targeting one or more additional epitopes and/or antigens), for example if the hematopoietic malignancy relapses.

ii. Immune Cells Expressing Chimeric Antigen Receptors

In some embodiments, the cytotoxic agent that targets an epitope of a specific cell-surface antigen as described herein is an immune cell that expresses a chimeric receptor, which comprises an epitope binding fragment (e.g., a single-chain antibody) capable of binding to the epitope of the cell surface protein (e.g., FLT3, CD123, or KIT). Recognition of a target cell (e.g., a cancer cell) having the epitope of the specific protein on its cell surface by the epitope binding fragment of the chimeric receptor transduces an activation signal to the signaling domain(s) (e.g., co-stimulatory signaling domain and/or the cytoplasmic signaling domain) of the chimeric receptor, which can activate an effector function in the immune cell expressing the chimeric receptor.

In some embodiments, the immune cell expresses more than one chimeric receptor (e.g., 2, 3, 4, 5 or more), referred to as a bispecific or multi-specific immune cell. In some embodiments, the immune cell expresses more than one chimeric receptor, at least one of which targets an epitope of a cell-surface antigen. In some embodiments, the immune cell expresses more than one chimeric receptor, each of which targets an epitope of a specific cell-surface antigen. In some embodiments, the immune cell expresses more than one chimeric receptor, at least one of which targets an epitope of a cell-surface antigen and at least one of which targets an epitope of an additional cell-surface antigen. In some embodiments, targeting of more than one cell-surface protein or a cell-surface protein and one or more additional cell-surface protein can reduce relapse of a hematopoietic malignancy. In some embodiments, the immune cell expresses a chimeric receptor that targets more than one epitope (e.g., more than one epitope of one antigen or epitopes of more than one antigen), referred to as a bispecific chimeric receptor.

In some embodiments, epitopes of two or more lineage-specific cell-surface proteins are targeted by cytotoxic agents. In some embodiments, two or more chimeric receptors are expressed in the same immune cell, e.g., bispecific chimeric receptors. Such cells can be used in any of the methods described herein. In some embodiments, cells expressing a chimeric receptor are “pooled”, i.e., two or more groups of cells express two or more different chimeric receptors. In some embodiments, two or more cells expressing different chimeric antigen receptors are administered concurrently. In some embodiments, two or more cells expressing different chimeric antigen receptors are administered sequentially. In some embodiments, epitopes of FLT3, CD123, and/or KIT are targeted by cytotoxic agents. In some embodiments, the chimeric receptors targeting FLT3, CD123, and/or KIT are expressed in the same immune cell (i.e., a bispecific immune cell). Such cells can be used in any of the methods described herein. In some embodiments, cells expressing chimeric receptors targeting FLT3, CD123, and/or KIT “pooled”, i.e., two or more groups of cells express two or more different chimeric receptors. In some embodiments, two or more groups of cells expressing chimeric receptors targeting FLT3, CD123, and/or KIT are administered concurrently. In some embodiments, two or more groups of cells expressing chimeric receptors targeting FLT3, CD123, and/or KIT are administered sequentially.

As used herein, a chimeric receptor refers to a non-naturally occurring molecule that can be expressed on the surface of a host cell and comprises binding domain that provides specificity of the chimeric receptor (e.g., an epitope binding fragment that binds to an epitope of a cell-surface lineage-specific protein). In general, chimeric receptors comprise at least two domains that are derived from different molecules. In addition to the epitope-binding fragment described herein, the chimeric receptor may further comprise one or more of the following: a hinge domain (e.g., CD28 hinge, IgG4 hinge, or CD8α hinge), a transmembrane domain (e.g., CD28 TM, CD8α TM, 4-1BB TM), a co-stimulatory domain (e.g., CD28z, 4-1BB, ICOS, OX40), a cytoplasmic signaling domain (e.g., CD3z), and combinations thereof.

In some embodiments, the chimeric receptors described herein comprise one or more hinge domain(s). In some embodiments, the hinge domain may be located between the epitope binding fragment and a transmembrane domain. A hinge domain is an amino acid segment that is generally found between two domains of a protein and may allow for flexibility of the protein and movement of one or both of the domains relative to one another. Any amino acid sequence that provides such flexibility and movement of the epitope binding fragment relative to another domain of the chimeric receptor can be used.

The hinge domain may contain about 10-200 amino acids, e.g., 15-150 amino acids, 20-100 amino acids, or 30-60 amino acids. In some embodiments, the hinge domain may be of about 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or 200 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is of CD8α or CD28. In some embodiments, the hinge domain is a portion of the hinge domain of CD8a, e.g., a fragment containing at least 15 (e.g., 20, 25, 30, 35, or 40) consecutive amino acids of the hinge domain of CD8α or CD28.

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

In some embodiments, the chimeric receptors described herein may comprise one or more transmembrane domain(s). The transmembrane domain for use in the chimeric receptors can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally occurring protein. Alternatively, the transmembrane domain may be a synthetic, non-naturally occurring protein segment, e.g., a hydrophobic protein segment that is thermodynamically stable in a cell membrane.

Transmembrane domains are classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). In some embodiments, the transmembrane domain is a single-pass transmembrane domain. In some embodiments, the transmembrane domain is a single-pass transmembrane domain that orients the N terminus of the chimeric receptor to the extracellular side of the cell and the C terminus of the chimeric receptor to the intracellular side of the cell. In some embodiments, the transmembrane domain is obtained from a single pass transmembrane protein. In some embodiments, the transmembrane domain is of CD28 or 4-1BB or CD8a.

In some embodiments, the chimeric receptors described herein comprise one or more costimulatory signaling domains. The term “co-stimulatory signaling domain,” as used herein, refers to at least a portion of a protein that mediates signal transduction within a cell to induce an immune response, such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils.

In some embodiments, the chimeric receptor comprises more than one (at least 2, 3, 4, or more) co-stimulatory signaling domains. In some embodiments, the chimeric receptor comprises more than one co-stimulatory signaling domains obtained from different costimulatory proteins. In some embodiments, the chimeric receptor does not comprise a co-stimulatory signaling domain.

In general, many immune cells require co-stimulation, in addition to stimulation of an antigen-specific signal, to promote cell proliferation, differentiation and survival, and to activate effector functions of the cell. Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory protein may be compatible for use in the chimeric receptors described herein. The type(s) of co-stimulatory signaling domain is selected based on factors such as the type of the immune cells in which the chimeric receptors would be expressed (e.g., primary T cells, T cell lines, NK cell lines) and the desired immune effector function (e.g., cytotoxicity). Examples of co-stimulatory signaling domains for use in the chimeric receptors can be the cytoplasmic signaling domain of co-stimulatory proteins, including, without limitation, CD27, CD28ζ (CD28z), 4-1BB, OX40, CD30, ICOS, CD2, CD7, LIGHT, NKG2C, B7-H3.

In some embodiments, the chimeric receptors described herein comprise one or more cytoplasmic signaling domain(s). Any cytoplasmic signaling domain can be used in the chimeric receptors described herein. In general, a cytoplasmic signaling domain relays a signal, such as interaction of an extracellular ligand-binding domain with its ligand, to stimulate a cellular response, such as inducing an effector function of the cell (e.g., cytotoxicity). In some embodiments, the cytoplasmic signaling domain is from CD3ζ (CD3z).

In some embodiments, provided herein a chimeric receptor construct targeting FLT3, CD123, FLT3+CD123, KIT, FLT3+KIT or KIT+CD123. The construct can further include at least a hinge domain (e.g., from CD28, CD8α, or an antibody), a transmembrane domain (e.g., from CD28), one or more co-stimulatory domains (from one or more of CD28z) and a cytoplasmic signaling domain (e.g., from CD3z), or a combination thereof. In some examples, the methods described herein involve administering to a subject a population of genetically engineered hematopoietic cells (engineered to have a mutant FLT3, CD123, FLT3+CD123, KIT, FLT3+KIT or CD123+KIT) and an immune cell expressing a chimeric receptor that targets FLT3, CD123, FLT3+CD123, KIT, FLT3+KIT or CD123+KIT respectively, which may further comprise at least a hinge domain (e.g., from CD28, CD8α, or an antibody), a transmembrane domain (e.g., from CD28), one or more co-stimulatory domains (from one or more of CD28z) and a cytoplasmic signaling domain (e.g., from CD3z), or combination thereof. In some embodiments, the administered immunotherapeutic product is a combination of immune cells expressing individual chimeric receptor that targets FLT3, CD123, and/or KIT.

Any of the chimeric receptors described herein can be prepared by routine methods, such as recombinant technology. Methods for preparing the chimeric receptors herein involve generation of a nucleic acid that encodes a polypeptide comprising each of the domains of the chimeric receptors, including the epitope binding fragment and optionally, the hinge domain, the transmembrane domain, at least one co-stimulatory signaling domain, and the cytoplasmic signaling domain. In some embodiments, nucleic acids encoding the components of a chimeric receptor are joined together using recombinant technology.

Additionally, any of the chimeric receptors can be expressed in immune cells and administered to a subject (e.g., a human subject) by routine methods. For example, T cells can be either derived from T cells in a subject's own blood (autologous) or derived from the T cells of another healthy donor (allogeneic). Once isolated from a subject, these T cells are genetically engineered to express a specific CAR, which programs them to target an antigen that is present on the surface of tumors. The CAR-T cells are then infused, by customary practice, into the subject.

In some embodiments, the CAR is at least 80%, 85%, 90%, 91%, 92%, 93%, 94%, 95%, 96%, 97%, 98%, 99%, or 100% identical to the amino acid sequence of any one of SEQ ID NOs: 69, 71, 73, 75, 77, 79, 86, or 87, wherein the CAR retains its ability to bind to its respective cell-surface lineage-specific protein (e.g., KIT, CD123, FLT3, or a combination thereof).

In some embodiments, the cell-surface lineage-specific protein is KIT and the epitope binding fragment comprises the following CDR sequences: GFNISVYMMH (SEQ ID NO: 88), SIYPYSGYTYYADSVKG (SEQ ID NO: 89), ARYVYHALDY (SEQ ID NO: 90), RASQRGLRNVAVA (SEQ ID NO: 91), SASSLYS (SEQ ID NO: 92), and QQWAVHSLIT (SEQ ID NO: 93). In some instances, the epitope binding fragment comprises a sequence that is at least 80%, 85%, 90%, 91%, 92%, 93%, 94%, 95%, 96%, 97%, 98%, 99%, or 100% identical to GFNISVYMMHWVRQAPGKGLEWVASIYPYSGYTYYADSVKGRFTISADTSKNT AYLQMNSLRAEDTAVYYCARYVYHALDY (SEQ ID NO: 94), wherein the epitope binding fragment retains its ability to bind to its respective KIT epitope. In some instances, the epitope binding fragment comprises a sequence that is at least 80%, 85%, 90%, 91%, 92%, 93%, 94%, 95%, 96%, 97%, 98%, 99%, or 100% identical to RASQRGLRNVAVAWYQQKPGKAPKLLIYSASSLYSGVPSRFSGSRSGTDFTLTIS SLQPEDFATYYCQQWAVHSLIT (SEQ ID NO: 95), wherein the epitope binding fragment retains its ability to bind to its respective KIT epitope. In some instances, the epitope binding fragment comprises a sequence that is at least 80%, 85%, 90%, 91%, 92%, 93%, 94%, 95%, 96%, 97%, 98%, 99%, or 100% identical to SEQ ID NO: 94 and comprises a sequence that is at least 80%, 85%, 90%, 91%, 92%, 93%, 94%, 95%, 96%, 97%, 98%, 99%, or 100% identical to SEQ ID NO: 95. In some embodiments, the epitope binding fragment comprises both SEQ ID NOs 94 and 95.

In some embodiments, the cell-surface lineage-specific protein is FLT3 and the epitope binding fragment comprises the following CDR sequences: GYTFTSYWMH (SEQ ID NO: 96), EIDPSDSYKDYNQKFK (SEQ ID NO: 97), RAITTTPFDF (SEQ ID NO: 98), RASQSISNNLH (SEQ ID NO: 99), YASQSIS (SEQ ID NO: 100), and QQSNTWPYT (SEQ ID NO: 101). In some instances, the epitope binding fragment comprises a sequence that is at least 80%, 85%, 90%, 91%, 92%, 93%, 94%, 95%, 96%, 97%, 98%, 99%, or 100% identical to GYTFTSYWMHWVRQRPGHGLEWIGEIDPSDSYKDYNQKFKDKATLTVDRSSNT AYMHLSSLTSDDSAVYYCARAITTTPFDF (SEQ ID NO: 102), wherein the epitope binding fragment retains its ability to bind to its respective FLT3 epitope. In some instances, the epitope binding fragment comprises a sequence that is at least 80%, 85%, 90%, 91%, 92%, 93%, 94%, 95%, 96%, 97%, 98%, 99%, or 100% identical to RASQSISNNLHWYQQKSHESPRLLIKYASQSISGIPSRFSGSGSGTDFTLSINSVETE DFGVYFCQQSNTWPYT (SEQ ID NO: 103), wherein the epitope binding fragment retains its ability to bind to its respective FLT3 epitope. In some instances, the epitope binding fragment comprises a sequence that is at least 80%, 85%, 90%, 91%, 92%, 93%, 94%, 95%, 96%, 97%, 98%, 99%, or 100% identical to SEQ ID NO: 102 and comprises a sequence that is at least 80%, 85%, 90%, 91%, 92%, 93%, 94%, 95%, 96%, 97%, 98%, 99%, or 100% identical to SEQ ID NO: 103. In some embodiments, the epitope binding fragment comprises both SEQ ID NOs 102 and 103.

In some embodiments, the cell-surface lineage-specific protein is CD123 and the epitope binding fragment comprises the following CDR sequences: GYSFTDYYMK (SEQ ID NO: 104), DIIPSNGATFYNQKFKG (SEQ ID NO: 105), ARSHLLRASWFAY (SEQ ID NO: 106), SQSLLNSGNQKNYLT (SEQ ID NO: 107), WASTRES (SEQ ID NO: 108), and QNDYSYPYT (SEQ ID NO: 109). In some instances, the epitope binding fragment comprises a sequence that is at least 80%, 85%, 90%, 91%, 92%, 93%, 94%, 95%, 96%, 97%, 98%, 99%, or 100% identical to GYSFTDYYMKWARQMPGKGLEWMGDIIPSNGATFYNQKFKGQVTISADKSISTT YLQWSSLKASDTAMYYCARSHLLRASWFAY (SEQ ID NO: 110), wherein the epitope binding fragment retains its ability to bind to its respective CD123 epitope. In some instances, the epitope binding fragment comprises a sequence that is at least 80%, 85%, 90%, 91%, 92%, 93%, 94%, 95%, 96%, 97%, 98%, 99%, or 100% identical to ESSQSLLNSGNQKNYLTWYQQKPGQPPKPLIYWASTRESGVPDRFSGSGSGTDFT LTISSLQAEDVAVYYCONDYSYPYT (SEQ ID NO: 111), wherein the epitope binding fragment retains its ability to bind to its respective CD123 epitope. In some instances, the epitope binding fragment comprises a sequence that is at least 80%, 85%, 90%, 91%, 92%, 93%, 94%, 95%, 96%, 97%, 98%, 99%, or 100% identical to SEQ ID NO: 110 and comprises a sequence that is at least 80%, 85%, 90%, 91%, 92%, 93%, 94%, 95%, 96%, 97%, 98%, 99%, or 100% identical to SEQ ID NO: 111. In some embodiments, the epitope binding fragment comprises both SEQ ID NOs 110 and 111.

In some embodiments, the cell-surface lineage-specific proteins are FLT3 and CD123 and the epitope binding fragment comprises the following CDR sequences: GYTFTSYWMH (SEQ ID NO: 96), EIDPSDSYKDYNQKFK (SEQ ID NO: 97), RAITTTPFDF (SEQ ID NO: 98), RASQSISNNLH (SEQ ID NO: 99), YASQSIS (SEQ ID NO: 100), QQSNTWPYT (SEQ ID NO: 101), GYSFTDYYMK (SEQ ID NO: 104), DIIPSNGATFYNQKFKG (SEQ ID NO: 105), ARSHLLRASWFAY (SEQ ID NO: 106), SQSLLNSGNQKNYLT (SEQ ID NO: 107), WASTRES (SEQ ID NO: 108), and QNDYSYPYT (SEQ ID NO: 109). In some instances, the epitope binding fragment comprises a sequence that is at least 80%, 85%, 90%, 91%, 92%, 93%, 94%, 95%, 96%, 97%, 98%, 99%, or 100% identical to GYTFTSYWMHWVRQRPGHGLEWIGEIDPSDSYKDYNQKFKDKATLTVDRSSNT AYMHLSSLTSDDSAVYYCARAITTTPFDF (SEQ ID NO: 102), wherein the epitope binding fragment retains its ability to bind to its respective FLT3 epitope. In some instances, the epitope binding fragment comprises a sequence that is at least 80%, 85%, 90%, 91%, 92%, 93%, 94%, 95%, 96%, 97%, 98%, 99%, or 100% identical to RASQSISNNLHWYQQKSHESPRLLIKYASQSISGIPSRFSGSGSGTDFTLSINSVETE DFGVYFCQQSNTWPYT (SEQ ID NO: 103), wherein the epitope binding fragment retains its ability to bind to its respective FLT3 epitope. In some instances, the epitope binding fragment comprises a sequence that is at least 80%, 85%, 90%, 91%, 92%, 93%, 94%, 95%, 96%, 97%, 98%, 99%, or 100% identical to GYSFTDYYMKWARQMPGKGLEWMGDIIPSNGATFYNQKFKGQVTISADKSISTT YLQWSSLKASDTAMYYCARSHLLRASWFAY (SEQ ID NO: 110), wherein the epitope binding fragment retains its ability to bind to its respective CD123 epitope. In some instances, the epitope binding fragment comprises a sequence that is at least 80%, 85%, 90%, 91%, 92%, 93%, 94%, 95%, 96%, 97%, 98%, 99%, or 100% identical to ESSQSLLNSGNQKNYLTWYQQKPGQPPKPLIYWASTRESGVPDRFSGSGSGTDFT LTISSLQAEDVAVYYCONDYSYPYT (SEQ ID NO: 111), wherein the epitope binding fragment retains its ability to bind to its respective CD123 epitope. In some instances, the epitope binding fragment comprises a sequence that is at least 80%, 85%, 90%, 91%, 92%, 93%, 94%, 95%, 96%, 97%, 98%, 99%, or 100% identical to SEQ ID NO: 102, a sequence that is at least 80%, 85%, 90%, 91%, 92%, 93%, 94%, 95%, 96%, 97%, 98%, 99%, or 100% identical to SEQ ID NO: 103, a sequence that is at least 80%, 85%, 90%, 91%, 92%, 93%, 94%, 95%, 96%, 97%, 98%, 99%, or 100% identical to SEQ ID NO: 110, and a sequence that is at least 80%, 85%, 90%, 91%, 92%, 93%, 94%, 95%, 96%, 97%, 98%, 99%, or 100% identical to SEQ ID NO: 111. In some embodiments, the epitope binding fragment comprises all four of SEQ ID NOs 102, 103, 110, and 111.

In some embodiments, the cell-surface lineage-specific protein is CD123 and the epitope binding fragment comprises the following CDR sequences: DIIPSNGATFYNQKFKG (SEQ ID NO: 105), SQSLLNSGNQKNYLT (SEQ ID NO: 107), WASTRES (SEQ ID NO: 108), and QNDYSYPYT (SEQ ID NO: 109).

### Exemplary CAR Sequences are Provided Below:

-Fab79D-CAR (CD28 hinge, CD28 TM, CD28z, CD3z) amino acid sequence
targeting KIT domain 4, variant I (epitope binding regions are italicized,
CDRs are bolded)
SEQ ID NO: 69
MLLLVTSLLLCELPHPAFLLIPEVQLVESGGGLVQPGGSLRLSCAASGFNISVYMMHWV
RQAPGKGLEWVASIYPYSGYTYYADSVKGRFTISADTSKNTAYLQMNSLRAEDTAVYYC
ARYVYHALDYWGQGTLVTVSSGSTSGSGKPGSSEGSTKGDIQMTQSPSSLSASVGDRVT
ITCRASQRGLRNVAVAWYQQKPGKAPKLLIYSASSLYSGVPSRFSGSRSGTDFTLTISS
LQPEDFATYYCQQWAVHSLITFGQGTKVEIKRAAIEVMYPPPYLDNEKSNGTIIHVKGK
HLCPSPLFPGPSKPFWVLVVVGGVLACYSLLVTVAFIIFWVRSKRSRGGHSDYMNMTPR
RPGPTRKHYQPYAPPRDFAAYRSRVKFSRSADAPAYQQGQNQLYNELNLGRREEYDVLD
KRRGRDPEMGGKPRRKNPQEGLYNELQKDKMAEAYSEIGMKGERRRGKGHDGLYQGLST
ATKDTYDALHMQALPPR
-Fab79D- CAR (CD28 hinge, CD28 TM, CD28z, CD3z) nucleotide sequence
targeting KIT domain 4, variant I
SEQ ID NO: 70
ATGCTTCTCCTGGTTACGTCACTGCTCTTGTGCGAGCTGCCTCACCCTGCGTTTTTGTT
GATCCCAGAAGTACAGCTTGTCGAGTCCGGCGGCGGACTCGTGCAGCCTGGCGGCTCCC
TTCGCCTCTCCTGCGCTGCCAGTGGATTCAACATCTCTGTCTATATGATGCATTGGGTG
CGGCAAGCACCGGGCAAAGGCTTGGAGTGGGTGGCCTCTATTTATCCGTATTCTGGCTA
TACGTATTATGCGGATAGCGTTAAAGGGCGGTTCACCATATCCGCAGACACCTCTAAGA
ATACAGCTTATTTGCAGATGAACAGTTTGCGAGCGGAAGACACGGCCGTTTATTATTGC
GCCCGATACGTATATCATGCCCTCGACTATTGGGGTCAGGGAACACTCGTCACCGTCTC
ATCTGGGTCAACCAGTGGGTCTGGTAAACCCGGCTCAAGCGAGGGCAGTACCAAAGGAG
ACATCCAGATGACCCAGAGCCCTTCCTCATTGTCAGCATCCGTCGGGGACAGAGTAACC
ATAACCTGCCGGGCCAGTCAGCGCGGCCTCCGGAACGTAGCAGTGGCATGGTATCAACA
AAAACCTGGCAAGGCCCCAAAACTCCTTATATATTCAGCTTCTTCCCTTTATTCTGGCG
TGCCCTCCAGGTTCTCCGGGTCACGATCCGGTACCGATTTCACCCTGACTATCAGTTCT
CTCCAGCCCGAAGACTTTGCAACGTACTACTGCCAACAATGGGCAGTTCACAGTCTTAT
AACATTTGGGCAGGGGACGAAAGTCGAGATCAAACGAGCCGCCATAGAAGTAATGTACC
CACCACCGTACCTGGATAACGAGAAAAGTAACGGTACTATCATACATGTTAAAGGTAAA
CACCTTTGCCCATCCCCACTCTTCCCTGGACCTTCGAAACCGTTCTGGGTTTTGGTGGT
GGTAGGTGGTGTCCTTGCTTGTTATTCCCTGCTGGTCACCGTTGCGTTTATCATCTTCT
GGGTTCGCAGCAAGAGGTCACGTGGTGGGCACTCCGACTACATGAACATGACGCCCCGA
CGCCCAGGTCCTACGAGAAAGCATTACCAACCGTACGCTCCACCCCGCGACTTTGCTGC
CTACCGGTCCAGAGTAAAATTCAGTCGGTCTGCTGATGCCCCTGCTTACCAACAGGGCC
AGAATCAGCTCTACAATGAGCTTAATCTGGGACGACGAGAGGAATATGACGTTTTGGAC
AAGCGAAGGGGCCGAGACCCTGAGATGGGCGGTAAACCGCGGAGAAAGAACCCCCAGGA
GGGGTTGTATAATGAATTGCAAAAGGACAAAATGGCGGAGGCGTATTCCGAAATAGGGA
TGAAGGGTGAAAGGCGGCGAGGCAAGGGACATGATGGTCTCTACCAAGGGCTGTCTACT
GCCACGAAGGACACCTATGATGCGCTTCATATGCAGGCCTTGCCTCCGCGA
-Fab79D-CAR (CD28 hinge, CD28 TM, CD28z, CD3z) amino acid sequence
targeting KIT domain 4, variant II (epitope binding regions are italicized,
CDRs are bolded)
SEQ ID NO: 71
MLRLLLALNLFPSIQVTGGSSDIQMTQSPSSLSASVGDRVTITCRASQRGLRNVAVAWY
QQKPGKAPKLLIYSASSLYSGVPSRFSGSRSGTDFTLTISSLQPEDFATYYCQQWAVHS
LITFGQGTKVEIKGSTSGSGKPGSGEGSTKGEVQLVESGGGLVQPGGSLRLSCAASGFN
ISVYMMHWVRQAPGKGLEWVASIYPYSGYTYYADSVKGRFTISADTSKNTAYLQMNSLR
AEDTAVYYCARYVYHALDYWGQGTLVTVSSIEVMYPPPYLDNEKSNGTIIHVKGKHLCP
SPLFPGPSKPFWVLVVVGGVLACYSLLVTVAFIIFWVRSKRSRGGHSDYMNMTPRRPGP
TRKHYQPYAPPRDFAAYRSRVKFSRSADAPAYQQGQNQLYNELNLGRREEYDVLDKRRG
RDPEMGGKPRRKNPQEGLYNELQKDKMAEAYSEIGMKGERRRGKGHDGLYQGLSTATKD
TYDALHMQALPPR
-Fab79D-CAR (CD28 hinge, CD28 TM, CD28z, CD3z) nucleotide sequence
targeting KIT domain 4, variant II
SEQ ID NO: 72
ATGCTTCGCCTCTTGCTGGCATTGAATCTTTTTCCGAGTATTCAGGTTACAGGAGGCTC
CTCAGACATCCAGATGACCCAATCCCCCTCATCTCTTTCTGCGTCTGTTGGAGACCGAG
TAACGATTACTTGCCGGGCAAGCCAAAGGGGACTCCGCAATGTGGCTGTCGCTTGGTAT
CAACAGAAGCCAGGTAAGGCACCAAAACTTTTGATCTACTCTGCTTCTTCCCTTTACTC
AGGTGTACCGAGCCGCTTCAGTGGCTCCCGGTCCGGAACGGACTTCACTTTGACGATAA
GTTCACTGCAGCCGGAAGACTTCGCCACATATTACTGCCAGCAATGGGCGGTCCACAGC
TTGATTACATTCGGACAAGGAACAAAGGTGGAAATAAAGGGCAGCACGAGTGGTAGCGG
CAAGCCTGGGTCAGGGGAGGGTTCAACGAAAGGGGAGGTCCAACTGGTCGAGTCAGGGG
GCGGACTTGTTCAGCCGGGTGGGAGCCTCCGACTGAGTTGCGCGGCATCCGGTTTTAAT
ATCTCAGTCTATATGATGCACTGGGTTAGACAAGCACCCGGCAAGGGTTTGGAATGGGT
AGCGTCAATATACCCCTATTCTGGCTACACCTATTACGCAGACTCTGTCAAAGGCAGGT
TTACAATCAGCGCGGACACCAGCAAAAACACCGCGTATCTCCAAATGAACAGCCTGCGG
GCAGAGGACACCGCTGTCTACTACTGCGCACGCTATGTCTACCACGCTTTGGATTATTG
GGGTCAAGGGACGCTGGTCACCGTATCAAGCATCGAGGTAATGTATCCTCCTCCCTATT
TGGATAACGAGAAGTCTAACGGAACTATTATTCACGTAAAGGGTAAACATCTCTGCCCC
AGCCCTTTGTTTCCGGGACCTTCAAAACCTTTTTGGGTTCTGGTGGTGGTTGGGGGAGT
CTTGGCGTGTTATTCCCTTCTCGTGACGGTGGCGTTCATAATATTCTGGGTCCGGAGCA
AAAGATCACGTGGCGGTCACTCAGATTACATGAATATGACGCCGAGGAGGCCGGGTCCT
ACCCGAAAGCATTATCAACCTTACGCACCACCGCGCGACTTCGCAGCTTACCGGTCCAG
GGTGAAGTTTAGCAGAAGTGCTGACGCCCCAGCCTACCAACAGGGCCAAAATCAACTGT
ACAACGAGCTGAACCTGGGACGAAGAGAGGAATACGACGTCCTGGACAAGAGGAGGGGG
CGCGACCCCGAGATGGGGGGGAAGCCGAGGCGCAAGAACCCCCAGGAGGGTCTTTATAA
TGAACTGCAGAAGGATAAGATGGCCGAAGCCTATTCTGAAATCGGAATGAAGGGTGAGC
GGCGCAGAGGTAAAGGGCACGACGGCCTCTATCAGGGACTTTCTACGGCAACGAAAGAT
ACGTATGACGCACTTCATATGCAAGCCCTTCCGCCCAGA
-4G8-CAR (IgG4 hinge, CD28 TM, CD28z, CD3z) amino acid sequence
targeting FLT3 domain 4 (epitope binding regions are italicized; CDRs are
bolded)
SEQ ID NO: 73
MLLLVTSLLLCELPHPAFLLIPQVQLQQPGAELVKPGASLKLSCKSSGYTFTSYWMHWV
RQRPGHGLEWIGEIDPSDSYKDYNQKFKDKATLTVDRSSNTAYMHLSSLTSDDSAVYYC
ARAITTTPFDFWGQGTTLTVSSGGGGSGGGGSGGGGSDIVLTQSPATLSVTPGDSVSLS
CRASQSISNNLHWYQQKSHESPRLLIKYASQSISGIPSRFSGSGSGTDFTLSINSVETE
DFGVYFCQQSNTWPYTFGGGTKLEIKRESKYGPPCPPCPASMFWVLVVVGGVLACYSLL
VTVAFIIFWVRSKRSRGGHSDYMNMTPRRPGPTRKHYQPYAPPRDFAAYRSRVKESRSA
DAPAYQQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
-4G8-CAR (IgG4 hinge, CD28 TM, CD28z, CD3z) nucleotide sequence targeting
FLT3 domain 4
SEQ ID NO: 74
ATGCTCTTGTTGGTGACGAGTCTCCTGCTGTGTGAACTGCCGCACCCAGCATTTCTTTT
GATTCCGCAGGTGCAGCTGCAGCAGCCTGGCGCCGAACTCGTGAAACCTGGCGCCTCTC
TGAAGCTGAGCTGCAAGAGCAGCGGCTACACCTTCACCAGCTACTGGATGCACTGGGTG
CGCCAGAGGCCTGGCCACGGACTGGAATGGATCGGCGAGATCGACCCCAGCGACAGCTA
CAAGGACTACAACCAGAAGTTCAAGGACAAGGCCACCCTGACCGTGGACAGAAGCAGCA
ACACCGCCTACATGCACCTGTCCAGCCTGACCAGCGACGACAGCGCCGTGTACTACTGT
GCCAGAGCCATCACAACCACCCCCTTCGATTTCTGGGGCCAGGGCACAACCCTGACAGT
GTCTAGCGGAGGCGGAGGCTCCGGTGGGGGAGGATCTGGGGGAGGCGGAAGCGATATTG
TGCTGACCCAGAGCCCTGCCACACTGAGCGTGACACCAGGCGATAGCGTGTCCCTGTCC
TGCAGAGCCAGCCAGAGCATCTCCAACAACCTGCACTGGTATCAGCAGAAGTCCCACGA
GAGCCCCAGACTGCTGATTAAGTACGCCAGCCAGTCCATCAGCGGCATCCCCAGCAGAT
TTTCCGGCAGCGGCTCCGGCACCGACTTCACCCTGAGCATCAACAGCGTGGAAACCGAG
GACTTCGGCGTGTACTTCTGCCAGCAGAGCAACACCTGGCCTTACACCTTCGGCGGAGG
CACCAAGCTGGAAATCAAGAGAGAGTCTAAGTACGGACCGCCCTGCCCCCCTTGCCCTG
CTAGCATGTTCTGGGTGCTGGTGGTGGTCGGAGGCGTGCTGGCCTGCTACAGCCTGCTG
GTCACCGTGGCCTTCATCATCTTTTGGGTCCGCAGCAAGCGGAGCAGAGGCGGCCACAG
CGACTACATGAACATGACCCCTAGACGGCCTGGCCCCACCAGAAAGCACTACCAGCCCT
ACGCCCCTCCCCGGGACTTTGCCGCCTACAGAAGCCGGGTGAAGTTCAGCAGAAGCGCC
GACGCCCCTGCCTACCAGCAGGGCCAGAATCAGCTGTACAACGAGCTGAACCTGGGCAG
AAGGGAAGAGTACGACGTCCTGGATAAGCGGAGAGGCCGGGACCCTGAGATGGGCGGCA
AGCCTCGGCGGAAGAACCCCCAGGAAGGCCTGTATAACGAACTGCAGAAAGACAAGATG
GCCGAGGCCTACAGCGAGATCGGCATGAAGGGCGAACGGAGGCGGGGCAAGGGCCACGA
CGGCCTGTATCAGGGCCTGTCCACCGCCACCAAGGATACCTACGACGCCCTGCACATGC
AGGCCCTGCCCCCAAGG
-CSL362-CAR (CD8a hinge, CD28 TM, CD28z, CD3z) amino acid sequence
targeting CD123 N-terminal domain (epitope binding regions are italicized,
CDRs are bolded)
SEQ ID NO: 75
MLLLVTSLLLCELPHPAFLLIPEVQLVQSGAEVKKPGESLKISCKGSGYSFTDYYMKWA
RQMPGKGLEWMGDIIPSNGATFYNQKFKGQVTISADKSISTTYLQWSSLKASDTAMYYC
ARSHLLRASWFAYWGQGTMVTVSSGSTSGSGKPGSSEGSTKGDIVMTQSPDSLAVSLGE
RATINCESSQSLLNSGNQKNYLTWYQQKPGQPPKPLIYWASTRESGVPDRFSGSGSGTD
FTLTISSLQAEDVAVYYCQNDYSYPYTFGQGTKLEIKTTTPAPRPPTPAPTIASQPLSL
RPEACRPAAGGAVHTRGLDFACDIYIWAPLAGTCGVLLLSLVITLYCNHRNRCKRSRGG
HSDYMNMTPRRPGPTRKHYQPYAPPRDFAAYRSRVKESRSADAPAYQQGQNQLYNELNL
GRREEYDVLDKRRGRDPEMGGKPRRKNPQEGLYNELQKDKMAEAYSEIGMKGERRRGKG
HDGLYQGLSTATKDTYDALHMQALPPR
-CSL362-CAR (CD8a hinge, CD28 TM, CD28z, CD3z) nucleotide sequence
targeting CD123 N-terminal domain
SEQ ID NO: 76
ATGCTTCTCCTGGTTACCTCCTTGCTGTTGTGTGAGCTGCCTCATCCAGCATTTCTCTT
GATACCAGAGGTTCAGCTCGTACAGAGCGGGGCCGAGGTGAAAAAACCCGGCGAGTCTT
TGAAAATATCATGCAAAGGCAGTGGCTATTCCTTTACGGATTACTACATGAAGTGGGCG
AGGCAGATGCCAGGGAAGGGACTCGAATGGATGGGAGACATCATACCGAGCAATGGTGC
GACCTTCTATAACCAAAAATTCAAAGGGCAGGTCACTATTTCCGCTGACAAAAGCATCA
GTACAACGTACTTGCAATGGTCTTCTCTTAAAGCCTCCGACACTGCGATGTACTATTGT
GCGAGATCACATCTCCTGAGAGCGAGTTGGTTCGCCTACTGGGGCCAAGGAACCATGGT
CACGGTCTCAAGTGGCAGCACCTCCGGTTCTGGAAAGCCGGGATCCTCAGAAGGCAGTA
CAAAAGGTGATATTGTCATGACCCAATCCCCCGATAGTCTGGCTGTATCTCTCGGTGAA
AGGGCCACGATAAACTGTGAGTCCTCACAATCCCTTCTGAACTCAGGGAATCAAAAGAA
CTATCTTACTTGGTATCAGCAAAAACCTGGACAACCTCCGAAGCCACTCATATACTGGG
CCTCCACCAGGGAAAGTGGAGTCCCAGACCGATTCTCAGGATCCGGTTCCGGGACCGAC
TTCACGCTCACCATTAGTAGCCTTCAAGCAGAGGATGTAGCAGTGTACTATTGTCAAAA
TGATTATAGTTACCCTTACACCTTCGGGCAAGGCACGAAACTGGAAATCAAAACCACGA
CCCCGGCTCCACGACCTCCCACCCCCGCCCCCACAATAGCGAGCCAACCACTTAGTTTG
AGACCGGAAGCTTGCCGGCCTGCCGCGGGTGGTGCTGTTCATACAAGAGGCCTGGACTT
CGCTTGCGATATATACATCTGGGCGCCACTCGCCGGCACGTGTGGTGTCCTTCTTCTCT
CTCTGGTAATTACACTCTATTGTAACCATAGAAACCGGTGTAAAAGAAGCCGAGGCGGT
CACAGTGACTATATGAATATGACACCGCGCCGACCCGGACCAACAAGAAAACATTATCA
ACCGTACGCTCCACCCCGCGACTTTGCTGCCTACCGGTCCAGAGTAAAATTCAGTCGGT
CTGCTGATGCCCCTGCTTACCAACAGGGCCAGAATCAGCTCTACAATGAGCTTAATCTG
GGACGACGAGAGGAATATGACGTTTTGGACAAGCGAAGGGGCCGAGACCCTGAGATGGG
CGGTAAACCGCGGAGAAAGAACCCCCAGGAGGGGTTGTATAATGAATTGCAAAAGGACA
AAATGGCGGAGGCGTATTCCGAAATAGGGATGAAGGGTGAAAGGCGGCGAGGCAAGGGA
CATGATGGTCTCTACCAAGGGCTGTCTACTGCCACGAAGGACACCTATGATGCGCTTCA
TATGCAGGCCTTGCCTCCGCGA
-4G8-CSL362-bispecific CAR (CD8a hinge, CD28 TM, CD28z, CD3z) amino
acid sequence targeting both FLT3 and CD123, variant I (epitope binding
regions are italicized, CDRs are bolded)
SEQ ID NO: 77
MLLLVTSLLLCELPHPAFLLIPQVQLQQPGAELVKPGASLKLSCKSSGYTFTSYWMHWV
RQRPGHGLEWIGEIDPSDSYKDYNQKFKDKATLTVDRSSNTAYMHLSSLTSDDSAVYYC
ARAITTTPFDFWGQGTTLTVSSGSTSGSGKPGSSEGSTKGDIVLTQSPATLSVTPGDSV
SLSCRASQSISNNLHWYQQKSHESPRLLIKYASQSISGIPSRFSGSGSGTDFTLSINSV
ETEDFGVYFCQQSNTWPYTFGGGTKLEIKRGGGGSGGGGSGGGGSGGGGSGGGGSDIVM
TQSPDSLAVSLGERATINCESSQSLLNSGNQKNYLTWYQQKPGQPPKPLIYWASTRESG
VPDRFSGSGSGTDFTLTISSLQAEDVAVYYCQNDYSYPYTFGQGTKLEIKSGSTSGSGK
PGSSEGSTKGEVQLVQSGAEVKKPGESLKISCKGSGYSFTDYYMKWARQMPGKGLEWMG
DIIPSNGATFYNQKFKGQVTISADKSISTTYLQWSSLKASDTAMYYCARSHLLRASWFA
YWGQGTMVTVSTTTPAPRPPTPAPTIASQPLSLRPEACRPAAGGAVHTRGLDFACDMFW
VLVVVGGVLACYSLLVTVAFIIFWVRSKRSRGGHSDYMNMTPRRPGPTRKHYQPYAPPR
DFAAYRSRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPP
R
-4G8-CSL362-bispecific CAR (CD8a hinge, CD28 TM, CD28z, CD3z)
nucleotide sequence targeting both FLT3 and CD123, variant I
SEQ ID NO: 78
ATGCTGCTTCTGGTGACTAGCCTGCTCCTGTGCGAGCTGCCCCACCCCGCGTTCCTGCT
CATCCCCCAGGTGCAGCTGCAGCAGCCAGGGGCCGAGCTTGTAAAGCCTGGGGCTTCCC
TGAAGCTGTCTTGTAAGTCTTCCGGCTACACGTTCACCTCCTATTGGATGCACTGGGTG
CGCCAGCGTCCGGGGCATGGTCTGGAGTGGATTGGAGAAATTGACCCGAGTGACAGCTA
CAAGGACTACAACCAAAAATTTAAAGACAAGGCCACTTTAACAGTTGACAGGTCCTCCA
ACACGGCTTACATGCACCTGAGCAGTCTGACCAGCGATGACTCCGCCGTGTACTACTGT
GCCCGCGCCATCACCACCACACCCTTCGACTTCTGGGGCCAGGGCACGACCCTTACCGT
GTCGTCGGGATCTACCAGCGGTTCCGGCAAACCGGGTTCGTCGGAGGGCTCGACCAAGG
GGGACATTGTCCTGACCCAGAGCCCGGCTACCCTGTCTGTCACCCCTGGTGATTCTGTT
TCATTGTCCTGTCGGGCATCACAGTCGATCTCTAACAACCTGCACTGGTACCAGCAGAA
AAGCCACGAGTCCCCGCGCCTGCTAATCAAGTATGCATCTCAGAGTATCTCTGGCATCC
CCTCTCGCTTTAGCGGTTCCGGTTCCGGCACAGATTTCACCTTAAGTATTAACTCCGTG
GAGACCGAGGATTTCGGAGTGTATTTCTGCCAGCAGTCAAATACCTGGCCCTACACCTT
TGGCGGCGGCACCAAGCTGGAGATCAAGCGGGGAGGCGGCGGTAGCGGCGGTGGCGGCT
CCGGCGGAGGGGGATCTGGTGGAGGCGGGTCGGGCGGCGGCGGCTCCGACATCGTGATG
ACCCAGAGTCCCGACTCTCTTGCCGTCTCCTTAGGGGAGCGCGCCACCATAAACTGCGA
GTCTTCCCAGTCCCTCCTGAACTCCGGCAACCAGAAAAACTACCTAACCTGGTACCAAC
AGAAGCCCGGCCAGCCACCCAAGCCACTTATCTACTGGGCCTCCACTCGCGAATCTGGC
GTCCCTGACCGCTTCTCTGGCTCCGGTTCCGGCACCGACTTCACCCTCACTATCTCCTC
TCTGCAGGCCGAGGATGTGGCCGTGTACTACTGTCAGAACGACTACAGCTATCCCTACA
CCTTCGGCCAGGGTACTAAGTTGGAAATTAAGTCCGGTTCTACTAGCGGGTCCGGTAAA
CCGGGCAGTTCCGAGGGCTCGACCAAGGGCGAGGTACAACTGGTGCAGAGCGGCGCGGA
GGTGAAGAAGCCTGGCGAGAGCCTGAAGATCTCATGCAAGGGTTCCGGCTACTCCTTCA
CGGACTATTACATGAAATGGGCCCGCCAGATGCCCGGCAAGGGGCTCGAATGGATGGGG
GACATCATCCCTAGCAACGGCGCCACGTTCTACAATCAGAAGTTCAAGGGACAGGTCAC
AATTTCCGCGGACAAAAGCATCAGCACTACTTACCTGCAGTGGTCTTCCCTCAAGGCTA
GCGACACCGCCATGTATTACTGCGCGAGAAGTCACTTGCTGCGCGCTAGTTGGTTCGCC
TATTGGGGCCAGGGCACCATGGTGACAGTGTCCACGACCACGCCTGCGCCGCGACCGCC
TACTCCCGCGCCAACCATCGCTTCCCAACCCCTGTCCTTGAGGCCCGAGGCCTGCAGAC
CTGCGGCTGGCGGGGCTGTTCACACTCGAGGTTTGGATTTTGCTTGCGACATGTTTTGG
GTGCTGGTGGTGGTCGGAGGTGTCCTGGCTTGTTACTCTCTGCTGGTCACCGTCGCGTT
CATCATCTTCTGGGTGCGGTCTAAGCGCTCACGTGGTGGGCACTCCGACTACATGAACA
TGACGCCCCGACGCCCAGGTCCTACGAGAAAGCATTACCAACCGTACGCTCCACCCCGC
GACTTTGCTGCCTACCGGTCCAGAGTAAAATTCAGTCGGTCTGCTGATGCCCCTGCTTA
CCAACAGGGCCAGAATCAGCTCTACAATGAGCTTAATCTGGGACGACGAGAGGAATATG
ACGTTTTGGACAAGCGAAGGGGCCGAGACCCTGAGATGGGCGGTAAACCGCGGAGAAAG
AACCCCCAGGAGGGGTTGTATAATGAATTGCAAAAGGACAAAATGGCGGAGGCGTATTC
CGAAATAGGGATGAAGGGTGAAAGGCGGCGAGGCAAGGGACATGATGGTCTCTACCAAG
GGCTGTCTACTGCCACGAAGGACACCTATGATGCGCTTCATATGCAGGCCTTGCCTCCG
CGA
-4G8-CSL362-bispecific CAR (CD8a hinge, CD28 TM, CD28z, CD3z) amino
acid sequence targeting both FLT3 and CD123, variant II (epitope binding
regions are italicized, CDRs are bolded)
SEQ ID NO: 79
MLLLVTSLLLCELPHPAFLLIPDIVLTQSPATLSVTPGDSVSLSCRASQSISNNLHWYQ
QKSHESPRLLIKYASQSISGIPSRFSGSGSGTDFTLSINSVETEDFGVYFCQQSNTWPY
TFGGGTKLEIKRGSTSGSGKPGSSEGSTKGQVQLQQPGAELVKPGASLKLSCKSSGYTF
TSYWMHWVRQRPGHGLEWIGEIDPSDSYKDYNQKFKDKATLTVDRSSNTAYMHLSSLTS
DDSAVYYCARAITTTPFDFWGQGTTLTVSSGGGGSGGGGSGGGGSGGGGSGGGGSEVQL
VQSGAEVKKPGESLKISCKGSGYSFTDYYMKWARQMPGKGLEWMGDIIPSNGATFYNQK
FKGQVTISADKSISTTYLQWSSLKASDTAMYYCARSHLLRASWFAYWGQGTMVTVSSGS
TSGSGKPGSSEGSTKGDIVMTQSPDSLAVSLGERATINCESSQSLLNSGNQKNYLTWYQ
QKPGQPPKPLIYWASTRESGVPDRFSGSGSGTDFTLTISSLQAEDVAVYYCQNDYSYPY
TFGQGTKLEIKTTTPAPRPPTPAPTIASQPLSLRPEACRPAAGGAVHTRGLDFACDMFW
VLVVVGGVLACYSLLVTVAFIIFWVRSKRSRGGHSDYMNMTPRRPGPTRKHYQPYAPPR
DFAAYRSRVKFSRSADAPAYQQGQNQLYNELNLGRREEYDVLDKRRGRDPEMGGKPRRK
NPQEGLYNELQKDKMAEAYSEIGMKGERRRGKGHDGLYQGLSTATKDTYDALHMQALPP
R
-4G8-CSL362-bispecific CAR (CD8a hinge, CD28 TM, CD28z, CD3z)
nucleotide sequence targeting both FLT3 and CD123, variant II
SEQ ID NO: 80
ATGCTCCTGCTGGTGACCTCACTGCTGCTTTGCGAGCTGCCCCACCCCGCGTTCCTGCT
GATTCCCGACATCGTGCTTACCCAGTCCCCCGCCACCCTGTCCGTCACCCCCGGCGATT
CTGTCAGCTTAAGTTGTCGCGCTTCCCAGAGCATTAGCAACAACCTGCACTGGTACCAA
CAAAAGTCTCATGAGAGCCCACGGCTGCTGATTAAGTACGCATCACAATCTATCTCTGG
CATCCCCTCGCGCTTTTCTGGGTCCGGCAGTGGAACTGATTTCACCCTCTCCATCAATT
CCGTCGAGACCGAGGACTTTGGGGTGTACTTCTGCCAGCAGTCAAATACCTGGCCTTAC
ACCTTCGGCGGGGGTACGAAGCTGGAGATTAAGCGTGGGTCCACATCCGGCAGCGGTAA
ACCCGGCTCCTCAGAGGGCTCCACAAAAGGCCAGGTTCAGCTGCAGCAGCCTGGCGCGG
AACTGGTTAAGCCCGGCGCAAGTCTGAAGCTGTCTTGTAAGTCTTCGGGTTACACGTTT
ACTTCGTACTGGATGCACTGGGTTCGCCAGCGTCCTGGCCACGGACTGGAATGGATCGG
CGAGATCGATCCTTCCGATTCCTACAAGGACTACAACCAGAAATTTAAGGATAAAGCTA
CTCTAACTGTGGACCGCTCTAGCAACACCGCTTACATGCACCTCAGCTCGCTTACCAGC
GATGACTCTGCGGTGTACTACTGTGCCCGCGCCATCACCACTACGCCATTCGACTTCTG
GGGCCAGGGCACTACTCTTACCGTGTCGTCGGGTGGAGGCGGCTCTGGAGGAGGCGGAA
GCGGAGGAGGTGGTTCTGGAGGCGGAGGCTCTGGTGGAGGCGGATCTGAGGTGCAGCTC
GTCCAGAGCGGGGCAGAGGTGAAGAAGCCTGGTGAGTCCCTTAAGATTTCCTGTAAGGG
CTCGGGCTACTCCTTCACCGACTACTACATGAAATGGGCGCGTCAGATGCCCGGAAAGG
GACTGGAGTGGATGGGGGACATCATCCCGTCCAATGGCGCTACCTTCTACAACCAGAAG
TTCAAGGGCCAGGTGACCATCAGCGCGGACAAATCCATCTCCACCACGTACCTGCAGTG
GTCTAGTCTGAAGGCCTCTGACACAGCCATGTATTATTGCGCCCGCAGCCACTTGCTGC
GCGCGAGTTGGTTCGCCTATTGGGGCCAGGGTACTATGGTGACTGTGAGCTCCGGTTCG
ACTTCCGGAAGTGGAAAACCGGGATCATCCGAAGGCAGCACCAAGGGTGACATAGTGAT
GACCCAGTCCCCGGATAGCCTTGCCGTGTCCCTGGGCGAGCGCGCCACAATCAACTGCG
AGAGCAGCCAGAGCCTGCTCAACTCCGGCAACCAGAAGAACTACCTGACCTGGTACCAA
CAGAAGCCCGGCCAGCCACCTAAGCCCCTGATCTATTGGGCTAGTACGAGGGAGTCCGG
CGTCCCTGATCGCTTCTCTGGCTCTGGGAGCGGTACAGATTTCACCCTGACTATTTCGA
GCCTGCAAGCTGAGGACGTGGCCGTGTACTATTGCCAGAACGACTACAGCTATCCTTAC
ACCTTCGGCCAGGGCACCAAGCTGGAGATCAAGACGACCACGCCTGCGCCGCGACCGCC
TACTCCCGCGCCAACCATCGCTTCCCAACCCCTGTCCTTGAGGCCCGAGGCCTGCAGAC
CTGCGGCTGGCGGGGCTGTTCACACTCGAGGTTTGGATTTTGCTTGCGACATGTTTTGG
GTGCTGGTGGTGGTCGGAGGTGTCCTGGCTTGTTACTCTCTGCTGGTCACCGTCGCGTT
CATCATCTTCTGGGTGCGGTCTAAGCGCTCACGTGGTGGGCACTCCGACTACATGAACA
TGACGCCCCGACGCCCAGGTCCTACGAGAAAGCATTACCAACCGTACGCTCCACCCCGC
GACTTTGCTGCCTACCGGTCCAGAGTAAAATTCAGTCGGTCTGCTGATGCCCCTGCTTA
CCAACAGGGCCAGAATCAGCTCTACAATGAGCTTAATCTGGGACGACGAGAGGAATATG
ACGTTTTGGACAAGCGAAGGGGCCGAGACCCTGAGATGGGCGGTAAACCGCGGAGAAAG
AACCCCCAGGAGGGGTTGTATAATGAATTGCAAAAGGACAAAATGGCGGAGGCGTATTC
CGAAATAGGGATGAAGGGTGAAAGGCGGCGAGGCAAGGGACATGATGGTCTCTACCAAG
GGCTGTCTACTGCCACGAAGGACACCTATGATGCGCTTCATATGCAGGCCTTGCCTCCG
CGA
-CSL362-CAR 2nd variant (IgG4 hinge, CD28 TM, CD28z, CD3z) amino
acid sequence targeting CD123 N-terminal domain (CDRs are bolded)
SEQ ID NO: 86
MLLLVTSLLLCELPHPAFLLIPEVQLQQSGPELVKPGASVKMSCKASGYTFTDYYMKWV
KQSHGKSLEWIGDIIPSNGATFYNqKFKGKATLTVDRSSSTAYMHLNSLTSEDSAVYYC
TRSHLLRASWFAYWGQGTLVTVSSGGGGSGGGGSGGGGSDFVMTqSPSSLTVTAGEKVT
MSCKSSQSLLNSGNQKNYLTWYLqKPGqPPKLLIYWASTRESGVPDRFTGSGSGTDETL
TISSVQAEDLAVYYCqNDYSYPYTFGGGTKLEIKRESKYGPPCPPCPASMFWVLVVVGG
VLACYSLLVTVAFIIFWVRSKRSRGGHSDYMNMTPRRPGPTRKHYQPYAPPRDFAAYRS
RVKFSRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR
-CSL362-CAR 3rd variant (IgG4 hinge, CD28 TM, CD28z, CD3z)
amino acid sequence targeting CD123 N-terminal domain (CDRs are bolded)
SEQ ID NO: 87
MLLLVTSLLLCELPHPAFLLIPEVQLVQSGAEVKKPGESLKISCKGSGYSFTDYYMKWA
RQMPGKGLEWMGDIIPSNGATFYNQKFKGQVTISADKSISTTYLQWSSLKASDTAMYYC
ARSHLLRASWFAYWGQGTMVTVSSGGGGSGGGGSGGGGSDIVMTQSPDSLAVSLGERAT
INCESSQSLLNSGNQKNYLTWYQQKPGQPPKPLIYWASTRESGVPDRFSGSGSGTDETL
TISSLQAEDVAVYYCQNDYSYPYTFGQGTKLEIKRESKYGPPCPPCPASMFWVLVVVGG
VLACYSLLVTVAFIIFWVRSKRSRGGHSDYMNMTPRRPGPTRKHYQPYAPPRDFAAYRS
RVKFSRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPR

### III. Methods of Treating a Subject

The genetically engineered hematopoietic cells such as HSCs can be administered to a subject in need of the treatment, either taken alone or in combination of one or more cytotoxic agents that target one or more cell-surface antigens as described herein. Since the hematopoietic cells are genetically edited in the genes of the one or more cell-surface antigens, the hematopoietic cells and/or descendant cells thereof would express the one or more cell-surface antigens in mutated form (e.g., but functional) such that they can escape being targeted by the cytotoxic agents, for example, CAR-T cells.

Thus, the present disclosure provides methods for treating a hematopoietic malignancy, the method comprising administering to a subject in need thereof (i) a population of the genetically engineered hematopoietic cells described herein, and optionally (ii) a cytotoxic agent such as CAR-T cells that target a cell-surface antigen, the gene of which is genetically edited in the hematopoietic cells such that the cytotoxic agent does not target hematopoietic cells or descendant cells thereof. The administration of (i) and (ii) can be concurrently or in any order. In some embodiments, the cytotoxic agents and/or the hematopoietic cells can be mixed with a pharmaceutically acceptable carrier to form a pharmaceutical composition, which is also within the scope of the present disclosure.

As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human patient having a hematopoietic malignancy.

To perform the methods described herein, an effective amount of the genetically engineered hematopoietic cells can be administered to a subject in need of the treatment. Optionally, the hematopoietic cells can be co-used with a cytotoxic agent as described herein. As used herein the term “effective amount” can be used interchangeably with the term “therapeutically effective amount” and refers to that quantity of a cytotoxic agent, hematopoietic cell population, or pharmaceutical composition (e.g., a composition comprising cytotoxic agents and/or hematopoietic cells) that is sufficient to result in a desired activity upon administration to a subject in need thereof. Within the context of the present disclosure, the term “effective amount” refers to that quantity of a compound, cell population, or pharmaceutical composition that is sufficient to delay the manifestation, arrest the progression, relieve or alleviate at least one symptom of a disorder treated by the methods of the present disclosure. Note that when a combination of active ingredients is administered the effective amount of the combination can or cannot include amounts of each ingredient that would have been effective if administered individually.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject is a human patient having a hematopoietic malignancy.

As described herein, the hematopoietic cells and/or immune cells expressing chimeric receptors can be autologous to the subject, i.e., the cells are obtained from the subject in need of the treatment, manipulated such that the cells do not bind the cytotoxic agents, and then administered to the same subject. Administration of autologous cells to a subject can result in reduced rejection of the host cells as compared to administration of non-autologous cells. For example, HSPCs are obtained from a biological sample from a subject, the HSPCs are genetically engineered, and the genetically engineered HSPCs are administered to the same subject. In some instances, the HSPCs are obtained from a biological sample, wherein the biological sample is bone marrow cells, blood, cord blood cells, or mobilized peripheral blood-derived CD34+ hematopoietic stem and progenitor cells.

Alternatively, the host cells are allogeneic cells, i.e., the cells are obtained from a first subject, genetically engineered, and then administered to a second subject that is different from the first subject but of the same species. For example, allogeneic immune cells can be derived from a human donor and administered to a human recipient who is different from the donor. In some embodiments, the hematopoietic cells have been further genetically engineered to reduce host-versus-graft effects. For example, in some embodiments, immune cells and/or hematopoietic cells can be subjected to gene editing or silencing methods to reduce or eliminate expression of one or more proteins involved in inducing host immune responses.

A typical amount of cells, i.e., immune cells or hematopoietic cells, administered to a mammal (e.g., a human) can be, for example, in the range of about 10⁶ to 10¹¹ cells. In some embodiments it can be desirable to administer fewer than 10⁶ cells to the subject. In some embodiments, it can be desirable to administer more than 10¹¹ cells to the subject. In some embodiments, one or more doses of cells includes about 10⁶ cells to about 10¹¹ cells, about 10⁷ cells to about 10¹⁰ cells, about 10⁸ cells to about 10⁹ cells, about 10⁶ cells to about 10⁸ cells, about 10⁷ cells to about 10⁹ cells, about 10⁷ cells to about 10¹⁰ cells, about 10⁷ cells to about 10¹¹ cells, about 10⁸ cells to about 10¹⁰ cells, about 10⁸ cells to about 10¹¹ cells, about 10⁹ cells to about 10¹⁰ cells, about 10⁹ cells to about 10¹¹ cells, or about 10¹⁰ cells to about 10¹¹ cells.

In some embodiments, the methods described herein involve administering a population of genetically engineered hematopoietic cells to a subject and administering one or more immunotherapeutic agents (e.g., cytotoxic agents). As will be appreciated by one of ordinary skill in the art, the immunotherapeutic agents can be of the same or different type (e.g., therapeutic antibodies, populations of immune cells expressing chimeric antigen receptor(s), and/or antibody-drug conjugates).

In some embodiments, the cytotoxic agent comprising an epitope binding fragment that binds an epitope of a cell-surface protein (e.g., immune cells expressing a chimeric receptor as described herein) is administered prior to administration of the hematopoietic cells. In some embodiments, the agent comprising an epitope binding fragment that binds an epitope of a cell-surface protein (e.g., immune cells expressing a chimeric receptor as described herein) is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the hematopoietic cells.

Alternatively, in some embodiments, the hematopoietic cells are administered prior to the cytotoxic agent comprising an epitope binding fragment that binds an epitope of the cell-surface protein (e.g., immune cells expressing a chimeric receptor as described herein). In some embodiments, the population of hematopoietic cells is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the cytotoxic agent comprising an epitope binding fragment that binds to an epitope of the cell-surface protein.

In some embodiments, the cytotoxic agent targeting the cell-surface protein and the population of hematopoietic cells are administered at substantially the same time. In some embodiments, the cytotoxic agent targeting the cell-surface protein is administered and the patient is assessed for a period of time, after which the population of hematopoietic cells is administered. In some embodiments, the population of hematopoietic cells is administered and the patient is assessed for a period of time, after which the cytotoxic agent targeting the cell-surface protein is administered.

Also within the scope of the present disclosure are multiple administrations (e.g., doses) of the cytotoxic agents and/or populations of hematopoietic cells. In some embodiments, the cytotoxic agents and/or populations of hematopoietic cells are administered to the subject once. In some embodiments, cytotoxic agents and/or populations of hematopoietic cells are administered to the subject more than once (e.g., at least 2, 3, 4, 5, or more times). In some embodiments, the cytotoxic agents and/or populations of hematopoietic cells are administered to the subject at a regular interval, e.g., every six months.

Examples of routes of administration include intravenous, infusion, intradermal, subcutaneous, oral (e.g., inhalation), transdermal (topical), transmucosal, and rectal administration.

Any of the methods described herein can be for the treatment of a hematological malignancy in a subject. The term “treat” or “treatment” or “treating” or “to treat” as used herein refers to therapeutic measures that aim to relieve, slow down progression of, lessen symptoms of, and/or halt progression of a pathologic condition or disorder. Thus, those in need of treatment include those already with the disorder. For example, in some instances, treating a cancer means stabilizing progression of the cancer. In some instances, treating a cancer means slowing down progression of the cancer. In some instances, treating a cancer means halting progression of the cancer. In some instances, treating a cancer means shrinking the cancer size. In some instances, treating a cancer means increasing the overall survival of the subject diagnosed with the cancer. Methods of assessing the progression of a cancer are known in the art and include, for example, evaluation of target lesions using imaging (e.g., X-ray, computerized tomography scan, magnetic resonance imaging, caliper measurement, or positron emission tomography scan), cytology or histology, or expression of tumor marker(s) (see, e.g., Eisenhauer et al., 2009, European Journal of Cancer 45:228-247 and Schwartz et al., 2016, European Journal of Cancer 62:132-137; each of which is incorporated by reference herein in its entirety).

In some embodiments, the subject is a human subject having a hematopoietic malignancy. As used herein a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Exemplary leukemias include, without limitation, acute myeloid leukemia, acute lymphoid leukemia, chronic myelogenous leukemia, acute lymphoblastic leukemia or chronic lymphoblastic leukemia, and chronic lymphoid leukemia.

In some embodiments, cells involved in the hematopoietic malignancy are resistant to conventional or standard therapeutics used to treat the malignancy. For example, the cells (e.g., cancer cells) can be resistant to a chemotherapeutic agent and/or CAR T cells used to treat the malignancy.

In some instances, the hematopoietic malignancies include: B-lymphoblastic leukemia (B-ALL), acute myeloid leukemia (AML), T-cell acute lymphoblastic leukemia (T-ALL), or Blastic Plasmacytoid Dendritic Cell Leukemia (BPCDN).

### IV. Compositions and Kits

Any of the immune cells expressing chimeric receptors described herein can be administered in a pharmaceutically acceptable carrier or excipient as a pharmaceutical composition.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and can comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

Also within the scope of the present disclosure are kits for use in treating hematopoietic malignancy. Such a kit can comprise the genetically engineered hematopoietic cells such as HSPCs, and optionally one or more cytotoxic agents targeting cell-surface antigens, the genes of which are edited in the hematopoietic cells. Such kits can include a container comprising a first pharmaceutical composition that comprises any of the genetically engineered hematopoietic cells as described herein, and optionally one or more additional containers comprising one or more cytotoxic agents (e.g., immune cells expressing chimeric receptors described herein) targeting the cell-surface antigens as also described herein.

In some embodiments, the kit can comprise instructions for use in any of the methods described herein. The included instructions can comprise a description of administration of the genetically engineered hematopoietic cells and optionally descriptions of administration of the one or more cytotoxic agents to a subject to achieve the intended activity in a subject. The kit can further comprise a description of selecting a subject suitable for treatment based on identifying whether the subject is in need of the treatment. In some embodiments, the instructions comprise a description of administering the genetically engineered hematopoietic cells and optionally the one or more cytotoxic agents to a subject who is in need of the treatment.

The instructions relating to the use of the genetically engineered hematopoietic cells and optionally the cytotoxic agents described herein generally include information as to dosage, dosing schedule, and route of administration for the intended treatment. The containers can be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit can have a sterile access port (for example, the container can be an intravenous solution bag or a vial having a stopper pierceable by a hypodermic injection needle). The container can also have a sterile access port. At least one active agent in the pharmaceutical composition is a chimeric receptor variants as described herein.

Kits optionally can provide additional components such as buffers and interpretive information. Normally, the kit comprises a container and a label or package insert(s) on or associated with the container. In some embodiment, the disclosure provides articles of manufacture comprising contents of the kits described above.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: General Protocols Used in the Following Examples

### Plasmid Cloning

WT-Cas9 and NG-Cas9 base editor plasmids were obtained through Addgene (plasmid #138495, 138491). SpRY-ABE8e-V106W 3xNLS and other base editor variants (see complete list below) were cloned using NEB HiFi assembly master mix and synthesized dsDNA inserts (IDT gBlocks). Single amino acid changes (i.e., K918N) or deletions (Blackjack variants) were introduced through standard site-specific mutagenesis techniques. When required, sgRNAs were cloned in a pLentiguide-Puro backbone (Addgene) or a pLKO-mTagBFP2 backbone (cloned) using the BsmBI restriction enzyme and annealed and phosphorylated DNA oligos with desired spacer sequence. Plasmid maxipreps were purified with Mackarey Nagel NucleoBond Xtra Maxi kit.

TABLE 3

| Base | | | |
|---|---|---|---|
| Editor | Full name | Description | Origin |
| ABE | ABE8e(TadA-8e V106W) | NGG PAM, reduced RNA | Addgene |
| | | editing (V106W) | |
| ABE | NG-ABE8e | NG PAM | Addgene |
| ABE | SpRY -ABE8e-TadA-8e | NRN PAM, 3xNLS | cloned |
| | V106W 3xNLS | | |
| ABE | LbABE8e(TadA-8e | TTTV PAM, reduced RNA | Addgene |
| | V106W) | editing (V106W) | |
| ABE | enAsABE8e | TTTV PAM | Addgene |
| ABE | dABE8e TadA 8e V106W | NGG PAM, dCas9 | cloned |
| ABE | ABE8e-TadA-8e V106W | NGG PAM, 3xNLS | cloned |
| | 3xNLS | | |
| ABE | NG-ABE8e 3xNLS | NG PAM, 3xNLS | cloned |
| ABE | LbABE8e(TadA-8e | TTTV PAM, 3xNLS | cloned |
| | V106W) 3xNLS | | |
| ABE | RVRR-LbABE8e(TadA-8e | PAM-relaxed LbCas12a variant | cloned |
| | V106W) 2xNLS | | |
| ABE | SpRY-K918N-ABE8e- | NRN PAM, K918N, 3xNLS | cloned |
| | TadA-8e V106W 3xNLS | | |
| ABE | SpRY-Sniper-ABE8e- | NRN PAM, high fidelity Sniper | cloned |
| | TadA-8e V106W 3xNLS | variant, 3xNLS | |
| ABE | dSpRY-ABE8e-TadA-8e | NRN PAM, dCas9, 3xNLS | cloned |
| | V106W 3xNLS | | |
| ABE | SpRY-BlackJack-ABE8e- | NRN PAM, long gRNA-tolerant | cloned |
| | TadA-8e V106W 3xNLS | variant (Blackjack), 3xNLS | |
| ABE | SpRY-Sniper-BlackJack- | NRN PAM, high fidelity Sniper | cloned |
| | ABE8e V106W 3xNLS | variant, Blackjack, 3xNLS | |
| ABE | SpRY-Sniper-K918N- | NRN PAM, high fidelity Sniper | cloned |
| | ABE8e V106W 3xNLS | variant, K918N, 3xNLS | |
| ABE | SpRY-HF1-ABE8e-TadA- | NRN PAM, high fidelity HF1 | cloned |
| | 8e V106W 3xNLS | variant, 3xNLS | |
| ABE | SpRY-HF1-BlackJack- | NRN PAM, high fidelity HF1 | cloned |
| | ABE8e V106W 3xNLS | variant, Blackjack, 3xNLS | |
| CBE | evoAPOBEC1-BE4max | NGG PAM, evoAPOBEC1 | Addgene |
| | | deaminase | |
| CBE | NG-evoAPOBEC1- | NG PAM, evoAPOBEC1 | Addgene |
| | BE4max | deaminase | |
| CBE | evoAPOBEC1-BE4max | NGG PAM, evoAPOBEC1 | cloned |
| | 3xNLS | deaminase 3xNLS | |
| CBE | SpRY-evoAPOBEC1-BE4 | NRN PAM, evoAPOBEC1 | cloned |
| | 3xNLS | deaminase 3xNLS | |
| CBE | eA3A-BE5-NG | NG PAM, eAPOBEC3A | Addgene |
| | | deaminase, high purity | |
| CBE | EA-BE4max-NG | NG PAM, evoAPOBEC1 | Addgene |
| | | deaminase, high purity | |

### Flow Cytometry Ligand Affinity Assay

In order to evaluate the binding affinity of the mutated receptors for their ligand, a fluorescent ligand binding assay was developed. Human SCF and FLT3L (Peprotech) were conjugated with Alexa Fluor 488 Antibody Labeling Kit (Invitrogen cat. A20181) according to manufacturer's recommendations. Cells expressing either FLT3 or KIT variants were incubated at room temperature for 15 min with FcR-blocking reagent (Miltenyi 130-059-901), LIVE/DEAD Fixable Yellow Dead Cell Stain (Invitrogen L34967), a control antibody (KIT 104D2 PE-Cy7, Biolegend, or FLT3 BV10A4 PE-Cy7, Biolegend) and the respective AF488-conjugated ligand. Samples were washed with PBS+2% FBS and analyzed with a BD Fortessa flow cytometer.

### Reporter Cell Lines for FLT3 and CD123 Editing

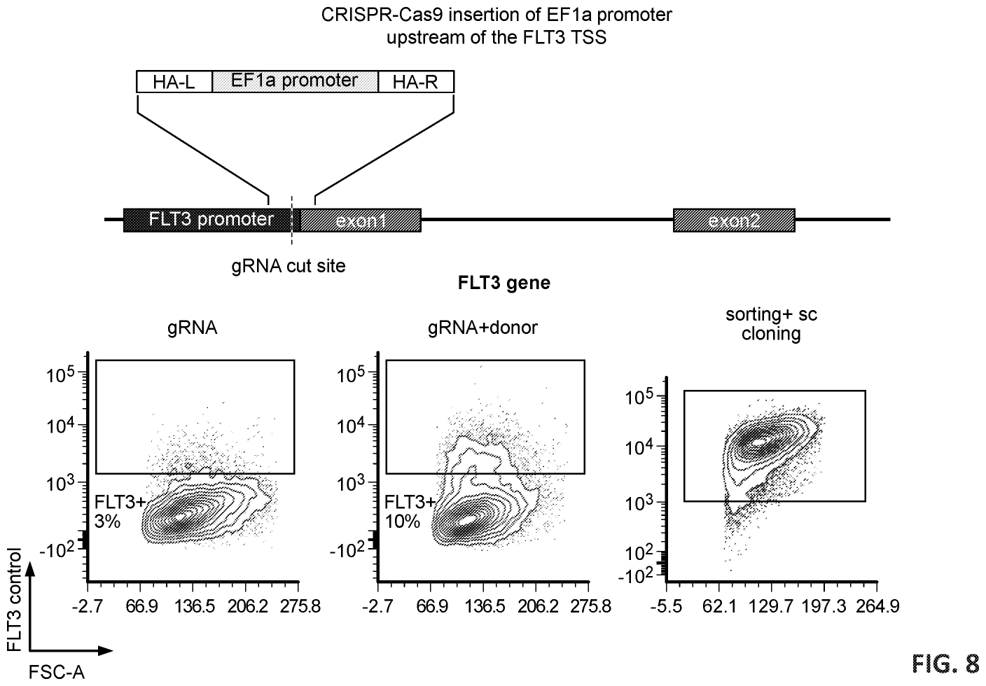

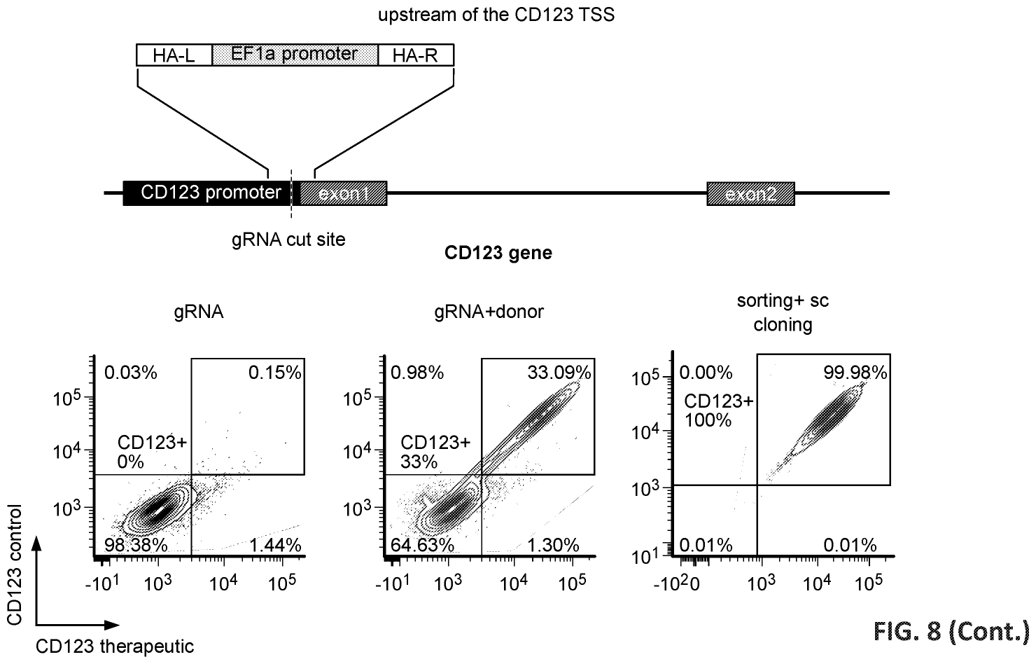

As K562 cells do not constitutively express FLT3 or CD123, these cells were engineered to over-express either FLT3, CD123, KIT or any combinations of the three genes from their endogenous genomic locus to serve as a model for gene editing approaches on these genes. In order to obtain constitutive strong expression of these genes, a full human EF1 alpha promoter was integrated upstream of the transcriptional start site of the FLT3, CD123 or KIT genes through CRISPR-Cas9 (FLT3, KIT) or CRISPR-AsCas12a (CD123) gene editing strategies (FIG. 8). A dsDNA linear donor bearing 50-bp long homology arms for the targeted regions was prepared by PCR on a sleeping beauty plasmid encoding for the EF1 alpha promoter. Wild-type K562 cells were electroporated using the Lonza 4D-Nucleofector system with Cas9 (FLT3, KIT) or Cas12a (CD123) RNPs with gRNAs targeting the promoter region of the respective gene. 5 to 10 ug of dsDNA linear donor with the matched homology arms was included in the electroporation reaction to serve as template for homology directed repair integration. Cells were stained with FLT3 BV10A4 PE-Cy7 (Biolegend), KIT 104D2 PE-Cy (Biolegend) or CD123 9F5 BV421 and CD123 7G3 BV711 (BD) and evaluated by flow cytometry. The FLT3, CD123 or KIT-high population was FACS-sorted and single clones were isolated by limiting dilution (FIG. 8). The clones with the highest MFI by flow cytometry for FLT3 (clone AH11) and CD123 (clone 3D5) were selected for expansion.

### Western Blot

K562 cells overexpressing FLT3/CD123 variants or NIH-3T3 or HEK-293T cells overexpressing KIT were cultured overnight in medium without FBS (serum starvation) and then 1 million cells from each condition were either stimulated or not with 100 ng/ml FLT3L, IL3 or SCF (depending on the evaluated receptor) for 5′ at 37° C. Cells were then washed 3× with ice cold PBS, lysed and proteins were extracted for western blot (Cell Extraction Buffer, ThermoFisher FNN0011+1 mM PMSF). After quantification with a BCA assay, protein extracts were mixed with Laemmli loading dye (Biorad 161-0747) and run on a Novex Tris-Glycine Gel (Invitrogen). After transfer, membranes were blocked with 5% w/v BSA in TBST and incubated with primary antibodies recognizing pKIT (Y719, Cell Signaling 3391T) or pFLT3 (Tyr589/591 clone 30D4, Cell Signaling 3464S) overnight at 4° C. After washing, membranes were incubated with Anti-rabbit IgG, HRP-linked Antibody (Cell Signaling 7074) for 1 hour at room temperature and later with SuperSignal West Femto chemiluminescent HRP-substrate (Thermo Scientific 34096) and analyzed with an ImageQuant LAS4000. The same membranes were subsequently incubated with Restore (ThermoScientific #21059) stripping buffer for 20 minutes at room temperature for a secondary staining with anti-KIT (clone 1C5, Invitrogen MA5-15894) or anti FLT3 (clone OTI7D6, Origene TA808157) primary antibodies with the same procedure described above. Secondary Ab staining was performed with anti-rabbit or anti-mouse IgG HRP-conjugated Abs according to the primary Ab and membranes were developed and acquired as above. Anti-actin staining was used as internal control for loading normalization.

### Flow Cytometry Analysis (In Vitro Experiments)

Edited cell lines were evaluated by flow cytometry at 72 hours post editing and stained with antibodies clones either binding to the therapeutic epitope or an unrelated epitope to serve as control antibody for surface expression of the edited protein. For FLT3 editing, cells were incubated with FcR-blocking reagent (Miltenyi 130-059-901) 2/100 μL, LIVE/DEAD Fixable Yellow Dead Cell Stain (Invitrogen L34967) 1/1000, FLT3 BV10A4 PE-Cy7 2/100 (Biolegend 313314) and FLT3 4G8 BV711 (BD 563908). For CD123 editing, cells were incubated with FcR-blocking reagent (Miltenyi 130-059-901) 2/100 μL, LIVE/DEAD Fixable Yellow Dead Cell Stain (Invitrogen L34967) 1/1000, CD123 9F5 PE or BV421 (BD 555644) 1/100, CD123 7G3 BV711 or BV421 (BD 740722) 1/100. For KIT editing, cells were incubated with FcR-blocking reagent (Miltenyi 130-059-901) 2/100 μL, LIVE/DEAD Fixable Yellow Dead Cell Stain (Invitrogen L34967) 1/1000, KIT 104D2 PE-Cy7 or BV711 2/100 (Biolegend 313212), KIT Fab79D AF488 or PE (Creative Biolabs) 2/100. Staining was performed at 4° C. for 30 minutes with 100 μL/sample volume. To evaluate the stem cell phenotype of cultured human CD34+ HSPCs, cells were harvested, resuspended in 100 μL PBS and stained with FcR-blocking reagent (Miltenyi 130-059-901) 2/100 μL, LIVE/DEAD Fixable Yellow Dead Cell Stain (Invitrogen L34967) 1/1000, CD34 BV421 (Biolegend) 1.5/100, CD90 APC (BD) 3.5/100, CD45RA APC-Cy7 (Biolegend) 3.5/100, CD133/2 PE (Miltenyi) 4/100. Staining was performed at 4° C. for 30 minutes with 100 μL/sample volume. Samples were analyzed on a 4- or 5-laser BD Fortessa flow cytometer.

### Flow Cytometry Analysis (In Vivo Experiments)

Peripheral blood from xeno-transplanted NSG mice was collected in 1.5 mL Eppendorf tubes with 10 μL 0.5M EDTA and stained with FcR-blocking reagent (Miltenyi 130-059-901) 2/100 μL, LIVE/DEAD Fixable Yellow Dead Cell Stain (Invitrogen L34967) 1/1000, human CD45 BV786 (Biolegend) 1.5/100, mouse CD45 BV570 (Biolegend), CD34 BV421 (Biolegend) 1.5/100, CD19 BV650 (Biolegend) 3/100, CD3 BV711 (Biolegend) 2/100, CD33 BB515 (BD) 2/100 for 15 minutes at room temperature. Blood samples were then lysed with ACK reagent (StemCell technologies) for 5 minutes at room temperature and washed twice in PBS+2% FBS. Samples were analyzed on a 4-laser BD Fortessa flow cytometer.

Bone marrow from xeno-transplanted mice was obtained through crushing the hind limbs bones in a mortar, filtration through a 40-um cell strainer and resuspending the cells in Miltenyi MACS running buffer. A fraction of the cells was stained for flow cytometry analysis: with FcR-blocking reagent (Miltenyi 130-059-901) 2/100 uL, 7-AAD (BD Pharmigen) 3/100, human CD45 BV786 (Biolegend) 1.5/100, mouse CD45 BV570 (Biolegend), CD34 BV421 (Biolegend) 1.5/100, CD19 BV650 (Biolegend) or CD19 BV605 (Biolegend) 3/100, CD3 BV711 (Biolegend) or CD3 PE-Cy7 (Biolegend) 2/100CD33 BB515 (BD), CD38 BV480 (BD) 1.5/100 or CD38 BUV396 (BD) 2/100, FLT3 BV10A4 PE-Cy7 (Biolegend), CD123 9F5 PE (BD), CD90 APC (BD) 3.5/100, CD45RA APC-Cy7 (Biolegend) 3/100 for 30 minutes at 4° C. Spleens were smashed on a 40 μm cell strainer and resuspended in Miltenyi MACS running buffer. Retrieved cells were stained with FcR-blocking reagent (Miltenyi 130-059-901) 2/100 uL, LIVE/DEAD Fixable Yellow Dead Cell Stain (Invitrogen L34967) 1/1000, human CD45 Pacific Blue (Biolegend) 1.5/100, mouse CD45 BV570 (Biolegend), CD3 BV711 (Biolegend) 2/100, EGFR AF488 (R&D) 1.5/100, CD62L PE (BD) 2/100, CD4 APC (BD) 2/100, CD8 BV750 (Biolegend) 2/100, CD45RA APC-Cy7 (Biolegend) 3/100, CD69 PerCP-Cy5.5 (Biolegend) 3/100 for 30 minutes at 4° C. Samples were analyzed on a 4-laser BD Fortessa flow cytometer. In some experiments, BM cells were stained with hCD45 BV786, mCD45 PerCP-Cy5.5, CD3 PE-Cy5, CD7 AF700, CD10 BUV737, CD11c BUV661, CD14 BV510, CD19 BV605, CD33 PE-Cy7, CD38 BUV396, CD45RA APC-Cy7, CD56 BUV496, CD90 APC, FLT3 PE or BV711 and either KIT BV711 or CD123 PE antibodies with the addition of 50 uL/sample Brilliant stain buffer (BD cat.no. 659611).

gDNA Extraction, PCR Amplification and Sanger Sequencing

Genomic DNA was extracted from dry pellet samples using Qiagen DNeasy Blood & Tissue Kit or Lucigen QuickExtract reagent, quantified by Nanodrop 8000 and the sequences of interest were amplified by PCR using Promega GoTaq G2 and the respective primers. PCR products were purified using Promega SV Wizard Gel and PCR Clean-Up system and sent for Sanger sequencing through Genewiz. Base editing efficiencies were calculated from Sanger traces by deconvolution with the EditR R package using a custom script for high-throughput sample analysis.

### Colony Forming Assays

Colony forming Unit assays (CFU) were performed by plating 1000 CD34+ cells/well, for in vitro CD34+ HSPCs experiments, or 25000 total bone marrow cells/wells, for xeno-transplanted BM-derived assays, unless stated otherwise. Cells were resuspended in Methocult H4034 media (StemCell cat.no. 04034) and plated in SmartDish meniscus-free 6-well plates. Wells were imaged after 2 weeks using StemCell STEMvision system. For flow cytometry analysis, methylcellulose media was softened with warm PBS, collected and washed twice before analysis.

### Example 2: Receptor Variants Design

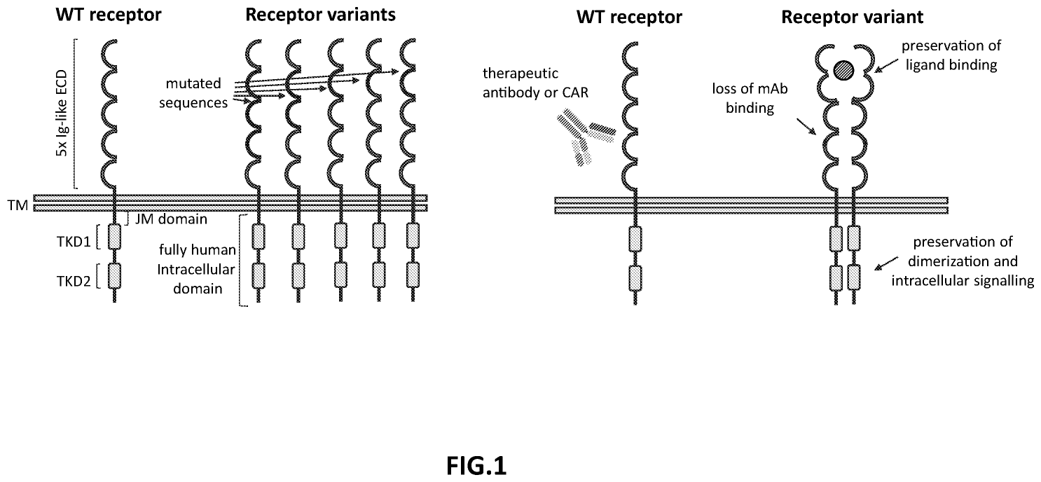

In order to design mutated receptor variants which would not be recognized by selected monoclonal antibody clones, the epitopes targeted by different mAb were identified according to available information in the literature or by screening individual mutated receptor variants or mutation libraries. The overall goal was the identification of minimally modified target variants that preserve surface expression, gene regulation and signal transduction functionality while lacking recognition by selected therapeutic antibody clones (FIG. 1). Cells bearing such modified surface targets were endowed with selective resistance when exposed to immunotherapies against the target molecule (including but not limited to naked monoclonal antibodies, toxin conjugated antibodies, bispecific antibodies constructs and chimeric antigen receptor cells).

### FLT3 Epitope Engineering:

FLT3 is a type III tyrosine kinase receptors composed by i) an extracellular ligand binding domain, characterized by the presence of five immunoglobulin-like domains; ii) a single spanning transmembrane region; iii) an intracellular part containing a split tyrosine kinase domain. The first 3 extracellular domains are involved in the binding with its dimeric ligand, FLT3-ligand (FLT3L), and this interaction induced the dimerization of the receptor. After dimerization, FLT3 activation is mediated by close positioning of the intracellular tyrosine kinase domains to each other, which facilitates their subsequent transphosphorylation.

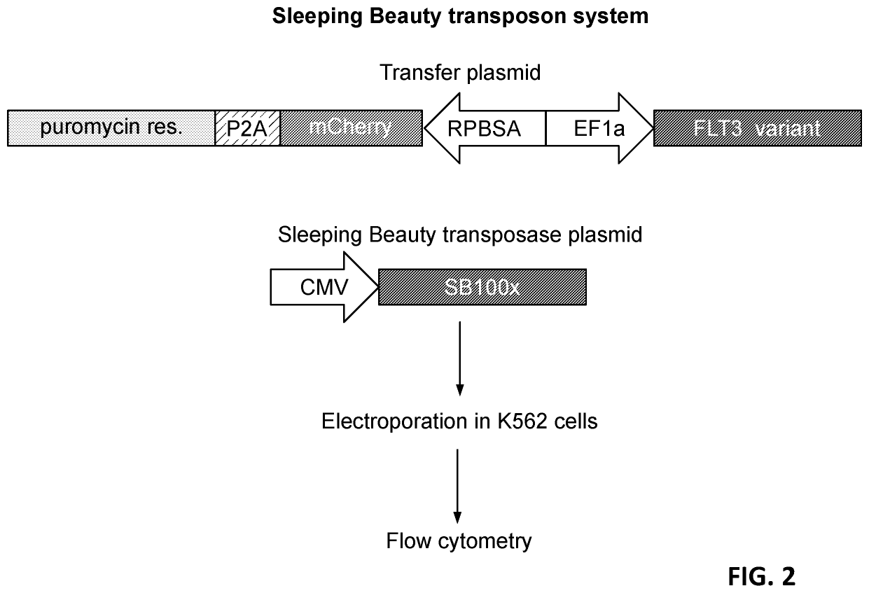

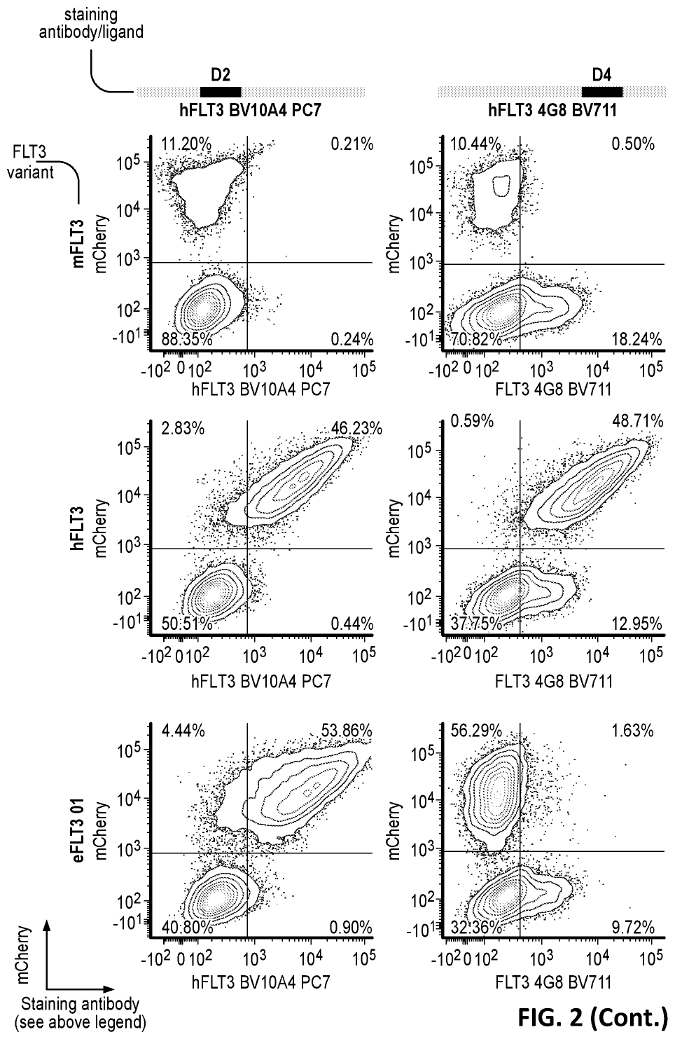

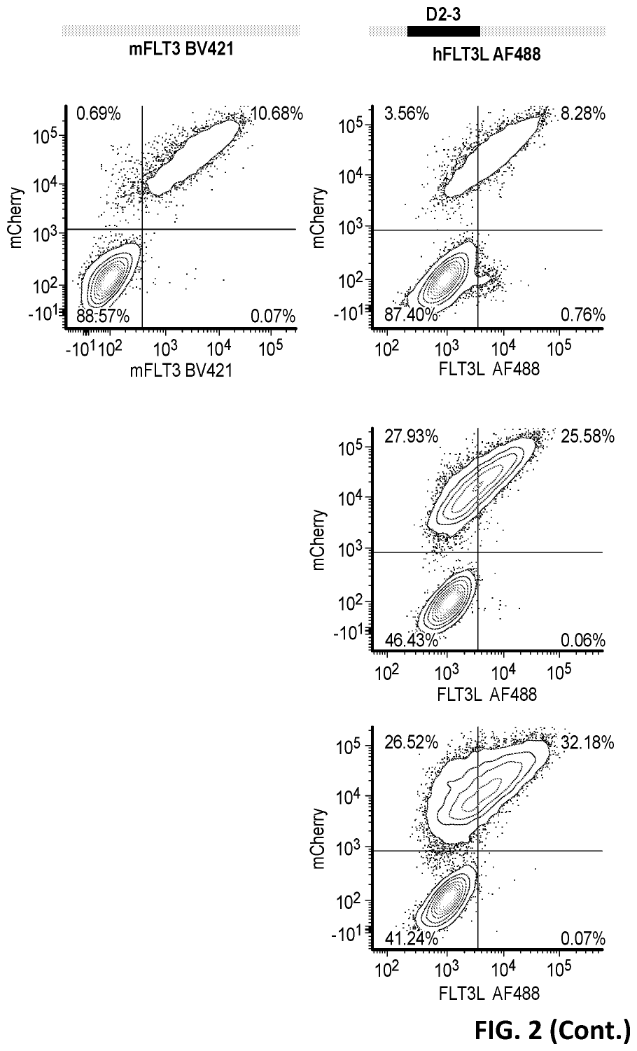

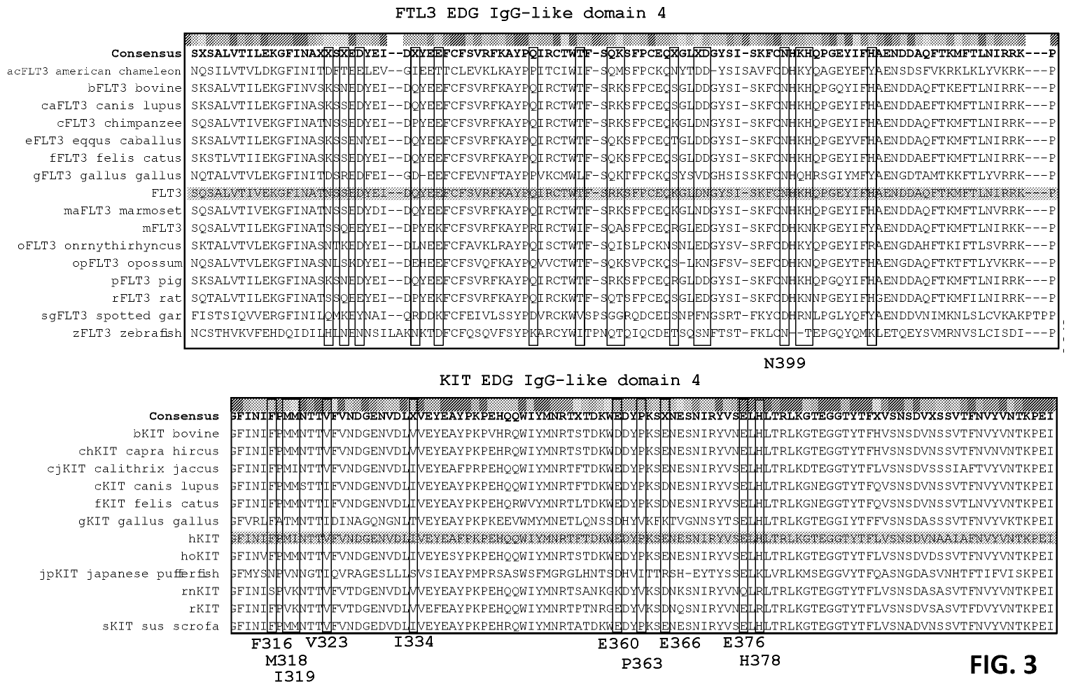

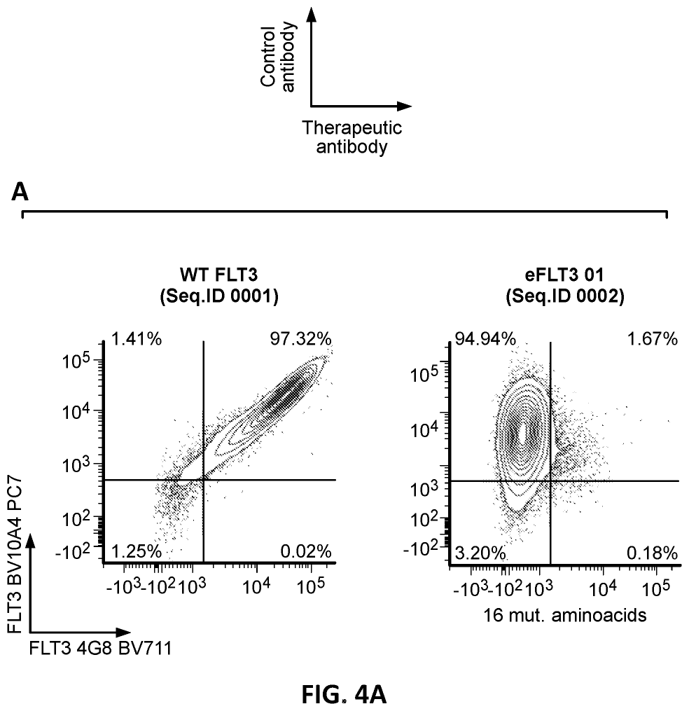

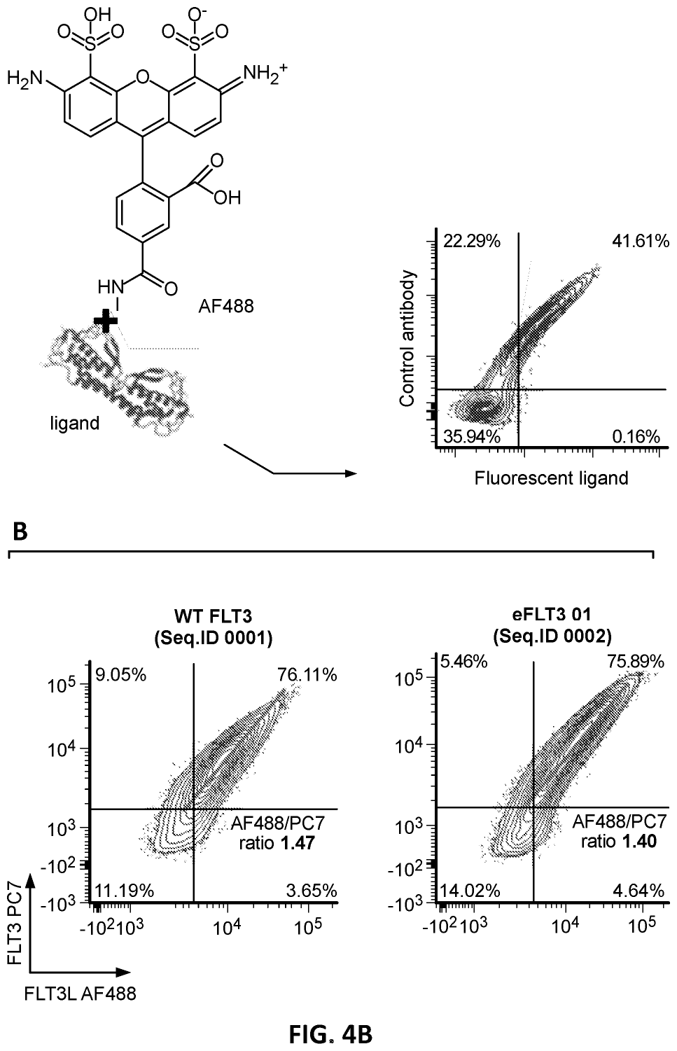

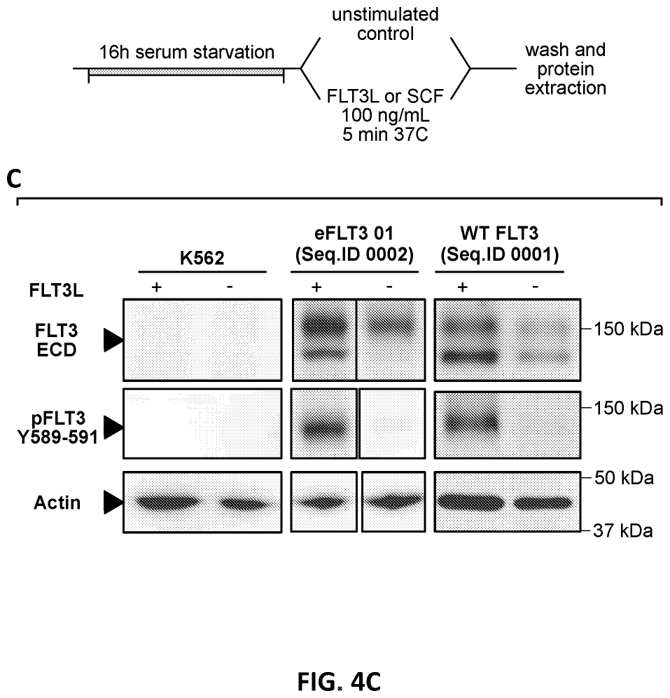

Alignments show that mouse and human FLT3/FLT3L are 85.5% identical at the amino-acid level and that mouse and human FLT3 IgG-like domain 4 are 82% identical (95.5% similar) at the amino-acid level (FIG. 3). Furthermore, mouse and human FLT3/FLT3L pairs are mutually cross-reactive. The anti-human FLT3 therapeutic antibody clone 4G8 specifically recognizes human FLT3 and the epitope is localized within extracellular domain 4. It was confirmed that clone 4G8 does not recognize other ortholog variants (i.e. mouse Flt3) by staining K562 cells overexpressing wild type human or murine FLT3 through a sleeping beauty transposon system in which either human FLT3 or murine Flt3 cDNA sequences were cloned downstream of a constitutive EF1α promoter (FIG. 2). An mCherry fluorescent reporter encoded by the sleeping beauty transposon was used to identify transduced cells. Based on this evidence, 16 residues located in the extracellular domain 4, which were relatively less preserved across ortholog sequences (FIG. 3), were substituted with the corresponding murine amino acids and the generated variant (eFLT3-01, SEQ ID NO: 49) was not recognized by clone 4G8 (FIG. 2). The generated variant still preserved surface expression (confirmed using control anti-FLT3 clone BV10A4, which binds extracellular domain 2), FLT3L binding (see FIGS. 4A and 4B) and intracellular signal transduction properties. (see FIG. 4C).

As a second step, the 16 mutations introduced to generate eFLT3-01 (SEQ ID NO: 49), were separated in two pools of mutations by their genomic localization within either FLT3 exon 9 or exon 10 (which together encode for FLT3 extracellular domain 4). Overexpression of these two FLT3 variants showed that the mutations restricted to FLT3 exon 9 (SEQ ID NO: 50) were sufficient to abrogate anti FLT3 clone 4G8, similarly to SEQ ID NO: 49. The FLT3 variant with mutations limited to exon 10, did not abrogate clone 4G8 binding.

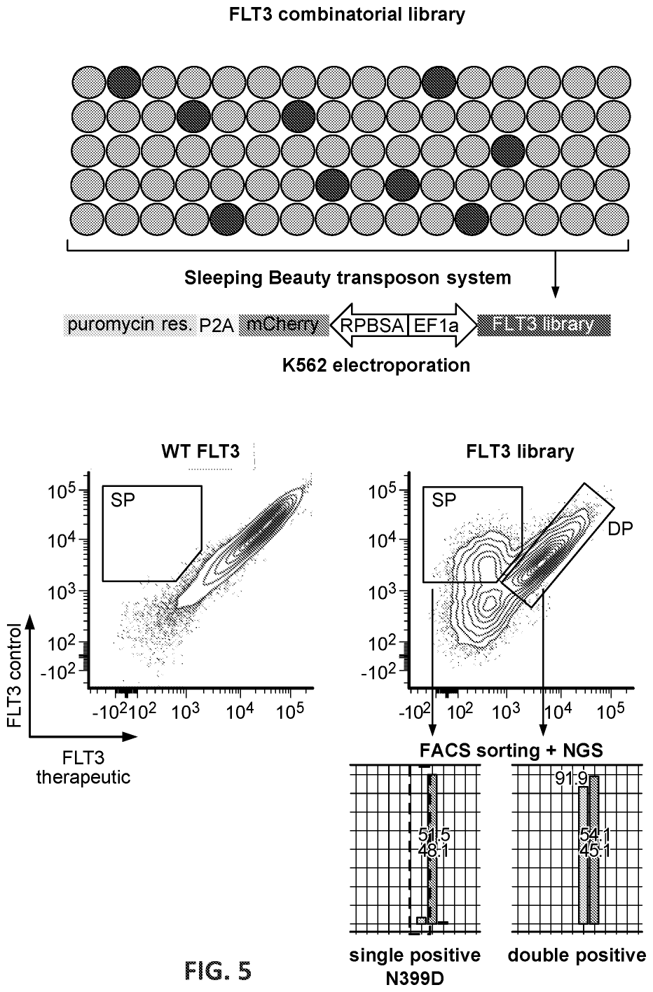

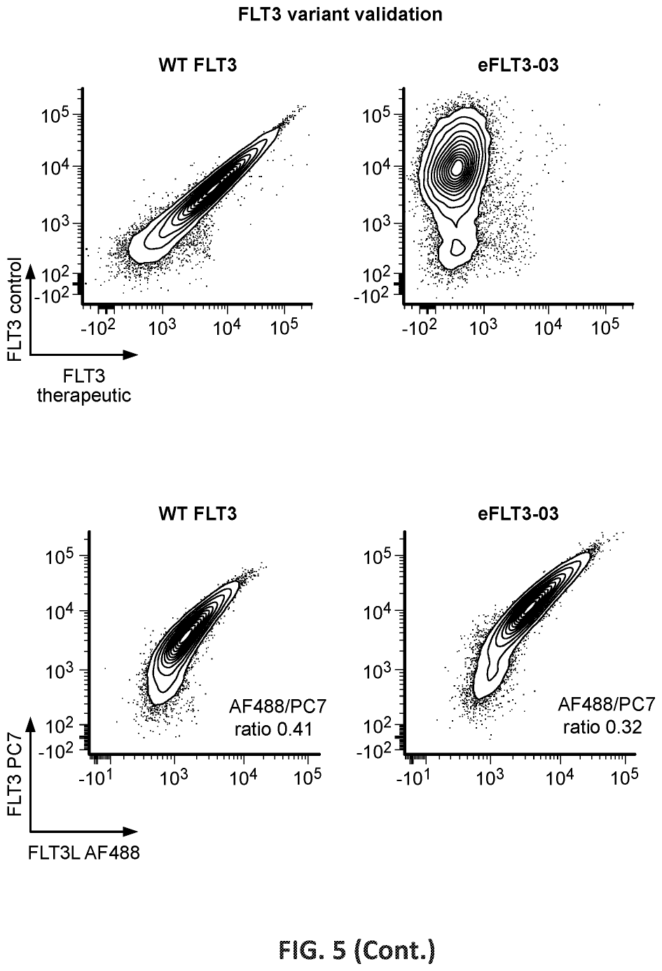

To identify the key residues involved in the anti-FLT3 antibody FLT3 4G8 clone recognition of FLT3 extracellular domain 4, a combinatorial library was designed with all 16 previously identified residues either wild-type or mutated and cloned in a sleeping beauty transposon transfer vector under a EF1 alpha promoter (GeneScript-FIG. 5). An antisense cassette expressing mCherry and puromycin resistance under a RPBSA promoter served as transduction marker and selection method. K562 cells were electroporated using Lonza 4D-Nucleofector system with 5, 10 or 100 ng of transfer vector and 500 ng of SB100x transposase-expressing plasmid. Nucleofected cells were selected with puromycin 1 ug/mL for 7 days and then evaluated by flow cytometry. Cells positive for the control antibody (FLT3 BV10A4 PE-Cy7) and negative for the therapeutic antibody (FLT3 4G8 BV711) were FACS-sorted with a BD Aria II sorter and expanded in vitro. The integrated FLT3 library region was PCR amplified from gDNA samples of unsorted, single positive sorted and double positive sorted cells and partial Illumina adapters were added to the amplicons and submitted for NGS on Illumina platforms (GeneWiz). By comparing the three samples, only one codon (N399) showed differential mutation enrichment in single positive vs double positive cells (FIG. 5). Validation of the candidate mutation (N399D) in a sleeping beauty overexpression system confirmed that it is sufficient for abrogation of 4G8 clone binding (FIG. 5). Incubation of K562 cells overexpressing the FLT3 N399D variant with fluorescent FLT3L conjugated with AF488 showed ligand binding comparable to wild type FLT3 (FIG. 5).

### KIT Epitope Engineering:

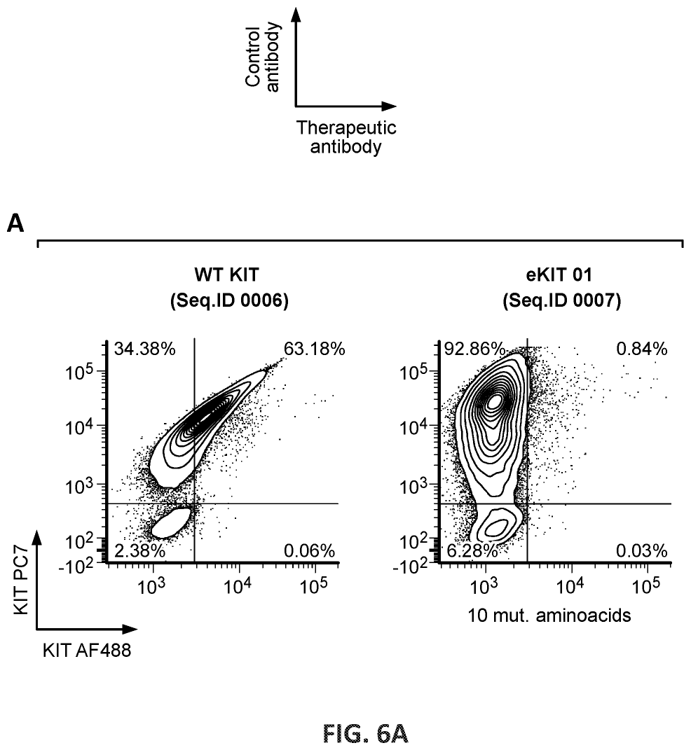

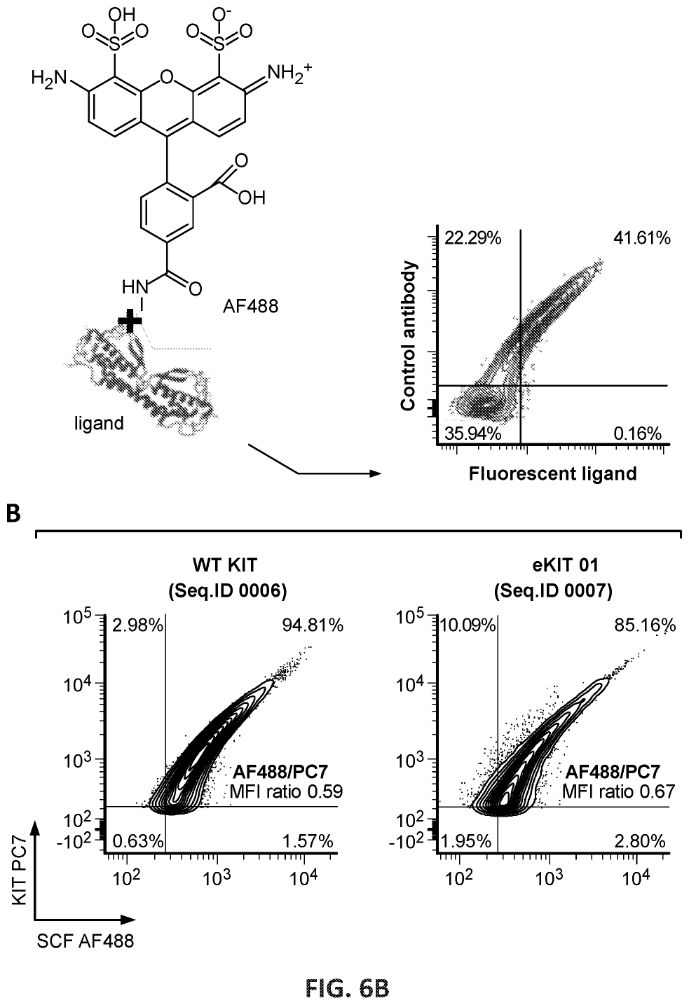

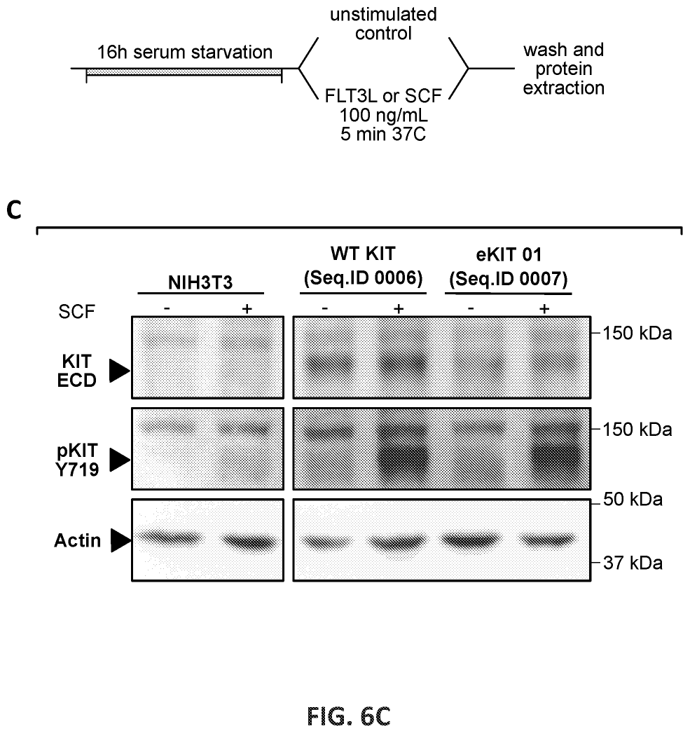

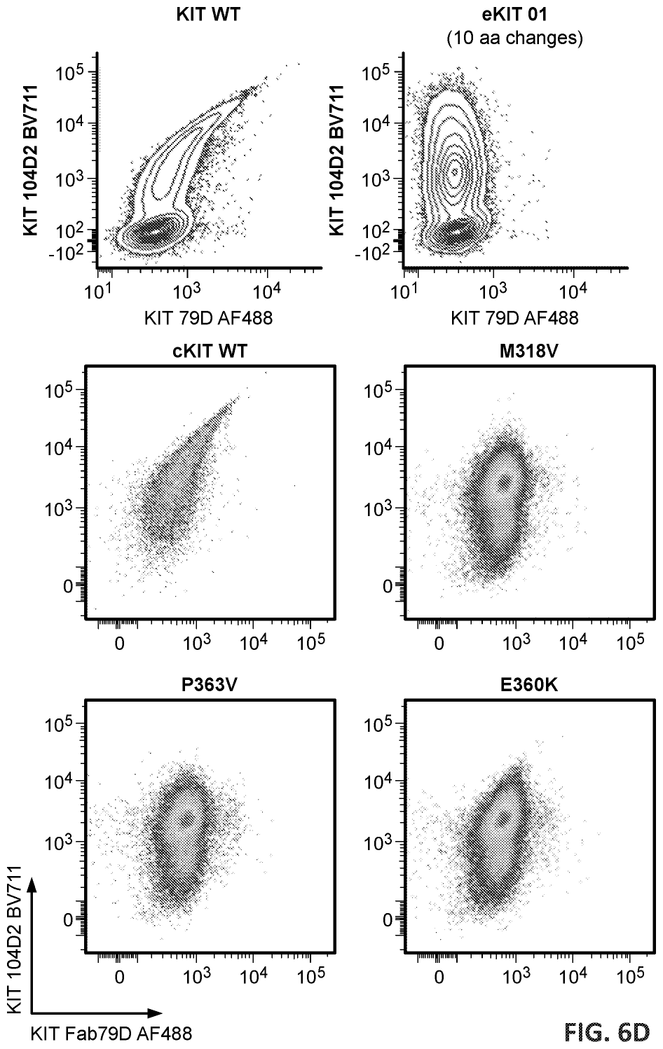

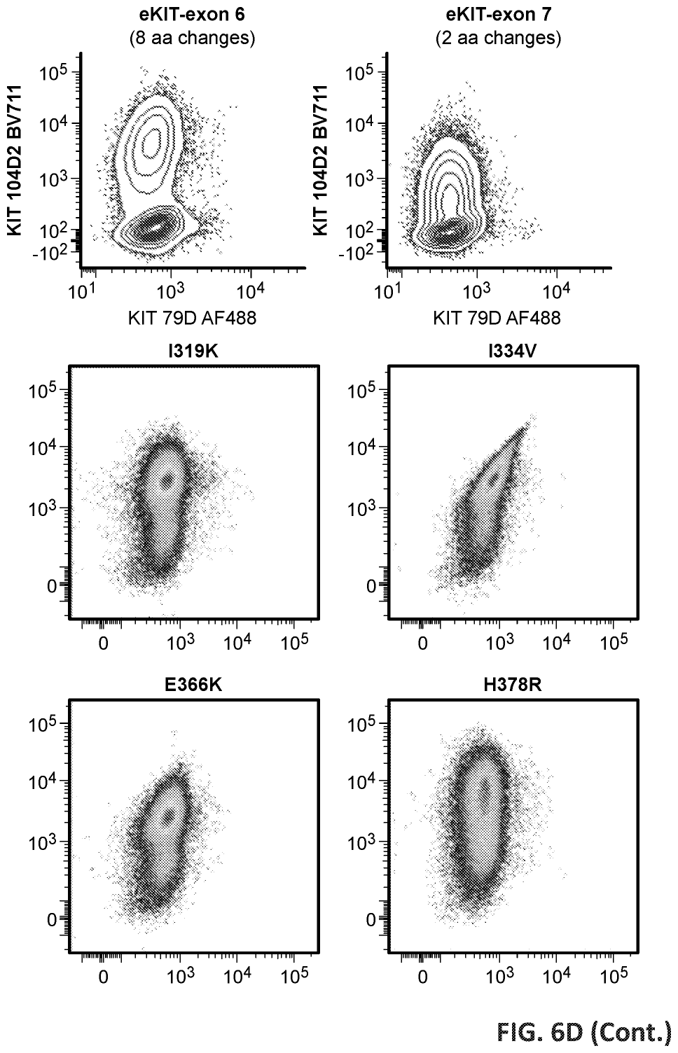

Based on the available literature, anti-KIT antibody clone Fab79D recognizes putative amino-acid contact points in the extracellular domain 4 of KIT (FIG. 3). As such, by substituting 10 of the predicted contact amino acids with lower preservation across ortholog sequences, a KIT variant (eKIT-01) that is not recognized by anti-KIT antibody clone Fab79D was generated (FIG. 6A). This variant preserved human stem cell factor (SCF) affinity (FIG. 6B) and intracellular domain phosphorylation upon SCF stimulation (FIG. 6C). To identify the specific amino-acids fundamental for clone Fab79D binding, each single amino acid change was cloned individually into sleeping beauty transfer vectors co-expressing mCherry and Puromycin resistance. 293T cells were electroporated using Lonza 4D-Nucleofector system in SF solution with 100 ng transfer vector and 500 ng plasmid expressing the SB100x transposase (FIG. 6D). Cells were selected with puromycin (2 ug/mL) and analyzed by flow cytometry staining with KIT Fab79D and KIT 104D2 control antibody. The mutation with lowest Fab79D MFI compared to KIT expression by clone 104D2 staining (KIT H378R) was selected as the candidate variant for base editing strategy development (FIG. 6D).

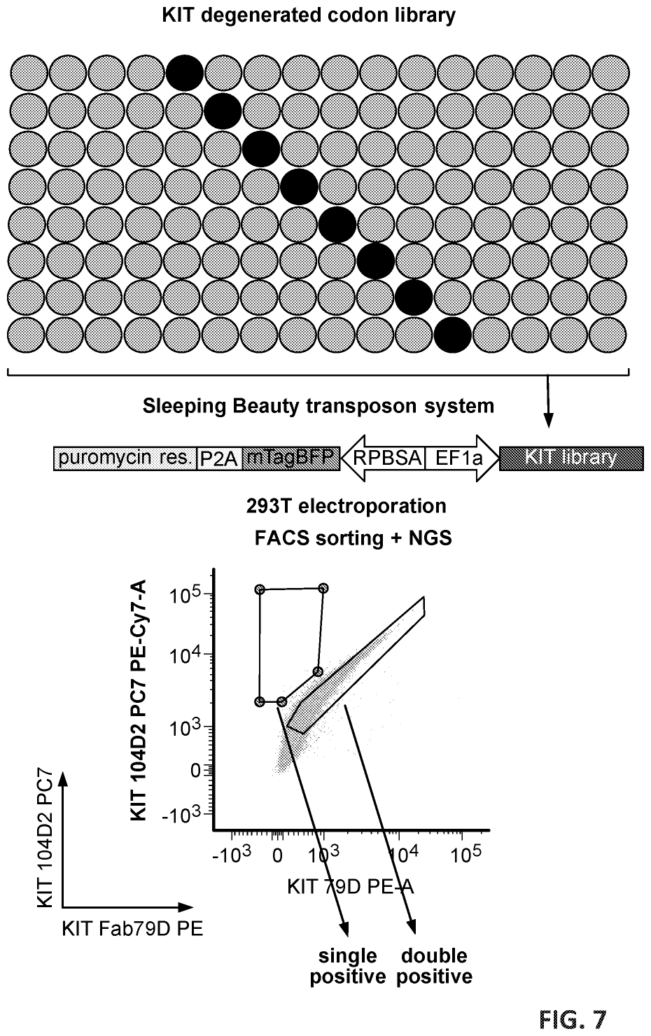

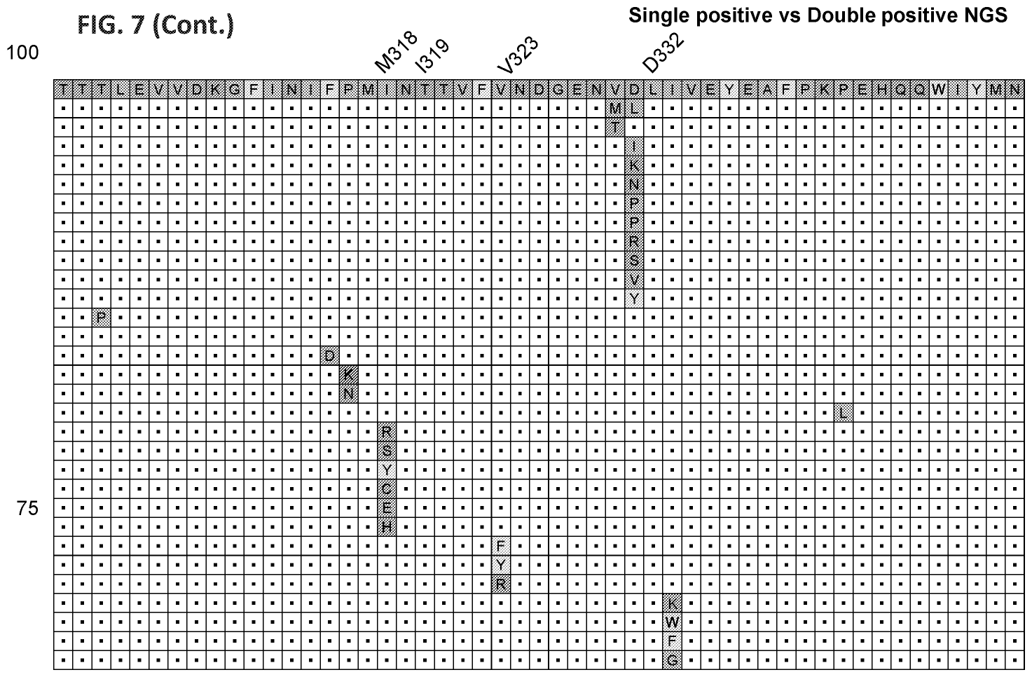

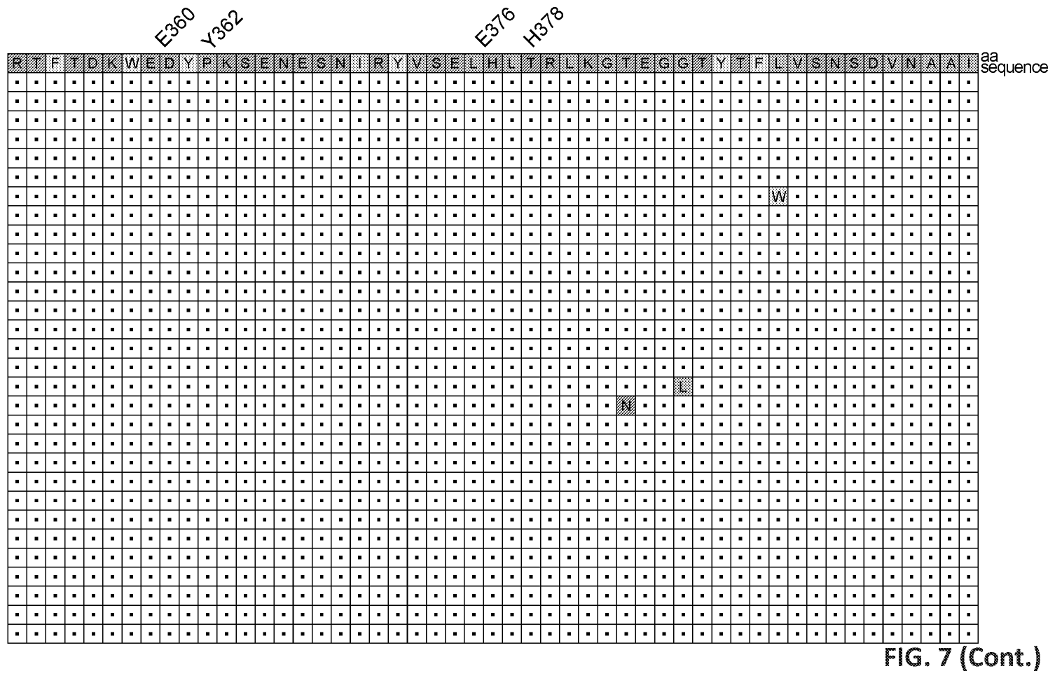

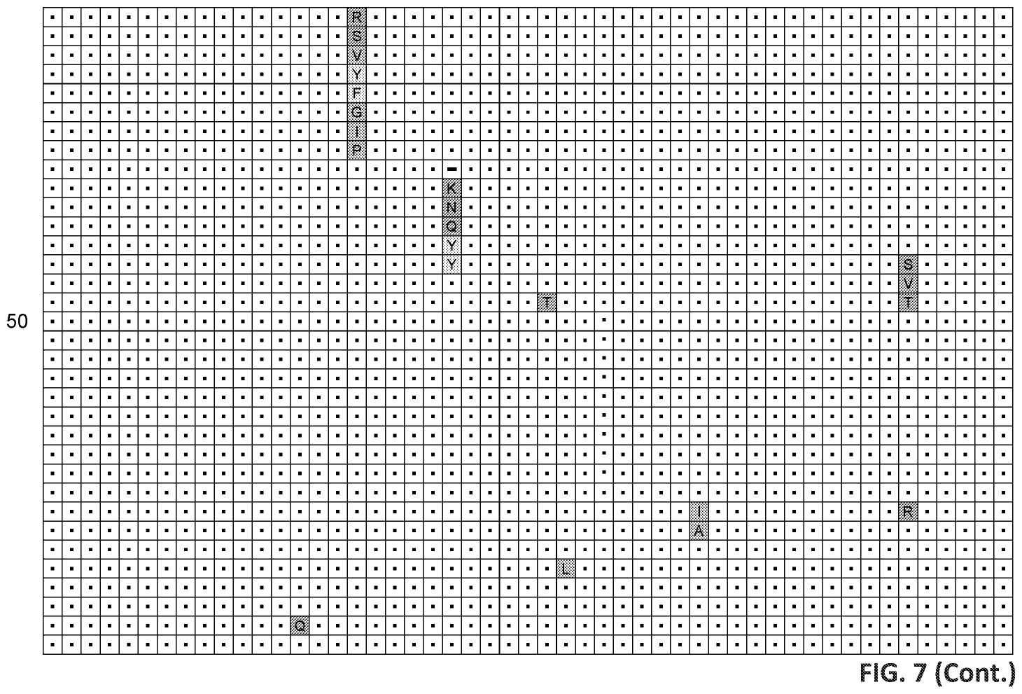

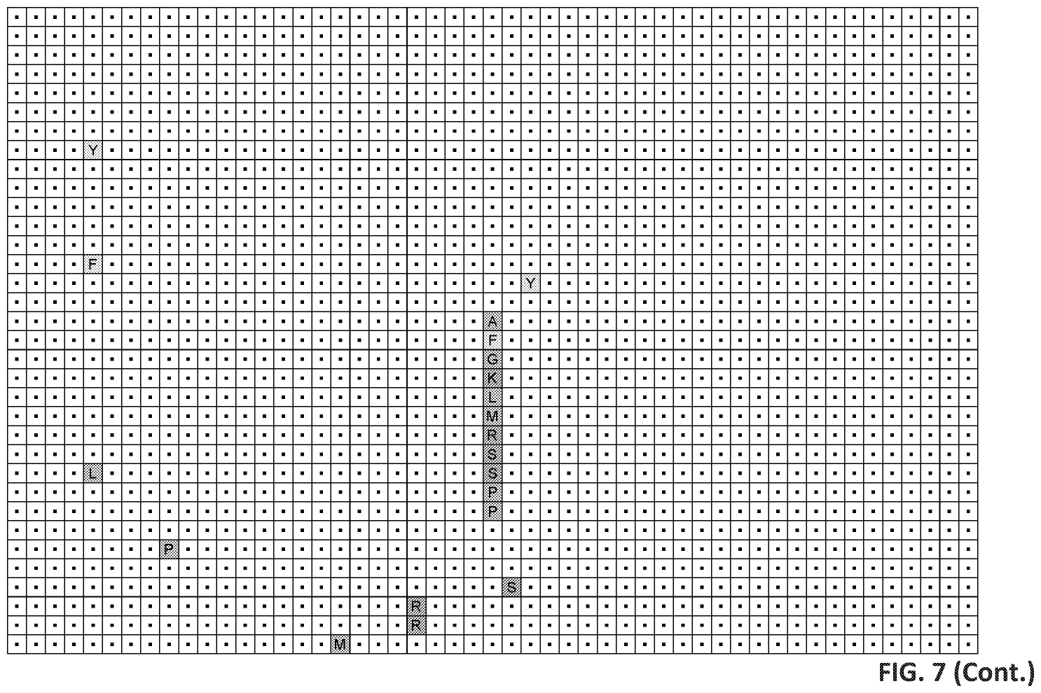

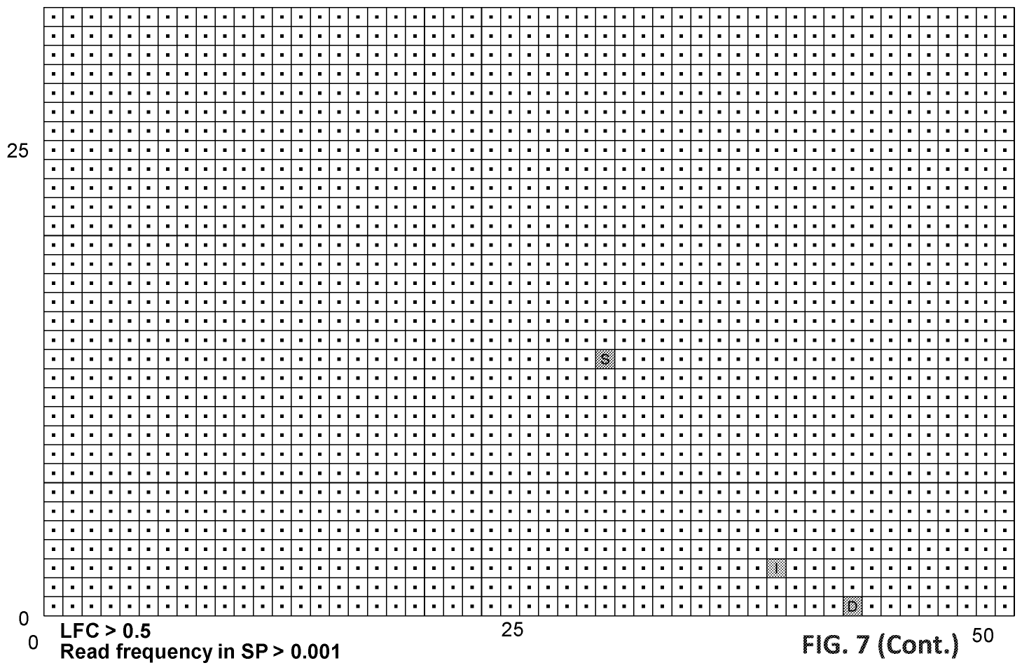

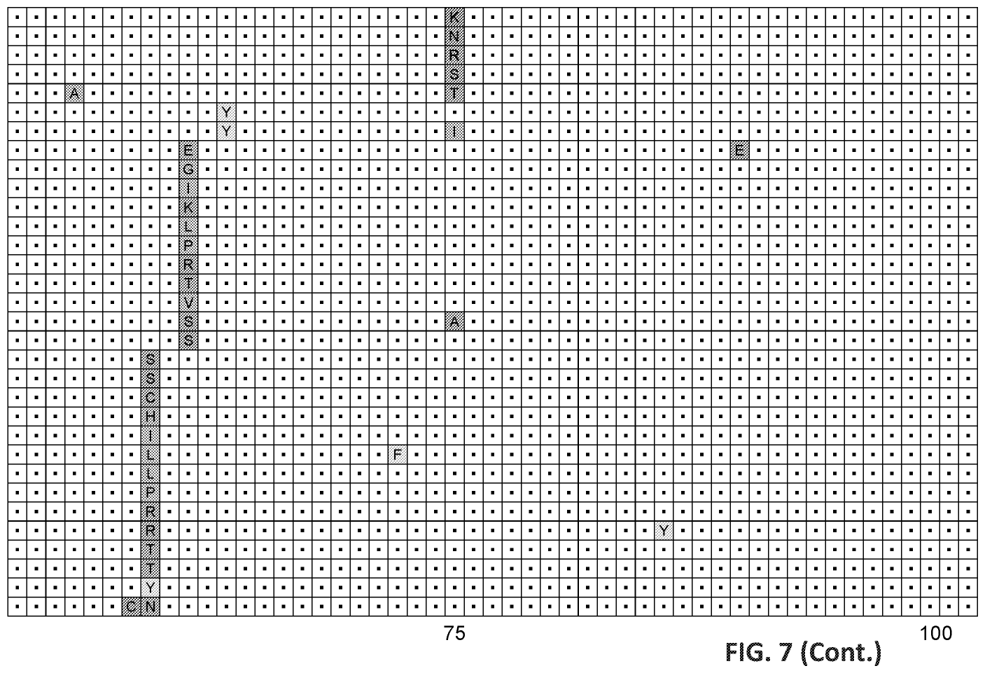

To expand these findings, a comprehensive library approach to further define alternative codons involved in Fab79D binding was designed. A degenerated library, where each codon within KIT extracellular domain 4 was composed of degenerated bases (NNN), was cloned in a sleeping beauty transfer plasmid expressing the human KIT cDNA, an mTagBFP reporter and puromycin resistance (FIG. 7). HEK-293T cells were electroporated with the library plasmid and a pSB100X transposase plasmid to allow stable integration of the transgene. After puromycin selection, the cells were FACS-sorted to obtain single positive and double positive populations. The library region was PCR amplified and NGS-sequenced. Comparison of the relative enrichment of the amino-acid variants within the single positive vs the double positive populations identified additional codons involved in Fab79D binding in addition to H378 (M318, 1319, V323, D332, E360, Y362, E376).

### CD123 Epitope Engineering:

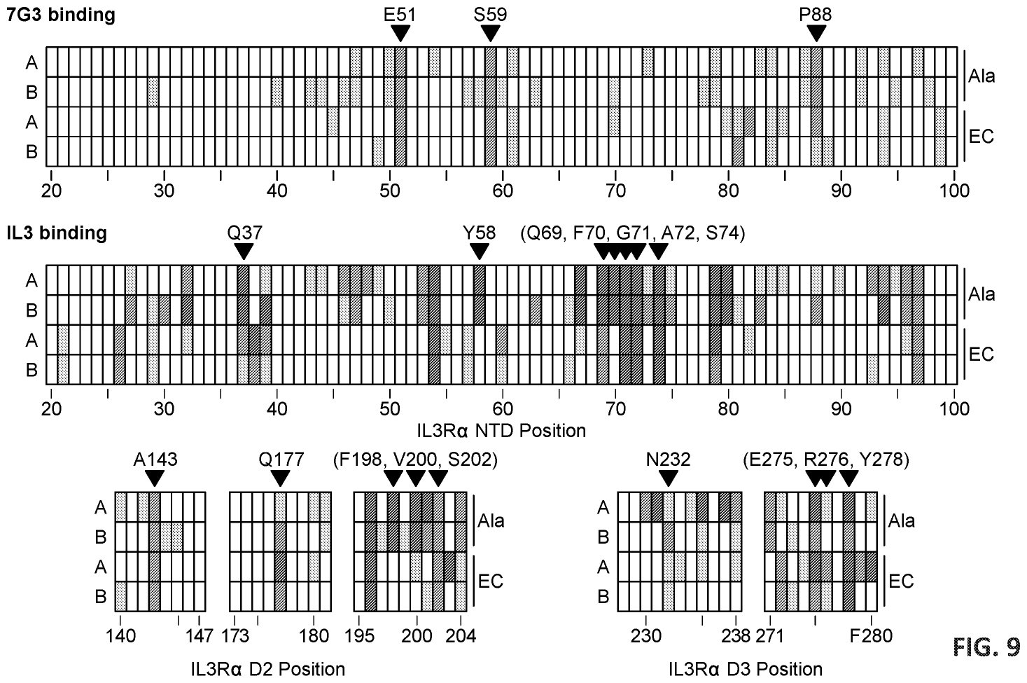

For CD123 (CD123), it has been reported in the literature that anti-CD123 antibody clones 7G3 and its humanized version, CSL362, recognize putative amino-acid contact points within CD123 N-terminal domain (FIG. 9). Similarly, other commercially available clones, 6H6 and S18016F are reported to bind the N-terminal domain with limited information on their contact points with CD123. IL3 is the ligand for CD123, and the CD123 residues important for IL3 binding have been mapped through Ala-scan or evolutionary conserved amino-acid changes (FIG. 9).

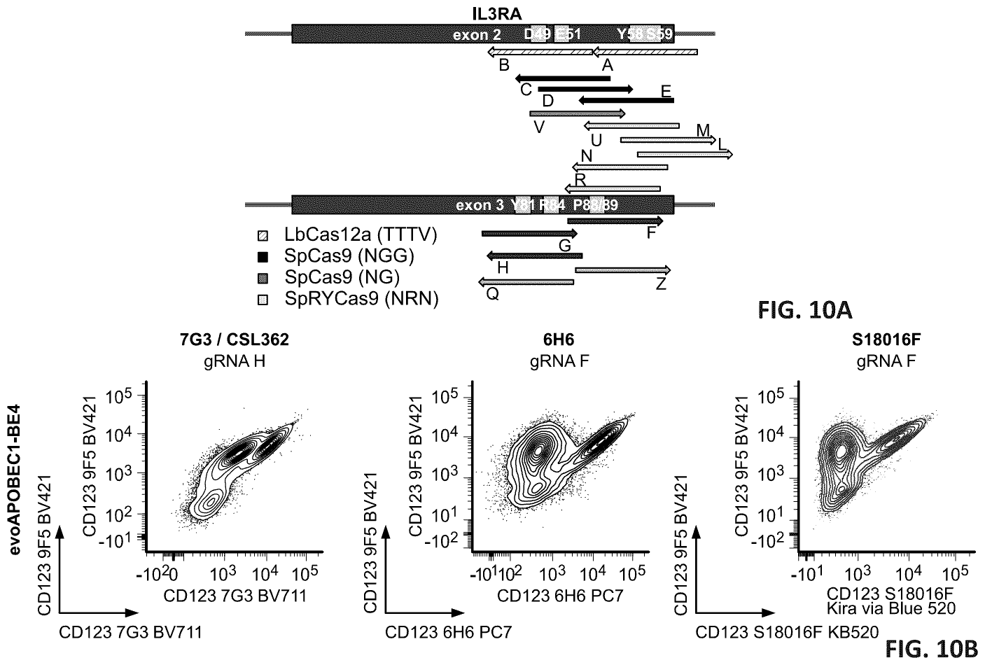

To engineer the target epitope within CD123 extracellular domain, a direct base editor screening approach was employed by designing a set of sgRNAs targeting 150, E51, Y58, S59, R84, P88 or P89 in the CD123 N-terminal domain (FIG. 10). These gRNAs were tested on K562 cells overexpressing CD123 with cytidine or adenine base editors (evo-APOBEC1-BE4, ABE8e-V106W). Briefly, 0.5 M cells were electroporated using Lonza 4D-Nucleofector system in SF solution according to the manufacturer instructions. 500 ng of base editor expression plasmid and 300-360 pmol of sgRNA (Integrated DNA Technologies) were included in the electroporation reaction. Cells were then cultured and samples for genomic DNA and flow cytometry analysis were harvested 72h after editing. The sgRNAs which generated CD123 mutants which lacked recognition by several mAb clones (7G3/CSL362, 6H6, S18016F) but retained surface expression by staining with control antibody clone 9F5 were selected for further development (FIG. 10, only the best performing candidate gRNAs are reported: SEQ ID NO: 24, SEQ ID NO: 30, SEQ ID NO: 32, SEQ ID NO: 34).

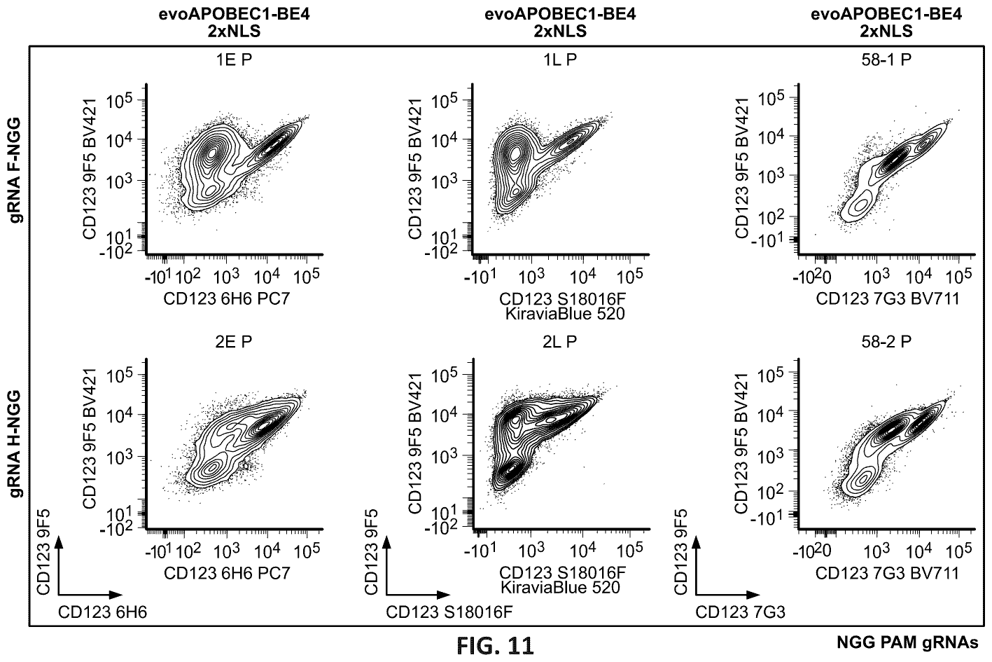

A second round of sgRNA screening was carried out including additional sequences in close proximity to the sgRNAs identified in the previous round and were tested with several BE, including variants with relaxed PAM specificity (evo-APOBEC1-BE4, NG-EA-BE4max, NG-A3A-BE5, SpRY-evo-APOBEC1-BE4-SEQ ID NO: 8 and SEQ ID NO: 11, NG-ABE8e, SpRY-ABE8e-V106E-SEQ ID NO: 7 and SEQ ID NO: 10, LbCas12a-ABE8e-FIG. 11). Flow cytometry evaluation of clones 7G3, 6H6 and S18016F binding was performed as previously described using clone 9F5 as normalizer for surface expression. gDNA from conditions showing desired loss of recognition was extracted and the CD123 exon 2 and 3 regions were Sanger sequenced to obtain the amino-acid change associated with the observed phenotype. For antibody clone 7G3 or its humanized counterpart, CSL362, CD123 S59P (introduced by gRNA-N, gRNA-R and related variants with ABE) and S59F (introduced by gRNA-L and variants with CBE) were the best candidate variants. A Y58H mutation was also introduced by gRNA-N and gRNA-R due to A to G conversion of a bystander adenine. To abrogate the binding of antibody clones 6H6 and S18016F, P88L/P89L (introduced by gRNA-F and related variants with CBE) were the best performing amino acid substitutions. Additional gRNAs (gRNA-H and related variants) which introduced a pool of other amino acid substitutions (R84Q, V85M, V85I, A86T) through cytidine base editing were identified and resulted in reduced affinity for clones 7G3, 6H6 and S18016F (FIG. 11).

### Example 3: FLT3 Variants are Resistant to FLT3-Targeting CAR-T Cells

To test if the FLT3 variants were selectively resistant to FLT3-targeting CAR-T cells, an in vitro killing assay was performed.

### CAR-T Cell Production

A III-generation lentiviral construct expressing a 2nd generation, FLT3-specific 4G8 clone chimeric antigen receptor with CD28 transmembrane region and CD28 co-stimulatory domain under a constitutive hPGK promoter was cloned using synthesized dsDNA fragments (IDT gBlocks). An antisense cassette expressing a truncated variant of the human EGFR cDNA under a minimal-CMV promoter was included to serve as marker of transduction and safety switch for in vivo depletion by using anti-EGFR antibody Cetuximab. VSV-G pseudotyped self-inactivating lentiviral particles were prepared according to published methods by calcium-phosphate transient co-transfection of 5 plasmids in HEK-293T cells (transfer vector, pMD2, pMDL-RRE, PREV and pAdvantage plasmids). Viral particles-containing supernatants were concentrated 500-fold by ultracentrifugation (20000 rpm at 20 C for 2 hours) and resuspended in PBS. Concentrated LVs were titrated by transducing 293T cells at different concentrations and calculating the transduction efficiency by flow cytometry or ddPCR.

Peripheral blood mononuclear cells (PBMC) were isolated by ficoll gradient separation from whole blood. After estimation of T cell fraction by flow cytometry, either freshly isolated or thawed PBMC were incubated with CD3-CD28 Dynabeads at 3:1 bead: T cell ratio (Gibco 11131D) for 45 min at room temperature on slow agitation and then magnetically separated (DynaMag-5 Magnet, Invitrogen 12303D). Positively selected cells were cultured with Dynabeads at 1 M/mL in IMDM supplemented with 10% FBS, 1% P/S, human IL-7 5 ng/mL (Peprotech) and human IL-15 5 ng/ml (Peprotech). 48h after the start of Dynabeads stimulation, T cells were transduced at MOI 5 to MOI 10 depending on the experiment with lentiviral particles encoding for the CAR of choice. Dynabeads were removed from culture by magnetic separation at day 7 since the start of the stimulation, and T cells were expanded for an additional 5-7 days in IMDM supplemented with 10% FBS, 1% P/S, human IL-7 5 ng/ml (Peprotech) and human IL-15 5 ng/ml (Peprotech). T cell phenotype and transduction efficiency (by EGFR surface staining) was evaluated periodically by flow cytometry. Expanded CAR-T cells or untransduced T cells were either used for killing assays, in vivo administration or vitally frozen after 12-14 days since the start of the stimulation.

K562 cells (either unmodified, base edited or overexpressing a receptor variant after sleeping-beauty transduction) were plated in a 96-well plate (25000 target cells/well). Anti-FLT3 CAR-T cells were generated by transducing peripheral blood mononuclear cells (PBMC with a lentiviral vector encoding for a 2nd generation 4G8-CAR construct with CD28 costimulatory domain, and co-expressing an EGFRt (truncated EGFR) safety switch (see protocol above). FLT3-targeting 4G8 CAR-T cells or untransduced T cells were marked with CellTrace yellow (Invitrogen C34567) according to the manufacturer's recommendations. FLT3-targeting 4G8 CAR-T cells or untransduced T cells were then co-plated at different effector: target ratios (E:T ratio), typically 10, 5, 2.5, 1.25, 0.625 in the same wells and incubated at 37° C. with 5% CO₂ in a humidified incubator. After 4 hours, 50% of the culture volume was harvested for flow cytometry analysis by staining with FcR-blocking reagent (Miltenyi 130-059-901) 2/100 μL, LIVE/DEAD Fixable Yellow Dead Cell Stain (Invitrogen L34967) 1/1000, FLT3 BV10A4 PE-Cy7 2/100 (Biolegend 313314), CD4 APC (BD) 2/100, CD8 BV750 (Biolegend) 2/100, CD107a BV711 (Biolegend) 2/100, CD69 PerCP-Cy5.5 (Biolegend) 2/100. The staining mix included flow counting beads to normalize cell counts (Biolegend Precision Count Beads). Cells were than washed and resuspended in AnnexinV binding buffer (Biolegend) supplemented with AnnexinV FITC (Biolegend) 3/100. Samples were analyzed on a 4- or 5-laser BD Fortessa flow cytometer. The remaining culture volume was evaluated at 48 hours post plating by flow cytometry stained with FcR-blocking reagent (Miltenyi 130-059-901) 2/100 μL, LIVE/DEAD Fixable Yellow Dead Cell Stain (Invitrogen L34967) 1/1000, FLT3 BV10A4 PE-Cy7 2/100 (Biolegend 313314), CD4 APC (BD) 2/100, CD8 BV750 (Biolegend) 2/100, CCR7 BV421 (Biolegend) 2/100, CD45RA APC-Cy7 (Biolegend) 3/100, CD33 PerCP-Cy5.5 (Biolegend). Cells were than washed and resuspended in AnnexinV binding buffer (Biolegend) supplemented with AnnexinV FITC (Biolegend) 3/100.

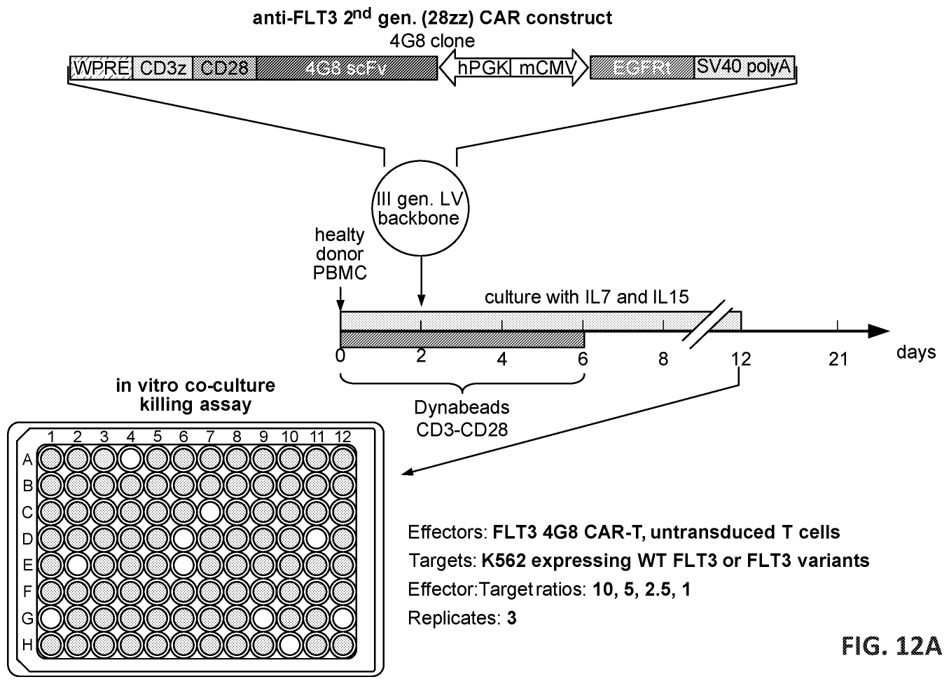

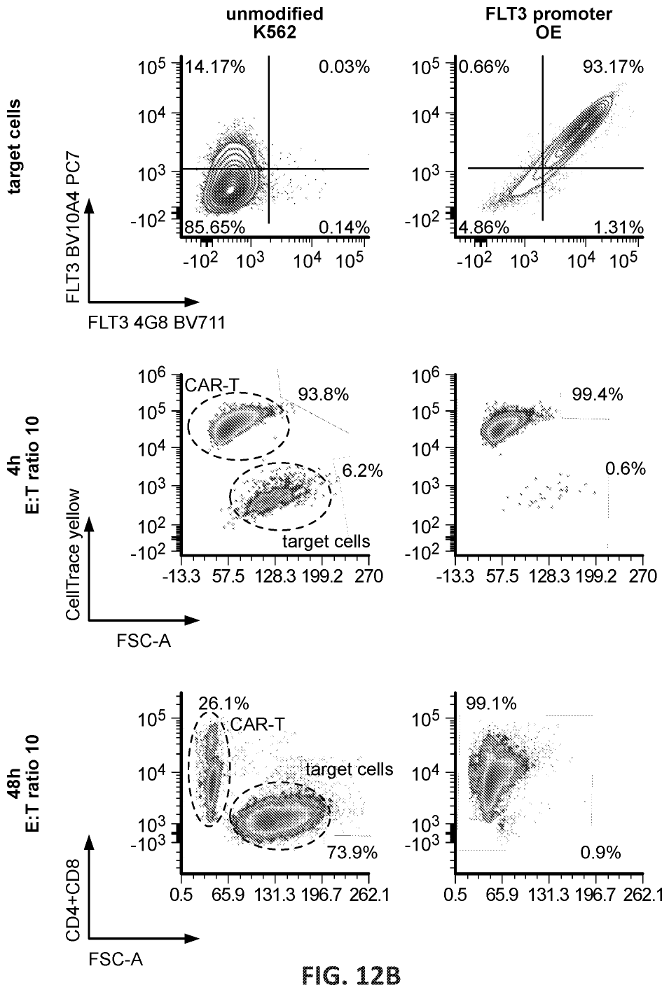

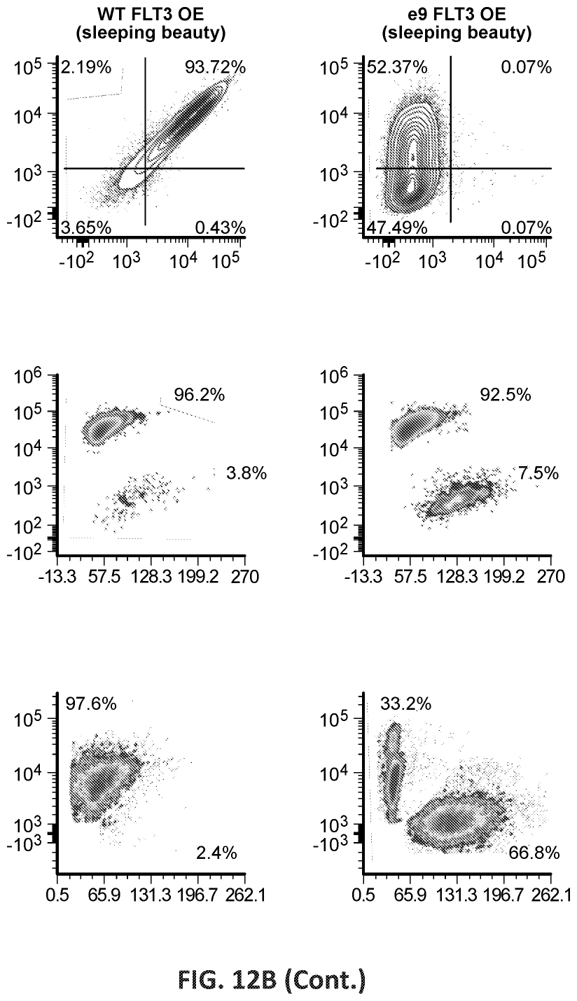

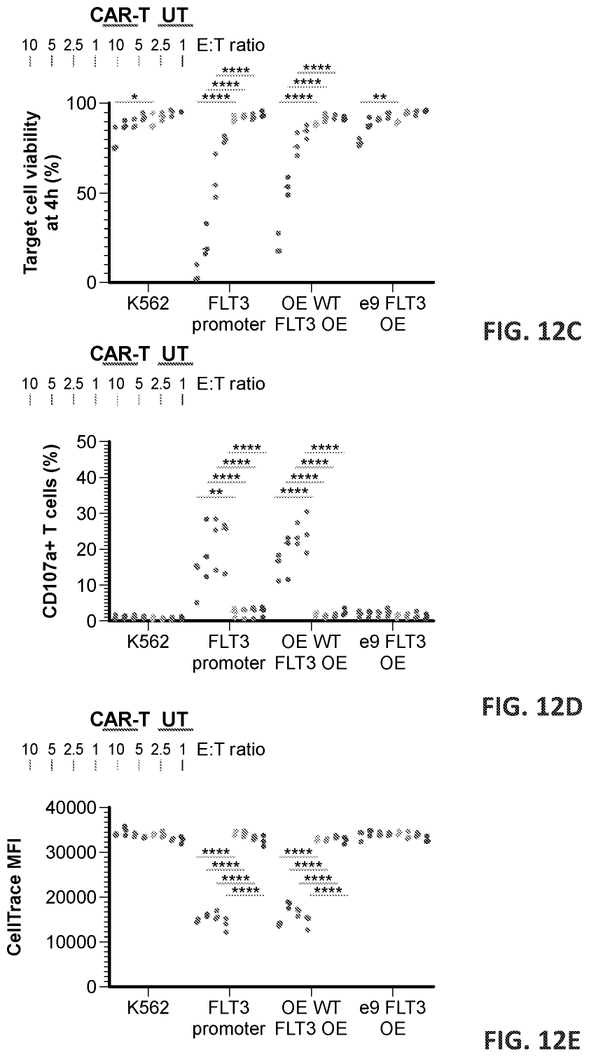

FIG. 12A shows the experimental design for co-culture killing assays to evaluate the resistance of modified FLT3 variants to CAR-T cell mediated killing. FIG. 12B are flow cytometry plots showing the co-culture composition of live cells at 4 hours and 48 hours after incubation. Top row shows FLT3 expression by flow cytometry on WT or engineered K562 cells. Cells expressing unmodified wild-type FLT3 are selectively killed, while engineered FLT3 (e9-FLT3) are spared. FIG. 12C shows target cell viability by AnnexinV and LiveDead yellow staining at 4 hours after incubation. FIG. 12D shows selective T cell degranulation by CD107a surface staining at 4 hours only for FLT3-CAR-T exposed to target cells expressing unmodified FLT3. FIG. 12E shows selective T cell proliferation by dye-dilution (CellTrace yellow) at 48 hours after co-culture only for FLT3-CAR-T exposed to target cells expressing unmodified FLT3. Unmodified K562: wild-type cells; WT FLT3 OE: K562 reporter cell line with FLT3 overexpression from its endogenous promoter (see reporter cell line generation); ‘WT FLT3 OE (sleeping beauty)’ and ‘e9 FLT3 OE (sleeping beauty)’: K562 cells transduced with a sleeping beauty transposon driving overexpression of FLT3 variant. The e9-FLT3 variant had 12 amino-acid changes compared to WT-FLT3, including N399D (SEQ ID NO: 50).

### Example 4: Homology Directed Repair to Introduce the N399D in FLT3

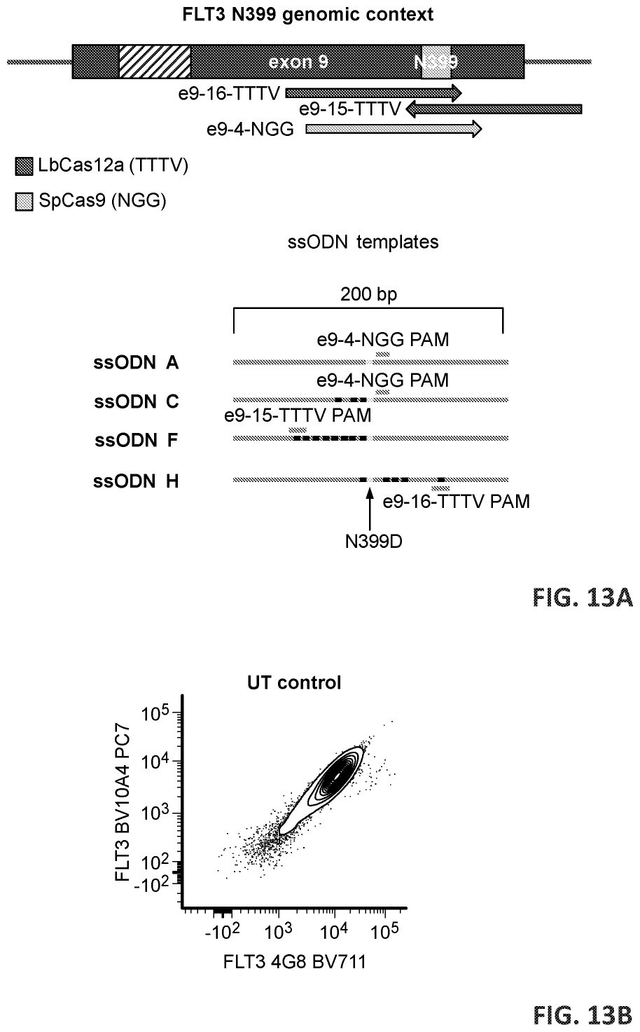

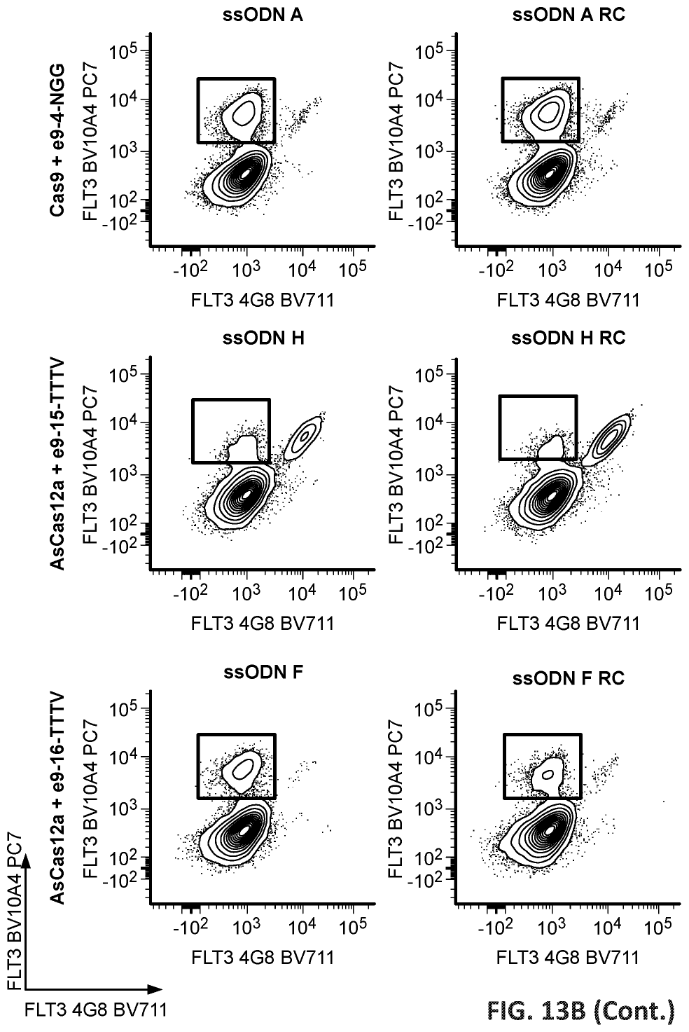

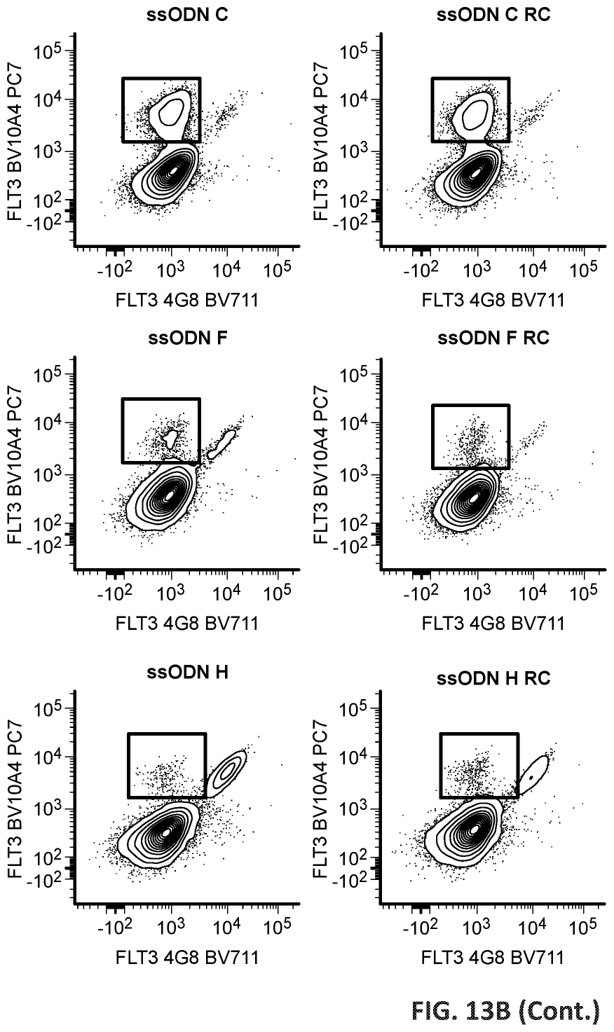

To test if FLT3 N399D can be introduced by homology directed repair, a CRISPR Cas homology directed repair (HDR) strategy was designed. SpCas9 or AsCas12a nucleases in combination with several gRNAs targeting the FLT3 exon 9 locus were tested in combination with and 200-nt long single strand oligo-deoxynucleotide (ssODN) as donors template for HDR. Each donor templates included selected silent mutations in bystander amino-acids to reduce the risk of re-cutting by the CRISPR-Cas9 RNP nuclease complex after successful DNA repair. The reverse complementary of each ssODN donors (termed A, C, H, F) were also tested. The sequences of the ssODN template donors are reported as SEQ ID NOS: 40 through 43 (see above in Table 2). K562 reporter cells overexpressing FLT3 by targeted integration of an EF1-a promoter upstream of the endogenous FLT3 locus were electroporated using Lonza 4D-Nucleofector system in SF solution supplemented with 50 pmol of 3xNLS Cas9 nuclease (IDT) complexed with 62.5 pmol annealed trRNA:gRNA or 50 pmol AsCas12a Ultra (IDT) complexed with 62.5 pmol sgRNA according to experimental condition. Cas9 gRNA e9-4-NGG was tested in combination with ssODN-A and C and their reverse complements (5 μM final concentration). Cas12a gRNAs e9-15-TTTV and e9-16-TTTV were tested in combination with ssODN-H and F and their reverse complements (5 uM final concentration). IDT HDR-enhancer 0.2 uL/20 uL was included in the electroporation reaction (30 uM). The outcome of the editing procedure was evaluated by flow cytometry 72 h after electroporation. FIG. 13A (top) shows the exon 9 of FLT3 with N399 residue highlighted and its relative position to 3 gRNAs (one SpCas9 gRNA (e9-4-NGG) and two Cas12a gRNAs (e9-15-TTTV and e9-16-TTTV)). FLT3 exon9 targeting gRNAs are reported as SEQ ID NOS: 13 through 16 (see above in Table 1). FIG. 13A (bottom) shows the single strand oligo-deoxynucleotide donor templates that were utilized to insert the N399D (arrow) mutation by homology directed repair. Additional silent single nucleotide changes were included in template design to reduce the rate of re-cutting by the Cas9-gRNA complex (dark squares).

FIG. 13B shows FACS plots 72 h after electroporation of K562 reporter cells stained with FLT3 clone 104D2 as normalizer for surface expression and clone 4G8 to evaluate the efficiency of N399D mutation. Successfully edited cells are highlighted by the black rectangle, showing that N399D mutation can be inserted in human cell lines through CRISPR-Cas homology directed repair resulting in loss of recognition by mAb clone 4G8.

### Example 5: FLT3 Mutations at N399 Position can be Introduced by CRISPR Adenine Base Editors

In order to introduce the desired single codon change in the FLT3 locus with high efficiency and low toxicity, without the introduction of double-strand DNA breaks, we tested CRISPR-Cas base editing. We designed a panel of sgRNAs that were predicted to introduce the N399D or N399G mutation in combination with adenine base editors. CRISPR-Cas9 base editor ABE8e (TadA-8e V106W) was selected for the development of the editing strategy and further optimized by mutating the Cas9 nickase protein to relax the PAM specificity in order to allow editing in the absence of conventional NGG PAM. To this end, both NG-SpCas9 and SpRY-Cas9 variants of the base editor were cloned. To further increase the efficiency, a 3rd nuclear localization site (NLS) was fused to the C-terminal portion of the protein. Unless stated otherwise, base editing experiments were performed by electroporation of reporter K562 overexpressing the FLT3 gene with 500 ng of base editor plasmid and either 300-360 pmol of sgRNA (Integrated DNA Technologies). Cells were then cultured and samples for genomic DNA and flow cytometry analysis were harvested 72 h after editing.

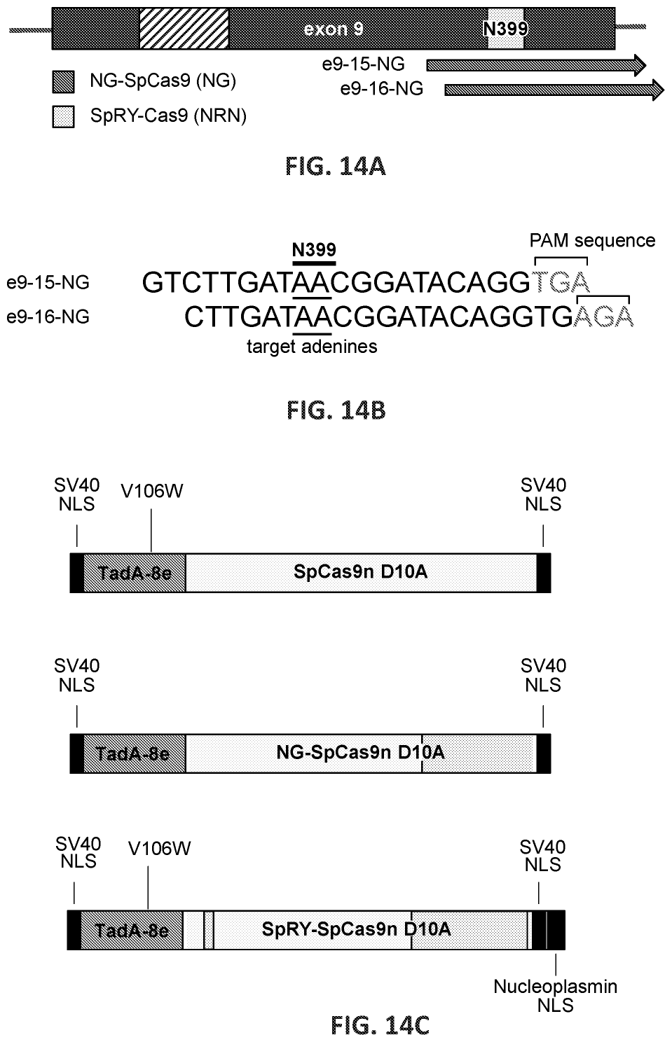

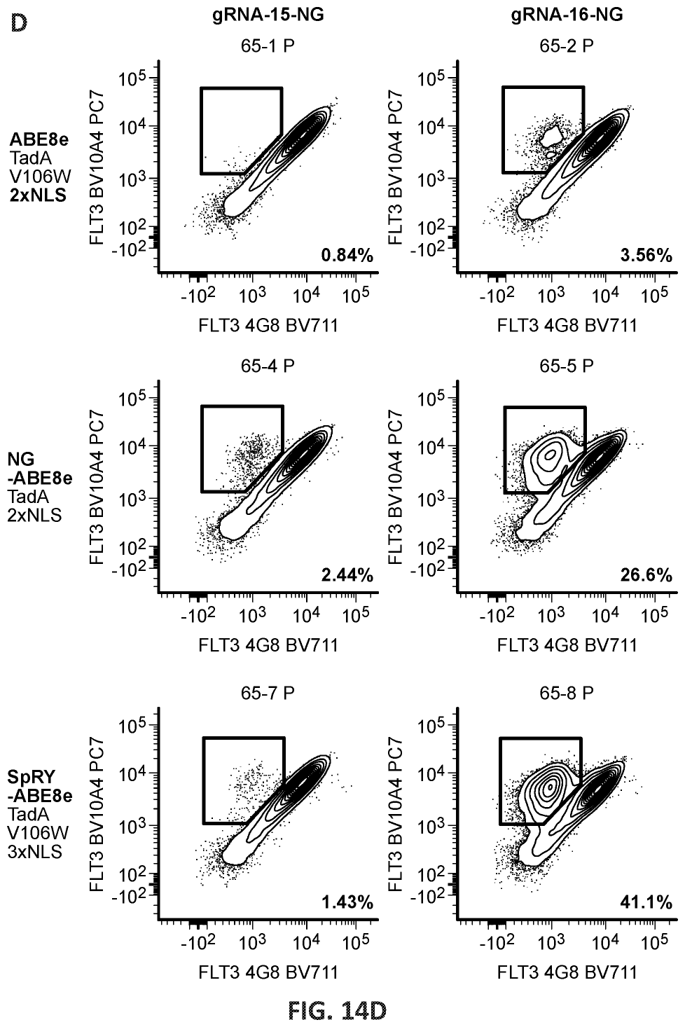

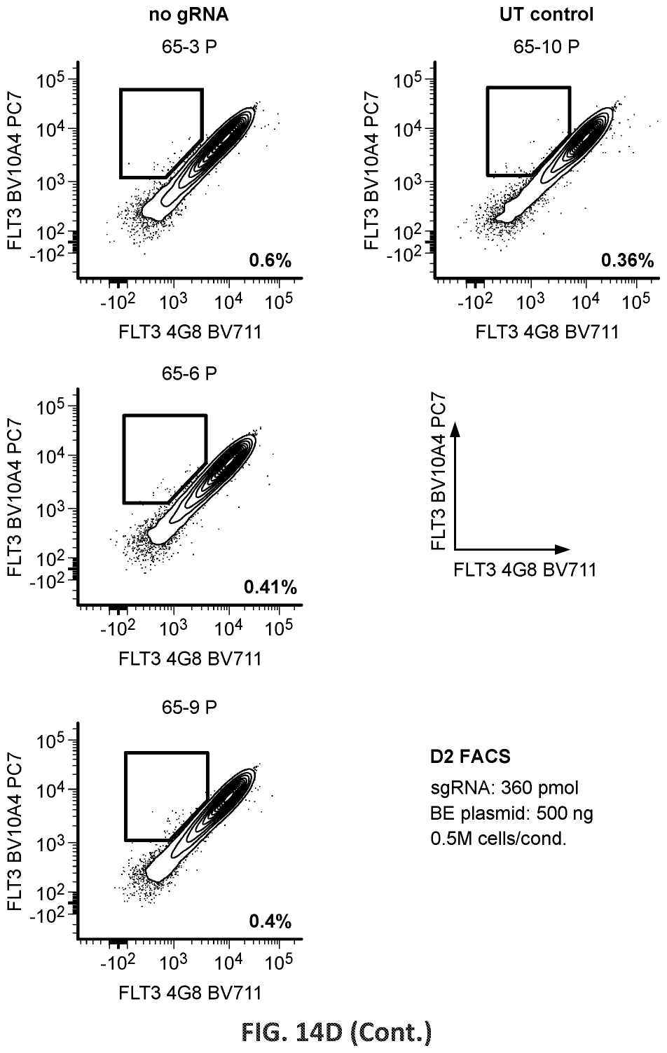

FIG. 14 shows a representative experiment exemplifying the PAM requirements for N399 base editing. Two SpCas9 sgRNAs with NG PAM (SEQ ID NO: 17 and SEQ ID NO: 18, see above in Table 1) were tested with WT-SpCas9 nickase (PAM requirement: NGG), NG-SpCas9 nickase (PAM requirement: NG), and SpRY-Cas9 nickase (PAM requirement: NRN), ABE constructs. FIG. 14A shows the relative position of the sgRNAs and N399 codon in the FLT3 gene. FIG. 14B the gRNA protospacer sequences with target adenines underlined. FIG. 14C shows the design of the three adenine base editors constructs (wild-type Cas9, NG-Cas9 or SpRY-Cas9 variants linked to the TadA deaminase domain with or without the V106W mutation). V106W mutation is associated with reduced RNA editing, a known undesired effect of adenine base editors. ABE8e-V106W and NG-ABE8e are also available through Addgene (catalog product #138495 and #138491, respectively). The cloned SpRY-ABE8e-V106W (SEQ ID NO: 7 and SEQ ID NO: 8) included an additional C-terminal Nucleoplasmin nuclear localization sequence (NLS). FACS plots of the base editing outcomes 72h after electroporation are represented in FIG. 14D, where K562 cells were stained with anti-FLT3 clone BV10A4 (normalizer) and 4G8 (therapeutic Ab). The percentage of edited cells is reported in the bottom right of each condition's FACS plot. Only base editor variants with relaxed PAM specificity (NG-ABE8e and SpRY-ABE8e-V106W) show efficient editing with up to 41.1% of cells becoming negative for clone 4G8 staining.

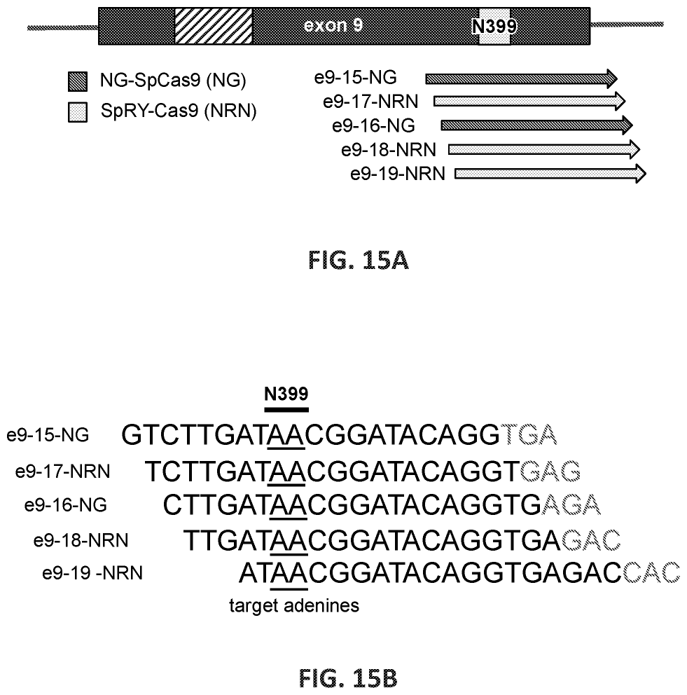

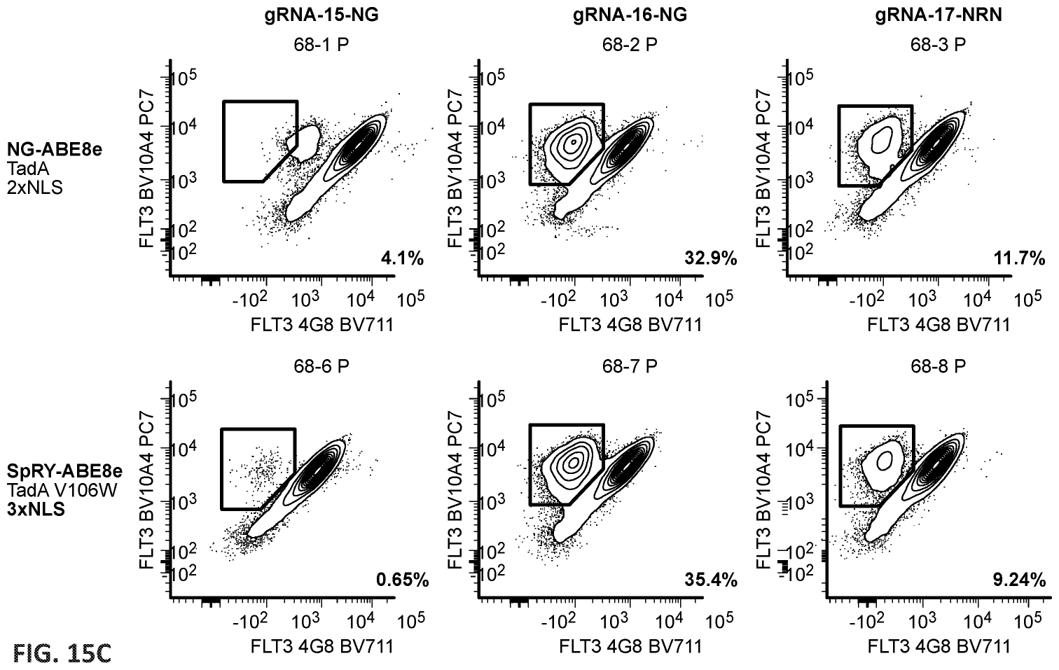

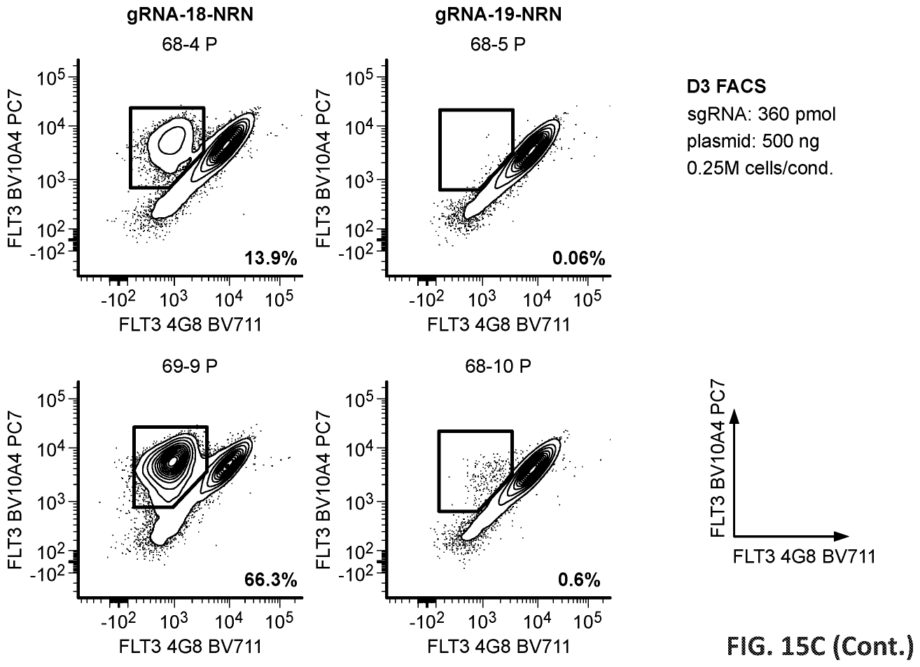

To improve efficiency of base-editing of the N399 codon, tailored editing window positioning was achieved by screening additional sgRNAs in combination with near PAM-less SpRY-Cas9 variant. FIG. 15A shows the genomic context of FLT3 N399 and its position relative to 5 sgRNAs, including the two from the previous example (SEQ ID NO: 17 and SEQ ID NO: 18) and three additional gRNA with NRN PAM (SEQ ID NO: 19, SEQ ID NO: 20, and SEQ ID NO: 23). FIG. 15B shows the sgRNA protospacer sequences with the target adenine underlined. FIG. 15C are FACS plots of a base editing experiment with electroporation of 500 ng base editor-expressing plasmid and 360 pmol of sgRNA into K562 cells. The percentage of edited cells is reported in the bottom right of each condition's FACS plot. SpRY-ABE8e-V106W in combination with sgRNA FLT3_e9 18 achieves the highest efficiency by flow cytometry, with up to 66.3% of edited cells being not recognized by clone 4G8.

### Example 6: FLT3 Mutations Introduced by ABE Preserve Protein Expression and Ligand Binding

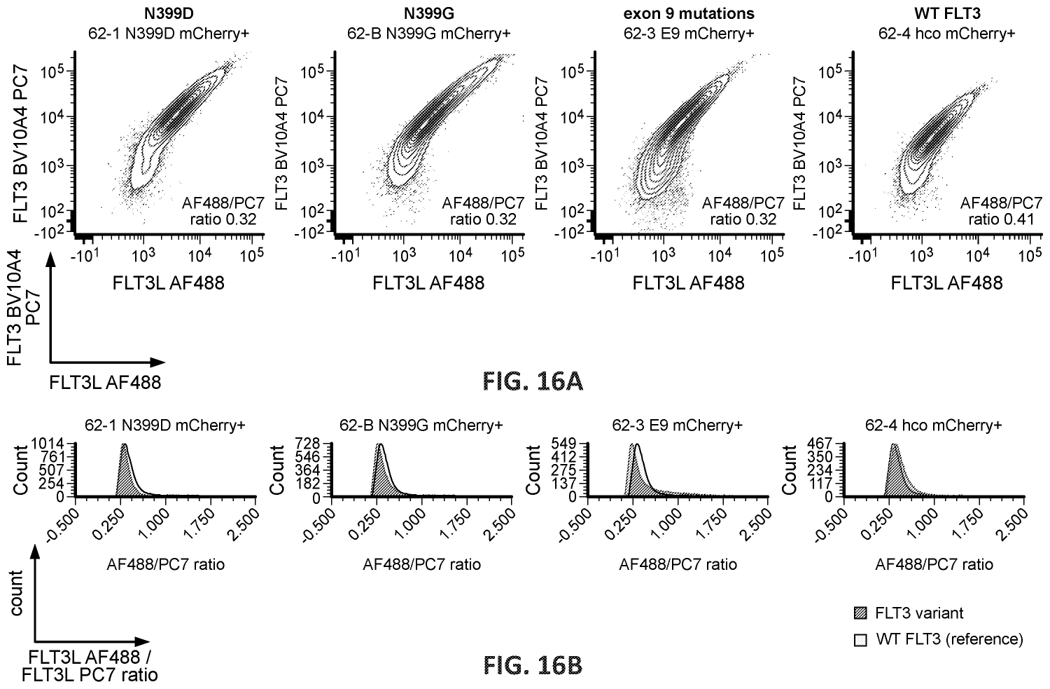

Sanger sequencing of base edited cells from Example 5 revealed A to G editing of both adenines within the N399 codon (sequence: AAC) with potential generation of either N399D or N399G mutations. To further test if FLT3 variants containing a mutation at N399 position still preserve physiological FLT3L binding, fluorescent ligand binding assays were performed. FIG. 16A shows a fluorescent ligand binding assay performed with K562 cells expressing various FLT3 variants: N339D (SEQ ID NO: 51), N399G (SEQ ID NO: 52), exon 9 mutations (SEQ ID NO: 50) or WT FLT3 (SEQ ID NO: 48). The fluorescence ratio between FLT3L AF488 and FLT3 BV10A4 PE-Cy7 is reported in each plot. FIG. 16B shows the distribution of fluorescence ratio between FLT3L AF488 and FLT3 BV10A4 PE-Cy7 for each variant (histograms). Data shows that N399D and N399G variants resulting from FLT3 adenine base editing preserve physiological FLT3L binding affinity.

### Example 7: Use of SpRY-ABE8e-V106W mRNA Results in Highly Efficient Base Editing in Human Leukemia Cell Lines and Human CD34+ HSPCs

In order to translate the base editing procedure to primary cells, a suitable delivery method for base editors needs to be developed, as bacterial plasmid transfection is reported to be toxic for stem cells.

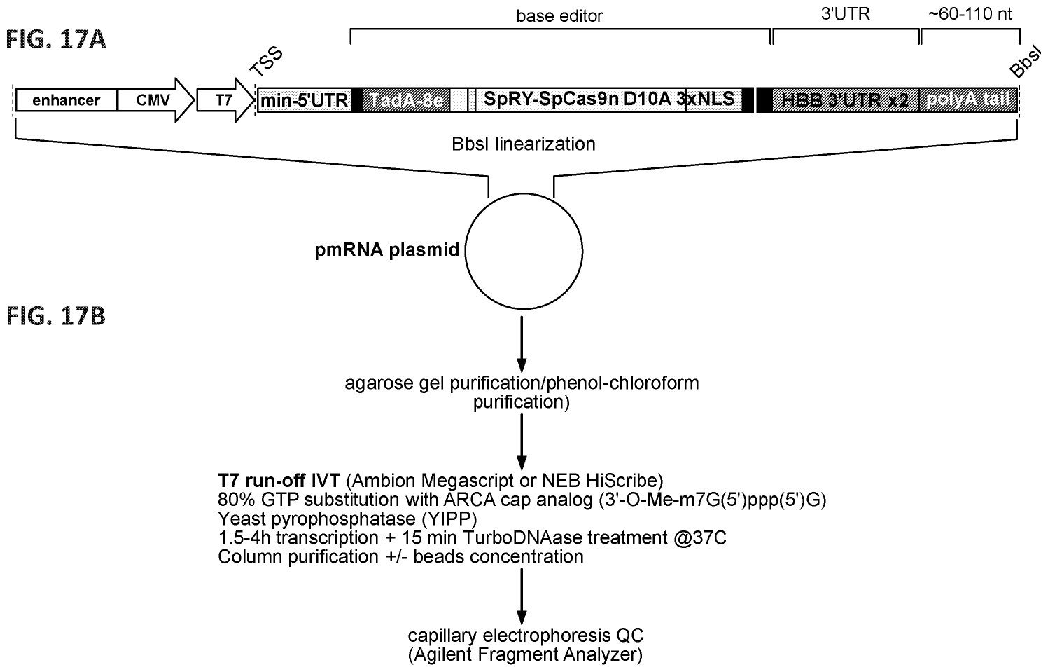

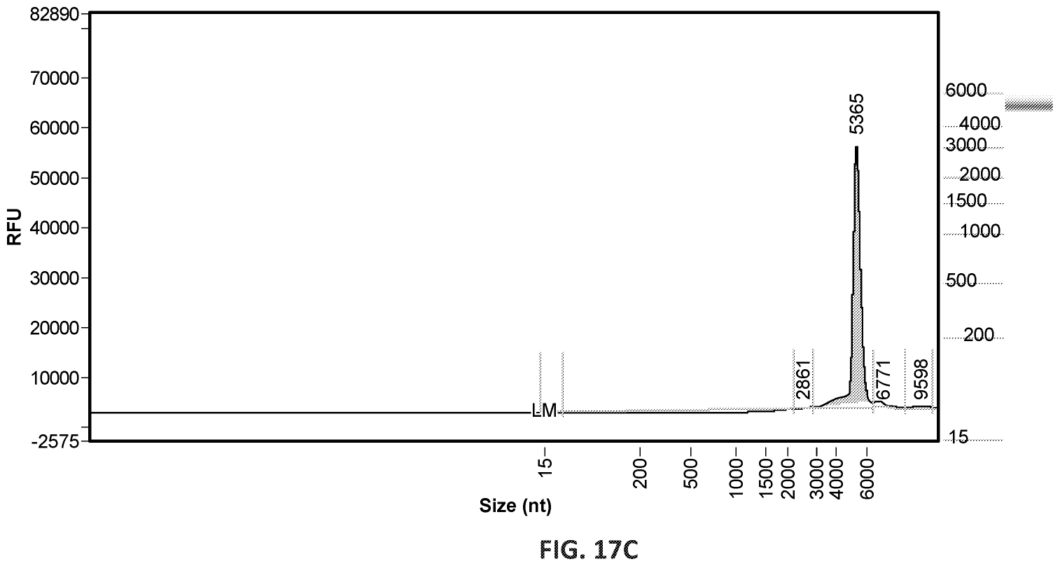

We employed base editor mRNA produced by in vitro transcription to translate the base editing protocol to human CD34+ HSPC. Functional mRNA encoding for adenine base editors (SpRY-ABE8e-V106W 3xNLS or SpRY-K918N-ABE8e-V106W 3xNLS) were produced by in vitro transcription (IVT) using MEGAscript T7 Transcription Kit (Invitrogen AM1333) or NEB T7 HiScribe kit (E2040S) and a custom plasmid (SEQ ID NO: 81; see FIG. 17A and below) template encoding the base editor reading frame downstream to a T7 promoter sequence, a minimal 5′ UTR and upstream to 2 copies of HBB (hemoglobin B) 3′UTR and a polyA sequence (60-120 bp long). FIG. 17B exemplifies the in vitro transcription workflow that was utilized for the generation of co-transcriptionally 5′-capped and 3′-polyadenylated mRNA for primary human CD34+ base editing. Co-transcriptional capping was achieved by substituting 80% of the GTP with 3′-O-Me-m7G (5′)ppp(5′) G RNA cap structure analog (NEB S1411). The IVT reaction products were purified using either Qiagen RNAesy mini kit or NEB Monarch mRNA CleanUp (T2050L), quantified by Nanodrop and analyzed by Agilent Fragment Analyzer for quality control (typical electropherogram shown in FIG. 17C.

-pmRNA plasmid for in vitro transcription of SpRY_ABE8e_V106W adenine
base editor, including 5′UTR, HBB 3′UTRx2 and 120 bp long polyA tails
SEQ ID NO: 81
atatgccaagtacgccccctattgacgtcaatgacggtaaatggcccgcctggcattat
gcccagtacatgaccttatgggactttcctacttggcagtacatctacgtattagtcat
cgctattaccatggtgatgcggttttggcagtacatcaatgggcgtggatagcggtttg
actcacggggatttccaagtctccaccccattgacgtcaatgggagtttgttttggcac
caaaatcaacgggactttccaaaatgtcgtaacaactccgccccattgacgcaaatggg
cggtaggcgtgtacggtgggaggtctatataagcagagctggtttagtgaaccgtcaga
tccgctagagatccgcggccgcAGCGCTGGTACCtaatacgactcactatagggagaCT
GCCAAGatgaaacggacagccgacggaagcgagttcgagtcaccaaagaagaagcggaa
agtctctgaggtggagttttcccacgagtactggatgagacatgccctgaccctggcca
agagggcacgggatgagagggaggtgcctgtgggagccgtgctggtgctgaacaataga
gtgatcggcgagggctggaacagagccatcggcctgcacgacccaacagcccatgccga
aattatggccctgagacagggcggcctggtcatgcagaactacagactgattgacgcca
ccctgtacgtgacattcgagccttgcgtgatgtgcgccggcgccatgatccactctagg
atcggccgcgtggtgtttggatggagaaattctaaaagaggcgccgcaggctccctgat
gaacgtgctgaactaccccggcatgaatcaccgcgtcgaaattaccgagggaatcctgg
cagatgaatgtgccgccctgctgtgcgatttctatcggatgcctagacaggtgttcaat
gctcagaagaaggcccagagctccatcaactccggaggatctagcggaggctcctctgg
ctctgagacacctggcacaagcgagagcgcaacacctgaaagcagcgggggcagcagcg
gggggtcagacaagaagtacagcatcggcctggccatcggcaccaactctgtgggctgg
gccgtgatcaccgacgagtacaaggtgcccagcaagaaattcaaggtgctgggcaacac
cgaccggcacagcatcaagaagaacctgatcggagccctgctgttcgacagcggcgaaa
cagccgagAGAacccggctgaagagaaccgccagaagaagatacaccagacggaagaac
cggatctgctatctgcaagagatcttcagcaacgagatggccaaggtggacgacagctt
cttccacagactggaagagtccttcctggtggaagaggataagaagcacgagcggcacc
ccatcttcggcaacatcgtggacgaggtggcctaccacgagaagtaccccaccatctac
cacctgagaaagaaactggtggacagcaccgacaaggccgacctgcggctgatctatct
ggccctggcccacatgatcaagttccggggccacttcctgatcgagggcgacctgaacc
ccgacaacagcgacgtggacaagctgttcatccagctggtgcagacctacaaccagctg
ttcgaggaaaaccccatcaacgccagcggcgtggacgccaaggccatcctgtctgccag
actgagcaagagcagacggctggaaaatctgatcgcccagctgcccggcgagaagaaga
atggcctgttcggaaacctgattgccctgagcctgggcctgacccccaacttcaagagc
aacttcgacctggccgaggatgccaaactgcagctgagcaaggacacctacgacgacga
cctggacaacctgctggcccagatcggcgaccagtacgccgacctgtttctggccgcca
agaacctgtccgacgccatcctgctgagcgacatcctgagagtgaacaccgagatcacc
aaggcccccctgagcgcctctatgatcaagagatacgacgagcaccaccaggacctgac
cctgctgaaagctctcgtgcggcagcagctgcctgagaagtacaaagagattttcttcg
accagagcaagaacggctacgccggctacattgacggcggagccagccaggaagagttc
tacaagttcatcaagcccatcctggaaaagatggacggcaccgaggaactgctcgtgaa
gctgaacagagaggacctgctgcggaagcagcggaccttcgacaacggcagcatccccc
accagatccacctgggagagctgcacgccattctgcggcggcaggaagatttttaccca
ttcctgaaggacaaccgggaaaagatcgagaagatcctgaccttccgcatcccctacta
cgtgggccctctggccaggggaaacagcagattcgcctggatgaccagaaagagcgagg
aaaccatcaccccctggaacttcgaggaagtggtggacaagggcgcttccgcccagagc
ttcatcgagcggatgaccaacttcgataagaacctgcccaacgagaaggtgctgcccaa
gcacagcctgctgtacgagtacttcaccgtgtataacgagctgaccaaagtgaaatacg
tgaccgagggaatgagaaagcccgccttcctgagcggcgagcagaaaaaggccatcgtg
gacctgctgttcaagaccaaccggaaagtgaccgtgaagcagctgaaagaggactactt
caagaaaatcgagtgcttcgactccgtggaaatctccggcgtggaagatcggttcaacg
cctccctgggcacataccacgatctgctgaaaattatcaaggacaaggacttcctggac
aatgaggaaaacgaggacattctggaagatatcgtgctgaccctgacactgtttgagga
cagagagatgatcgaggaacggctgaaaacctatgcccacctgttcgacgacaaagtga
tgaagcagctgaagcggcggagatacaccggctggggcaggctgagccggaagctgatc
aacggcatccgggacaagcagtccggcaagacaatcctggatttcctgaagtccgacgg
cttcgccaacagaaacttcatgcagctgatccacgacgacagcctgacctttaaagagg
acatccagaaagcccaggtgtccggccagggcgatagcctgcacgagcacattgccaat
ctggccggcagccccgccattaagaagggcatcctgcagacagtgaaggtggtggacga
gctcgtgaaagtgatgggccggcacaagcccgagaacatcgtgatcgaaatggccagag
agaaccagaccacccagaagggacagaagaacagccgcgagagaatgaagcggatcgaa
gagggcatcaaagagctgggcagccagatcctgaaagaacaccccgtggaaaacaccca
gctgcagaacgagaagctgtacctgtactacctgcagaatgggcgggatatgtacgtgg
accaggaactggacatcaaccggctgtccgactacgatgtggaccatatcgtgcctcag
agctttctgaaggacgactccatcgacaacaaggtgctgaccagaagcgacaagaaccg
gggcaagagcgacaacgtgccctccgaagaggtcgtgaagaagatgaagaactactggc
ggcagctgctgaacgccaagctgattacccagagaaagttcgacaatctgaccaaggcc
gagagaggcggcctgagcgaactggataaggccggcttcatcaagagacagctggtgga
aacccggcagatcacaaagcacgtggcacagatcctggactcccggatgaacactaagt
acgacgagaatgacaagctgatccgggaagtgaaagtgatcaccctgaagtccaagctg
gtgtccgatttccggaaggatttccagttttacaaagtgcgcgagatcaacaactacca
ccacgcccacgacgcctacctgaacgccgtcgtgggaaccgccctgatcaaaaagtacc
ctaagctggaaagcgagttcgtgtacggcgactacaaggtgtacgacgtgcggaagatg
atcgccaagagcgagcaggaaatcggcaaggctaccgccaagtacttcttctacagcaa
catcatgaactttttcaagaccgagattaccctggccaacggcgagatccggaagcggc
ctctgatcgagacaaacggcgaaaccggggagatcgtgtgggataagggccgggatttt
gccaccgtgcggaaagtgctgagcatgccccaagtgaatatcgtgaaaaagaccgaggt
gcagacaggcggcttcagcaaagagtctatcAGAcccaagaggaacagcgataagctga
tcgccagaaagaaggactgggaccctaagaagtacggcggcttcCTTTGGcccaccgtg
gcctattctgtgctggtggtggccaaagtggaaaagggcaagtccaagaaactgaagag
tgtgaaagagctgctggggatcaccatcatggaaagaagcagcttcgagaagaatccca
tcgactttctggaagccaagggctacaaagaagtgaaaaaggacctgatcatcaagctg
cctaagtactccctgttcgagctggaaaacggccggaagagaatgctggcctctgccAA
GCaactgcagaagggaaacgaactggccctgccctccaaatatgtgaacttcctgtacc
tggccagccactatgagaagctgaagggctcccccgaggataatgagcagaaacagctg
tttgtggaacagcacaagcactacctggacgagatcatcgagcagatcagcgagttctc
caagagagtgatcctggccgacgctaatctggacaaagtgctgtccgcctacaacaagc
accgggataagcccatcagagagcaggccgagaatatcatccacctgtttaccctgacc
aGActgggagcccctAGAgccttcaagtactttgacaccaccatcgaccCTaagCAAta
caGAagcaccaaagaggtgctggacgccaccctgatccaccagagcatcaccggcctgt
acgagacacggatcgacctgtctcagctgggaggtgactctggcggctcaaaaagaacc
gccgacggcagcgaattcgagcccaagaagaagaggaaagtcggcagcggaagcaaaag
gccggcggccacgaaaaaggccggccaggcaaaaaagaaaaagctcgagtaaaccggtC
CCGGGtctagaagctcgctttcttgctgtccaatttctattaaaggttcctttgttccc
taagtccaactactaaactgggggatattatgaagggccttgagcatctggattctgcc
taataaaaaacatttattttcattgctcATGCATatagaagctcgctttcttgctgtcc
aatttctattaaaggttcctttgttccctaagtccaactactaaactgggggatattat
gaagggccttgagcatctggattctgcctaataaaaaacatttattttcattgctcttA
GCtaataaaaaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaaaaaaaaaaaaaaa
aaaaaaaaGTCTTCactagtAAGCTGCTGATaaacccgctgatcagcctcgactgtgcc
ttctagttgccagccatctgttgtttgcccctcccccgtgccttccttgaccctggaag
gtgccactcccactgtcctttcctaataaaatgaggaaattgcatcgcattgtctgagt
aggtgtcattctattctggggggtggggtggggcaggacagcaagggggaggattggga
agtcaatagcaggcatgctggggatgcggtgggctctatggcttctgaggcggaaagaa
ccagctggggctcgataccgtcgacctctagctagagcttggcgtaatcatggtcatag
ctgtttcctgtgtgaaattgttatccgctcacaattccacacaacatacgagccggaag
cataaagtgtaaagcctagggtgcctaatgagtgagctaactcacattaattgcgttgc
gctcactgcccgctttccagtcgggaaacctgtcgtgccagctgcattaatgaatcggc
caacgcgcggggagaggcggtttgcgtattgggcgctcttccgcttcctcgctcactga
ctcgctgcgctcggtcgttcggctgcggcgagcggtatcagctcactcaaaggcggtaa
tacggttatccacagaatcaggggataacgcaggaaagaacatgtgagcaaaaggccag
caaaaggccaggaaccgtaaaaaggccgcgttgctggcgtttttccataggctccgccc
ccctgacgagcatcacaaaaatcgacgctcaagtcagaggtggcgaaacccgacaggac
tataaagataccaggcgtttccccctggaagctccctcgtgcgctctcctgttccgacc
ctgccgcttaccggatacctgtccgcctttctcccttcgggaagcgtggcgctttctca
tagctcacgctgtaggtatctcagttcggtgtaggtcgttcgctccaagctgggctgtg
tgcacgaaccccccgttcagcccgaccgctgcgccttatccggtaactatcgtcttgag
tccaacccggtaagacacgacttatcgccactggcagcagccactggtaacaggattag
cagagcgaggtatgtaggcggtgctacagagttcttgaagtggtggcctaactacggct
acactagaagaacagtatttggtatctgcgctctgctgaagccagttaccttcggaaaa
agagttggtagctcttgatccggcaaacaaaccaccgctggtagcggtggtttttttgt
ttgcaagcagcagattacgcgcagaaaaaaaggatctcaagaagatcctttgatctttt
ctacggggtctgacactcagtggaacgaaaactcacgttaagggattttggtcatgaga
ttatcaaaaaggatcttcacctagatccttttaaattaaaaatgaagttttaaatcaat
ctaaagtatatatgagtaaacttggtctgacagttaccaatgcttaatcagtgaggcac
ctatctcagcgatctgtctatttcgttcatccatagttgcctgactccccgtcgtgtag
ataactacgatacgggagggcttaccatctggccccagtgctgcaatgataccgcgaga
cccacgctcaccggctccagatttatcagcaataaaccagccagccggaagggccgagc
gcagaagtggtcctgcaactttatccgcctccatccagtctattaattgttgccgggaa
gctagagtaagtagttcgccagttaatagtttgcgcaacgttgttgccattgctacagg
catcgtggtgtcacgctcgtcgtttggtatggcttcattcagctccggttcccaacgat
caaggcgagttacatgatcccccatgttgtgcaaaaaagcggttagctccttcggtcct
ccgatcgttgtcagaagtaagttggccgcagtgttatcactcatggttatggcagcact
gcataattctcttactgtcatgccatccgtaagatgcttttctgtgactggtgagtact
caaccaagtcattctgagaatagtgtatgcggcgaccgagttgctcttgcccggcgtca
atacgggataataccgcgccacatagcagaactttaaaagtgctcatcattggaaaacg
ttcttcggggcgaaaactctcaaggatcttaccgctgttgagatccagttcgatgtaac
ccactcgtgcacccaactgatcttcagcatcttttactttcaccagcgtttctgggtga
gcaaaaacaggaaggcaaaatgccgcaaaaaagggaataagggcgacacggaaatgttg
aatactcatactcttcctttttcaatattattgaagcatttatcagggttattgtctca
tgagcggatacatatttgaatgtatttagaaaaataaacaaataggggttccgcgcaca
tttccccgaaaagtgccacctgacgtcgacggatcgggagatcgatctcccgatcccct
agggtcgactctcagtacaatctgctctgatgccgcatagttaagccagtatctgctcc
ctgcttgtgtgttggaggtcgctgagtagtgcgcgagcaaaatttaagctacaacaagg
caaggcttgaccgacaattgcatgaagaatctgcttagggttaggcgttttgcgctgct
tcgcgatgtacgggccagatatacgcgttgacattgattattgactagttattaatagt
aatcaattacggggtcattagttcatagcccatatattgagttccgcgttacataactt
acggtaaatggcccgcctggctgaccgcccaacgacccccgcccattgacgtcaataat
gacgtatgttcccatagtaacgccaatagggactttccattgacgtcaatgggtggagt
atttacggtaaactgcccacttggcagtacatcaagtgtatc

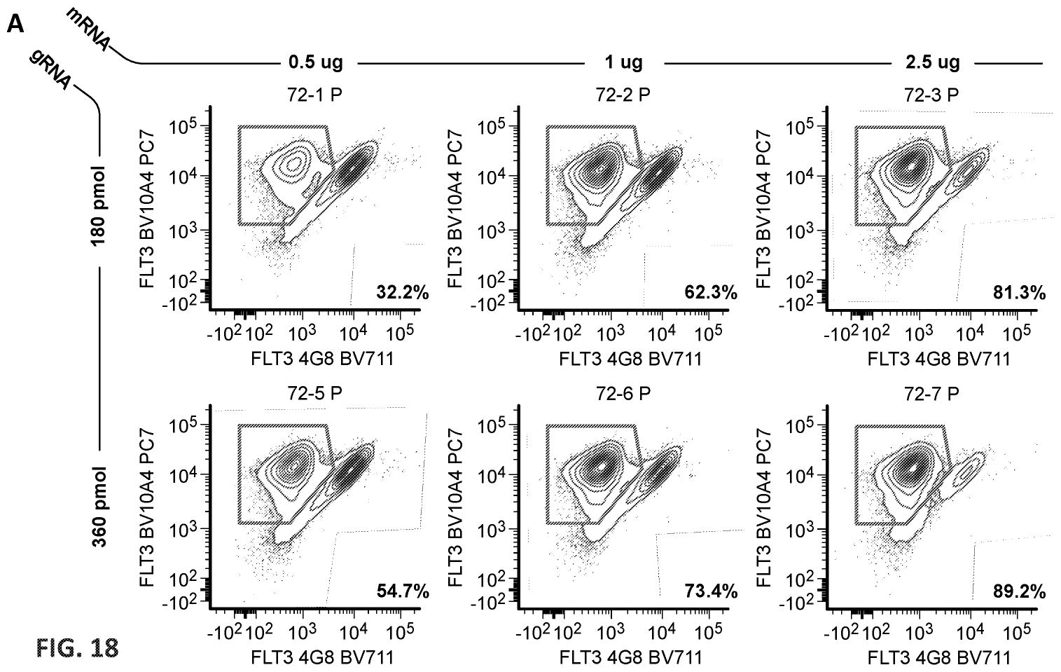

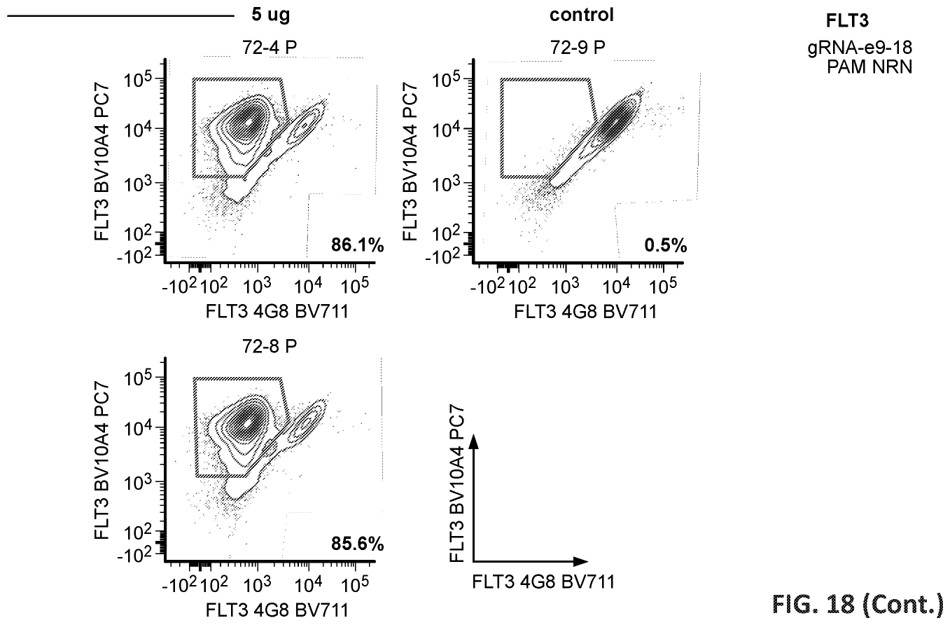

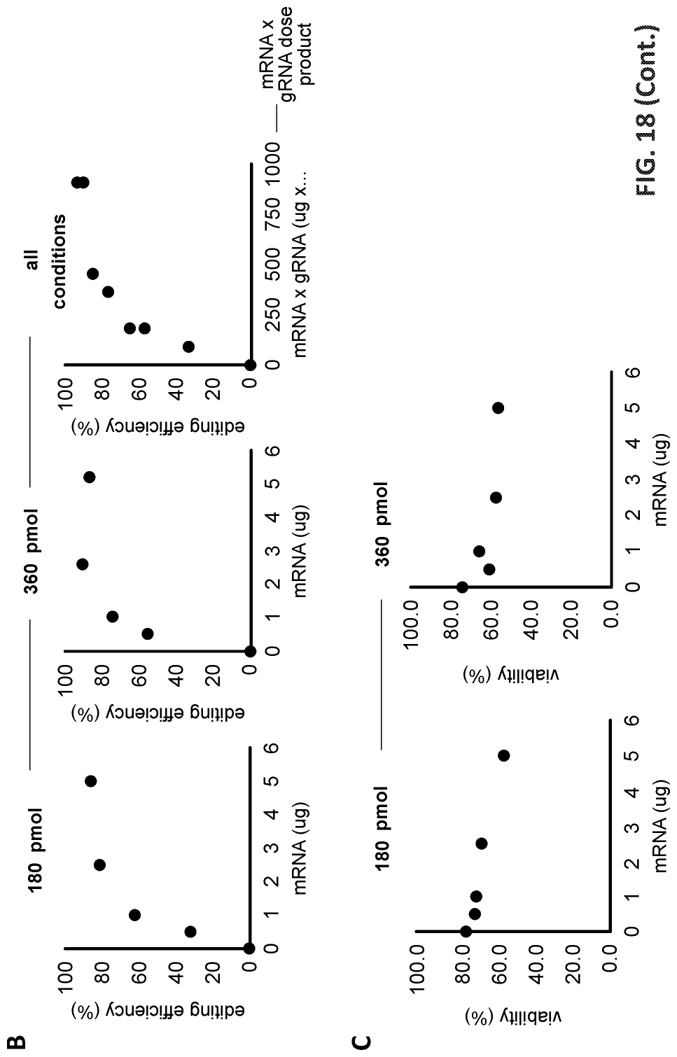

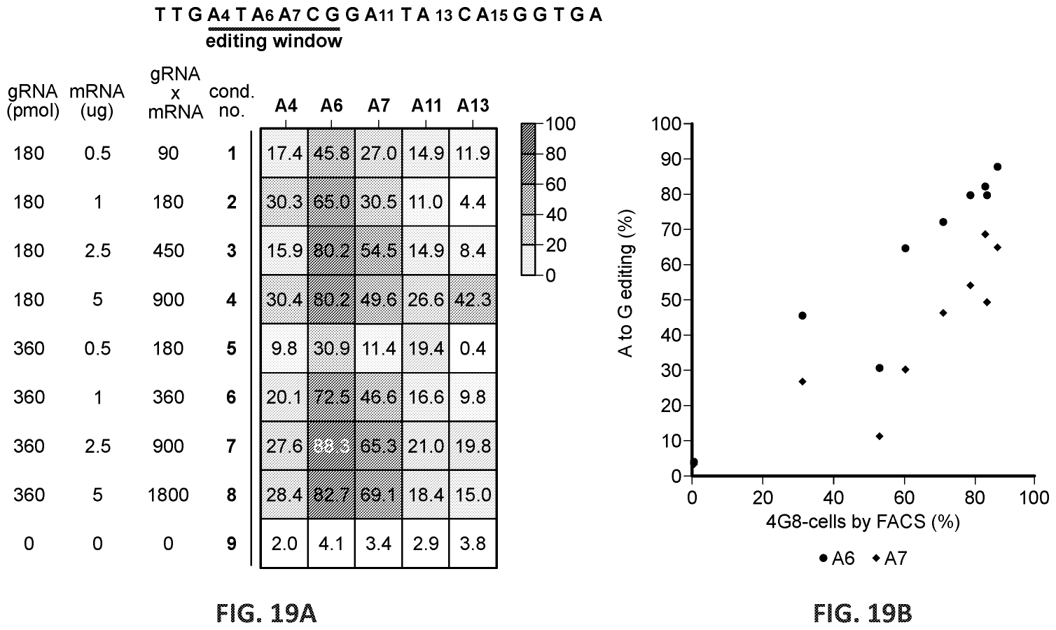

FIG. 18 exemplifies a base editing experiment on K562 reporter cells using electroporated IVT SpRY-ABE8e-V106W 3xNLS base editor mRNA at different doses (0.5 to 5 ug in 20 uL electroporation volume) and either 180 or 360 pmol of FLT3-e9-18 sgRNA. Edited cells were stained with 4G8 clone (targeting antibody) and BV10A4 clone for expression normalization (FIG. 18A). The percentage of edited cells by flow cytometry is reported in the bottom right of each plot. FIG. 18B shows the relationship between mRNA/gRNA doses and editing efficiency by flow cytometry, while FIG. 18C shows the relationship between mRNA/gRNA doses and cell viability by flow cytometry (LiveDead yellow staining). A heatmap showing the base editing efficiency by Sanger sequencing of PCR-amplified gDNA from samples of the experiment in FIG. 19A. The Sanger trace was deconvoluted using EditR package to calculate the A to G conversion efficiency. gRNA and mRNA doses are reported on the left, while the numbering of targeted adenine within the gRNA sequence are reported as columns (with the PAM corresponding to positions 21-23). FIG. 19B reports the relationship between editing efficiency by flow cytometry (4G8-cells) and Sanger sequencing.

To confirm that FLT3 base editing of primary human CD34+ hematopoietic stem and progenitor cells is feasible, an in vitro base editing and expansion culture experiment was performed. Mobilized peripheral blood-derived CD34+ HSPCs were thawed and cultured at 0.5-0.75 million/mL in StemCell SFEMII medium supplemented with 1% Penicillin/Streptomycin, SCF 100 ng/ml (Peprotech), FTL3L 100 ng/ml (Peprotech), TPO 50 ng/mL (Peprotech), Stemregenin-1 0.75 uM (StemCell technologies), UM171 35 nM (Selleckhem). 0.15-0.25 million (M) HSPCs were electroporated either 48 hours after thawing using Lonza 4D-Nucleofector system in P3 electroporation solution (Lonza) supplemented with 2.5-7.5 ug base editor mRNA (SpRY-ABE8e-V106W) and sgRNA (Integrated DNA Technologies) 250-450 pmol per 20 uL reaction. Cells were cultured in the aforementioned medium for 5-7 days. To test for specific resistance to CAR-T cell killing, edited or unedited CD34+ cells were expanded in vitro for 3 days post editing and then co-cultured with either anti-FLT3 CAR-T cells or untransduced T cells.

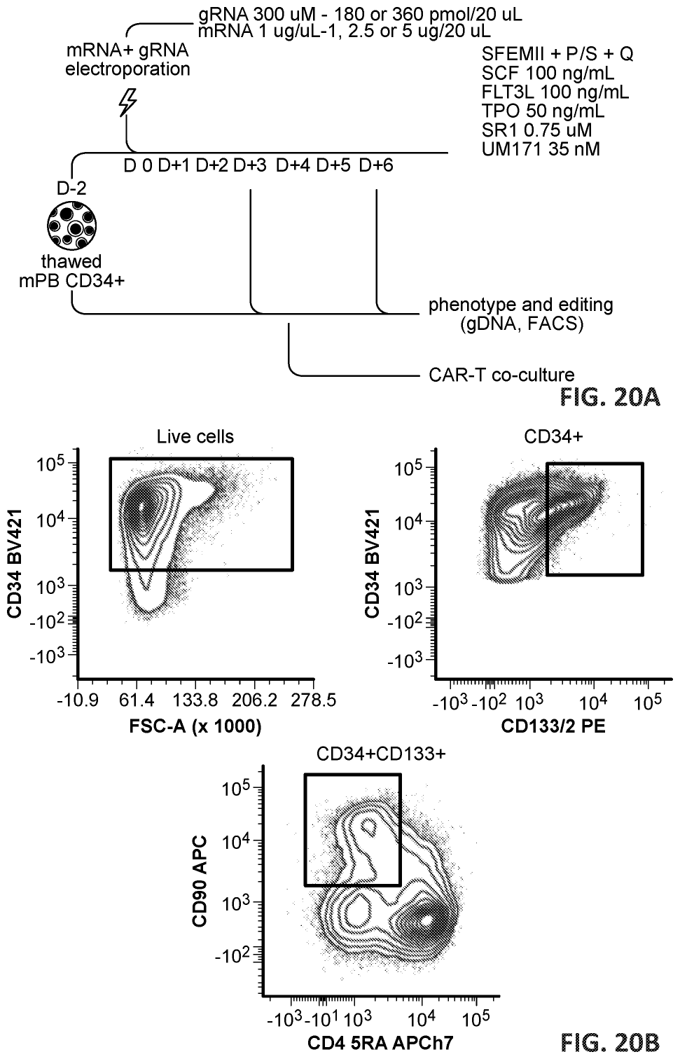

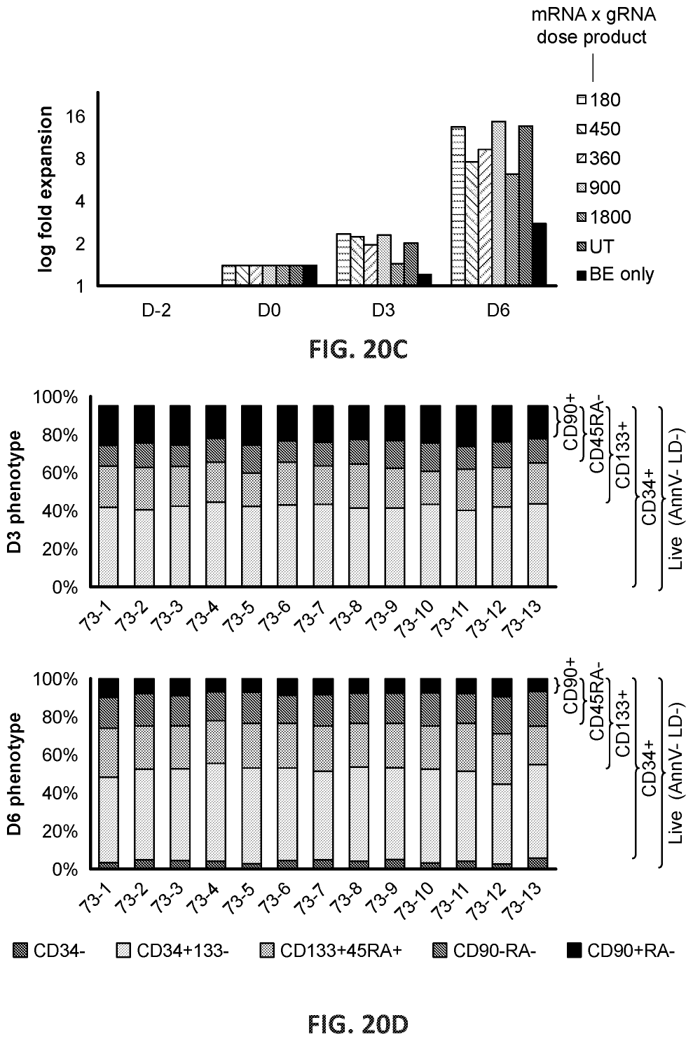

FIG. 20A shows the experimental layout and timeline for in vitro expansion culture and FLT3 base editing of mobilized peripheral blood-derived CD34+ HSPCs. Cell were electroporated with either 1, 2.5 or 5 ug of SpRY-ABE8e-V106W 3xNLS mRNA and 180 or 360 pmol of FLT3-e9-18 sgRNA. FIG. 20B exemplifies the gating strategy for flow cytometry immunophenotype of CD34+ cells during in vitro expansion culture. FIG. 20C shows the in vitro fold expansion of CD34+ cells relative to different base editing conditions, demonstrating limited toxicity of the base editing procedure, compared to the untreated control. FIG. 20D summarizes the compositions by flow cytometry of cultured CD34+at day 3 and day 6 post editing, showing no skewing of the stem cell and progenitor subsets upon electroporation and mRNA base editing

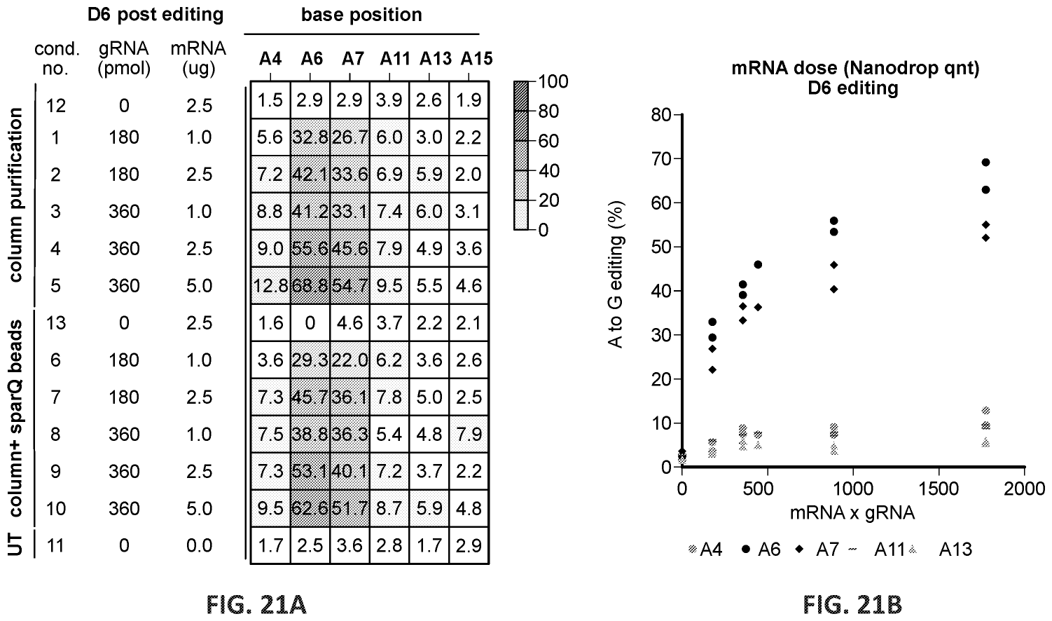

Editing efficiencies by Sanger sequencing at day6 post electroporation are reported in FIG. 21A. gRNA and mRNA doses are reported on the left, while the numbering of targeted adenine within the gRNA sequence are reported as columns (with the PAM corresponding to positions 21-23). The relationship between base editing efficiency and different gRNA×mRNA doses in human CD34+ HSPCs is reported as a scatter plot in FIG. 21B.

### Example 8: FLT3 Base Editing of Human FLT3-Expressing Leukemia Cell Lines or Human CD34+ HSPCs with SpRY-ABE8e-V106W mRNA Confers Resistance to 4G8 CAR-T Cells

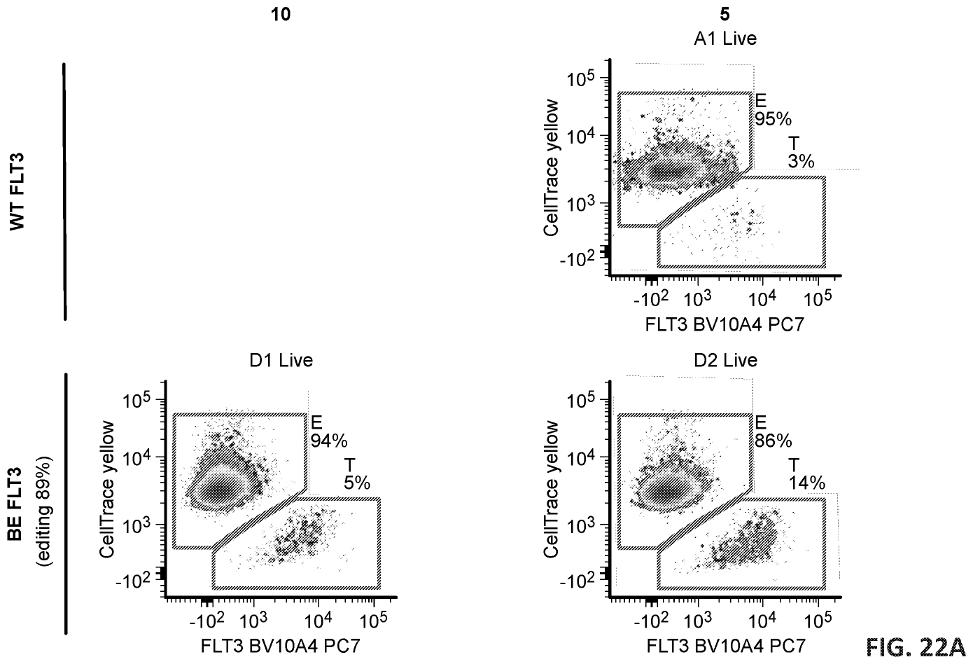

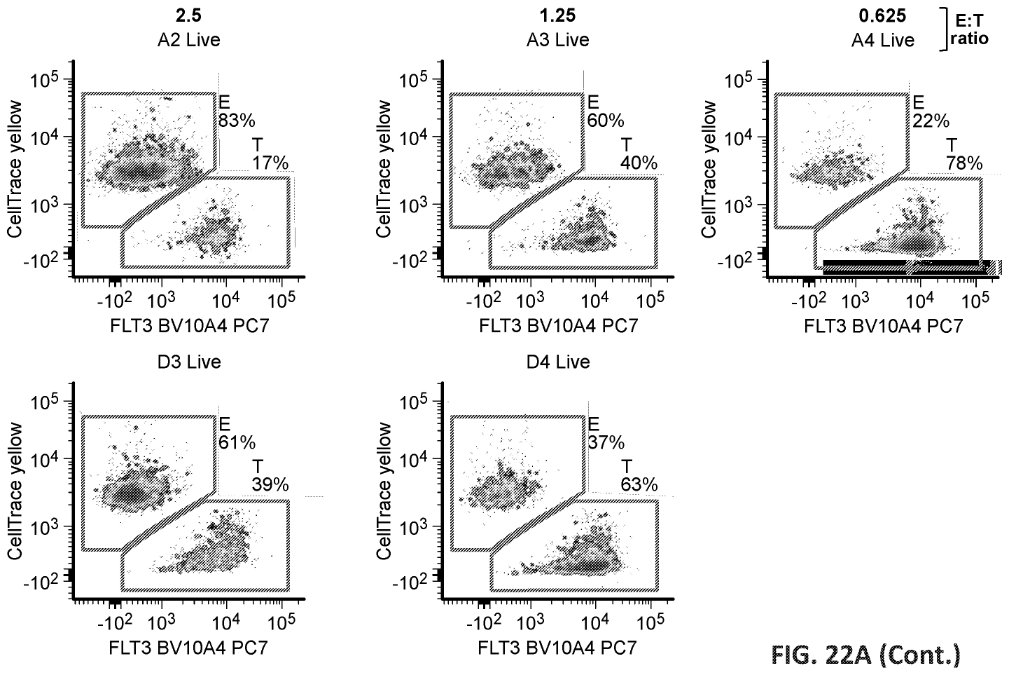

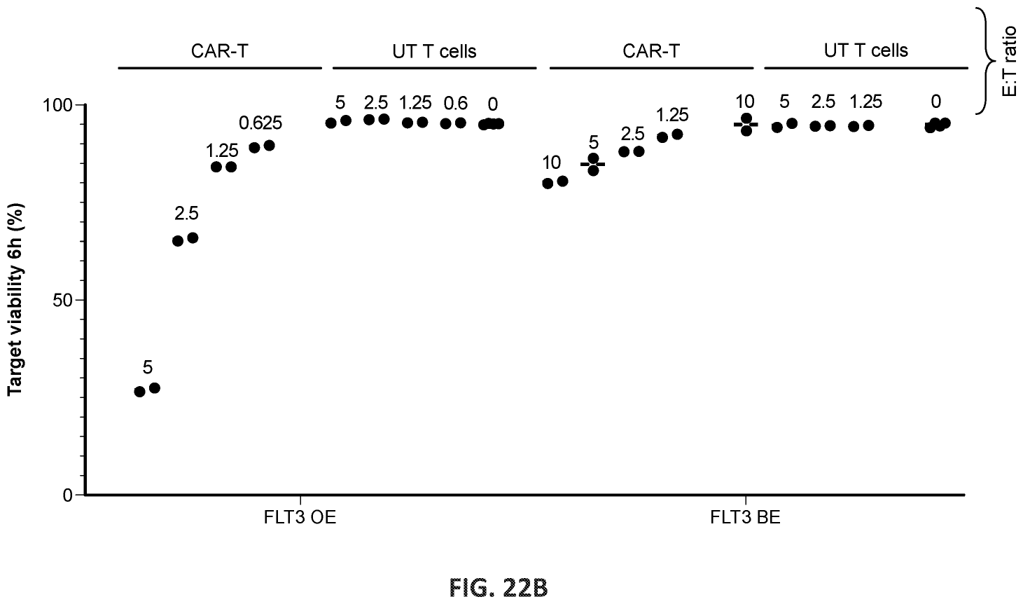

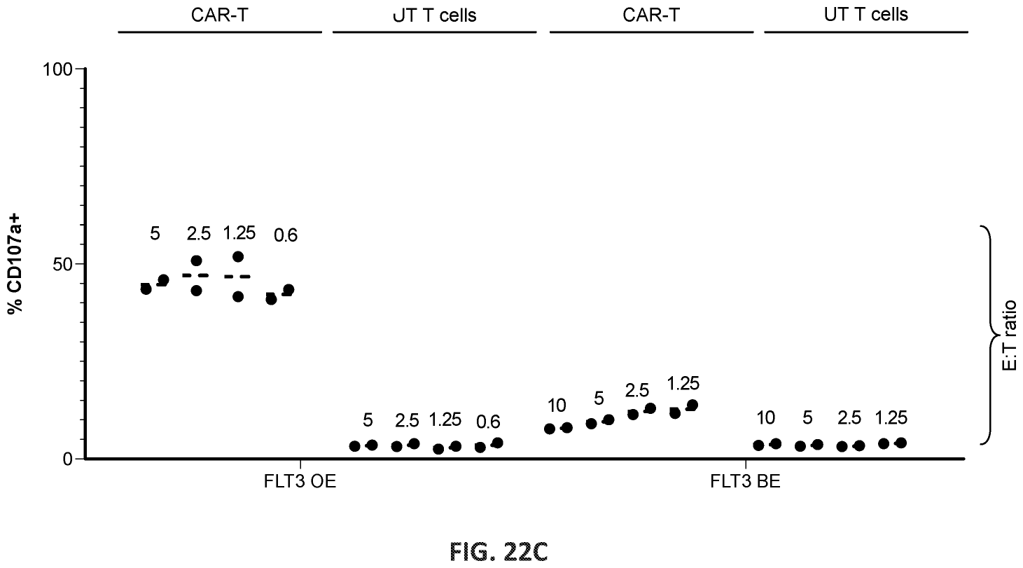

To test if base edited cells obtained from the experiments done in FIG. 18 are resistant to 4G8 CAR-T cells, a co-culture experiment was performed as previously reported. Flow cytometry plots of surviving cells at 4 hours after co-culture at different effector: target ratios are reported in FIG. 22A. Edited cells (editing efficiency ˜89%) are protected from CAR-T cell killing and survive even at higher E:T ratios. FIG. 22B reports target cell (unedited or FLT3 base edited K562) viability by AnnexinV and LiveDead yellow staining at 4 hours after co-culture with either 4G8 anti FLT3 CAR-T cells or untransduced T cells, highlighting selective killing of non-modified cells. CAR-T/T cell degranulation by surface CD107a staining at 4h of co-culture with either unedited or FLT3 base edited K562 cells is reported in FIG. 22C. Only CAR-T cells exposed to unmodified cells show significant degranulation.

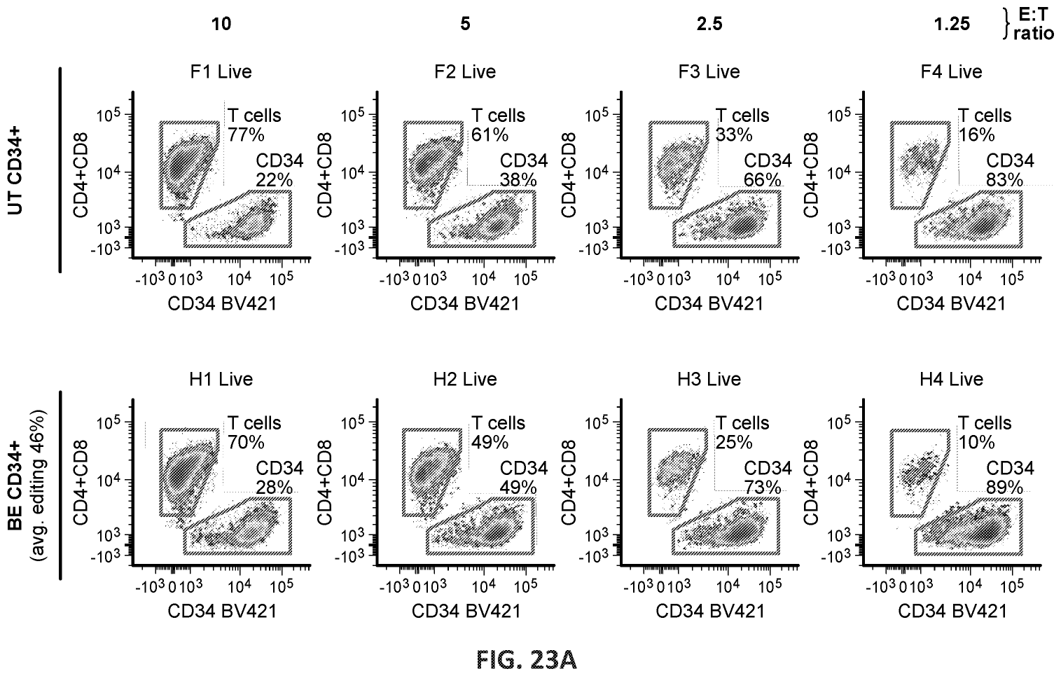

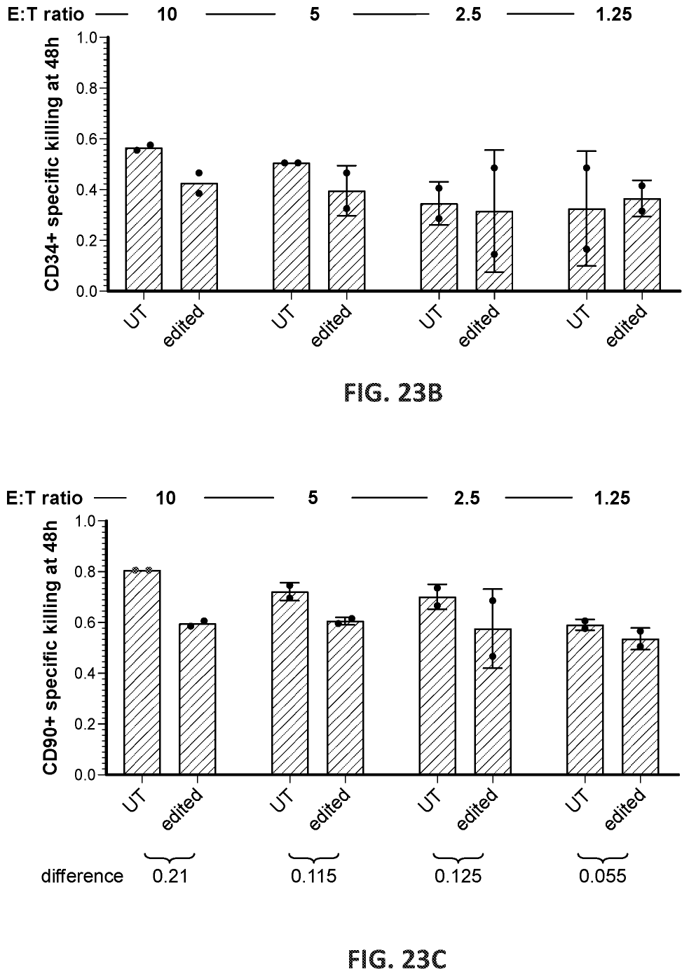

Similarly, to test if base edited human hematopoietic stem and progenitor cells are resistant to FLT3-targeting immunotherapies, CD34+ edited in the experiment performed in FIG. 20 were co-cultured with 4G8 CAR-T cells or untransduced T cells. FIG. 23 shows a CAR-T cell co-culture experiment with unmodified or FLT3 base edited mPB CD34+ cells (editing efficiency ˜46%) from day 3 culture of the experiment in FIG. 20. Flow cytometry plots of surviving cells at 48h of co-culture at different effector: target ratios with either 4G8 anti FLT3 CAR-T cells or untransduced T cells are reported in FIG. 23A. The specific killing of CD34+ cells co-cultured at different effector: target ratios with either 4G8 anti FLT3 CAR-T cells or untransduced T cells is reported in FIG. 23B. The specific killing for the stem cell enriched CD34+90+ subset is reported in FIG. 23C. While the differences in CAR-T mediated killing are less pronounced compared to experiments performed on cell lines, due to the relatively lower expression of FLT3 by cultured CD34+ HSPCs exposed to FLT3L, modified HSPCs show resistance conferred by FLT3 editing, with higher differences within the more stem-enriched CD90+ cells.

### Example 9: FLT3-Edited HSPC are Protected by CAR-T Cell Mediated Killing In Vivo

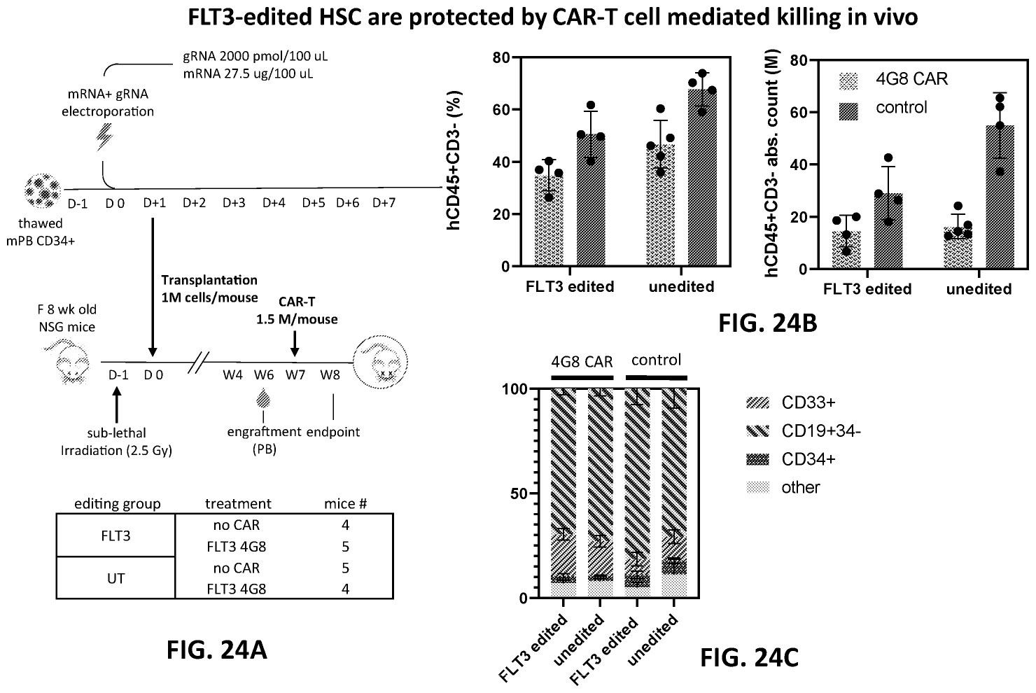

To further confirm the protective role against on-target killing mediated by anti-FLT3 CAR-T cells, an in vivo xenotransplantation experiment in immunodeficient NSG mice was performed (FIG. 24A shows the experimental design). Human mobilized peripheral blood derived CD34+ HSPCs were edited as previously exemplified and transplanted at 1 million per mouse by tail vein injection 24 h after sublethal irradiation (2.5 Gy). Mice xeno-transplanted with non-edited HSPCs served as control group. Each group was further divided in two treatment subsets, either vehicle (PBS) or 4G8-CAR T cells 1.5 million cells per mouse at week 7 post-transplant. The mice were euthanized at week 8 (one week after CAR-T cell treatment). Human bone marrow engraftment by flow cytometry (% of human CD45+ cells, excluding CD3+ cells) and human absolute CD45+ cell count are reported in FIG. 24B. CD3+ are excluded from the human engraftment as they are derived from injected CAR-T cells. More severe depletion of human engraftment is observed in mice transplanted with unedited cells. Human engraftment composition, CD3+ cells excluded, on bone marrow is reported in FIG. 24C, demonstrating multilineage engraftment generated by edited HSPCs.

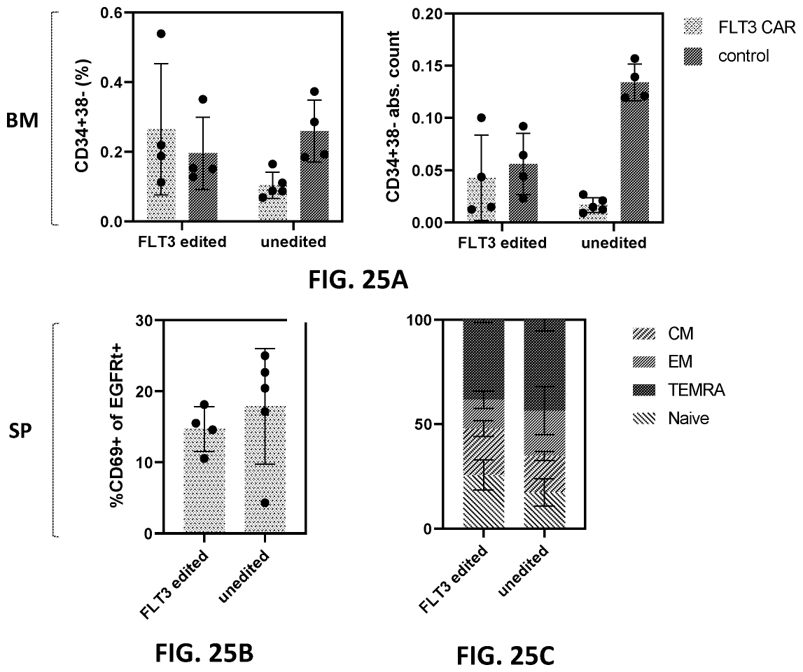

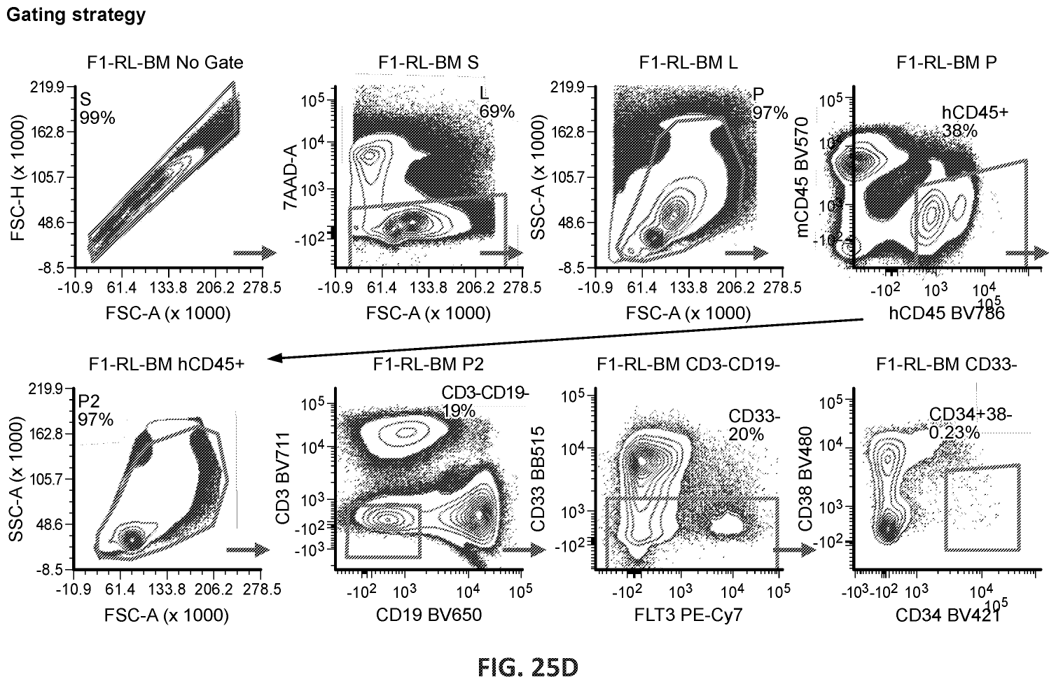

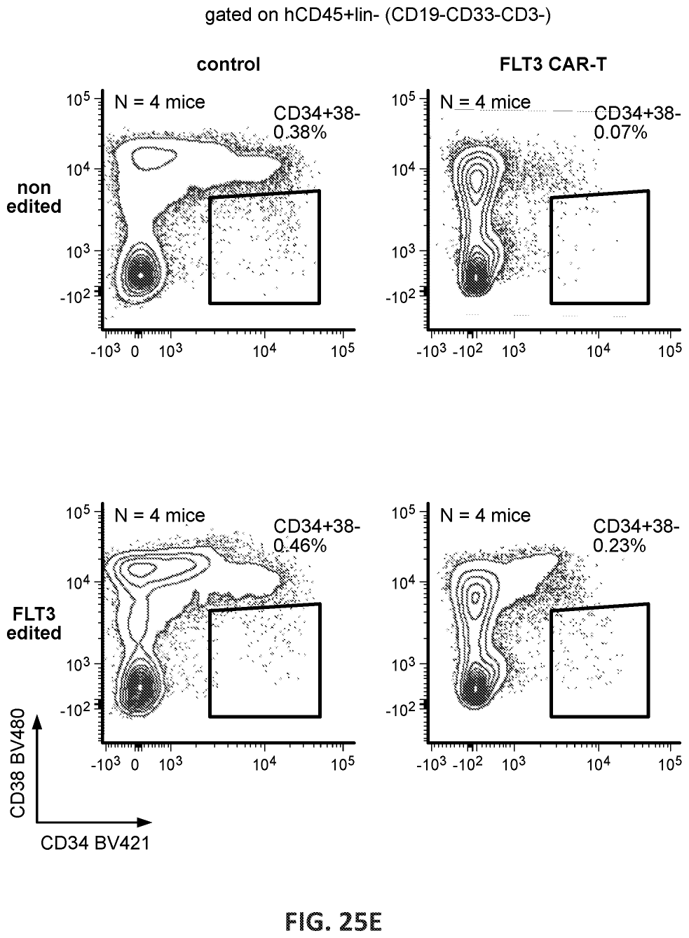

Hematopoietic stem and progenitor cell frequency (CD34+CD38−) and absolute abundance in the bone marrow at sacrifice are reported in FIG. 25A, demonstrating a protective effect of FLT3 base editing against CAR-T mediated killing. Exemplary flow cytometry plots resulting from pooled events from bone marrow analysis of mice from each group are reported in FIG. 25D (gating strategy) and FIG. 25E (fraction of lineage-CD34+38− persisting cells). Relative higher activation of CAR-T cells in the spleen of treated mice by surface CD69 staining is showed in FIG. 25B. CAR-T cells were identified by EGFR staining (co-expressed with the CAR as marker and safety switch). CAR-T cell phenotype by CD62L and CD45RA expression in the spleen of treated mice is reported in FIG. 25C. Naive: CD45RA+CD62L+, central memory (CM): CD45RA−CD62L+; effector memory (EM): CD45RA−CD62L−; terminally differentiated effector memory (TEMRA): CD45RA+CD62L−. A relative skewing towards effector subsets is evident in treated mice xeno-transplanted with unedited HSPCs.

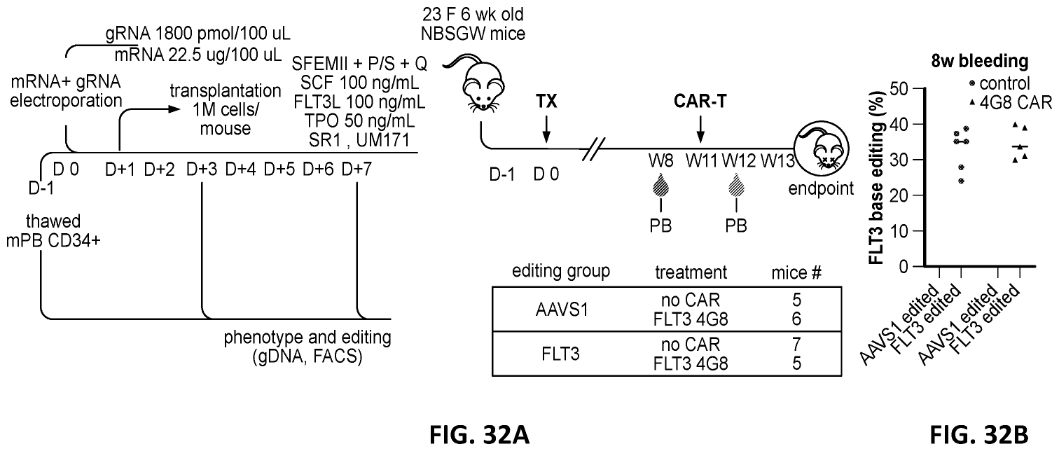

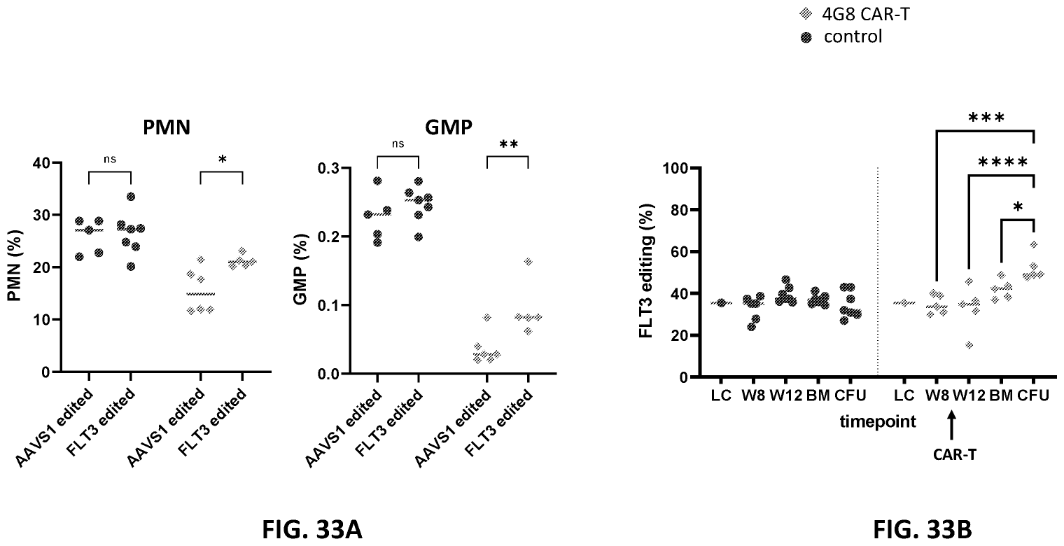

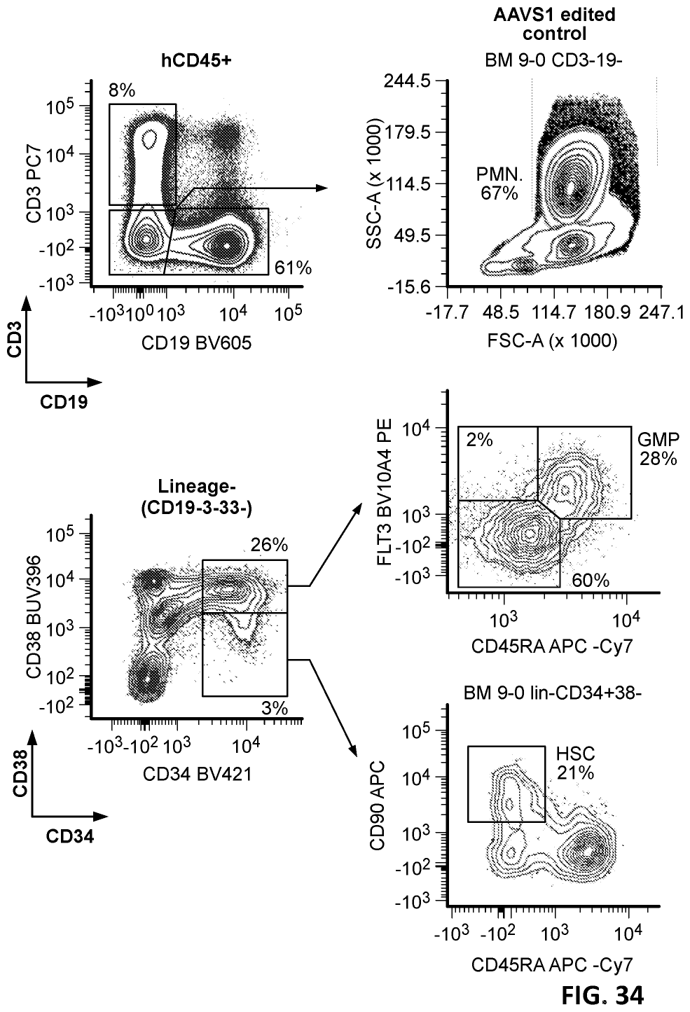

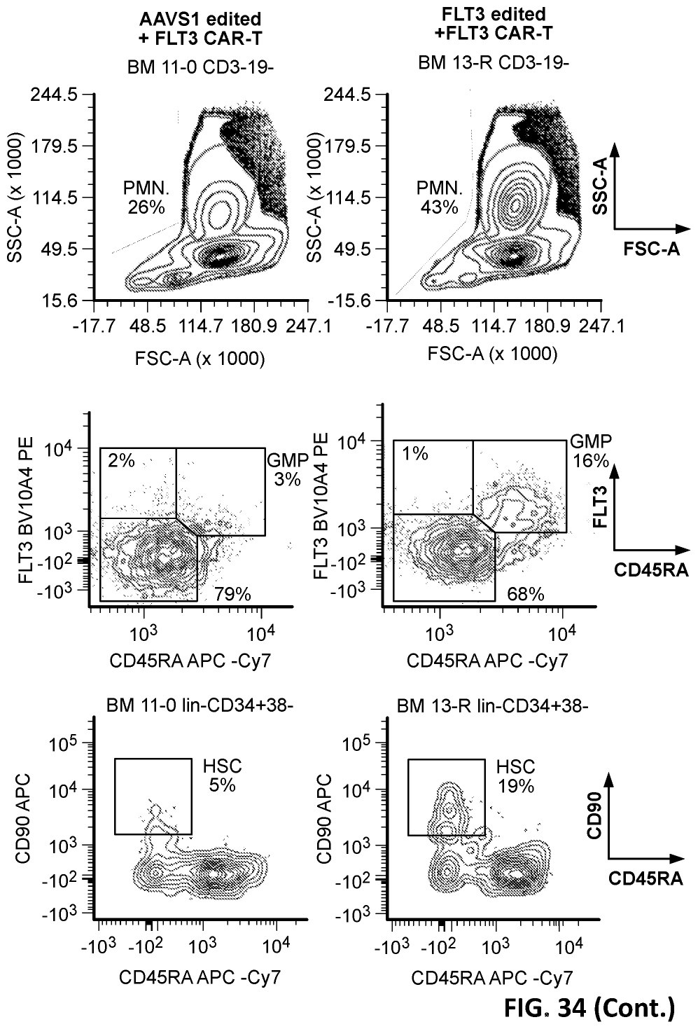

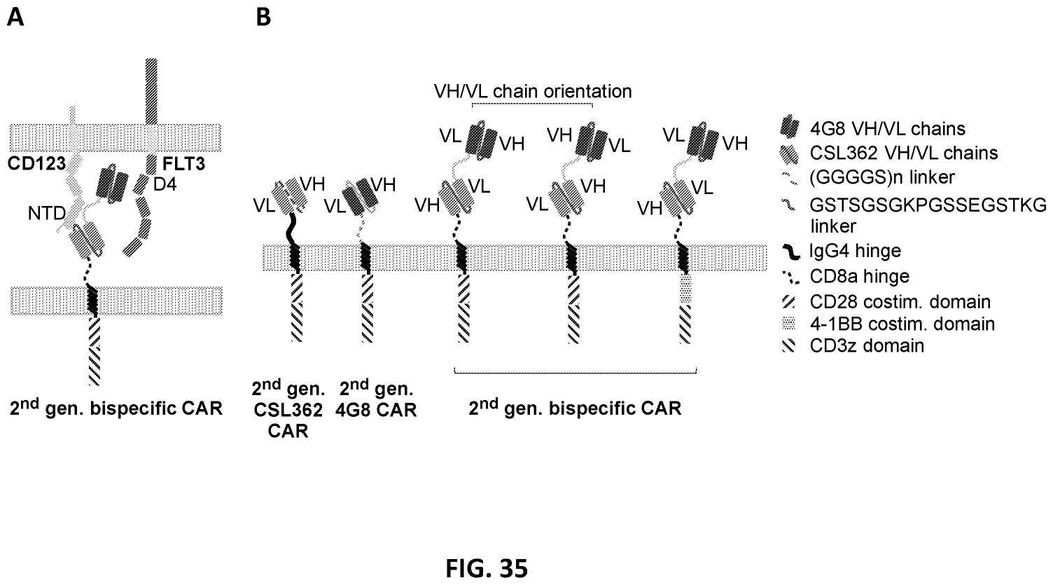

To further evaluate the protection of CD34+ HSPCs by FLT3 base editing of N399 against CAR-T cell killing, a second experiment in vivo experiment was performed in NBSGW mice, which allow xeno-transplantation and engraftment of human HSPCs without irradiation and, thanks to a hypomorphic Kit mutation in murine HSPCs, (homozygous W41 allele) higher levels of human hematopoietic reconstitution. FIG. 32 reports the experimental setup and FLT3 base editing efficiency on engraft human cells at week 8 bleeding. Treated groups received 2.5 million 4G8 CAR-T cells at week 11 post transplantation and were subsequently euthanized at week 13 post transplantation. This experimental setting allowed improved evaluation of myeloid progenitor and lineage protection conferred by FLT3 base editing. FIG. 33A shows the relative abundance of granulocytes (polymorphonucleated cells, PMN) by flow cytometry on bone marrow at sacrifice, with clear protection in mice transplanted with FLT3 edited HSPCs, compared to AAVS1-control edited cells. As mature granulocytes do not express FLT3, this indicates some degree of protection at the progenitor level. Consistent with this hypothesis, granulo-mono progenitors (GMP), which are defined as lineage-CD34+CD38+FLT3+CD45RA+, are selectively depleted by FLT3 CAR-T cells only in mice xeno-transplanted with mock-edited HSPCs (FIG. 33A). FIG. 33B reports the FLT3 base editing efficiency at several time-points in 4G8-CAR treated or untreated groups. Upon 4G8-CAR administration there is progressive negative selection of unmodified cells, resulting in selection of FLT3-edited cells (which is more evident in the progeny derived from colony forming unit assays plated with bone marrow samples of treated mice). Representative FACS plots showing the granulocyte, granulo-mono progenitors (GMP) and HSPC (lineage-CD34+CD38−CD90+CD45RA−) populations are reported in FIG. 34.

### Example 10: CD123 Base Editing of Human CD34+ HSPCs with SpRY-ABE8e-V106W mRNA is Feasible and can be Multiplex with FLT3 Base Editing

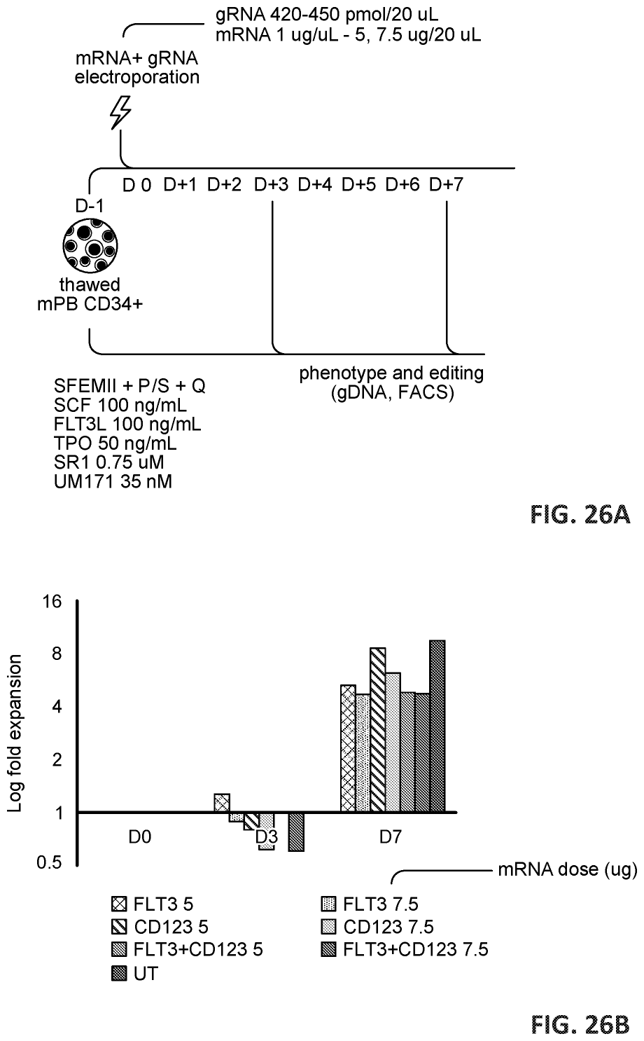

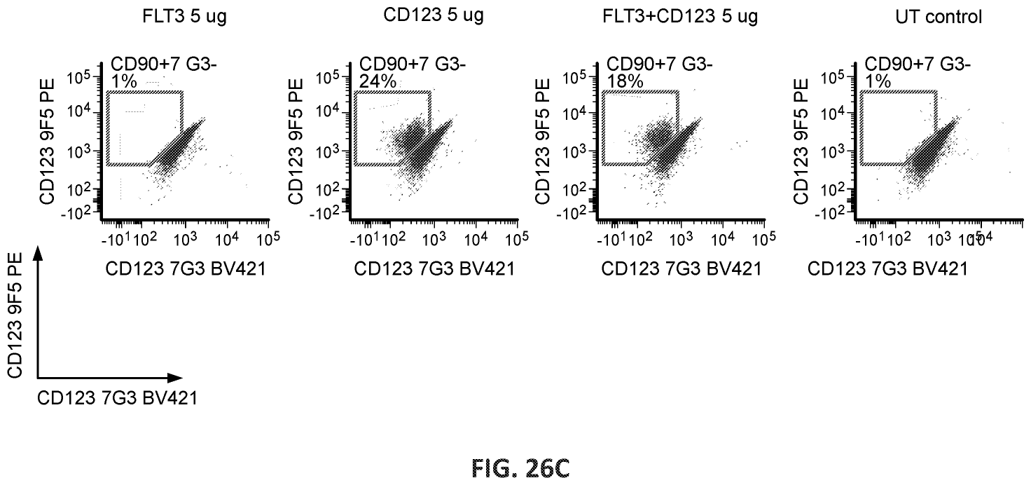

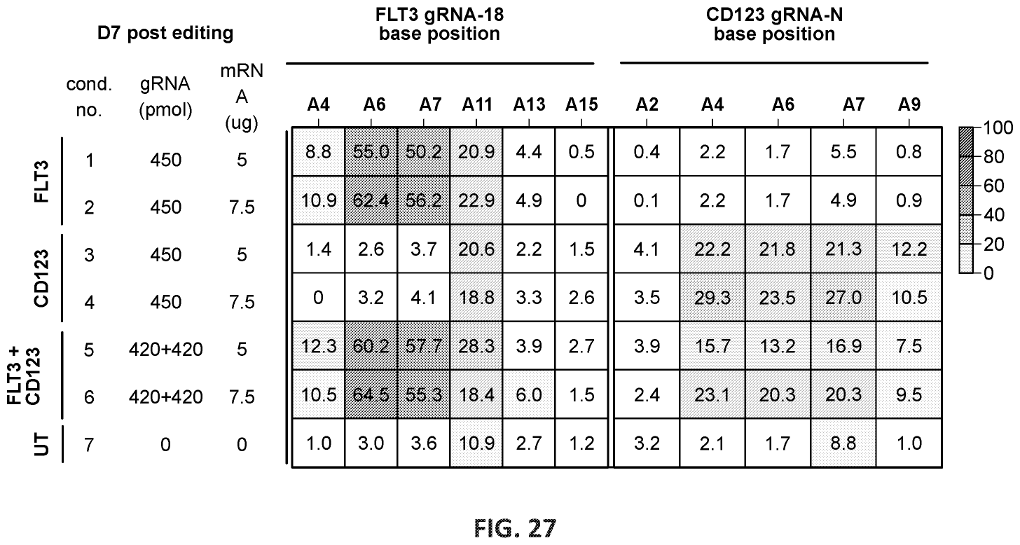

Similar to FLT3-base editing experiments, we introduced the S59P mutation using SpRY-ABE8e-V106W mRNA and CD123 gRNA-N in human CD34+ HSPCs, either alone or in combination with FLT3 N399 base editing. Mobilized peripheral blood-derived CD34+ HSPCs were thawed and cultured at 0.5-0.75 million/mL in StemCell SFEMII medium supplemented with 1% Penicillin/Streptomycin, SCF 100 ng/ml (Peprotech), FTL3L 100 ng/ml (Peprotech), TPO 50 ng/ml (Peprotech), Stemregenin-1 0.75 uM (StemCell technologies), UM171 35 nM (Selleckhem). 0.15-0.25 million (M) HSPCs were electroporated either 48 hours after thawing using Lonza 4D-Nucleofector system in P3 electroporation solution (Lonza) supplemented with 5-7.5 ug base editor mRNA (SpRY-ABE8e-V106W) and sgRNA (Integrated DNA Technologies) 420-450 pmol per 20 uL reaction. Cells were cultured in the aforementioned medium for 7 days. FIG. 26A exemplifies the experimental layout and culture media composition. FIG. 26B shows the in vitro expansion of cultured CD34+ HSPCs, while FIG. 26C shows the loss of 7G3 clone binding to successfully base edited CD34+ HSPCs. The flow cytometry plots are gated on the stem-enriched CD90+ subset, showing CD123 base editing of HSPCs by loss of 7G3 staining when normalized with clone 9F5 staining. The editing efficiencies of FLT3 single edited, CD123 single edited and FLT3+CD123 dual base edited conditions are reported in FIG. 27. Whereas it is feasible to introduce the S59P mutation through base editing, the efficiencies obtained in our setting are relatively low (up to 29.3%) compared to FLT3 N399 (>64%).

### Example 11: CD123 Base Editing Efficiency can be Improved by Optimization of sgRNA Position and Introduction of the K918N Substitution within the Cas9 Protein of the Adenine Base Editor

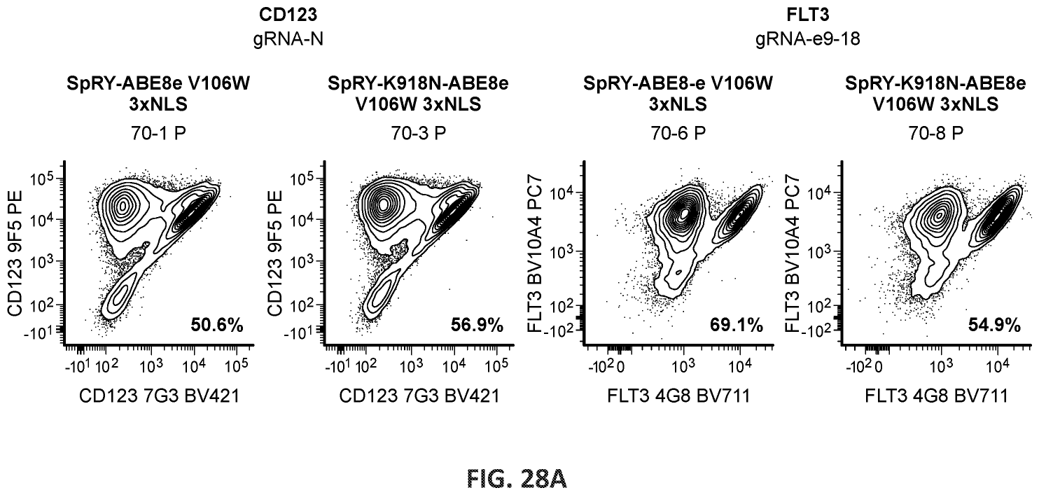

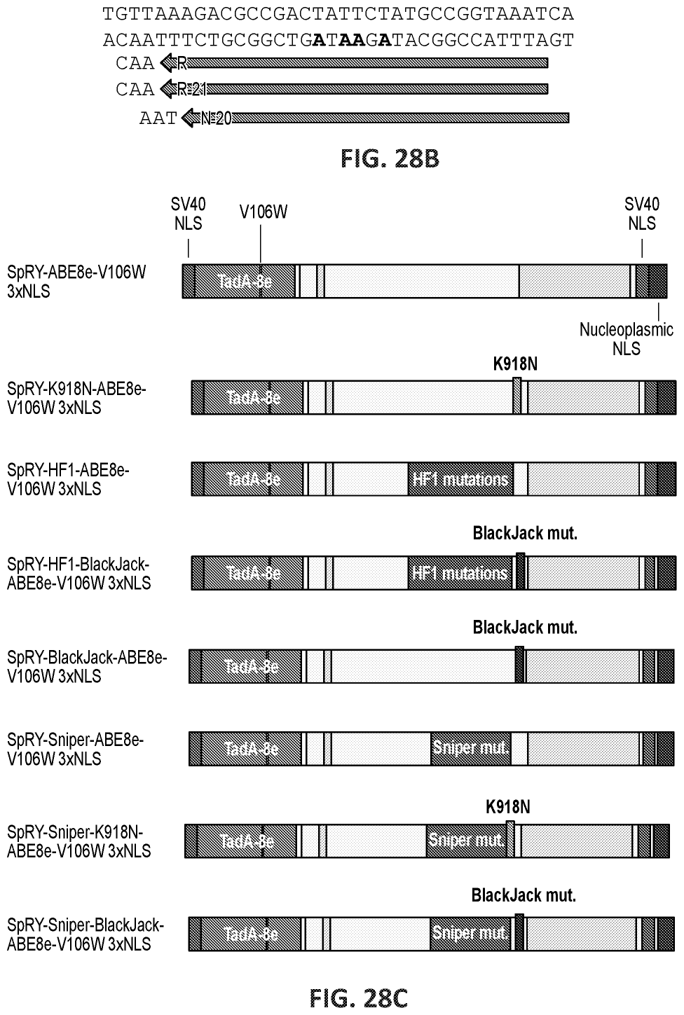

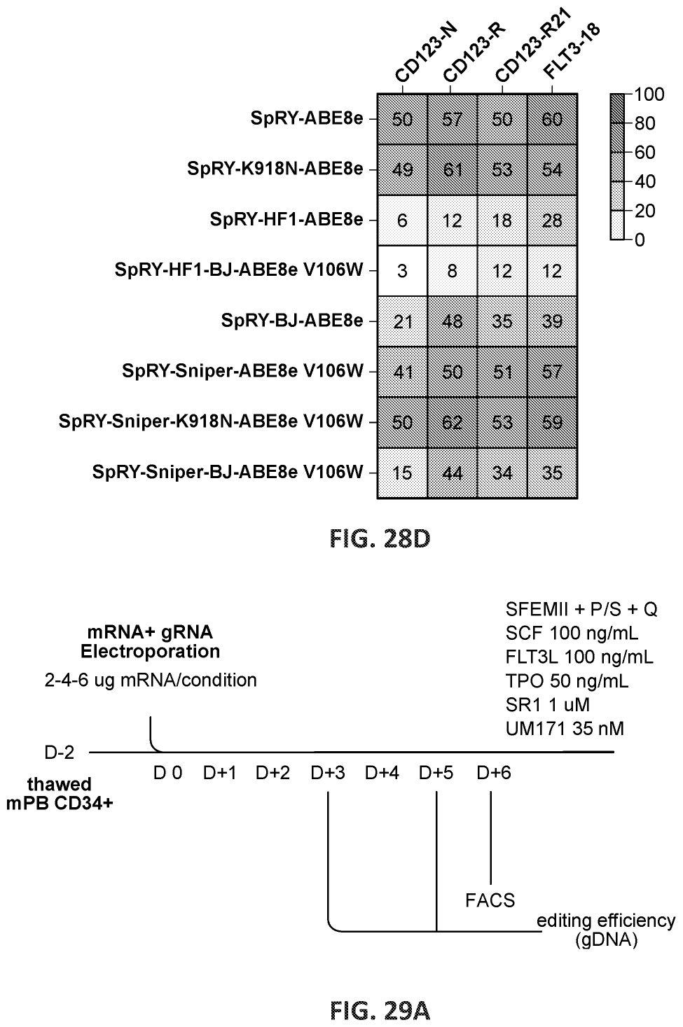

To see if editing efficiency of CD123 S59 codon could be improved, a Cas9 protein containing the K918N mutation, which has been associated with improved Cas9 catalytic activity was tested in combination with our SpRY-ABE8e design. As shown in FIG. 28A the introduction of K918N mutation resulted in sequence-specific effects on editing efficiency, with improvement for CD123-gRNA-N while FLT3-gRNA-18 editing was slightly reduced. FIG. 28A shows the base editing outcomes by flow cytometry at day 3 after electroporation of 500 ng base editor-expressing plasmid and 360 pmol of sgRNA into K562 reporter cells. Edited cells were stained with 7G3 (targeting antibody) plus 9F5 clone (normalization) for CD123. The gRNA CD123-N(CD123_gRNA_N, SEQ ID NO: 24), was tested in combination with SpRY-ABE8e-V106W-3xNLS BE, as in previous experiments, or with SpRY-K3918N-ABE8e-V106W-3xNLS (SEQ ID NO: 9 and SEQ ID NO: 12, sequences are above). The percentage of edited cells by flow cytometry is reported in the bottom right of each plot.

In order to further improve CD123 editing efficiency, two additional sgRNAs (CD123-gRNA-R and its 21-bp long version, CD123-gRNA-R21) were cloned alongside the benchmark CD123-gRNA-N and FLT3-gRNA-18 in a pHKO-mTagBFP2 plasmid under a human U6 promoter. FIG. 28B reports the relative positions and sequences of CD123 S59 targeting gRNAs. Furthermore, additional adenine base editor designs were cloned and tested alongside SpRY-ABE8e-V106W (FIG. 28C): SPRY-K918N-ABE8e-V106W, SpRY-HF1-ABE8e-V106W, SpRY-HF1-BlackJack-ABE8e-V106W, SpRY-BlackJack-ABE8e-V106W, SpRY-Sniper-ABE8e-V106W SpRY-Sniper-BlackJack-ABE8e-V106W. The HF1 variants include N497A, R661A, Q695A, Q926A substitutions associated with lower tolerance for gRNA spacer mismatched bases and lower off-target editing (High Fidelity). The Sniper variants include F539S, M763I, K890N substitutions, which produce another higher fidelity SpCas9 variant with preserved on-target efficiency. BlackJack is a Cas9 variant designed to be tolerant to 21-bp and longer sgRNAs, which is relatively more important for higher fidelity variants. Co-electroporation of 500 ng of base editor plasmid and 500 ng of sgRNA expressing plasmid in K562 reporter cells was performed for all combinations of sgRNA (CD123-gRNA-N, CD123-gRNA-R, CD123-gRNA-R21, FLT3-gRNA-18) and the outcome was evaluated 72h after editing by flow cytometry. FIG. 28D summarizes the result of the screening procedure, which highlights improved combinations for CD123 S59 base editing. In particular, excluding the HF1 variants, CD123-gRNA-R is on average 1.42× more efficient than CD123-gRNA-N, while its 21-bp long counterpart (CD123-gRNA-R21) is only 1.21× more efficient. Introduction of the K918N mutation improves CD123 editing by 1.08× for gRNA-R and 1.05× for gRNA-N. In combination with Sniper mutations, K918N improves efficiencies by 1.22× for gRNA-R and 1.16× for gRNA-N. In conclusion, switching to CD123-gRNA-R and introduction of K918N mutation would partially compensate for the lower efficiencies observed for CD123 editing compared to FLT3, which appears to be relatively unaffected by the K918N substitution.

### Example 12: Improved Duplex Base Editing Efficiency with SpRY-K918N-ABE8e-V106W mRNA in CD34+ HSPCs

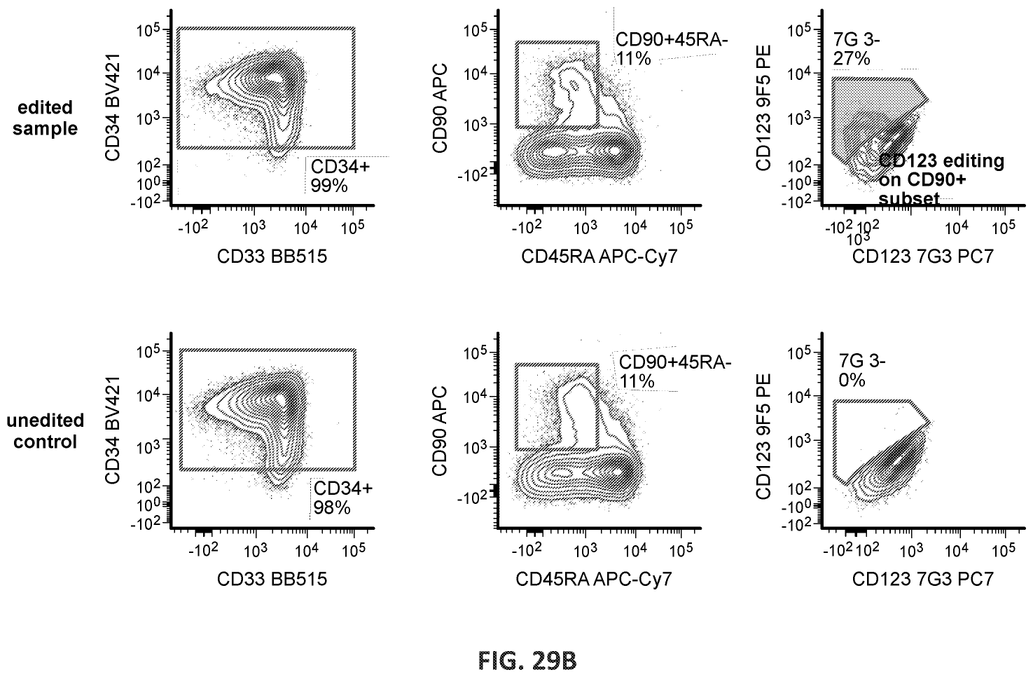

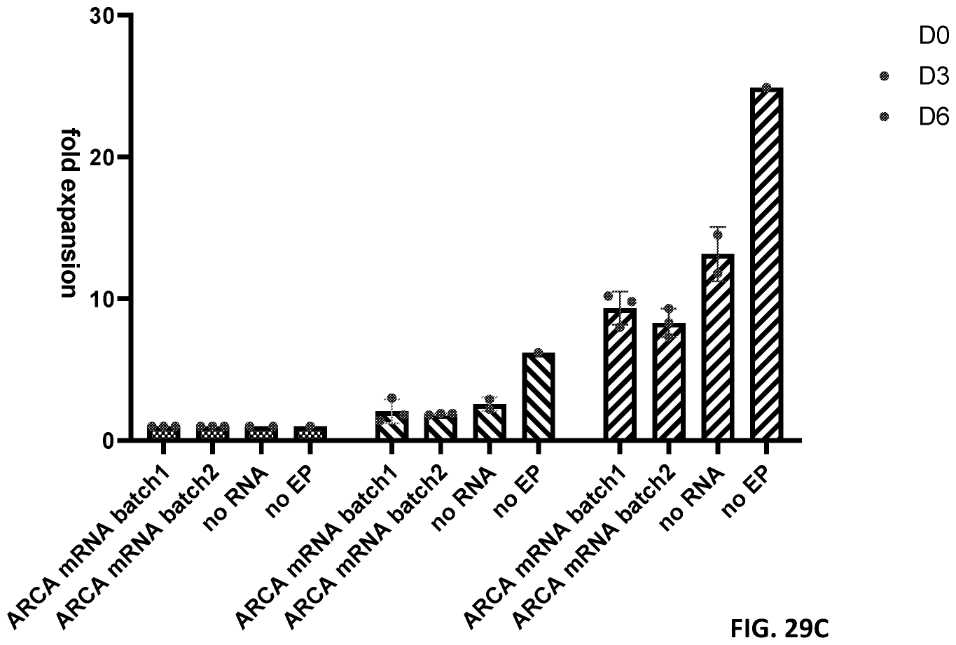

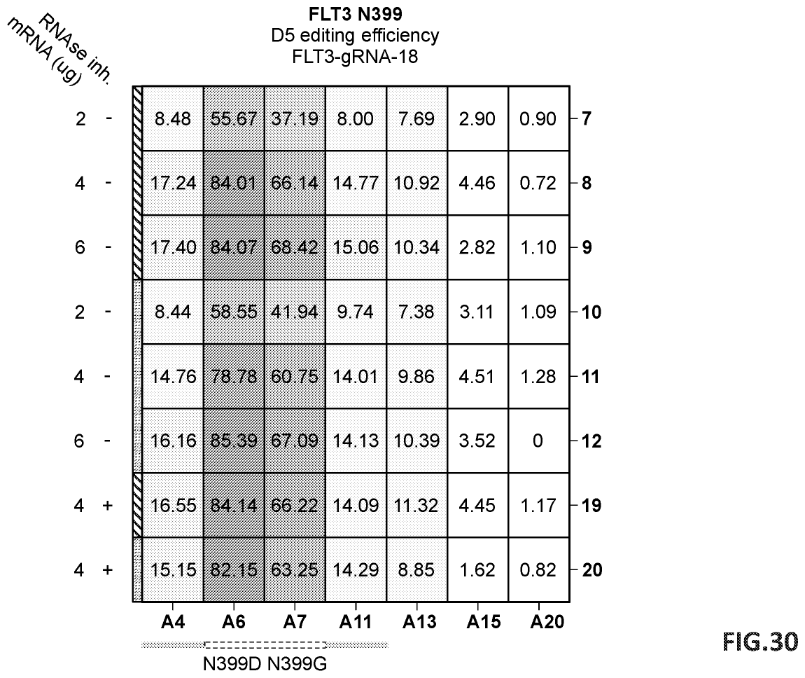

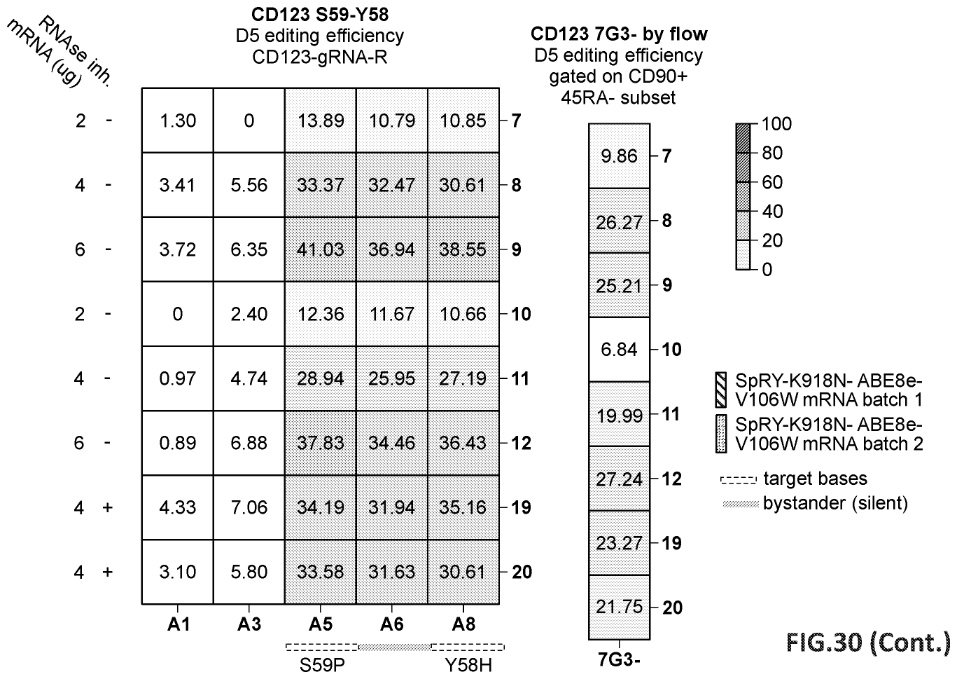

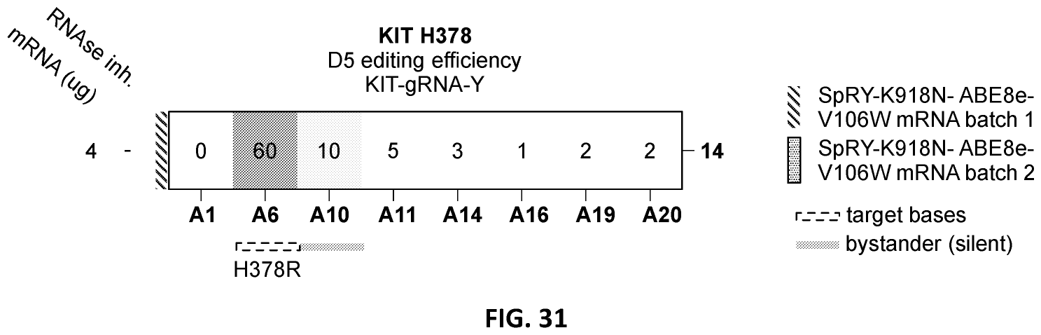

Improvement in CD34+ HSPCs base editing efficiencies for FLT3 and CD123 loci using SpRY-K918N-ABE8e-V106W in vitro transcribed mRNA was achieved through optimization of multiple parameters (mRNA preparation, K918N mutation introduction, sgRNA selection). An exemplary experiment is reported in FIG. 29. FIG. 29A summarizes the experimental design, FIG. 29B reports the gating strategy for CD123 editing evaluation by flow cytometry while FIG. 29C demonstrates similar expansion for FLT3+CD123 base edited cells when compared to untreated (electroporation only) control. Sanger sequencing revealed up to 85% for FLT3 N399 editing and up to 41% efficiency for CD123 S59 editing (FIG. 30).

### Example 13: KIT H378R Mutation can be Introduced Through Adenine Base Editing in CD34+ HSPCs

Using the same experimental setting as Example 12, we tested whether H378R mutation could be introduced through base editing in human CD34+ HSPCs. By electroporating SpRY-K918N-ABE8e-V106W in vitro transcribed mRNA (4 ug/20 μL electroporation volume) and KIT-gRNA-Y (SEQ ID NO: 37), editing efficiency up to 60% was achieved.

### Example 14: Generation of Anti-FLT3 and CD123 Bispecific Chimeric Antigen Receptors

Rationally designed anti-FLT3 and CD123 bispecific chimeric antigen receptors should result in potent antileukemia efficacy. FIG. 29A is a schematic of a putative 2^{nd} generation bispecific chimeric antigen receptor targeting both FLT3 domain 4 and CD123 N-terminal domain. FIG. 29B is the design of a 2nd generation single-specificity CARs targeting CD123 (CSL362 clone, SEQ ID NOs: 75-76) and FLT3 (4G8 clone, SEQ ID NOs: 73-74) and bispecific chimeric antigen receptor with different orientation of the scFv domains and or different extracellular linkers, transmembrane domains, intracellular co-stimulatory domains (SEQ ID NOs: 77-80).

### Example 15: Base Editing Generates Stealth Receptors

As targets for our epitope engineering strategy, we selected the cytokine receptors FLT3, KIT and CD123 (IL3RA). Fms-like tyrosine kinase 3 (FLT3, CD135) and proto-oncogene c-KIT (KIT, CD117) are class III receptor tyrosine kinase which are expressed, either in wild type (WT) or mutated form, in 93% and 85% of AML cases, respectively³²⁻³⁷. CD123 is the alpha subunit of the IL-3 receptor (IL3RA), a type I cytokine receptor found on the surface of >75% of AML cases and overexpressed on the surface of leukemic stem cells³⁸⁻⁴⁰. These genes are present at various stages of normal hematopoietic development and their overexpression on AML cells is associated with poor prognosis, with higher incidence of relapse after HSCT and a lower overall survival rate both in adult and pediatric patients^{33,36,41-43}. To develop our approach, we selected monoclonal antibodies (mAb) currently under evaluation for the development of anti-AML immunotherapies: clone 4G8^{22,44} (FLT3), Fab-79D^{21,45} (KIT) and 7G3⁴⁶⁻⁴⁸ (CD123). Previous studies reported that 4G8 recognizes FLT3 extracellular domain (ECD) 4, while clone BV10A4 recognizes an unrelated epitope within ECD2 and therefore can serve as control to assess FLT3 surface expression⁴⁴. Since 4G8 was generated by immunizing BALB/c mice with human FLT3 transfected cell lines, we reasoned that 4G8 recognizes a human-specific epitope, despite the high degree of homology (85.8% identity and 91.5% similarity) and FLT3 ligand (FLT3L) cross-reactivity between human and mouse FLT3⁴⁹.

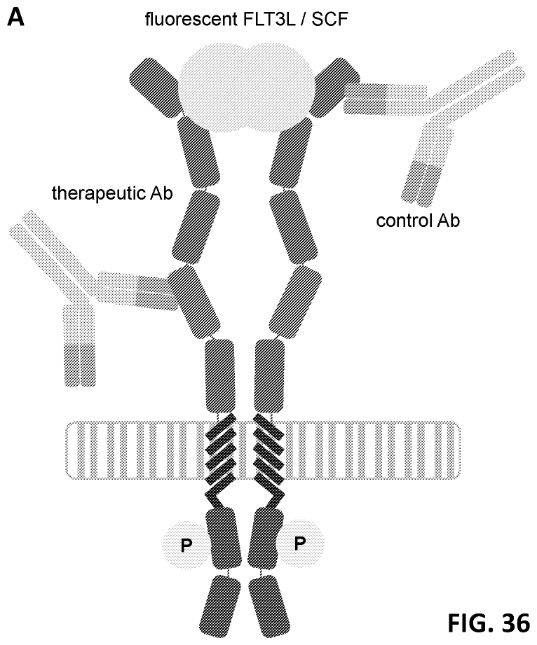

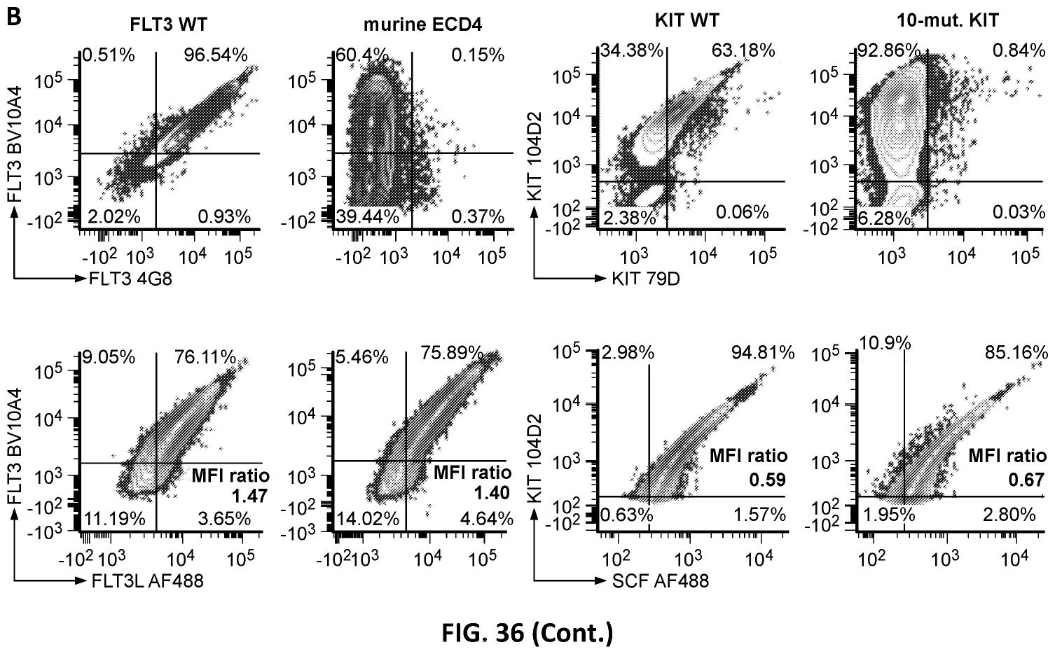

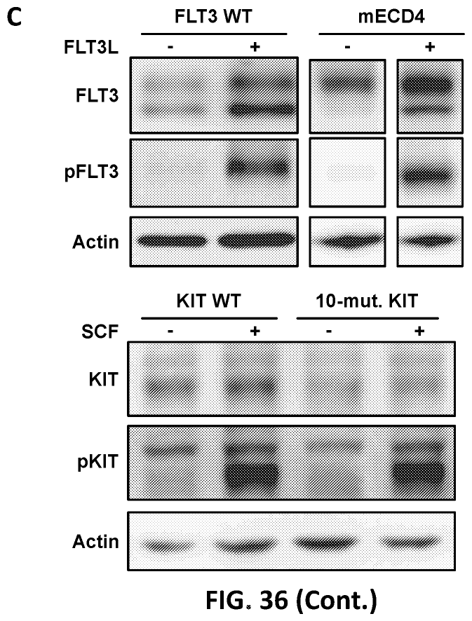

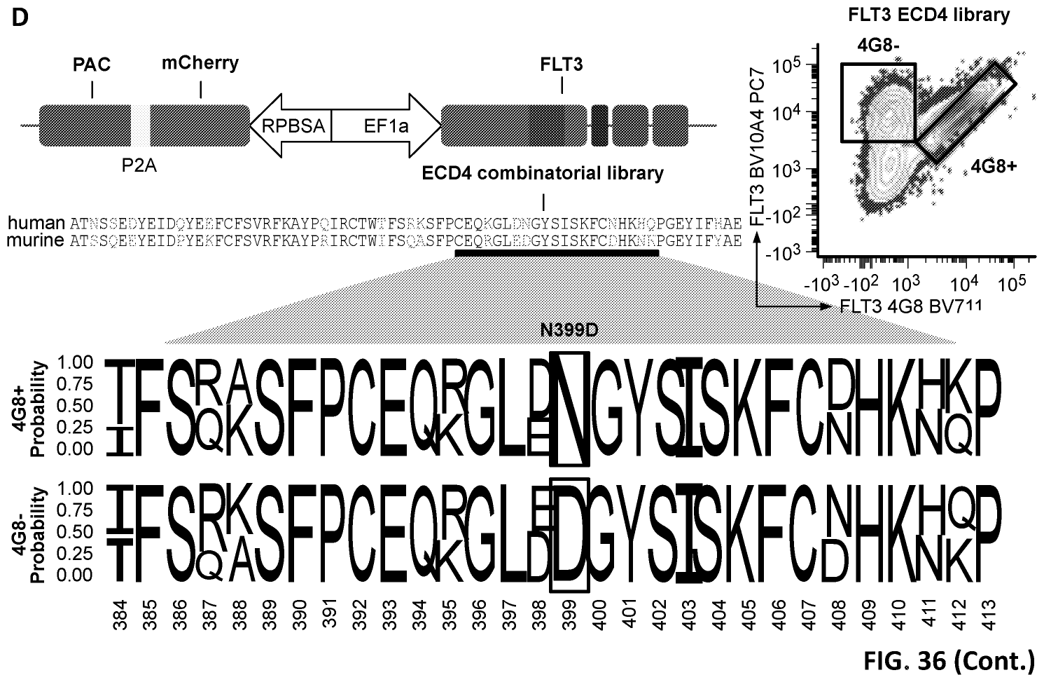

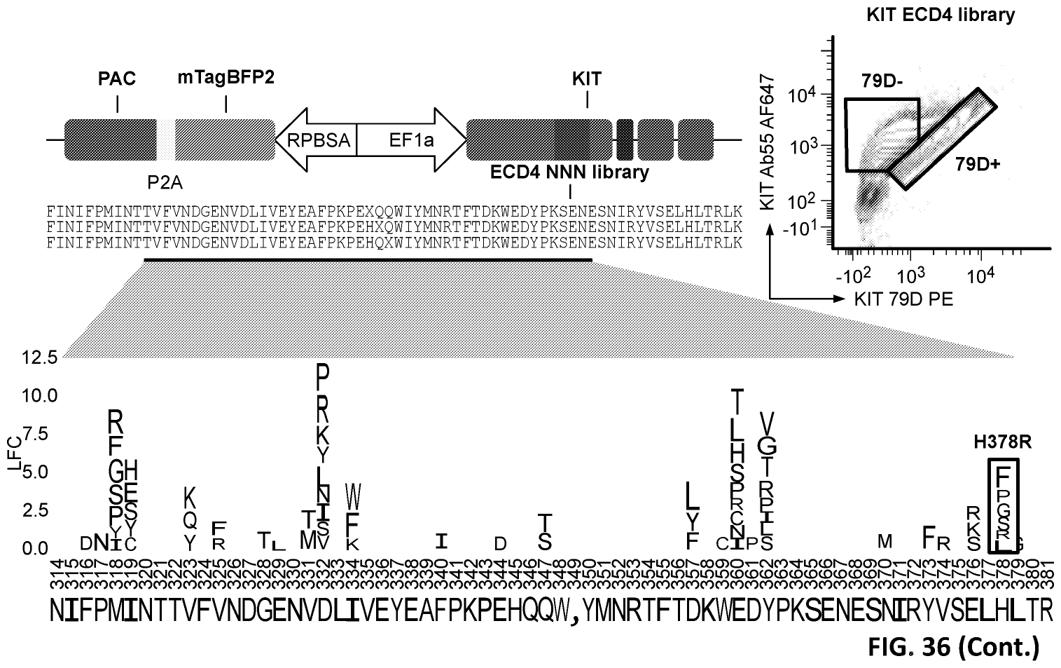

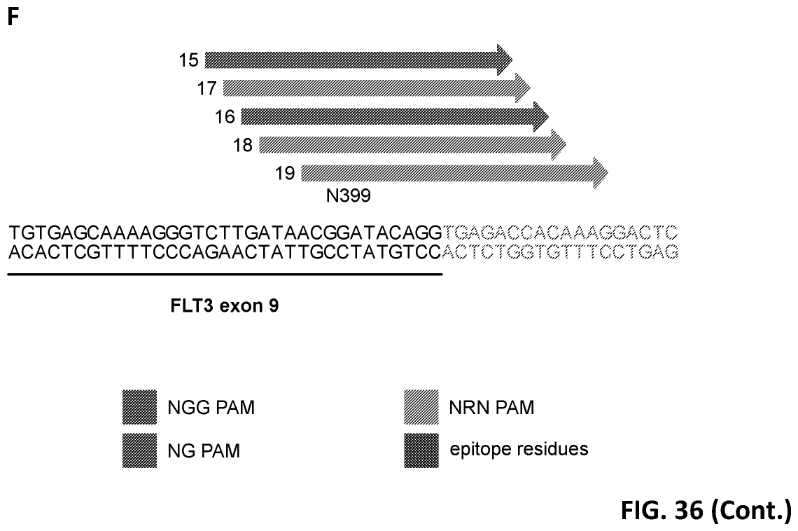

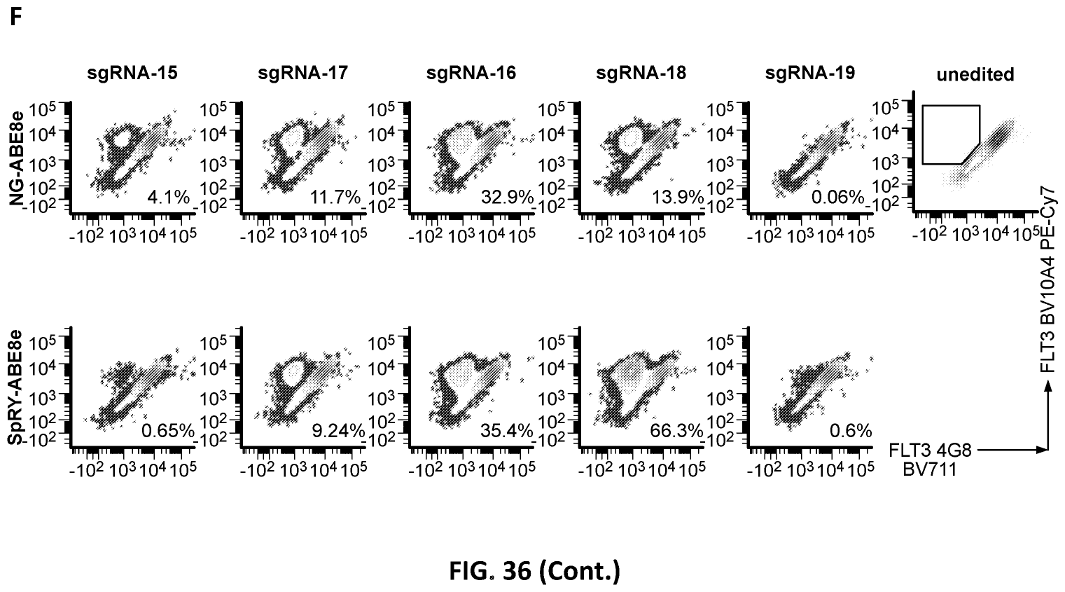

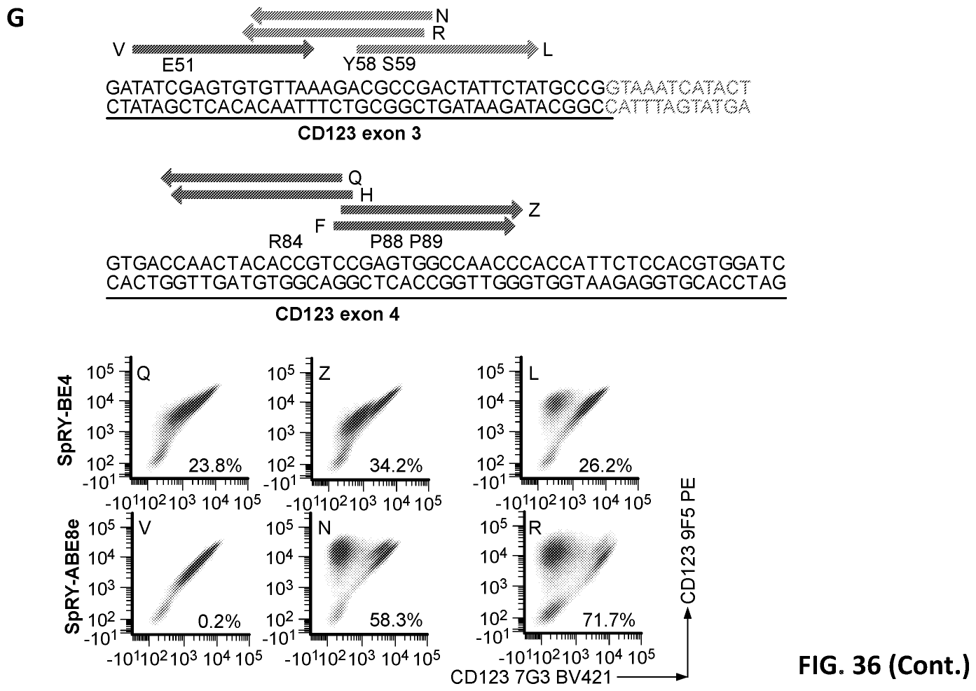

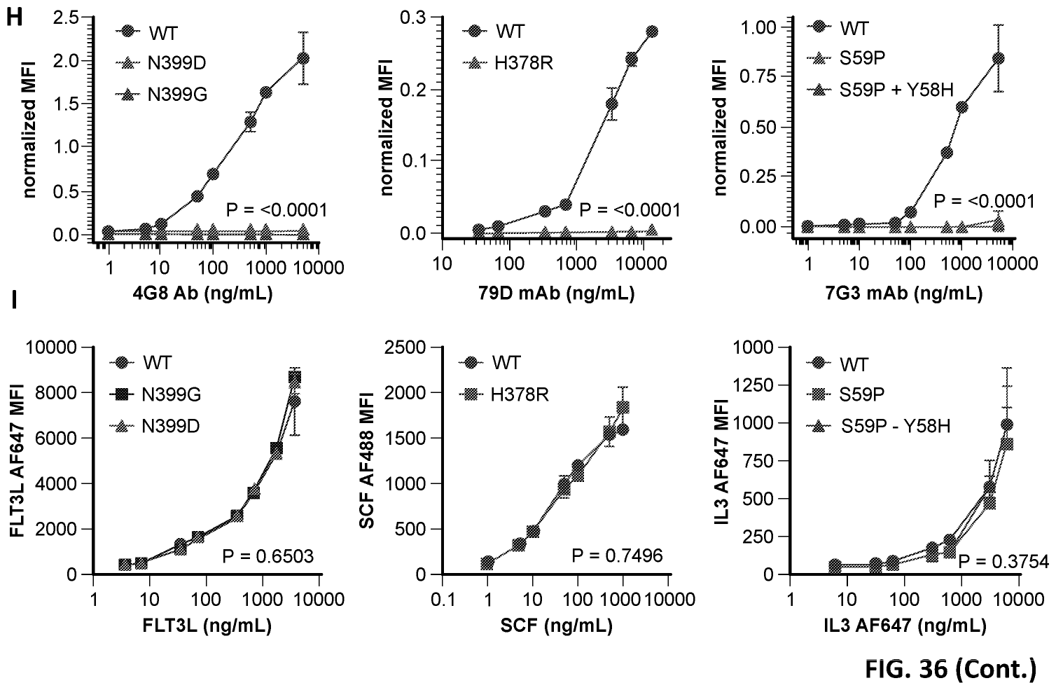

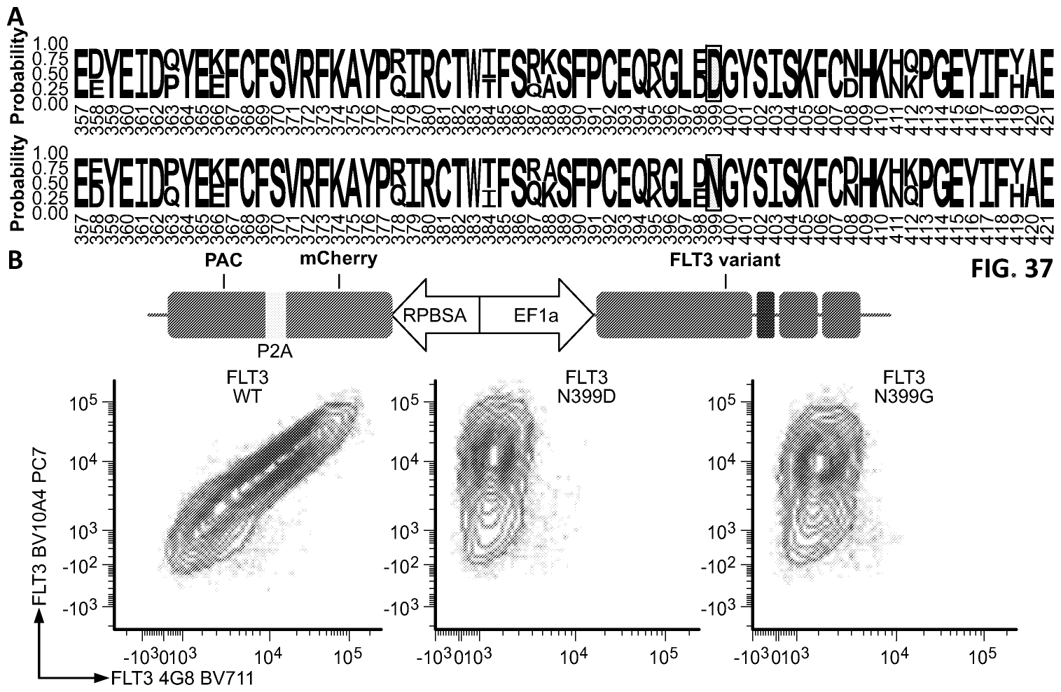

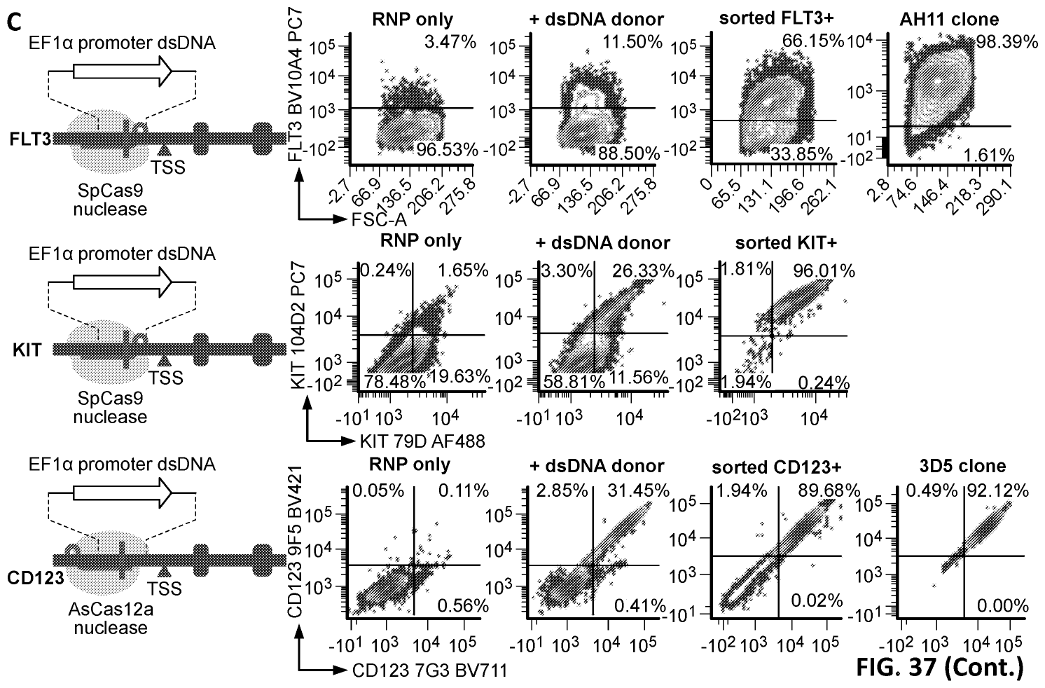

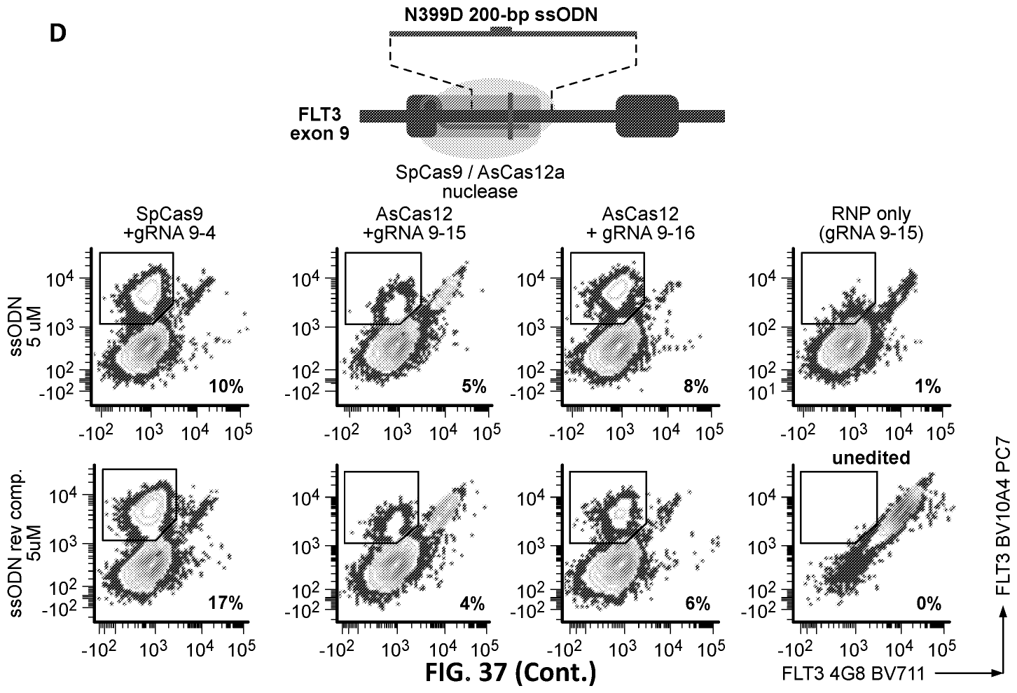

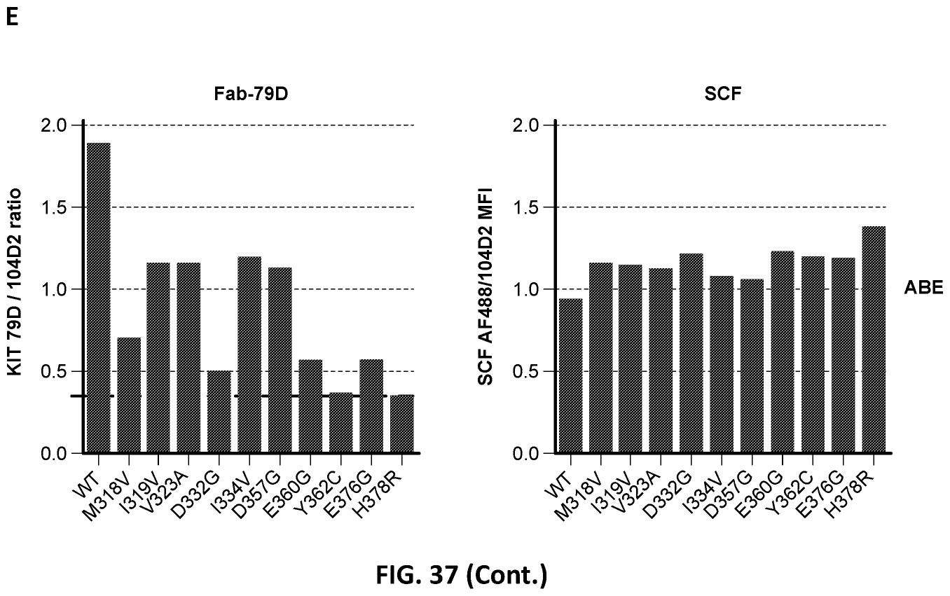

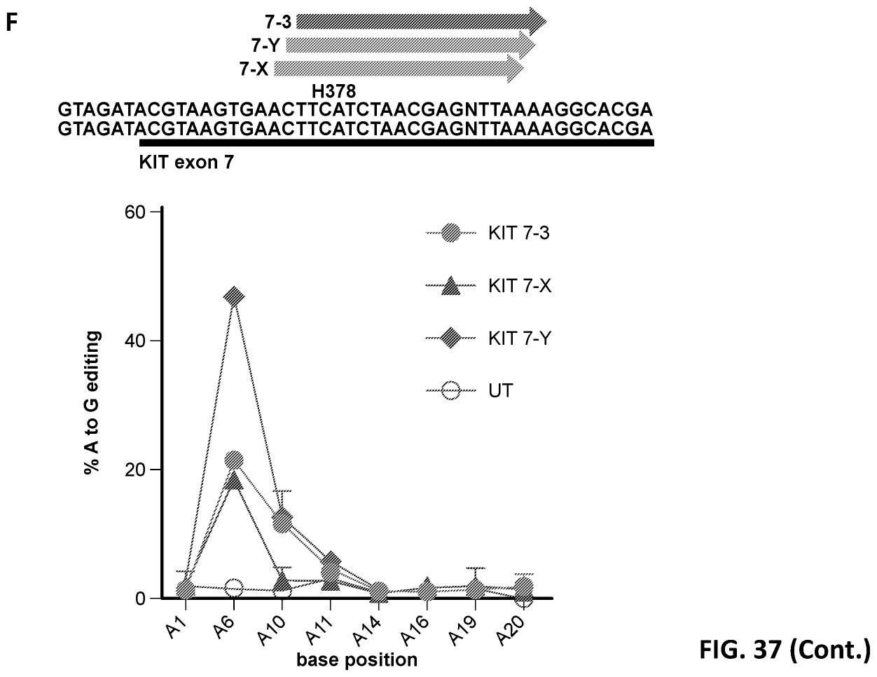

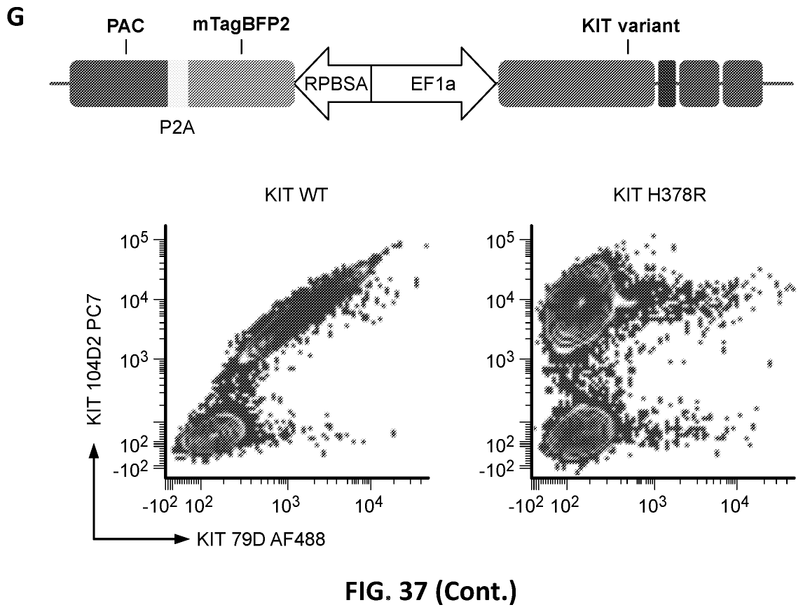

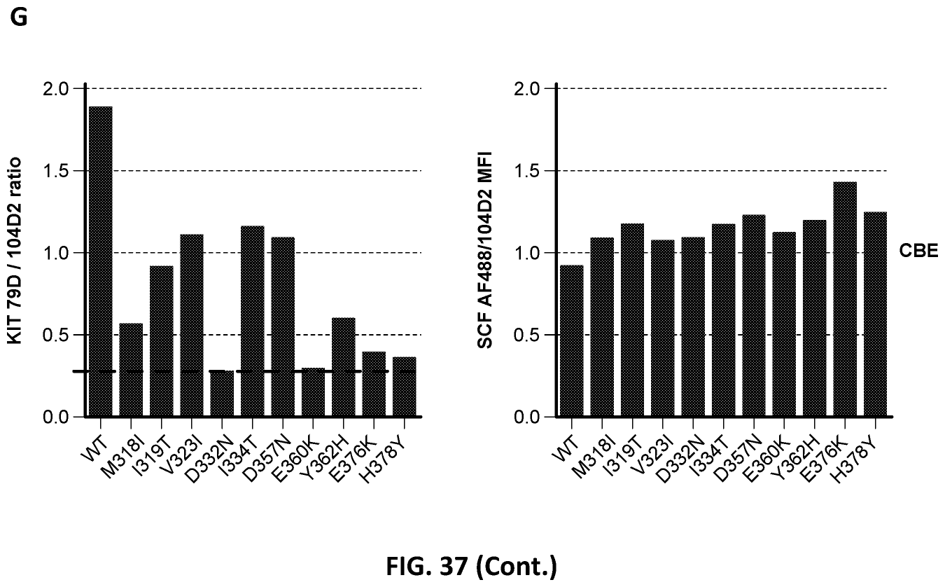

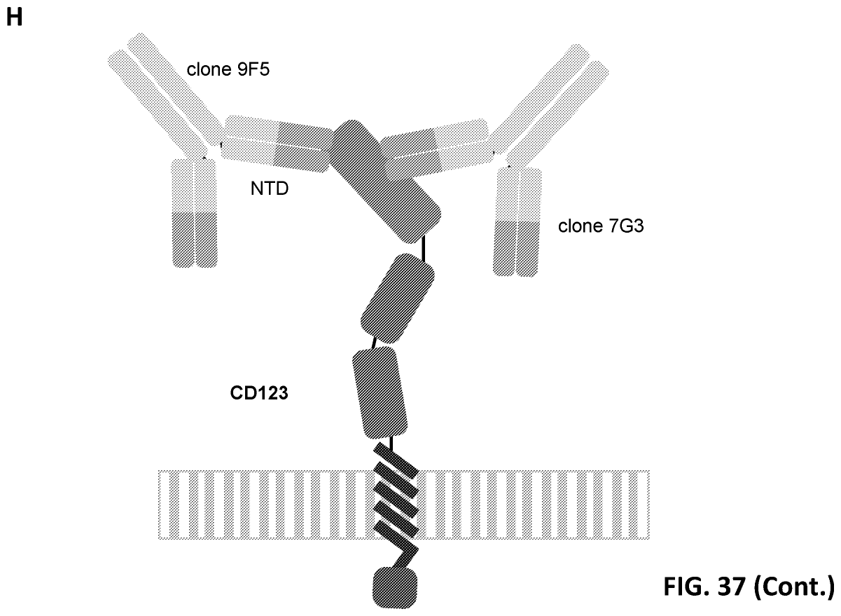

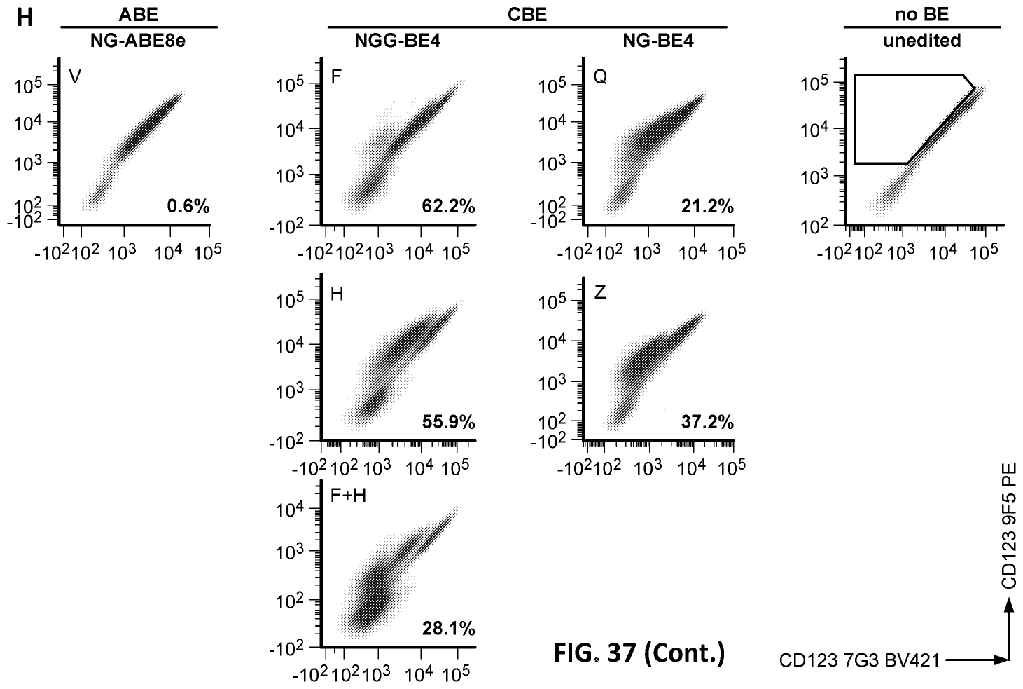

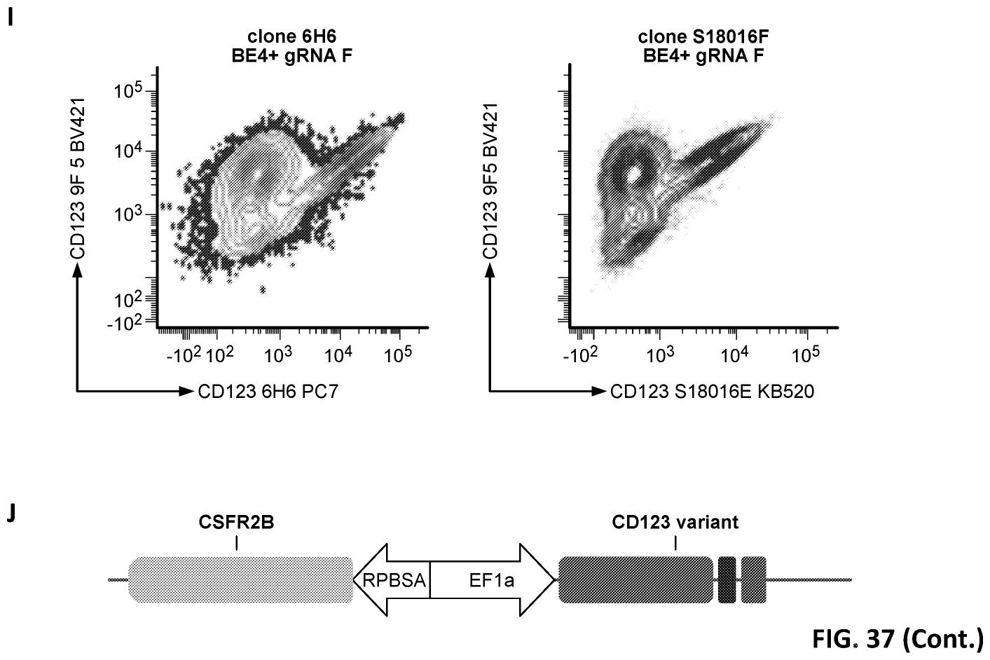

We first confirmed that a chimeric human FLT3 with substitution of ECD4 with its murine ortholog (16 codon changes) results in loss of 4G8 binding without affecting FLT3L binding and intracellular kinase phosphorylation (FIG. 36A, B left, C top). To identify the minimal number of residues involved in 4G8 binding, we designed a Sleeping Beauty transposon-based combinatorial library with the human or murine codons at each of the 16 mismatched positions within ECD4 (FIG. 36D, top left). Flow cytometry analysis of K562 cells transduced with this library showed a population of cells positive for the control antibody (BV10A4) and negative for 4G8 (FIG. 36D, top right). Comparison of the relative abundance of human vs murine codons at each position, measured by targeted deep sequencing of sorted single (BV10A4+4G8−) and double positive (BV10A4+4G8+) cells, revealed enrichment for a single amino acid substitution (N399D; FIG. 36D, bottom and FIG. 37A). To validate this result, we transduced K562 cells with the FLT3 N399D variant and confirmed surface expression of FLT3 at levels comparable to wild type, but with loss of 4G8 binding (FIG. 37B).

Next, we evaluated gene editing strategies to introduce the N399D substitution. To easily evaluate the outcomes of our genome engineering procedures on cells that do not depend on FLT3 signaling, we generated K562 reporter cells that express FLT3 from their endogenous locus by targeted integration of an EF1α constitutive promoter upstream to the transcriptional start site (FIG. 37C). We then confirmed that the N399D mutation can be inserted by homology directed repair (HDR) using either SpCas9 or AsCas12a nucleases and 200-bp ssODN donor templates and that successfully edited cells show loss of 4G8 binding while preserving FLT3 surface expression (FIG. 37D). Nonetheless, the use of nucleases bears the intrinsic risk of genotoxicity associated with DNA double strand breaks (DSB) and gene knock-out, which occurs in a large proportion of non-edited cells (FIG. 37D).

Since epitope engineering can be achieved by the introduction of single point mutations, we reasoned that base editing (BE) could be a suitable and safer option for epitope editing by avoiding the need for DSB. The Asparagine in position 399 is encoded by an AAC codon, which can be converted to GAC (Aspartate) or GGC (Glycine) by Adenine Base Editing (ABE). We tested this hypothesis by electroporating the FLT3-reporter cells with several sgRNAs (in a 1-bp staggered fashion, with the target Adenine in position 3 to 9 of the protospacer) in combination with the advanced generation TadA-8e deaminase, linked either with SpCas9 nickase (NGG PAM) or Cas9 variants with relaxed PAM-specificity (NG-SpCas9n and SpRY-Cas9n, FIG. 36F). Evaluation by flow cytometry showed successful epitope editing with loss of 4G8 recognition, with the highest efficiency achieved by SpRY-ABE8e in combination with FLT3-sgRNA-18 (66.3%, FIG. 36F). In contrast to the HDR-based strategy, both base-edited and non-base edited cells retained normal surface FLT3 expression, without significant gene knock-out. As both N399D and N399G are potential outcomes of our editing strategy, we included this mutation in all further validation analyses.

A similar strategy was applied for the epitope mapping of Fab-79D, a KIT-targeting mAb which is reported to bind to KIT ECD4 and to block ligand-induced dimerization⁴⁵. We first confirmed loss of Fab-79D binding by introducing 10 amino-acid changes (from KIT orthologs) at positions previously predicted as potential contact points with KIT ECD4 (F316S, M318V, I319K, V323I, 1334V, E360K, P363V, E366D, E376Q, H378R) and verified the preservation of stem cell factor (SCF) binding and intracellular kinase phosphorylation (FIG. 36B right, C bottom). To comprehensively screen the interaction between ECD4 mutations and clone Fab-79D we performed a screening using a degenerated codon library in which each position of KIT ECD4 was substituted by a random amino acid (FIG. 36E left). We transduced HEK-293T cells with the library and sorted the cells with reduced binding to Fab-79D but preserved KIT expression and cells with preserved Fab-79D staining (FIG. 36E right). NGS sequencing of the library region and comparison of the enrichment of specific codons at each position within single positive cells revealed several mutations capable of reducing the affinity of Fab-79D (FIG. 36E bottom). To validate these findings, we individually cloned 20 amino acid substitutions by selecting mutations which could be reproduced by Adenine- or Cytidine-base editing at 10 positions identified by the library (M318, I319, V323, D332, I334, D357, E360, E376 and H378) and selected the H378R for further development, as it showed the highest reduction of Fab-79D binding when normalized for the KIT control Ab (clone 104D2) while preserving binding to the SCF (FIG. 37E). Furthermore, H378R can be inserted by ABE, similarly to FLT3 N399D, thus allowing potential combination and dual epitope-engineering. As done for FLT3, we screened on K562 cells 3 different sgRNAs with the editing windows aimed at H378 (target A in position 5, 6 or 7) in combination with SpRY-ABE8e and identified KIT-Y as the combination with the highest efficiency (FIG. 37C,F).

For CD123, both the epitope and the amino acid substitutions affecting the binding of therapeutic clone 7G3 (or its humanized counterpart, CSL362—Talacotuzumab) have been previously reported⁴⁸. To develop our epitope-editing strategy, we designed a targeted BE screening on K562 reporter cells (FIG. 37E) by testing sgRNAs aimed at positions E51, Y58, S59, R84, P88 and P89 of CD123 N-terminal domain and rationally combined them with CBE (evo-APOBEC1-BE4 with NGG, NG and SpRY Cas variants) and ABE (ABE8e, NG and SpRY variants, FIG. 36G). While several combinations introduced mutations that reduced the affinity of 7G3 to CD123, only gRNAs CD123-N and CD123-R in combination with SpRY-ABE8e and gRNA CD123-L in combination with SpRY-evo-APOBEC1-BE4 could completely abrogate 7G3 binding (FIG. 36G and FIG. 37H). Sequencing of edited cells revealed that ABE and CBE resulted in S59P and S59F substitutions respectively. A bystander mutation (Y58H) could also be introduced, at higher efficiency with gRNA CD123-R than with CD123-N, by adenine base editing, and was therefore included in further validations. Due to the possibility of editing multiple epitopes in combination with FLT3 and KIT ABE, we selected S59P introduced by gRNA CD123-R with ABE for further development. Finally, by testing our base edited CD123 reporter cells with two additional CD123-targeting mAbs, we discovered that cytidine base editing of P88/P89 resulting in combinations of P88S/P89S or P88L/P89L) resulted in near-complete loss of recognition by clones 6H6 and S18016F, thus widening the pool of mAbs with potential application in combination with epitope-engineered cells (FIG. 37I).

To precisely quantify the loss of affinity of our selected Abs for the epitope-engineered receptor, we transduced K562 cells by Sleeping Beauty transposase with each receptor variant (FLT3 WT, N399D, N399G; KIT WT, H378R; CD123 WT, S59P and Y58H-S59P) and observed almost complete absence of recognition of all tested variants also at saturating concentration of mAb for the WT receptor (>5000 ng/ml, FIG. 36H). Notably, FLT3 N399G (introduced by editing of both adenines in the AAG codon in position 399) did not differ from N399D in its capacity to reduce 4G8 binding. Similarly, addition of the Y58H bystander mutation to CD123 S59P showed similar reduction in 7G3 binding, without affecting CD123 expression on the cell membrane.

Overall, we concluded that epitope engineering of FLT3, KIT and CD123 is feasible and can be achieved with high efficiencies without the need for DSB by selecting appropriate combinations of gRNA and base editing enzymes.

### Example 16: Epitope Editing Preserves Receptor Functionality

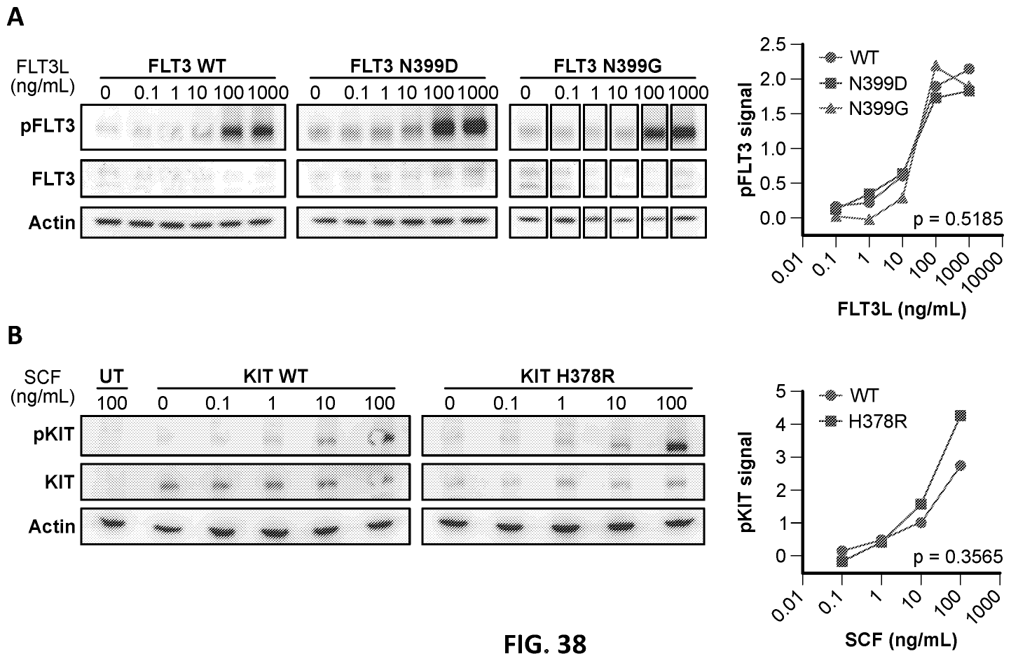

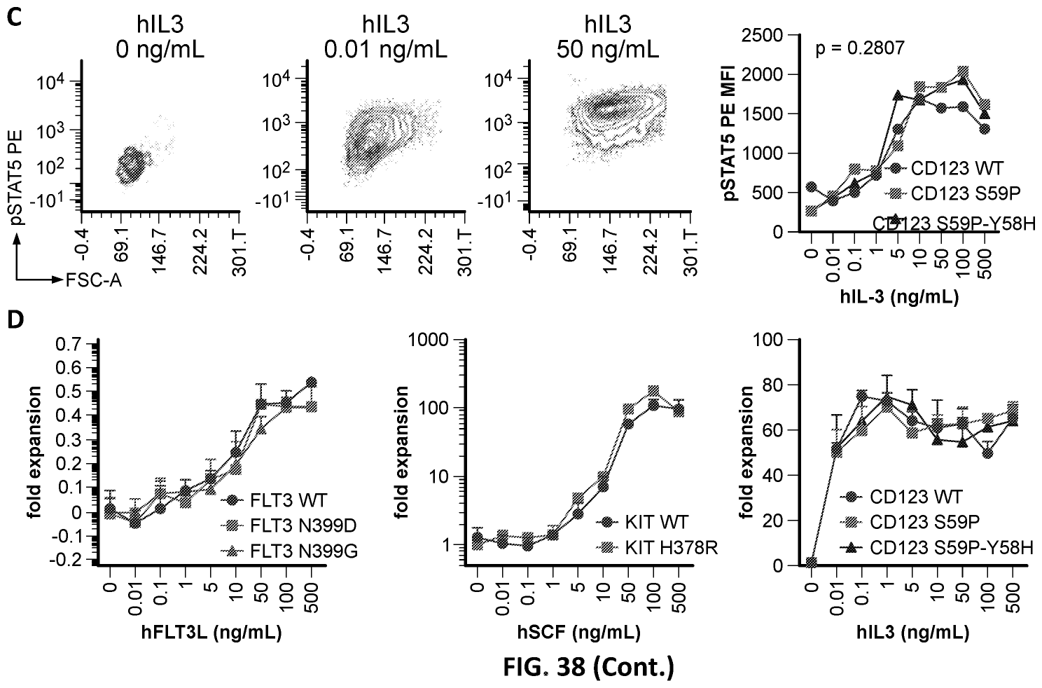

As the selected targets are fundamental cytokine/growth factor receptors expressed on human HSPCs and have relevant roles in stem cell maintenance and lineage differentiation, we stringently assessed if our engineering procedure would alter receptor functionality. By using fluorescently conjugated FLT3L, SCF and IL-3 ligands, we confirmed comparable binding to their respective WT or epitope engineered receptors across all tested concentrations (1 to >1000 ng/ml; FIG. 36I). CD123 variants were co-expressed with the common beta subunit (CSF2RB/CD131) to form the heterodimeric IL-3 receptor and allow signal transduction, as K562 cells do not express CSF2RB by flow cytometry (FIG. 37J). Activation of intracellular signaling cascade of FLT3 and KIT was confirmed by western blot, which showed comparable and dose dependent kinase phosphorylation upon ligand binding (FIG. 38A,B). For CD123, we confirmed ligand-mediated receptor activation by measuring phosphorylation of the downstream STAT5 signal transducer, which was equally activated in both WT and epitope engineered variants at all tested IL-3 concentrations (FIG. 38C). Finally, to confirm full preservation of ligand-dependent proliferative response, we performed a kinase complementation assay on BaF3 cells, a murine IL-3-dependent lymphoblastoid cell line which requires signaling through the STAT5 pathway for proliferation and survival⁵⁰. We confirmed comparable and dose dependent rescue of cell proliferation by WT and epitope engineered receptor variants after exposure to human FLT3L, SCF and human IL-3 during murine IL-3 starvation (FIG. 38D).

### Example 17: Stealth Receptors are Resistant to CAR-T Cells

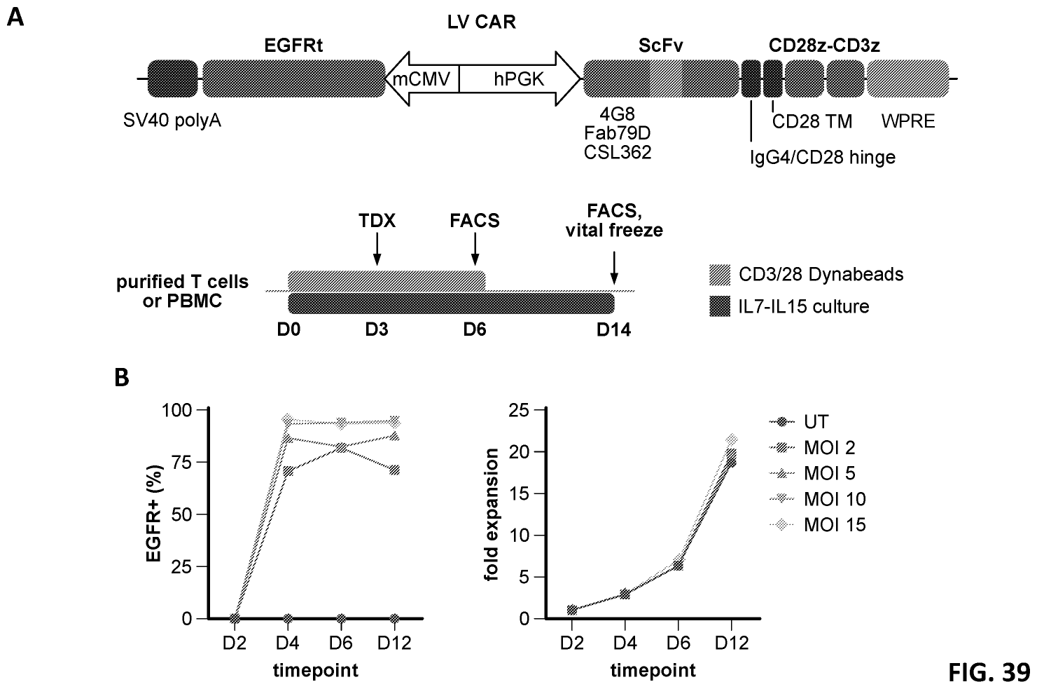

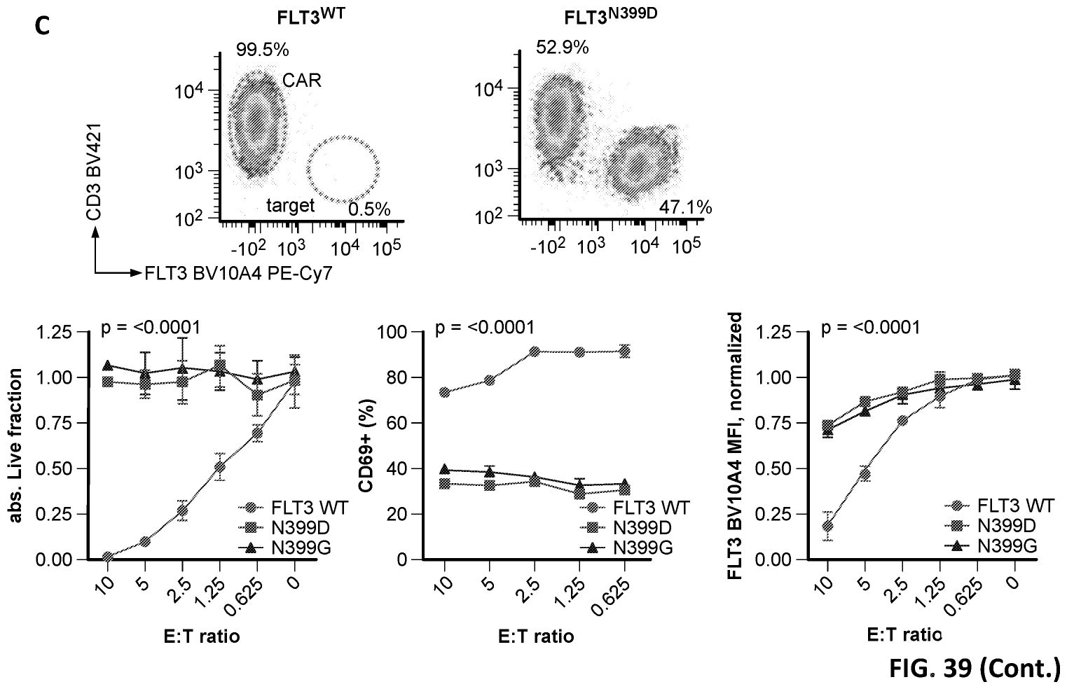

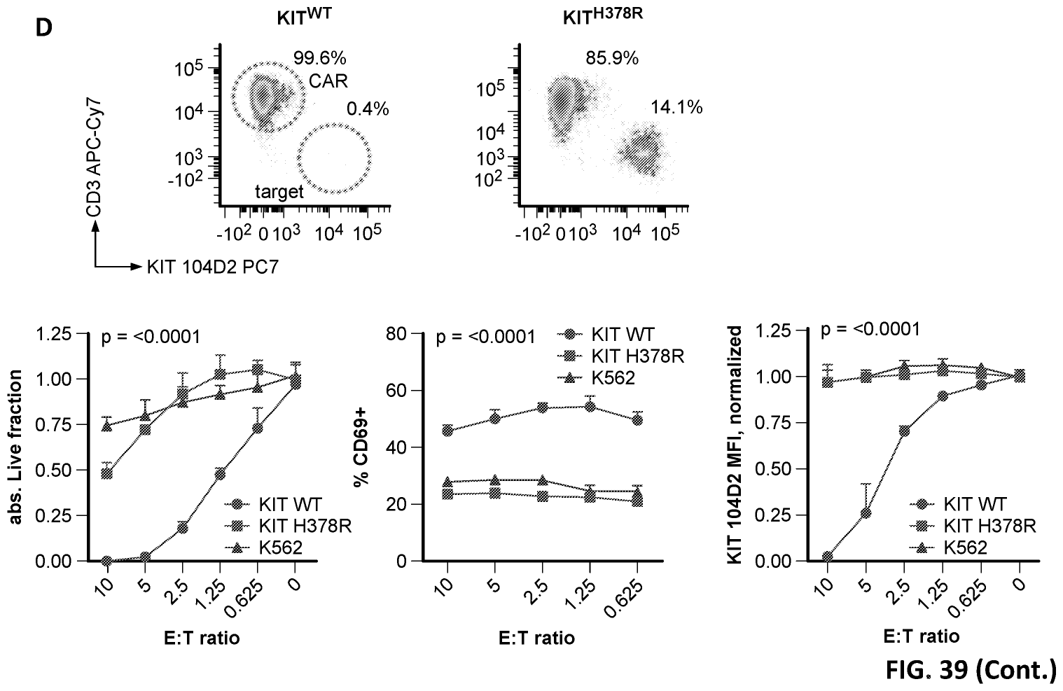

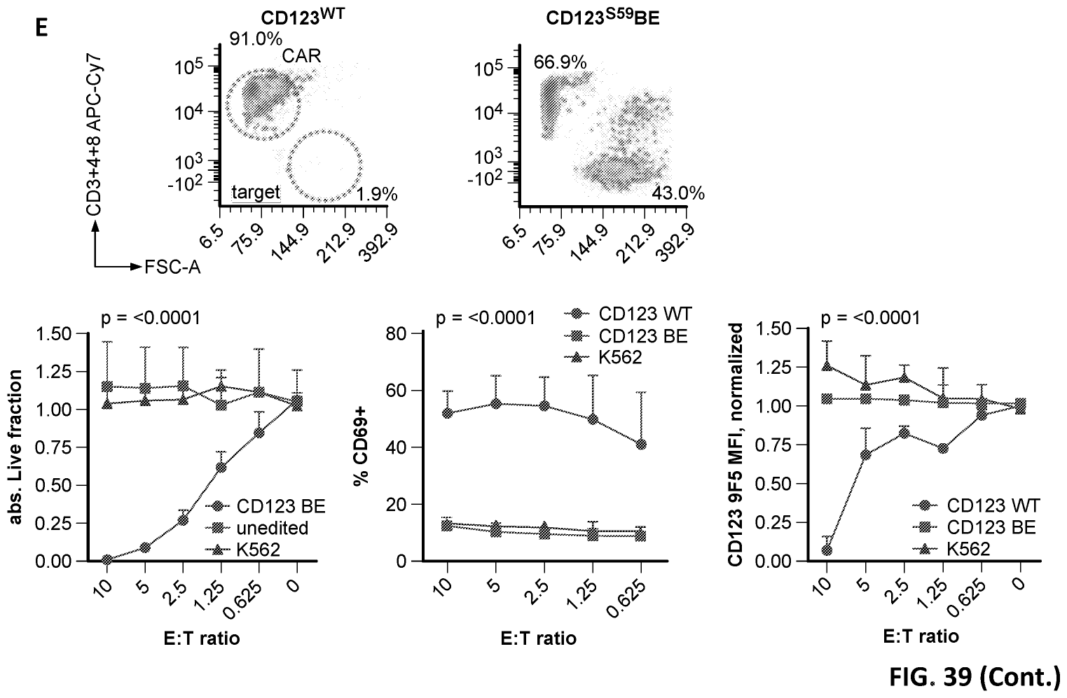

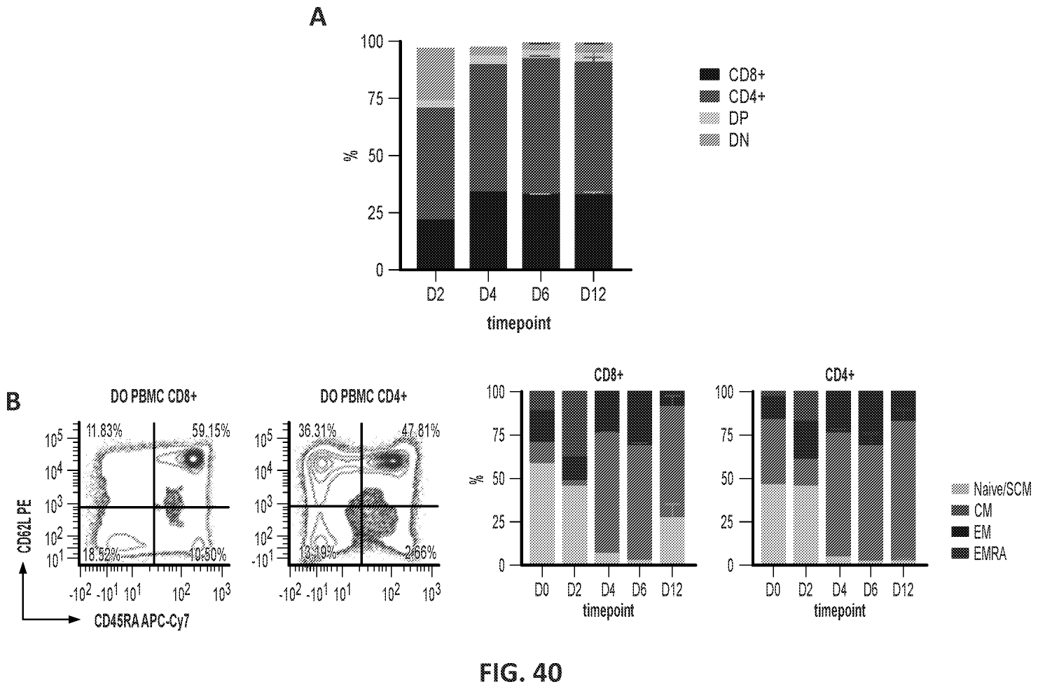

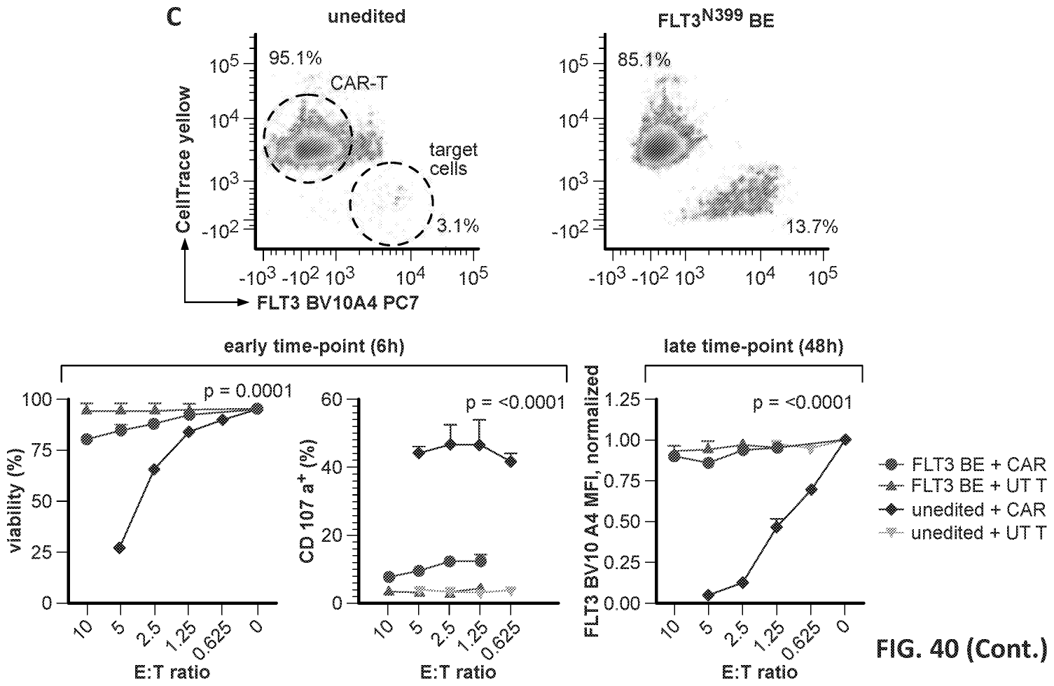

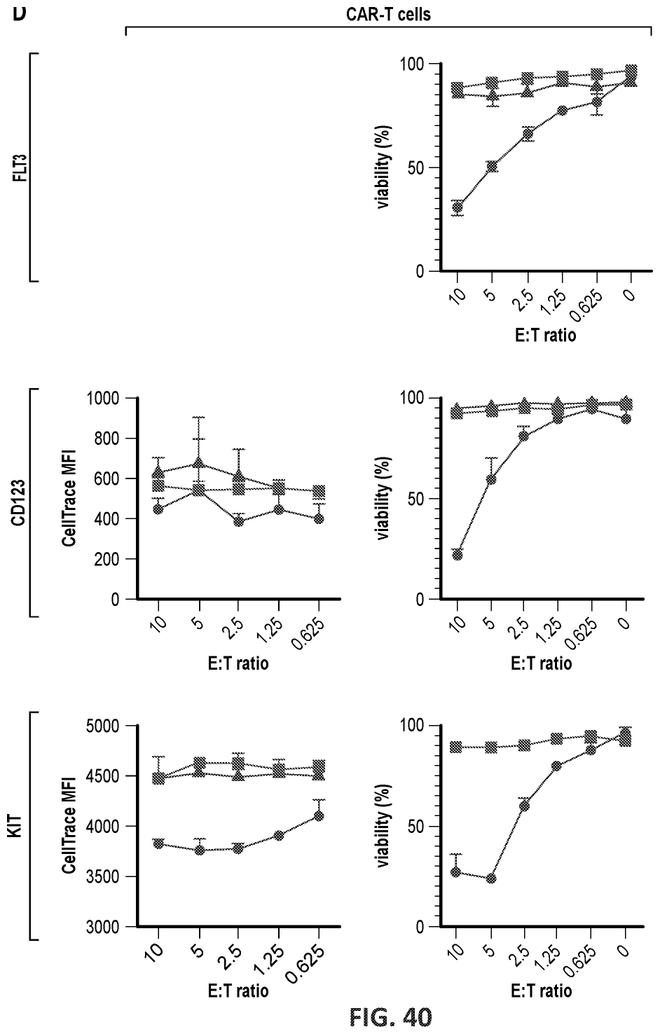

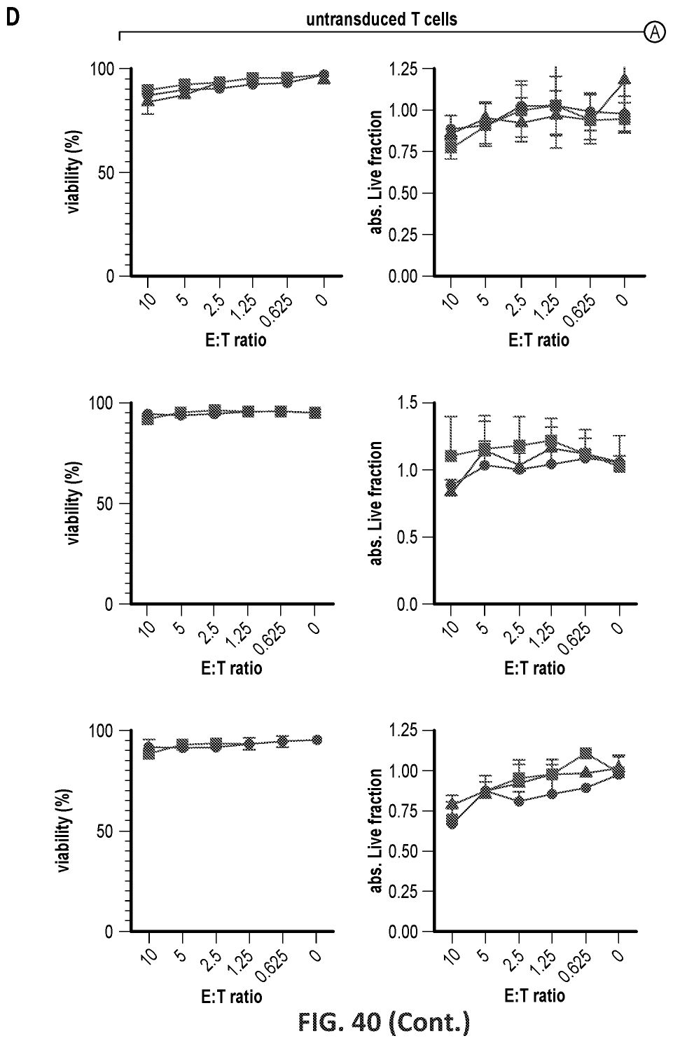

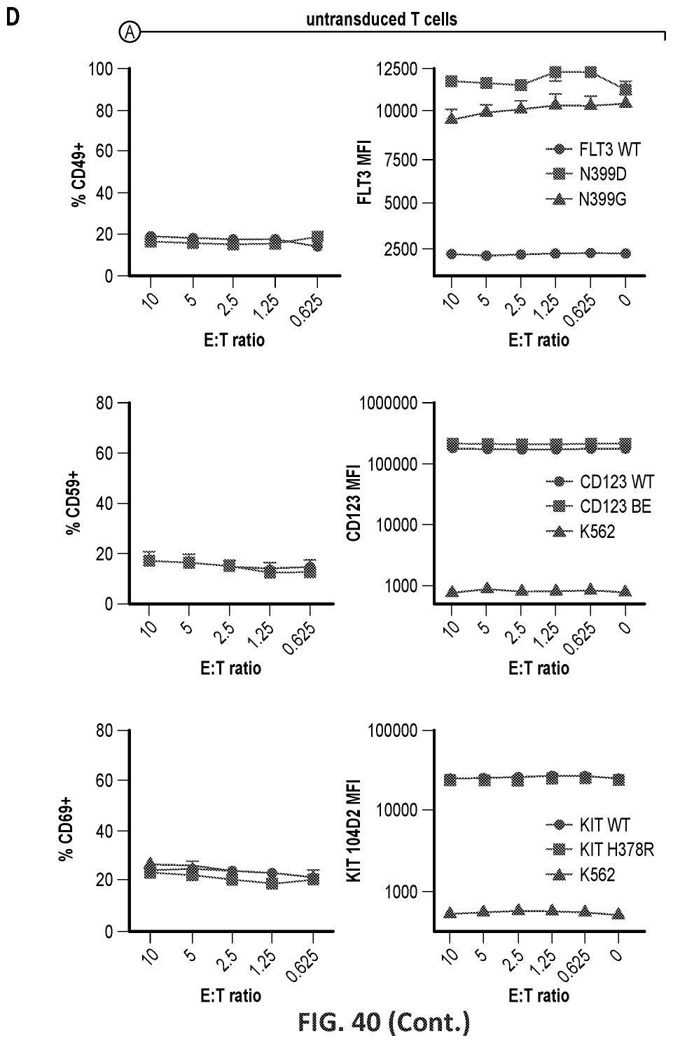

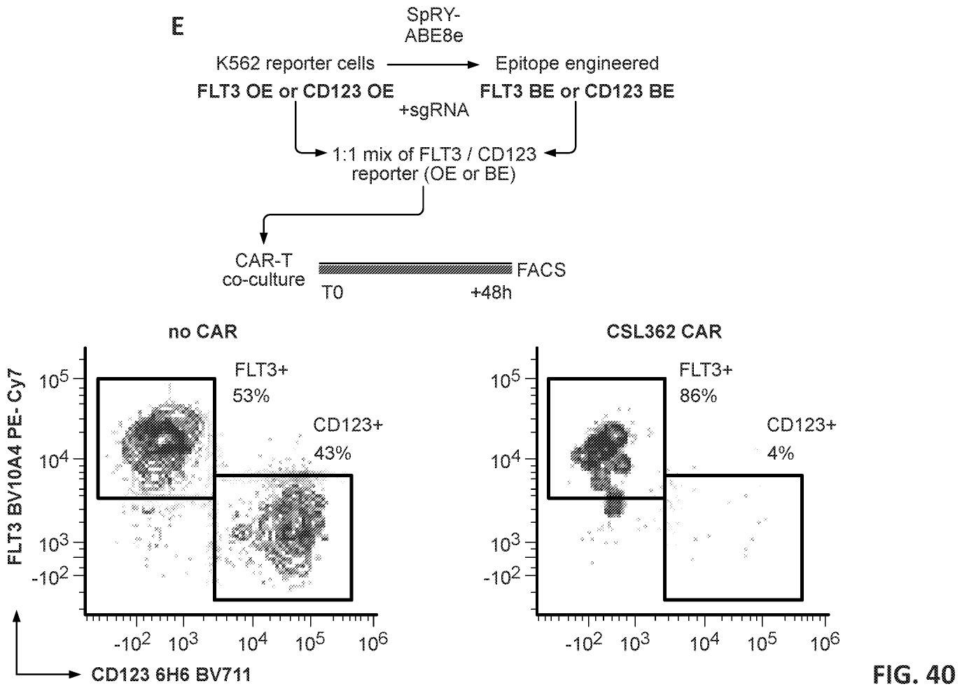

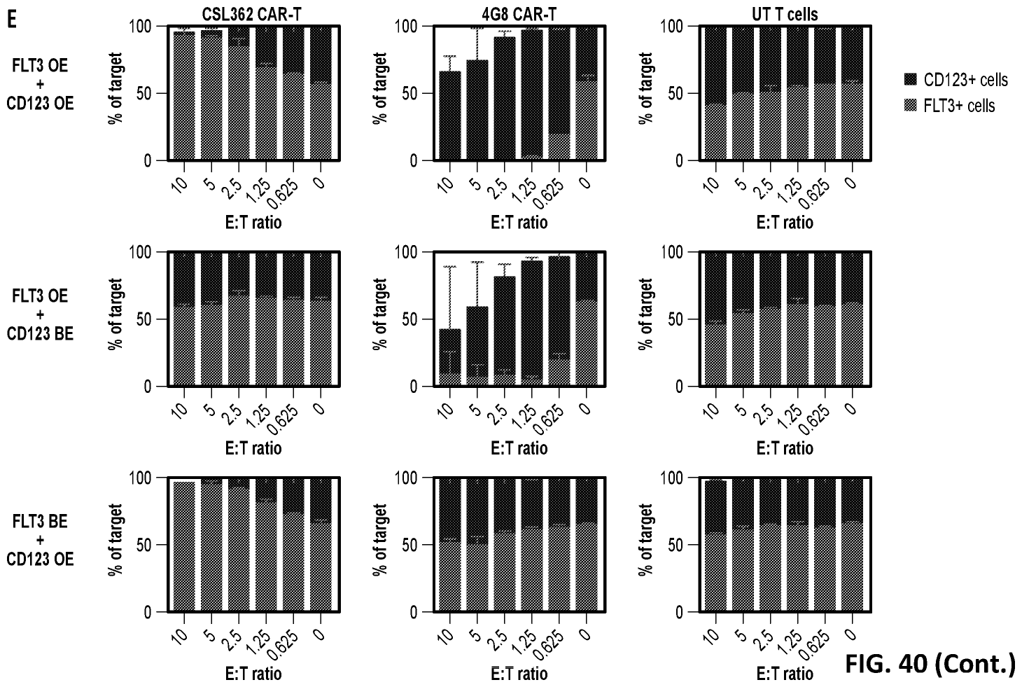

Recent studies have shown that CAR-T cells generated from the anti-FLT3 clone 4G8²², the anti-KIT Fab-79D²¹ or the anti-CD123 CSL362—the humanized variant of clone 7G3^{48,51}—mAbs have remarkable efficacy against human AML cells. To assess resistance of epitope engineered cells to targeted CAR-T therapy, we cloned the 4G8, Fab79D and CSL362 single-chain variable fragments (scFv) in a 2^{nd} generation CAR construct with a CD28 costimulatory domain and used a lentiviral vector with a bidirectional promoter to co-express an optimized⁵² truncated EGFR selection/depletion marker (tEGFR; FIG. 39A). For CAR-T cell production, we used a culture protocol based on CD3-CD28 bead stimulation in the presence of IL-7+IL-15, which expands cells with a T stem memory phenotype⁵³ (FIG. 39A, FIG. 40A,B). We obtained high CAR transduction efficiency (>85% by tEGFR staining) with in vitro expansion of >20 fold compared to culture start (FIG. 39B).

By performing an in vitro killing assay on K562 reporter cells we found that, while the majority of cells overexpressing WT FLT3, KIT or CD123 were killed by their respective specific CAR-T (at E:T=10, <2% surviving cells compared to E:T=0), K562 cells expressing epitope-engineered variants were resistant to CAR-T cell killing (both as absolute counts and cell viability) across different effector: target ratios (E:T 0.625 to 10) and survived up to experiment termination (FIG. 39C-E and FIG. 40C,D). T cell activation (CD69 expression) and degranulation (CD107a surface expression) were significantly higher in conditions cultured with cells expressing WT genes, which is consistent with lack of recognition of the epitope edited variants by the CAR-T (FIG. 40C,D). Moreover, the surviving K562 reporter cells still expressed the targeted receptor at levels comparable to untreated controls (FIG. 39C,D,E right). Untransduced T cells did not show target killing nor CD69 upregulation when cultured with all tested conditions (FIG. 40D). To further confirm stringent epitope-specific killing by CAR-T cells we co-cultured mixed populations of dual target cells (FLT3- and CD123-expressing K562 cells), either unmodified or base edited, and observed selective resistance of the epitope-edited population when plated with the corresponding CAR-T cells (FIG. 40E).

Overall, these data provide a stringent validation that cells over-expressing the epitope engineered FLT3, KIT and CD123 variants are resistant to CAR-T cell recognition and killing.

### Example 18: Efficient Epitope Editing of Human HSPC

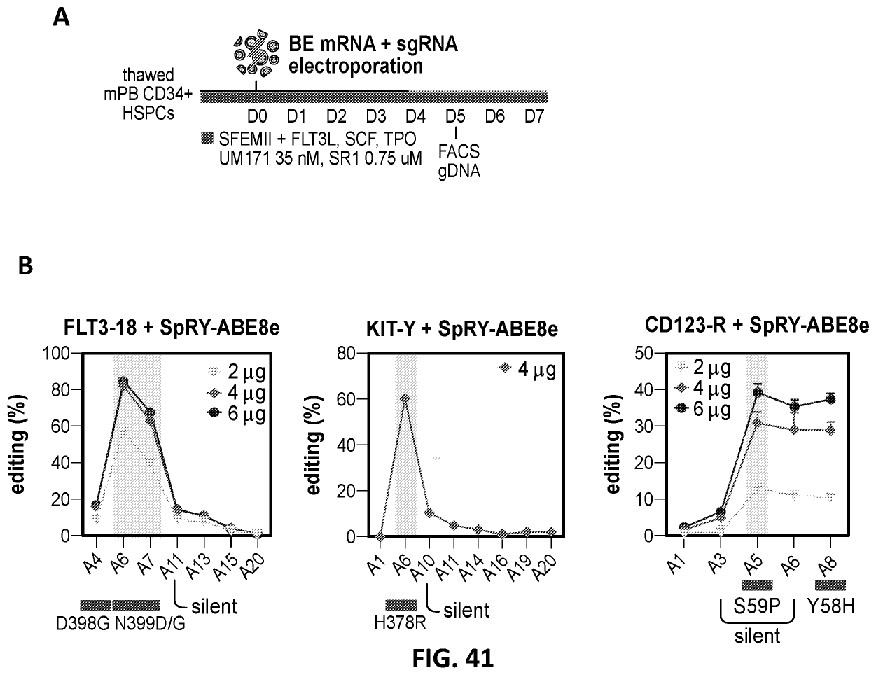

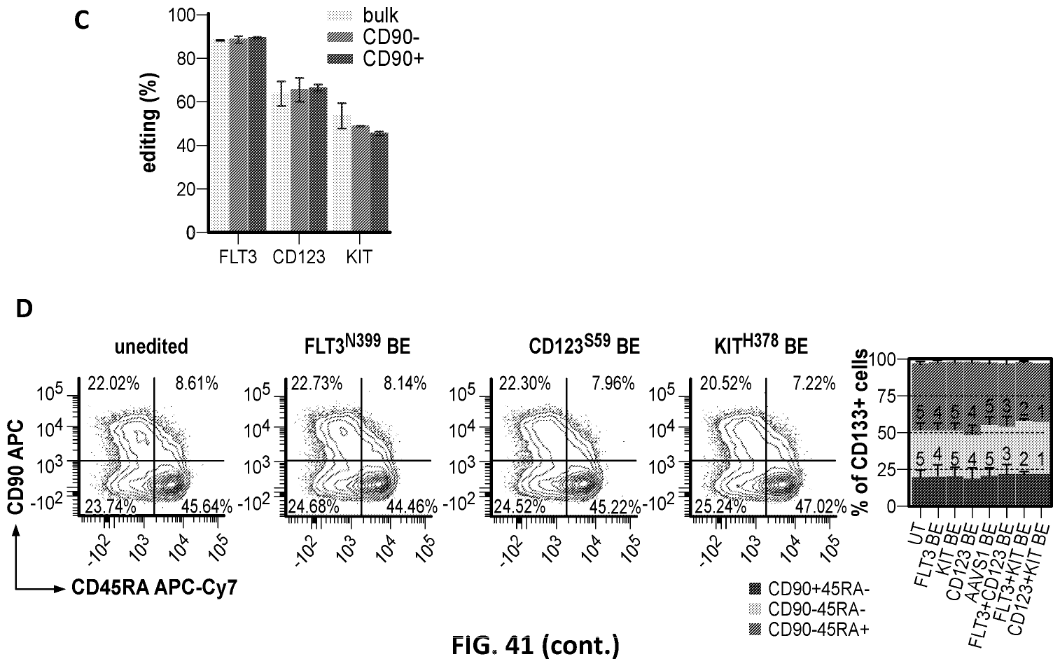

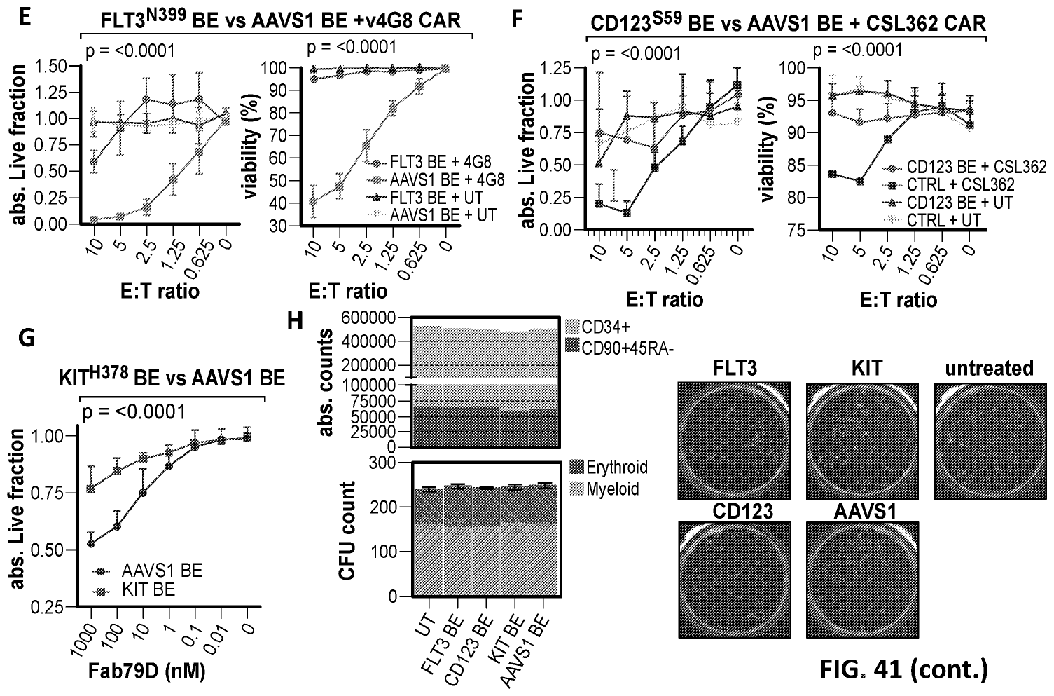

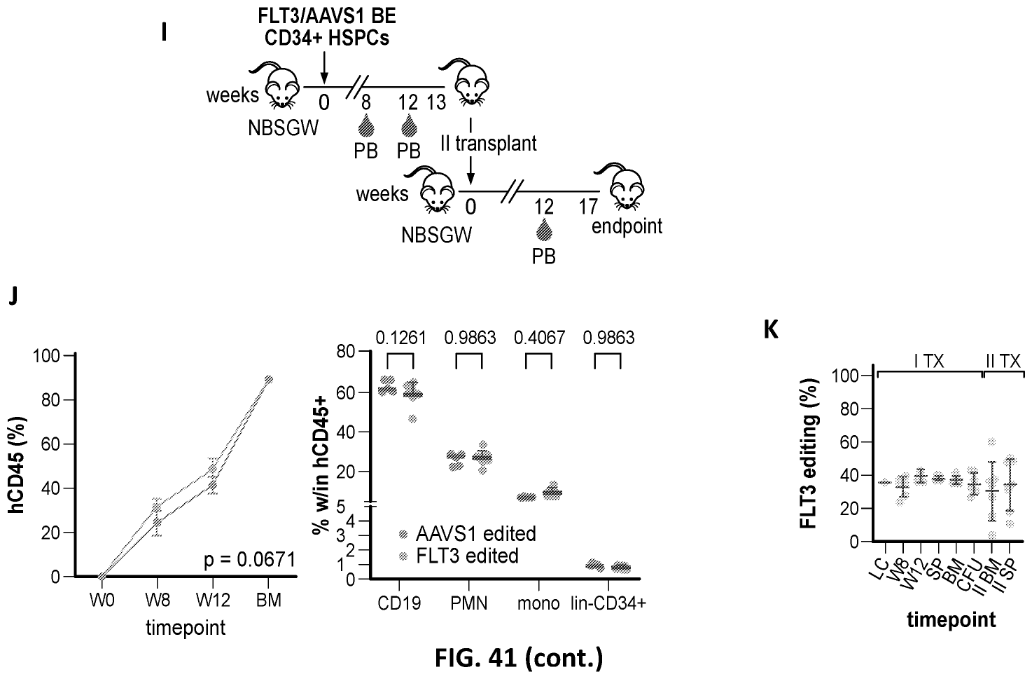

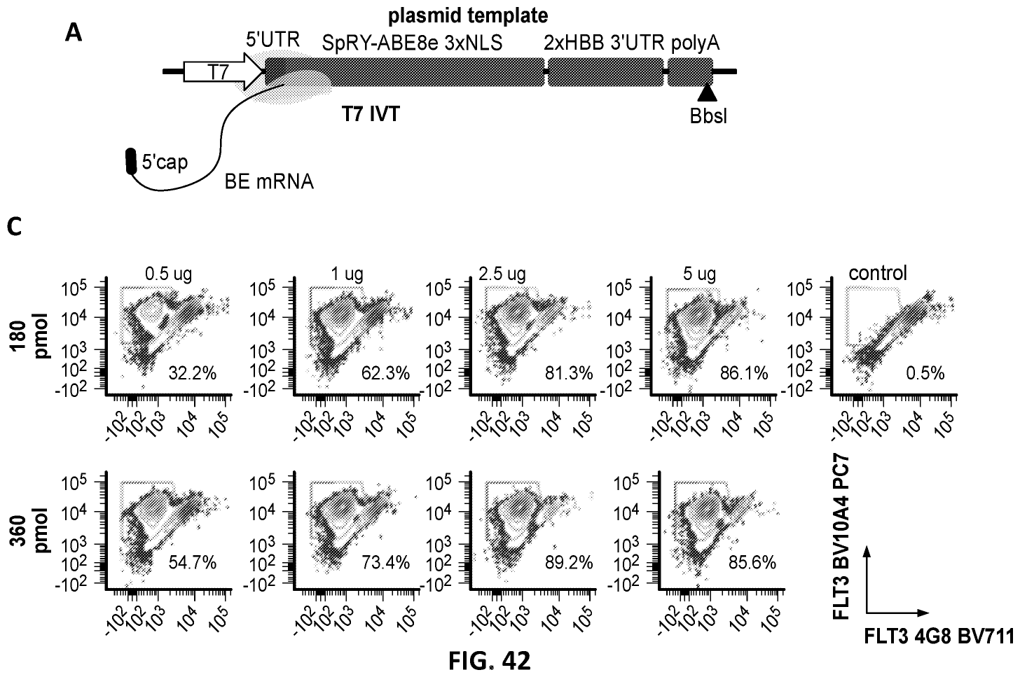

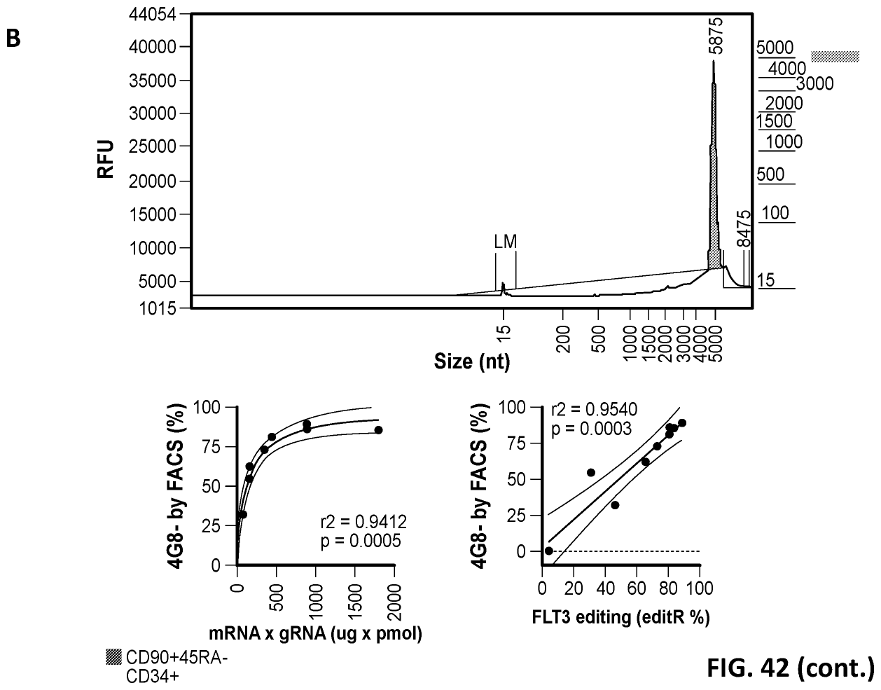

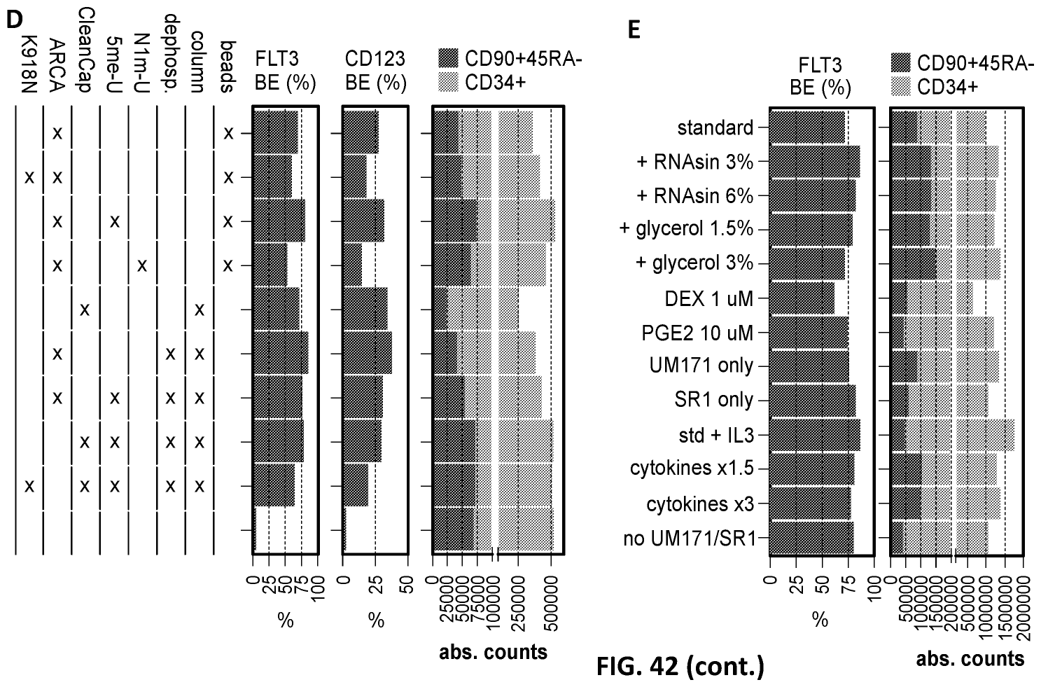

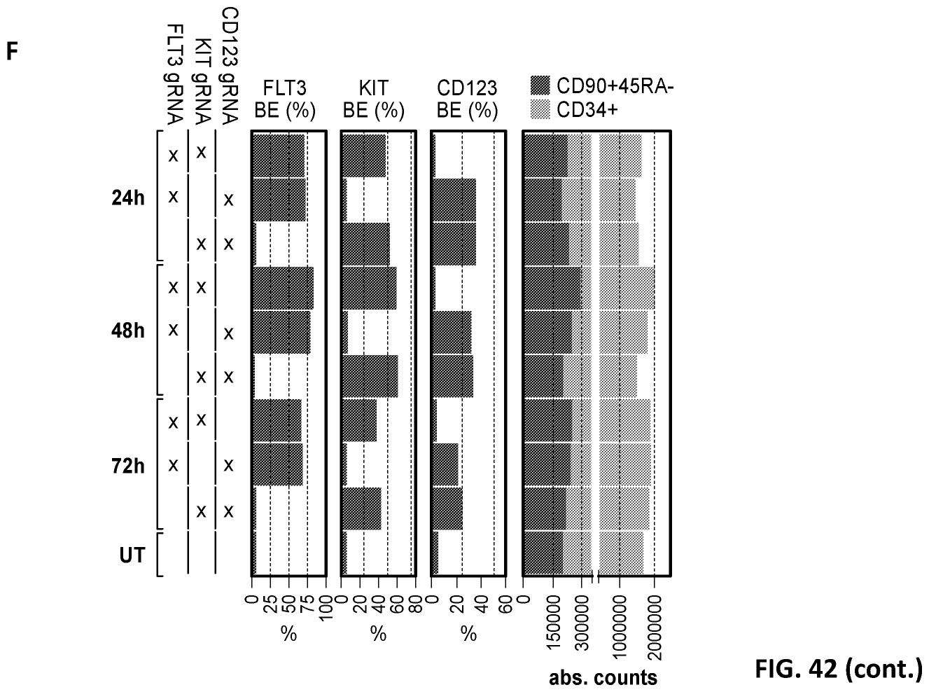

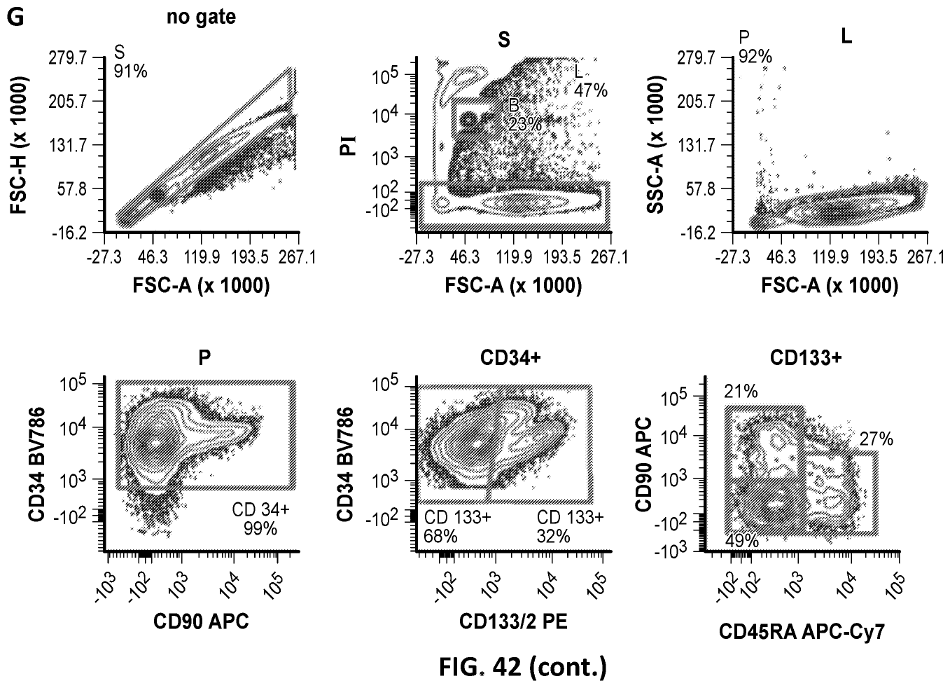

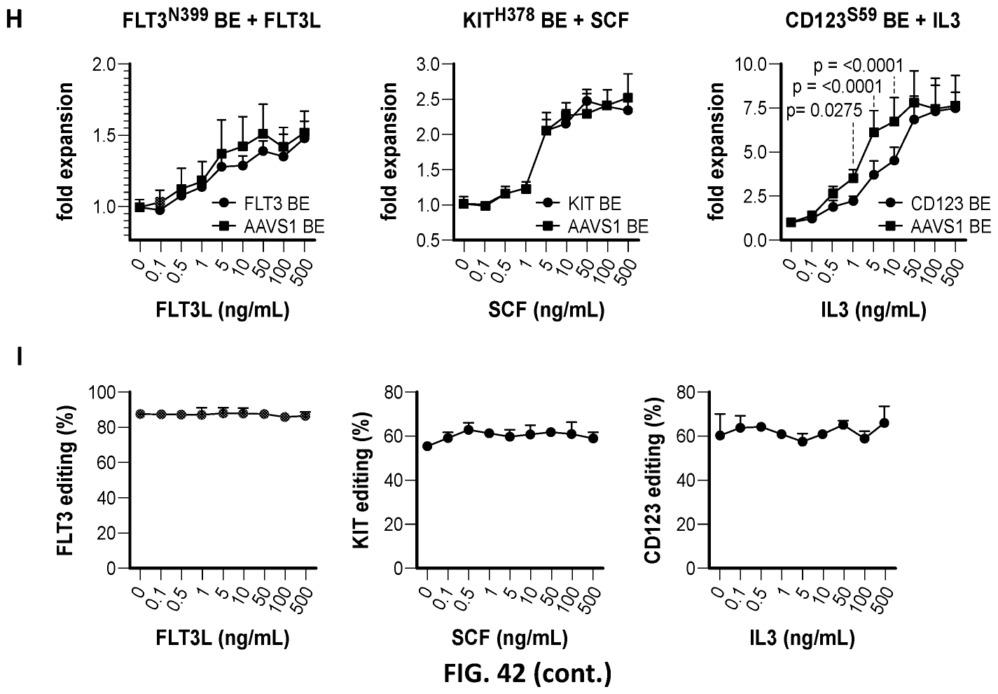

To effectively introduce our nucleotide variants into the endogenous genes of human primary HSPCs, we optimized a base editing protocol on mobilized-peripheral blood (mPB)-derived CD34+ cells based on co-electroporation of a chemically modified sgRNA and in vitro transcribed (IVT) SpRY-ABE8e mRNA (FIG. 41A and FIG. 42A-C). After optimization of mRNA in vitro transcription, culture and electroporation conditions and editing time point after HSPCs stimulation (FIG. 42D,E,F), we achieved up to 86.6%, 78.6% and 67.9% of target A>G conversion for FLT3, KIT and CD123, respectively. We were able to efficiently edit the target adenines within the windows of FLT3-18, KIT-Y and CD123-R sgRNAs (FIG. 41B). Analyses of treated cells showed no skewing of the composition of phenotypically identified progenitors (LMPP, CD90−45RA+; MPP, CD90−45RA−; HSC, CD90+45RA−) during in vitro culture in the presence of stem preserving compounds (StemRegenin, SR-1 and UM171; FIG. 41D and FIG. 42E). Contrary to what we previously observed with HDR-mediated editing⁵⁴, base editing efficiencies were equal in bulk cells, committed progenitors (CD90−) and in the more primitive HSPC subset (CD90+45RA−; FIG. 41C).

To confirm the resistance of FLT3^{N399} and CD123^{S59} epitope-engineered HSPCs to immunotherapies, we performed a killing assay by plating, 3-5 days after electroporation, edited HSPCs with 4G8 and CSL362 CAR-T cells at different E:T ratios. Specific killing by FLT3- and CD123 CAR-T was most pronounced within the CD45RA+ and the CD90+ subsets, respectively, which were thus used to evaluate the outcome of these experiments by absolute counts. While cells edited in a control site (AAVS1 safe genomic harbor) were eliminated by CAR-T cell co-culture, epitope-edited cells showed higher viability and absolute counts, similar to what we observed with K562 co-cultures (FIG. 41E,F). As KIT has known extra-hematopoietic expression in humans⁵⁵ we focused on the use of mAb instead of CAR-T cells, which might result in less severe on-target toxicities and can be used for non-genotoxic conditioning or in vivo selection of epitope-engineered cells. By plating edited HSPCs with increasing concentrations of the dimerization-blocking Fab-79D mAb, we observed preserved expansion kinetics of KIT^{H378} edited HSPCs in response to SCF while AAVS1^{BE} cells were inhibited in a dose dependent manner (FIG. 41G).

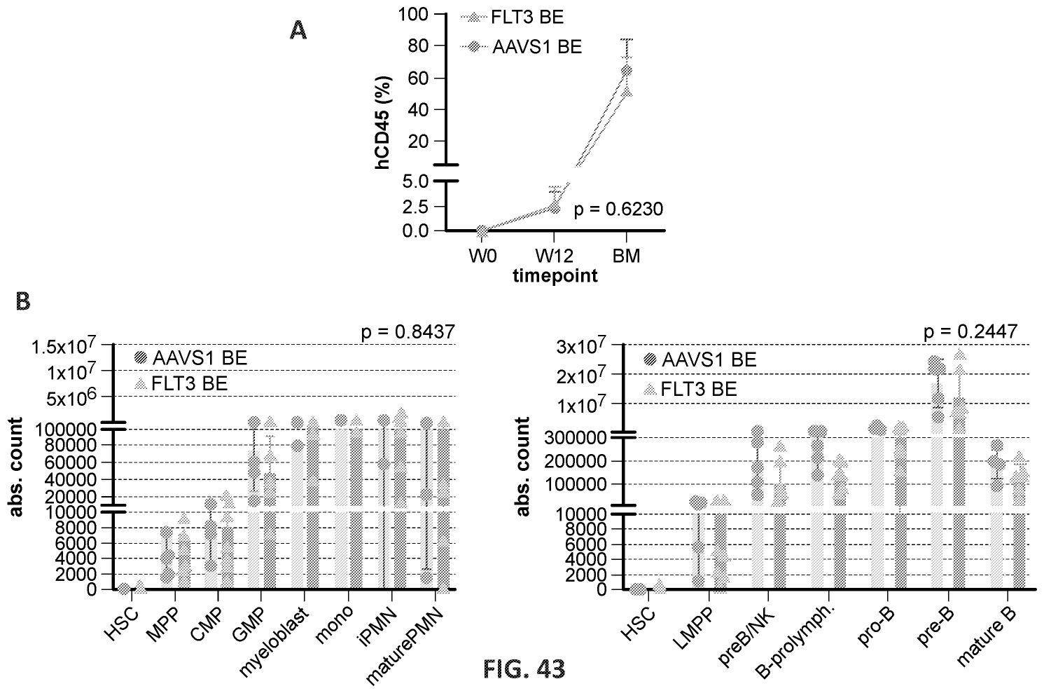

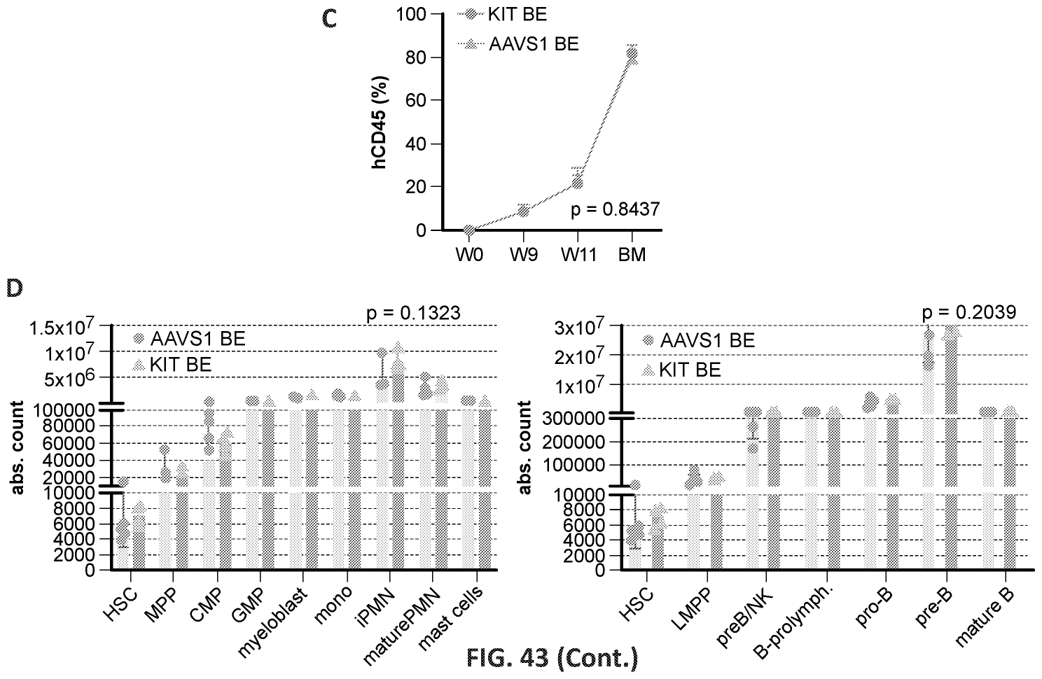

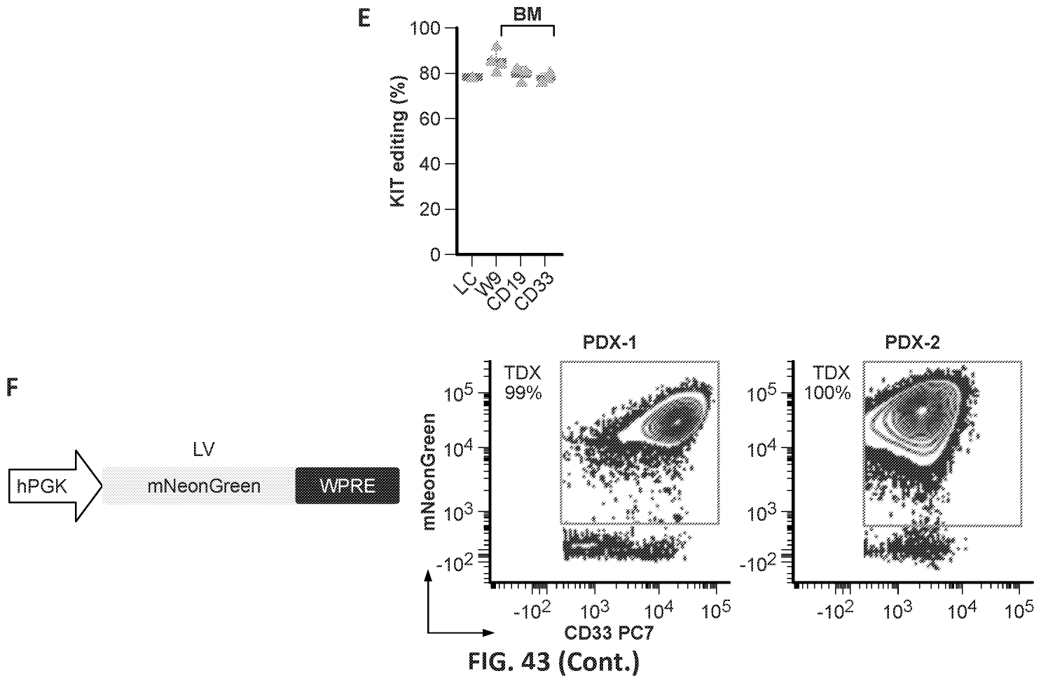

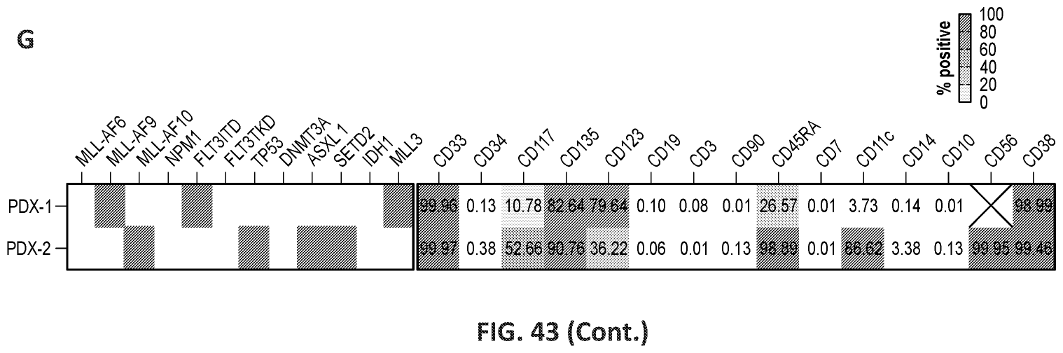

To confirm preservation of functionality of the receptor engineered CD34+ HSPCs, treated cells were cultured for 4 additional days with increasing concentrations of their respective ligand and without other cytokines. At the end of culture, we observed dose-dependent HSPCs proliferative expansion for all 3 targets, with no difference between receptor-edited and AAVS1-edited controls except for CD123^{S59} BE at IL-3 concentrations of 1-10 ng/ml (FIG. 42H). Nonetheless, we did not observe counter-selection of BE HSPCs, confirming uniform expansion of CD34+ cells regardless of CD123 epitope editing (FIG. 42I). To further confirm minimal impact on proliferation and assess the differentiation capacity of the receptor edited HSPCs, we performed cell culture and colony-forming unit (CFU) assays on HSPCs base edited for the FLT3, CD123, KIT or AAVS1 genes and observed absolute cell counts post in vitro expansion and numbers of myeloid and erythroid colonies comparable to untreated controls (FIG. 41H). Xeno-transplantation of the treated HSPCs into female NBSGW immunodeficient mice showed preserved engraftment, repopulation and multilineage differentiation capacity of FLT3^{N399} HSPCs (FIG. 41I,J), which were comparable to AAVS1 edited controls. Percentage of FLT3 editing were comparable to those measured in input cells (BE efficiency ˜35%) and stable up to 13 weeks post-transplant (FIG. 41K), confirming successful editing of the most primitive HSPC subset and no counterselection of the FLT3^{N399} cells. Transplantation of bone marrow (BM) cells into secondary recipients resulted in high human hematopoietic engraftment and no differences in lineage distribution up to 17 weeks after secondary transplant (FIG. 43A,B), and FLT3 editing levels remained comparable to those measured in primary recipients (FIG. 41K). Since murine Flt31 is cross-reactive with human FLT3, these results further confirm the functionality of the epitope engineered FLT3^{N399} variant. Similarly, the in vivo repopulating capacity and multilineage differentiation of KIT^{H378} edited HSPCs was comparable to matched AAVS1^{BE} controls (FIG. 43C-E), with no skewing of lineage differentiation and no counterselection of edited cells, further corroborating that the base editing procedure is not affecting the fitness and functionality of treated cells.

Overall, these data showed that epitope engineered HSPCs can be efficiently achieved by adenine base editor without affecting stem cell functionality and differentiation capacity.

### Example 19: FLT3BE HSPCs are Resistant to 4G8 CAR-T In Vivo Treatment

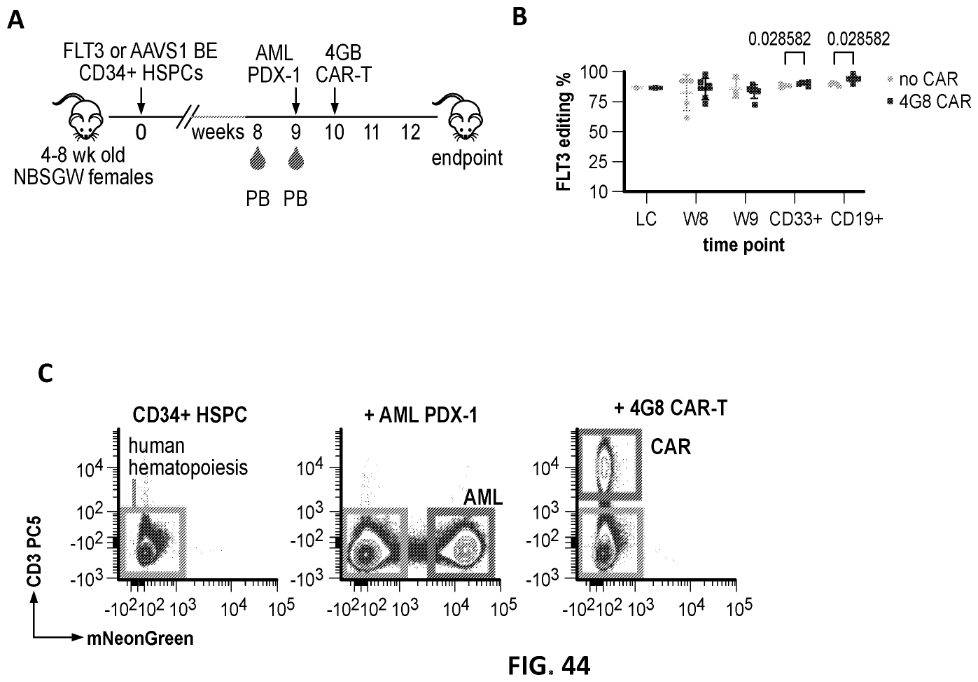

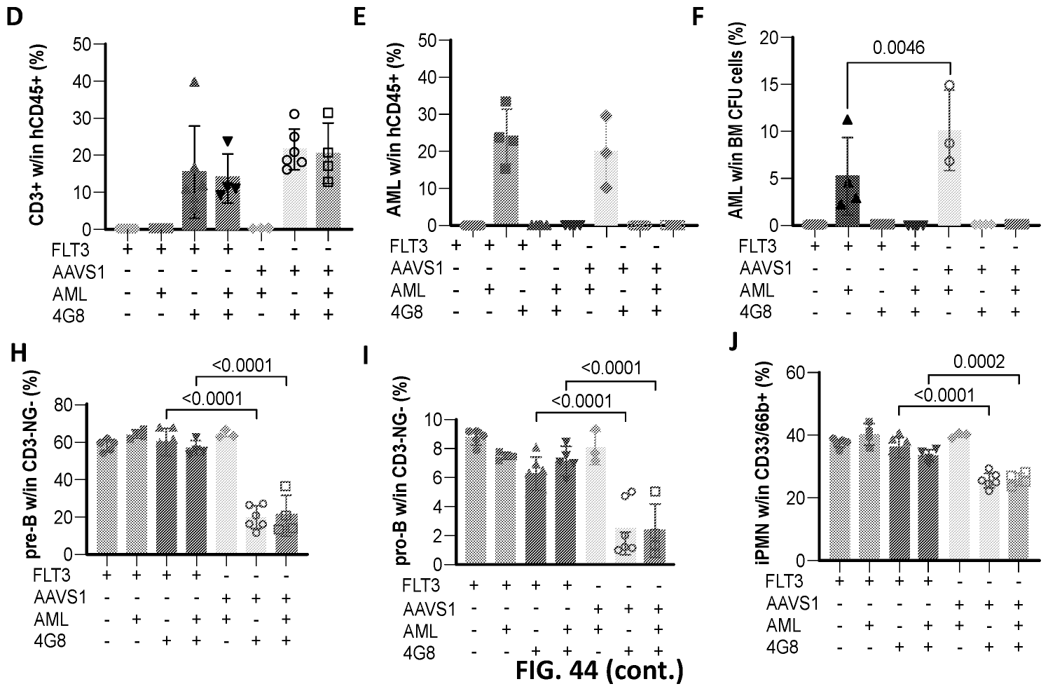

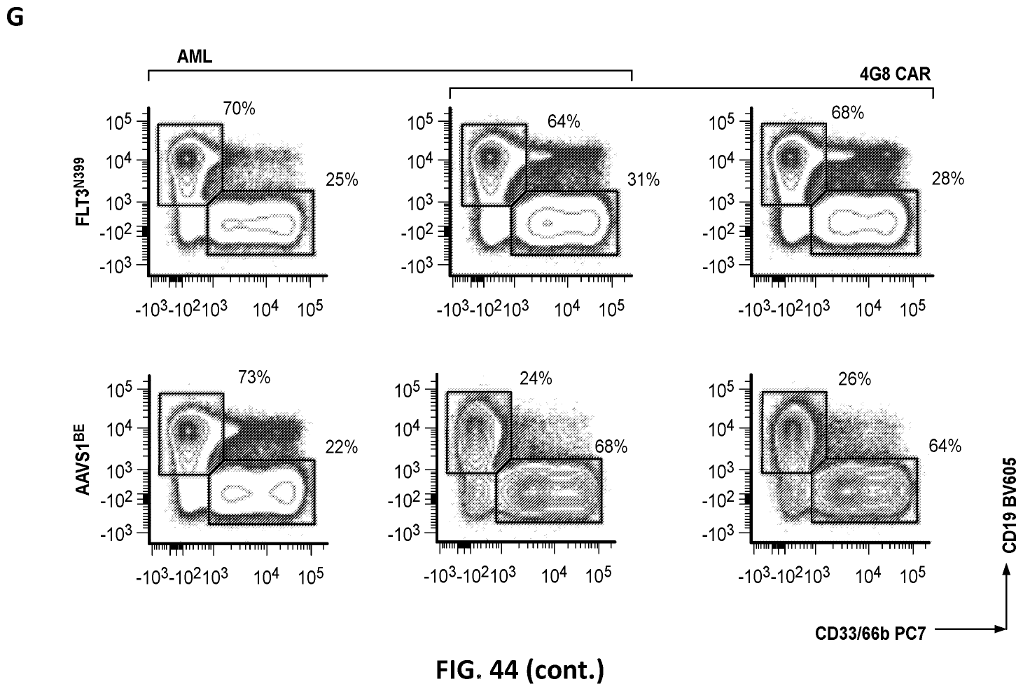

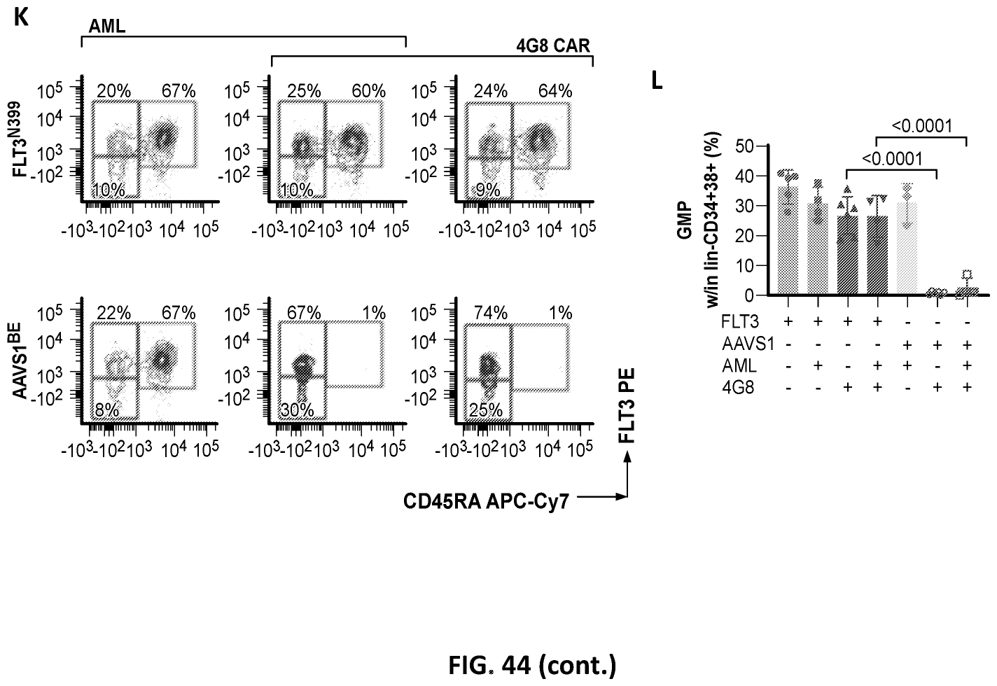

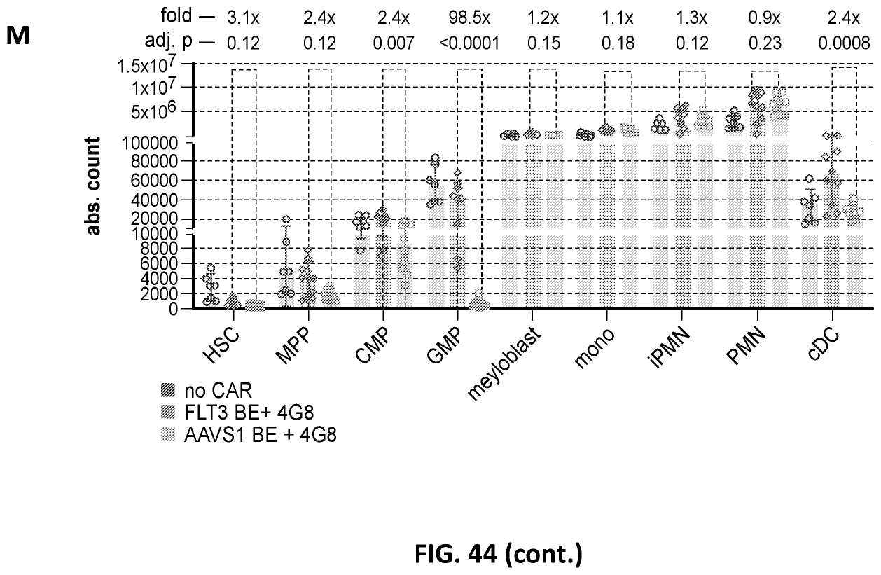

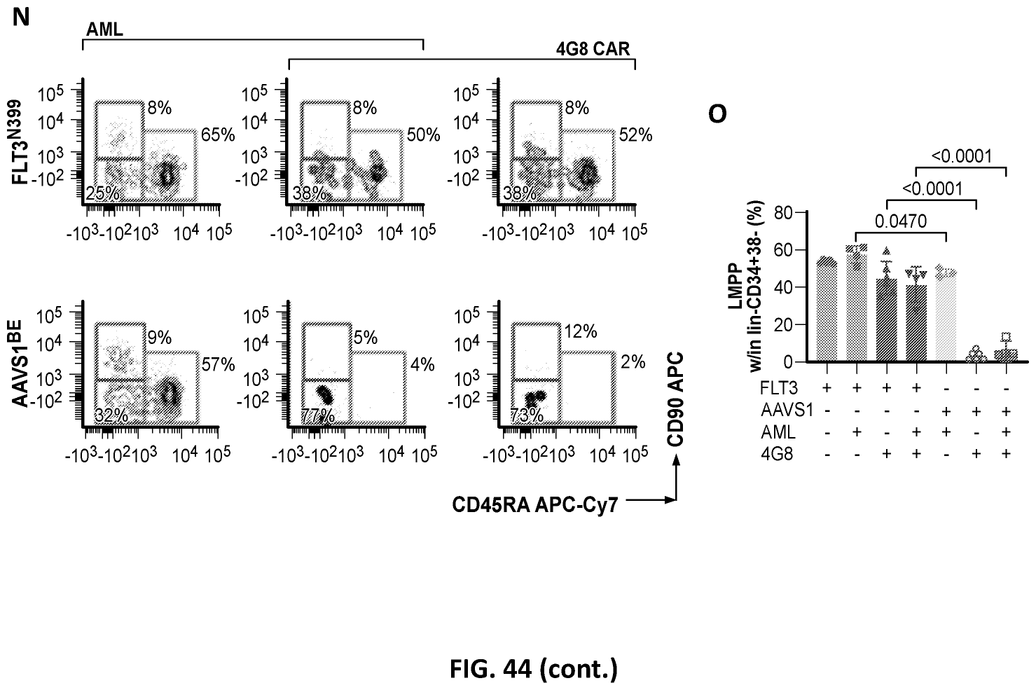

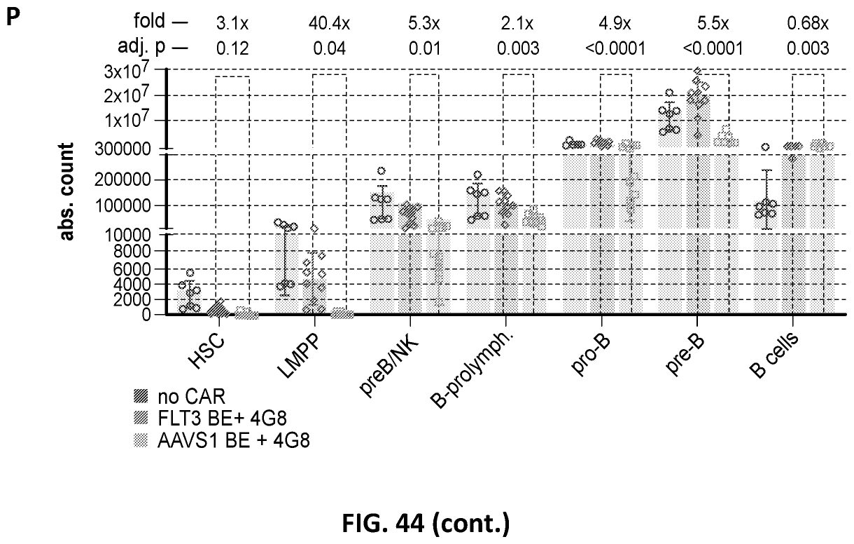

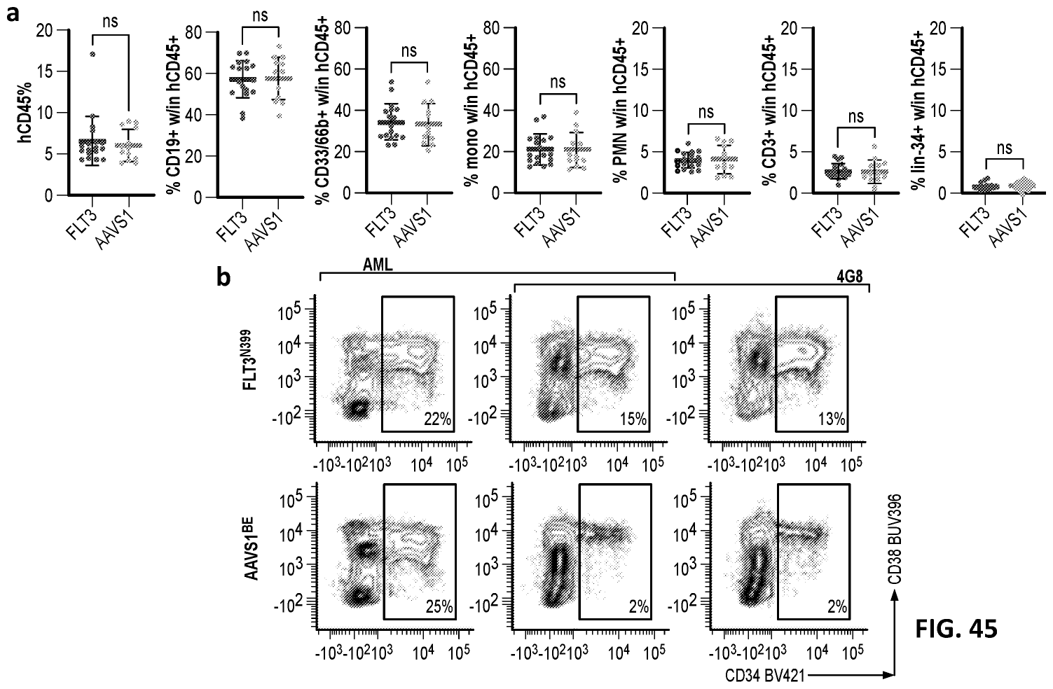

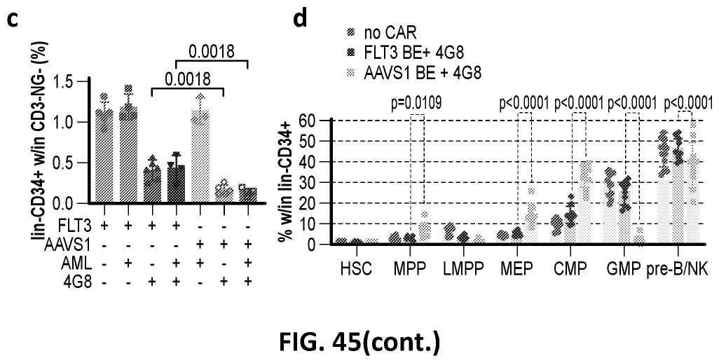

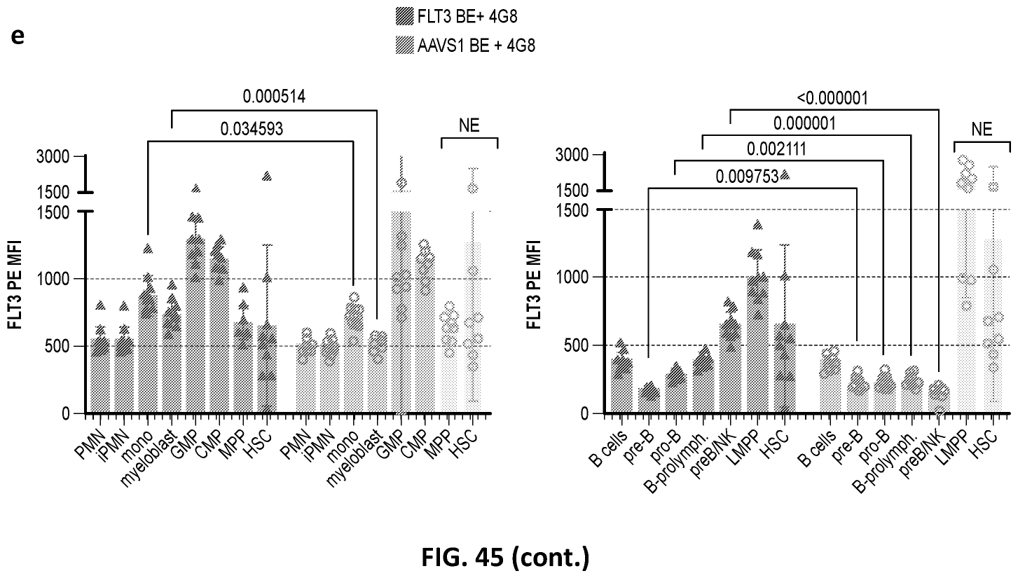

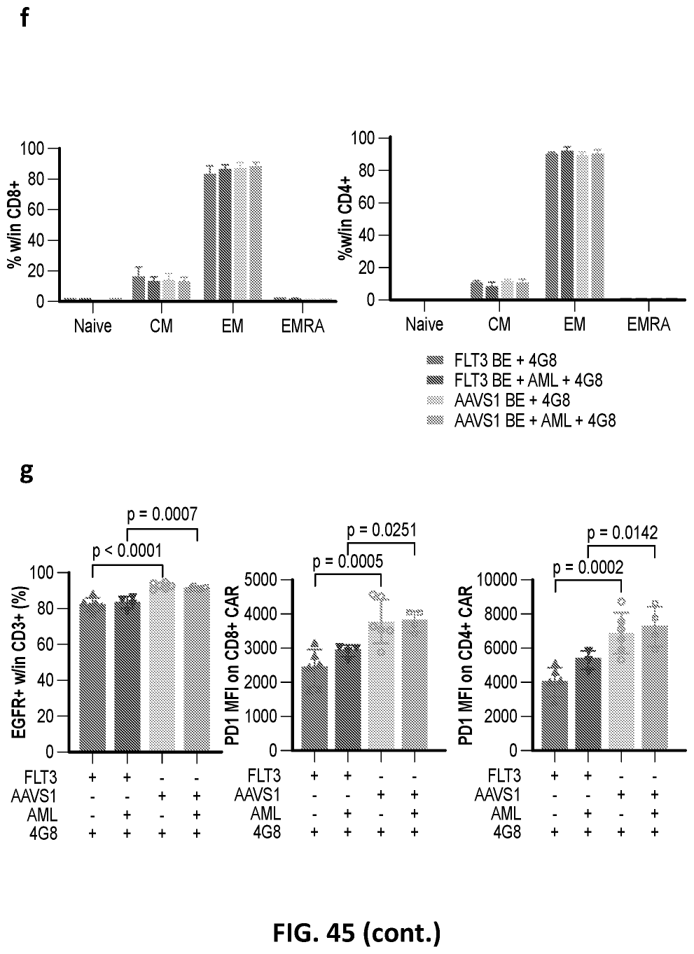

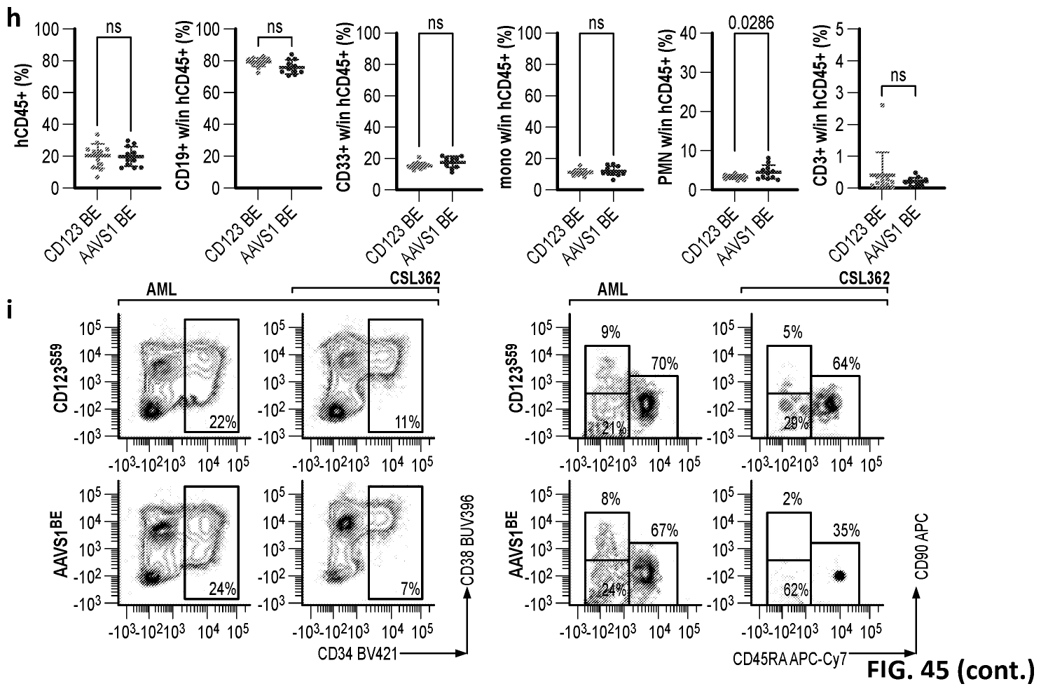

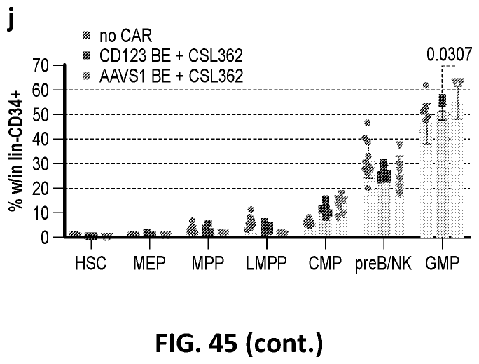

In order to assess if FLT3 CAR-T cells can be effectively used to treat AML while sparing FLT3^{N399} hematopoiesis, we sequentially engrafted NBSGW mice with CD34+ HSPCs (either FLT3^{BE} or AAVS1^{BE}) and a human patient-derived AML xenograft (PDX-1), characterized by MLL-AF9 and FLT3-ITD mutations and previously transduced with a reporter gene—mNeonGreen− to facilitate its detection within the mixed hematopoiesis (FIG. 43F,G). After 10 days from the PDX challenge, mice were treated with 4G8 CAR-T cells and their hematopoietic composition was monitored by cytofluorimetric analyses on serial blood samples and, at the end of the experiment, on hematopoietic organs (BM, spleen, SP; FIG. 44A). As observed in the previous experiment, before AML PDX or CAR-T cell administration the transplanted mice showed similar peripheral blood composition in both editing groups (FIG. 45A) and editing levels were comparable to input cells (˜85%), with no differences within the myeloid and lymphoid lineages (FACS-sorted CD33+ and CD19+ cells, respectively; FIG. 44B). Mice treated with 4G8 CAR-T showed CAR-T cell engraftment and complete AML eradication in both BM and SP (FIG. 44C-F), and a small but significant increase in the fraction of FLT3^{N399} cells in the BM (88% vs 90% within myeloid cells and 89% vs 94% within lymphoid cells; FIG. 44B). Multiparametric flow cytometry analysis of the BM revealed relative depletion of CD19+ B cells (pre-B and pro-B cells) only in the AAVS1^{BE} group treated with 4G8 CAR, while mice engrafted with FLT3^{N399} HPSC were protected (FIG. 44G-I). Within differentiated myeloid cells (CD33/66b+, excluded AML cells), the proportion of immature granulocytes (CD14−10−11c−SSC^{high})⁵⁶ was reduced in AAVS1^{BE} compared to FLT3^{N399} mice (FIG. 44J). FLT3^{N399} BE conferred selective resistance to lineage-negative progenitor cells (lin-CD34+, FIG. 45B-D) and in particular to granulo-mono progenitors (GMP, lin-CD34+38+45RA+FLT3+, FIG. 44K,L) and lymphoid-primed multipotent progenitors (LMPP, lin-CD34+38−45RA+90−10−, FIG. 44N,O), which instead were nearly completely eliminated in the AAVS1^{BE} group (1.4% vs 26.6% GMP w/in lin-CD34+38+ and 4.8% vs 43.3% LMPP w/in lin-CD34+38− in AAVS1^{BE} vs FLT3^{N399}, respectively). To more precisely identify hematopoietic subsets depleted by 4G8 CAR—and selectively protected by epitope-engineering-we compared the absolute counts of different lineages between treated FLT3^{N399} and AAVS1^{BE} groups. Absolute counts of common myeloid progenitors (CMP, lin-CD34+38+CD45RA−FLT3+), dendritic cells (CD33+14−11c+FLT3+SSC^{low}) and GMP were reduced in the AAVS1^{BE} condition (CMP 0.48×, GMP 0.01×, cDC 0.41× fold reduction in AAVS1^{BE} vs FLT3^{N399} FIG. 44M). Within lymphoid lineages and progenitors LMPP, pre-B/NK (lin-CD34+38+10+) and downstream subsets (B-prolymphocytes, pro-B and pre-B) were protected in the FLT3^{N399} vs AAVS1^{BE} group (LMPP 0.02×, pre-B/NK 0.19×, pro-B 0.2×, pre-B 0.18× fold change in AAVS1^{BE} vs FLT3^{N399}, FIG. 44P). An increase of mature B cells (which are FLT3−) in CAR-treated conditions likely reflects expansion in response to CAR-mediated cross-talk. Furthermore, the FLT3 median fluorescence intensity (MFI) of persisting pre-B/NK, B-prolymphocytes, pro-B and pre-B cells, monocytes and myeloblasts (CD33/66b+14−11c−34−SSC^{low}) in AAVS1^{BE} exposed to 4G8 CAR was lower than that measured in the same populations in the FLT3^{N399} edited group (FIG. 45E), providing additional evidence that FLT3^{N399} cells can retain FLT3 expression while avoiding CAR-mediated killing. Interestingly, while CAR-T cells detected at the end of the experiment showed a similar phenotype (mainly effector and central memory) in all groups (FIG. 45F), those exposed to FLT3^{N399} hematopoiesis displayed a lower expansion and a significant reduction in PD-1 expression compared to AAVS1^{BE} controls (FIG. 45G), suggesting an overall decrease in activation and/or exhaustion caused by the lower antigen burden to which CAR-T were exposed due to lack of recognition of epitope-engineered hematopoiesis. Importantly, FLT3^{N399} epitope editing provided the same protection against 4G8 CAR killing regardless of the presence of human PDX engraftment, highlighting the possibility to selectively eliminate AML cells while preserving hematopoietic reconstitution.

Overall, these data confirmed that in the NBSGW model, FLT3 CAR-T cell immunotherapy preferentially depletes B cell and progenitor subsets (GMP, LMPP) while FLT3^{N399} epitope editing confers protection to these subpopulations.

### Example 20: CD123BE HSPCs are Resistant to CSL362 CAR-T In Vivo Treatment

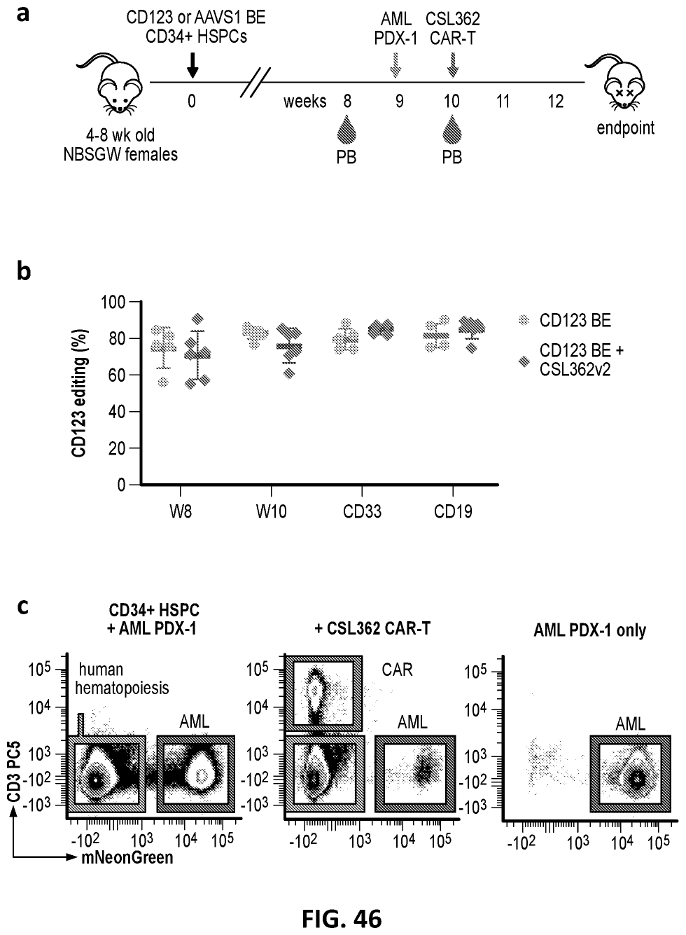

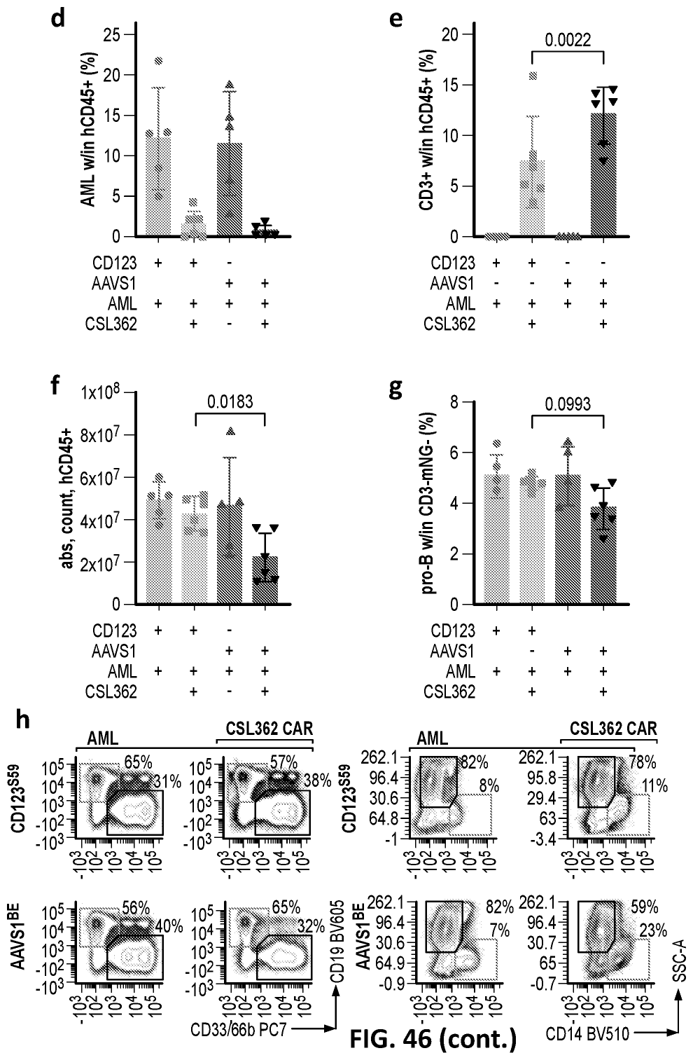

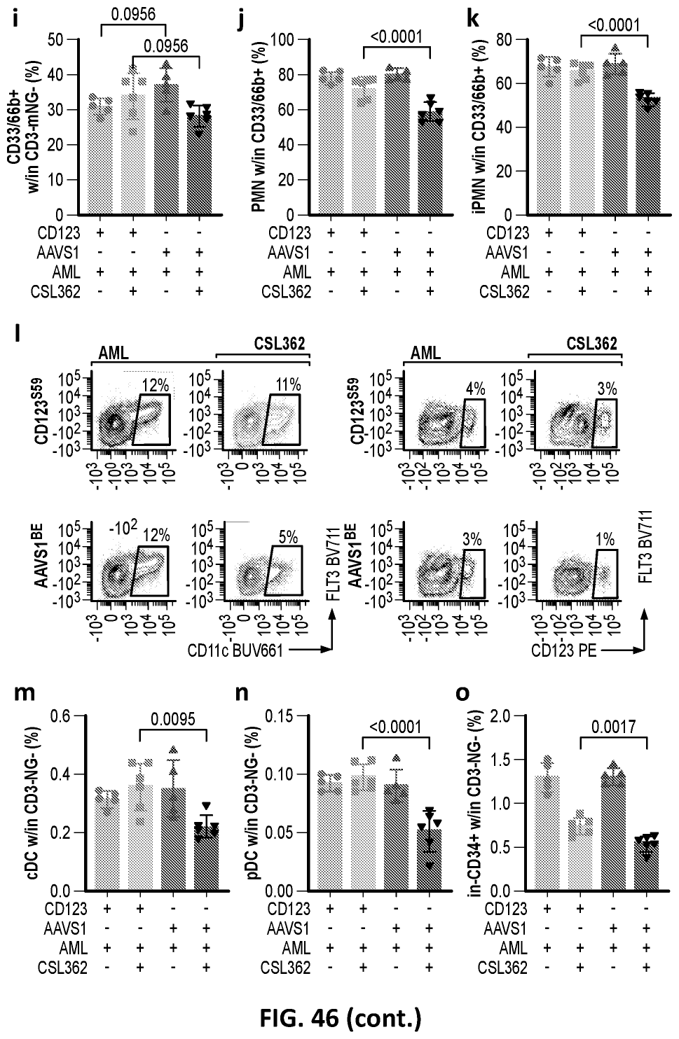

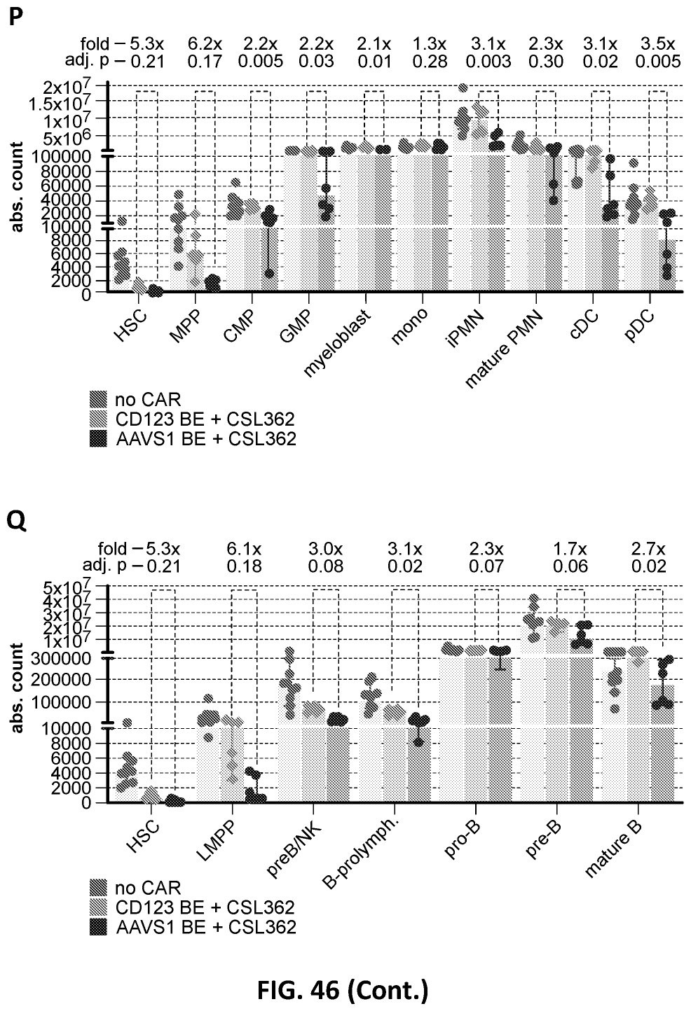

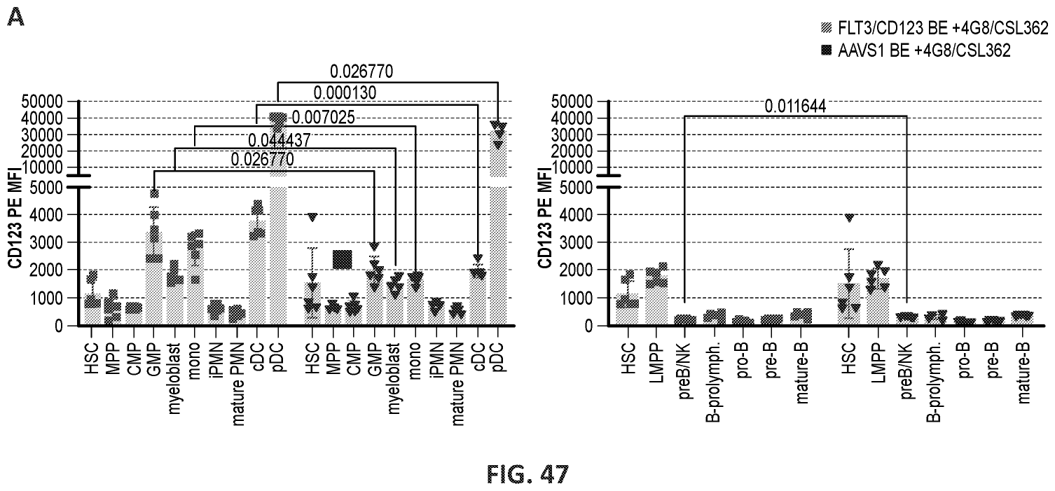

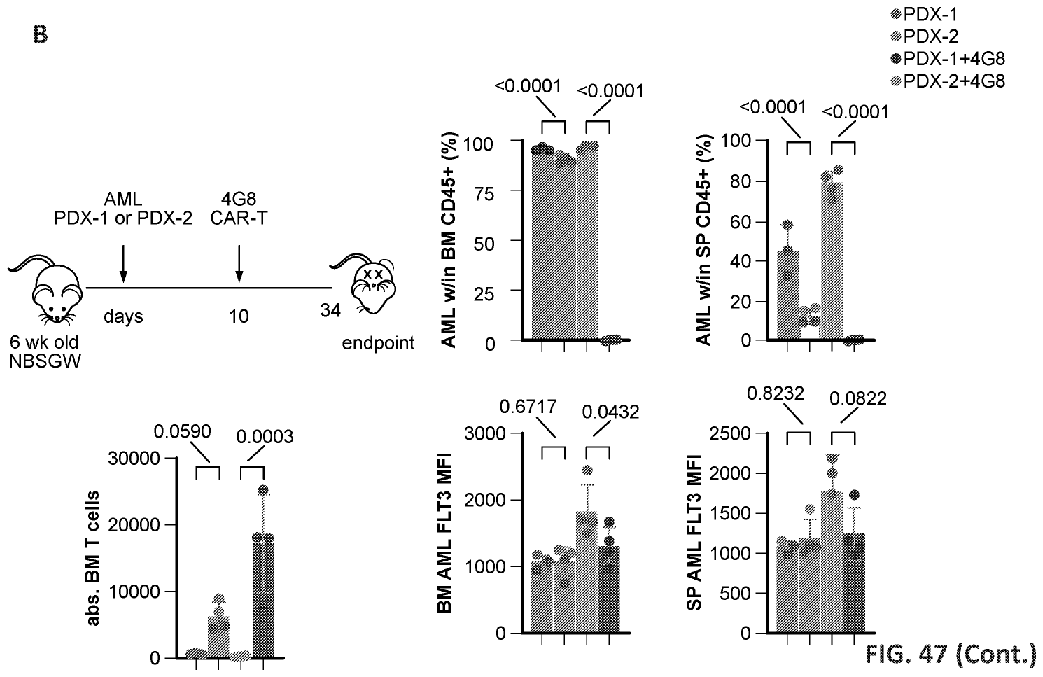

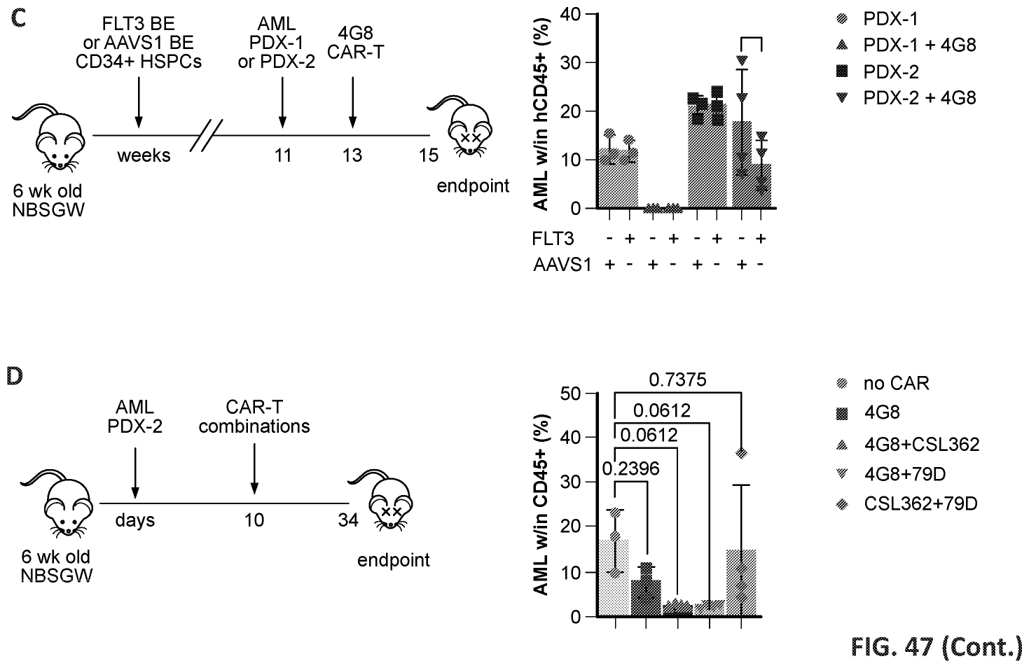

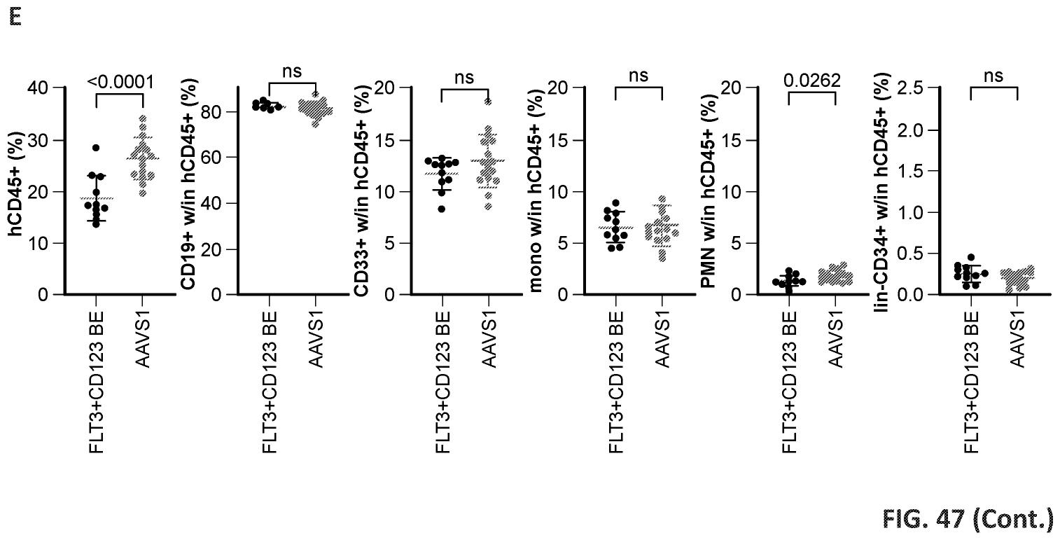

As done for FLT3 epitope editing, we performed xeno-transplantation of CD123^{S59} HSPCs into NBSGW mice and confirmed engraftment and multilineage repopulating capacity similar to AAVS1^{BE} HSPCs (FIG. 46A and FIG. 45H), with a high and stable fraction of edited cells (FIG. 46B). Transplanted mice were then injected with PDX-1—which also express CD123+ (FIG. 43G)—and treated with CSL362 CAR-T cells after 10 days. Similar to 4G8 CAR-T therapy, CSL362 CAR-T cells nearly completely eradicated AML cells (FIG. 46C,D) and display higher expansion in mice engrafted with AAVS1^{BE} HSPCs compared to CD123^{S59} (FIG. 46E). Flow cytometry analysis of BM at the end point highlighted significant reduction in the absolute counts of human hematopoietic cells (CD45+, after exclusion of AML and CAR T cells; FIG. 46F) and relative depletion of myeloid cells (CD33/66b+), including mature and immature granulocytes, in mice transplanted with AAVS1^{BE} HSPCs while the progeny of CD123^{S59} HSPCs was protected (FIG. 46H-K). Differently from the killing pattern observed with 4G8 CAR, within the lymphoid lineage, only the percentage of pro-B cells showed a trend toward reduction in CSL362 CAR-T treated AAVS1^{BE} mice (FIG. 46G). Dendritic cells (DC), including CD123^{high} plasmacytoid DC, were depleted by the treatment with CSL362 CAR-T, while they were preserved in the CD123^{S59} BE group (FIG. 46L-N). Similar to FLT3 epitope editing, lin-CD34+ progenitors were relatively preserved in the CD123^{S59} group (FIG. 46O and FIG. 45F,G). Absolute counts of myeloid populations including CMP, GMP, myeloblasts, granulocytes and DC subsets were significantly reduced in AAVS1^{BE} and protected by CD123^{S59} epitope editing. Among lymphoid cells, when comparing AAVS1^{BE} with CD123^{S59}, we observed partial depletion of B-prolymphocytes to mature B cells (FIG. 46P,Q). As observed for FLT3, CD123 median fluorescence intensity (MFI) of persisting GMP, myeloblasts, monocytes, cDC and pDC was higher in CD123^{S59} edited compared to the AAVS1^{BE} conditions treated with CSL362 CAR, while we did not observe relevant differences in lymphoid subsets (FIG. 47A).

Overall, these data showed a reduction of on-target toxicity induced by CD123 CAR-T cells on CD123^{S59} epitope edited hematopoiesis, which would otherwise result in depletion of the myeloid lineage, DCs and overall reduction in absolute counts of hematopoietic cells.

### Example 21: Multiplex Editing Enables More Effective AML Therapies

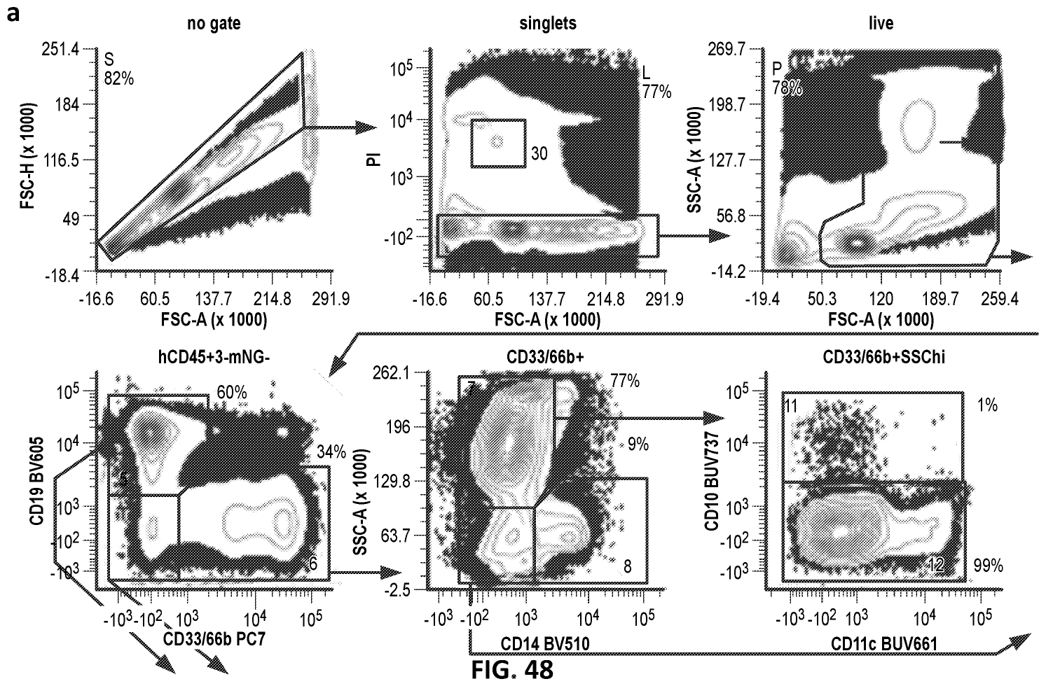

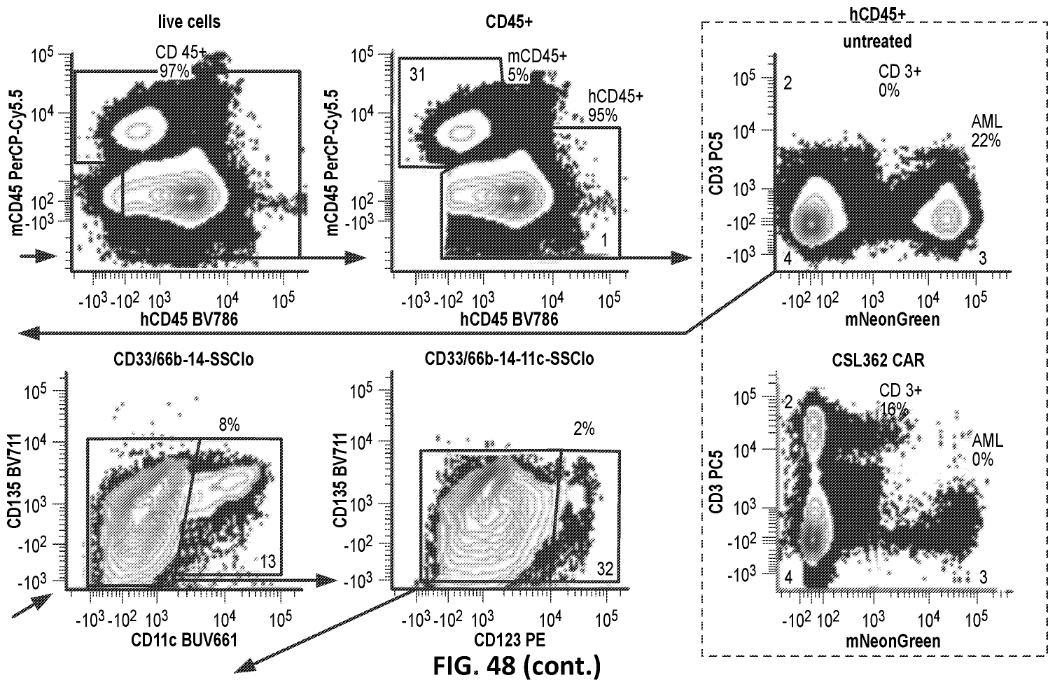

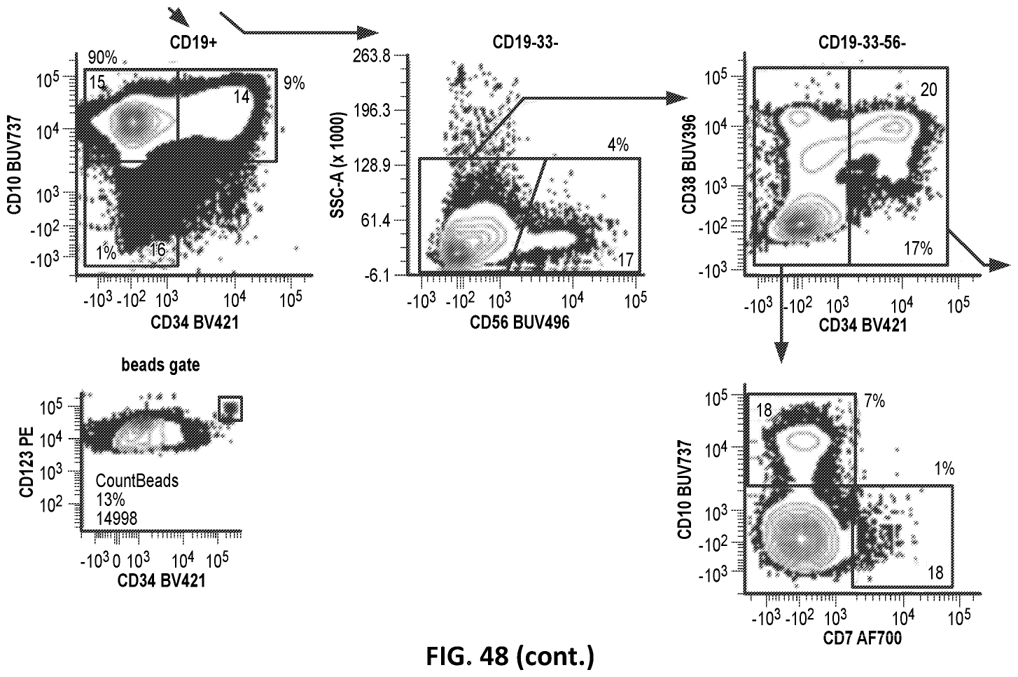

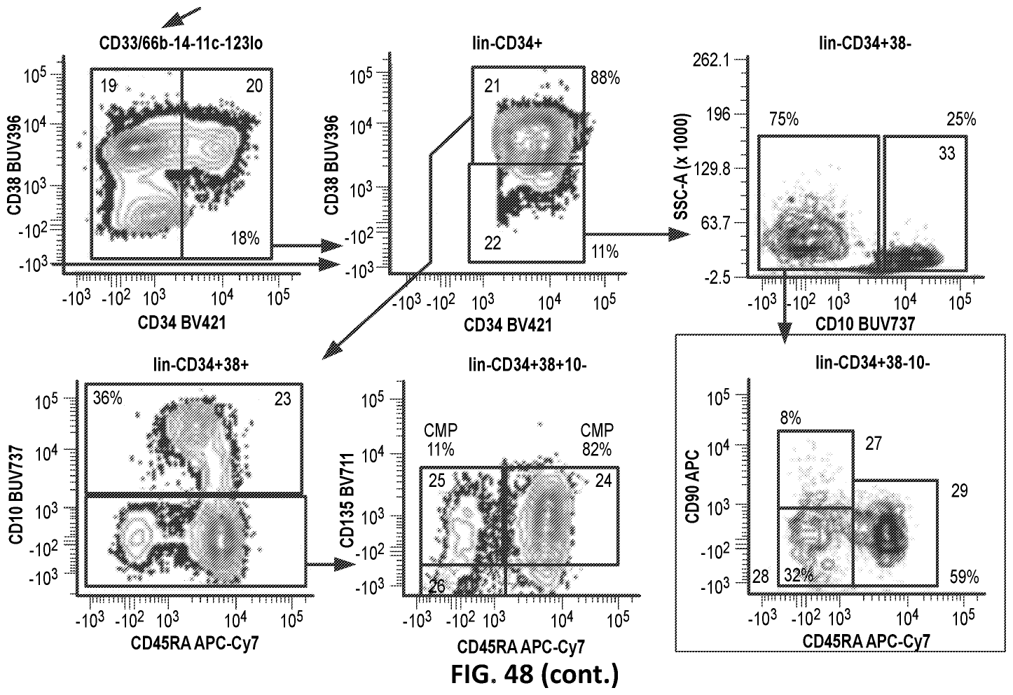

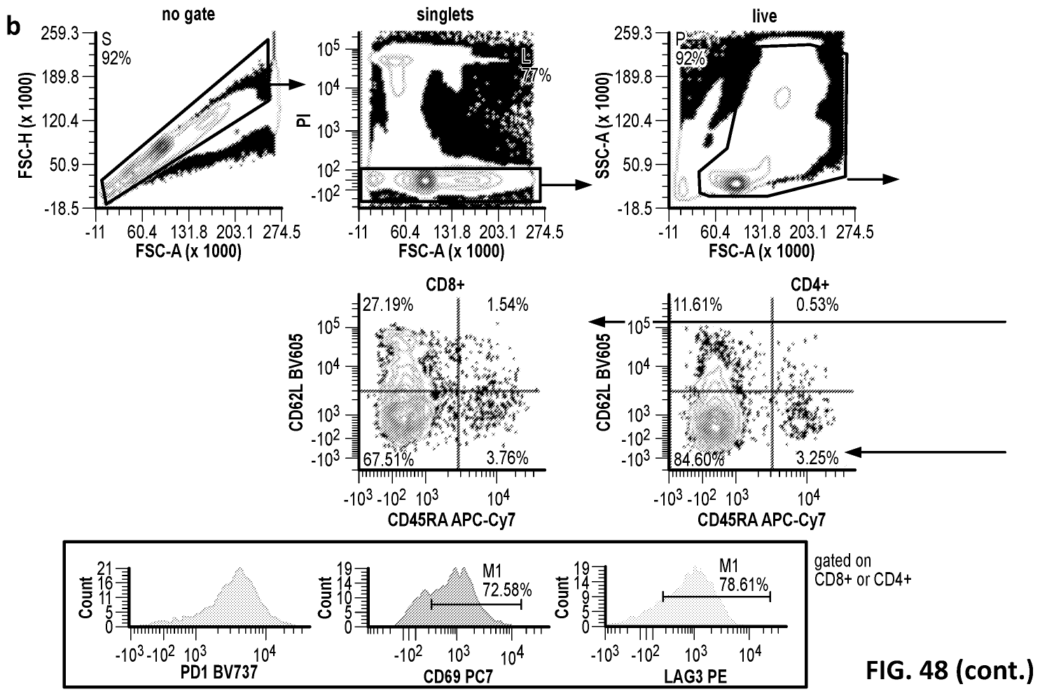

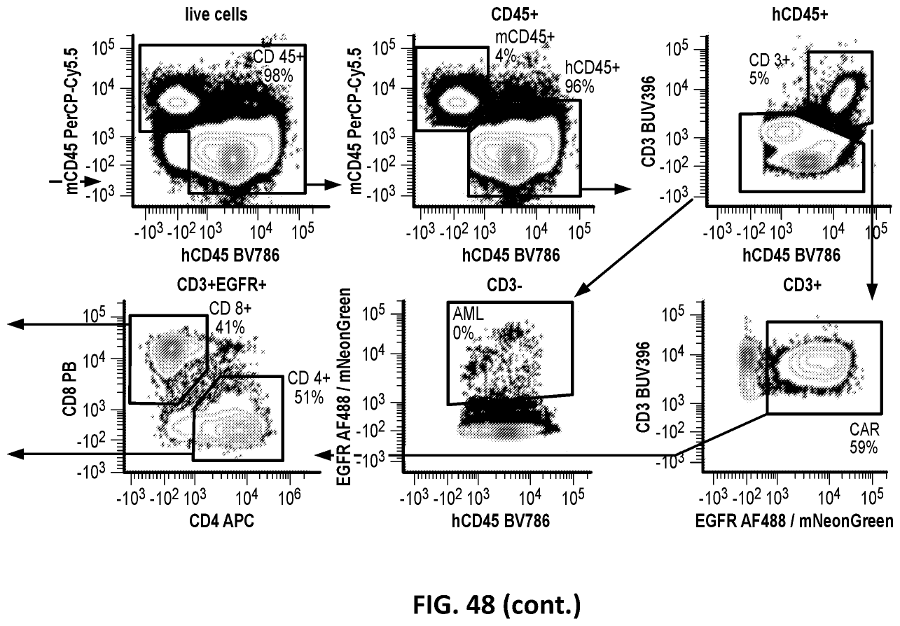

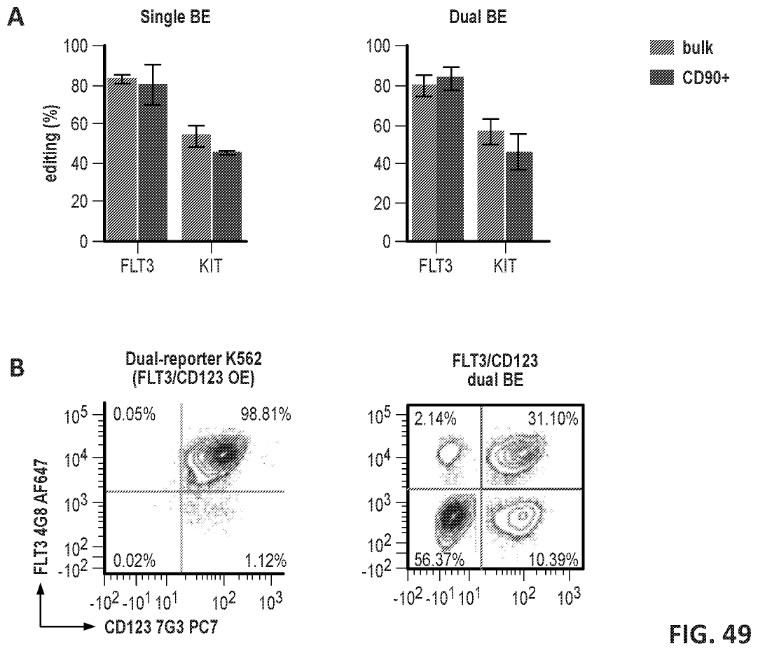

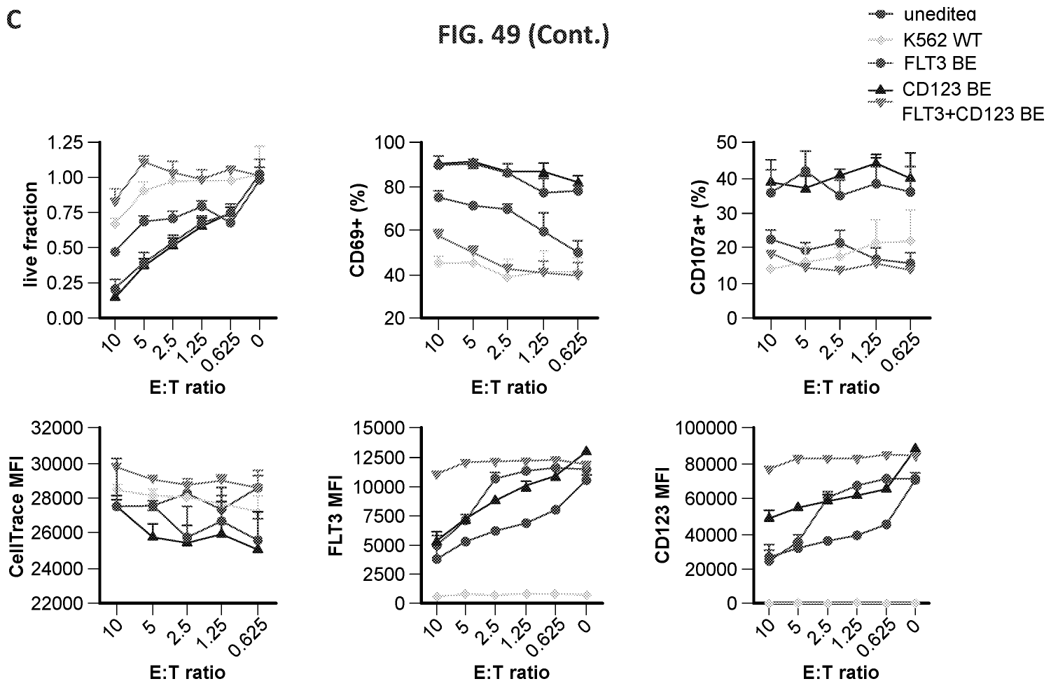

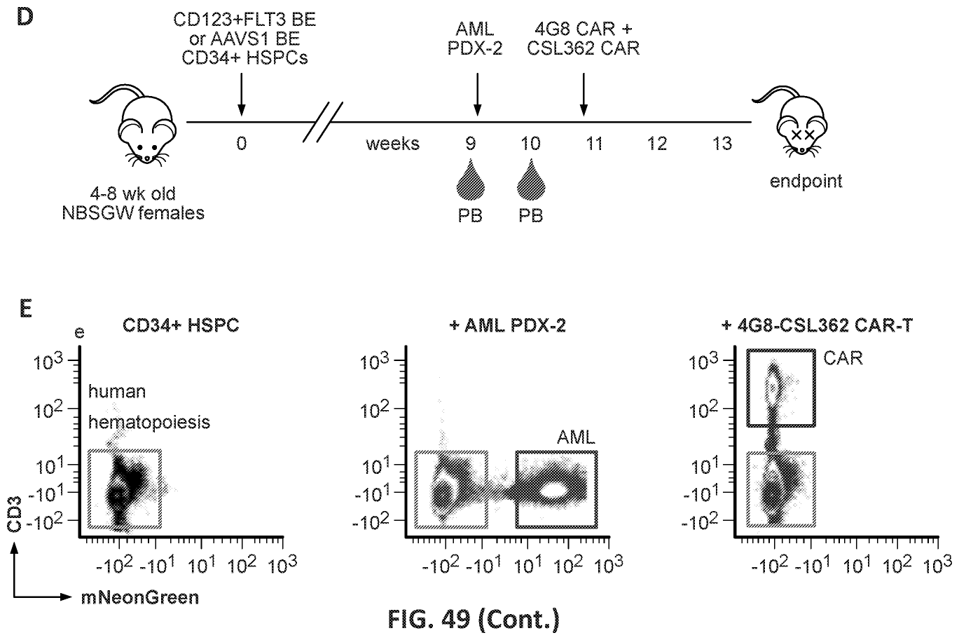

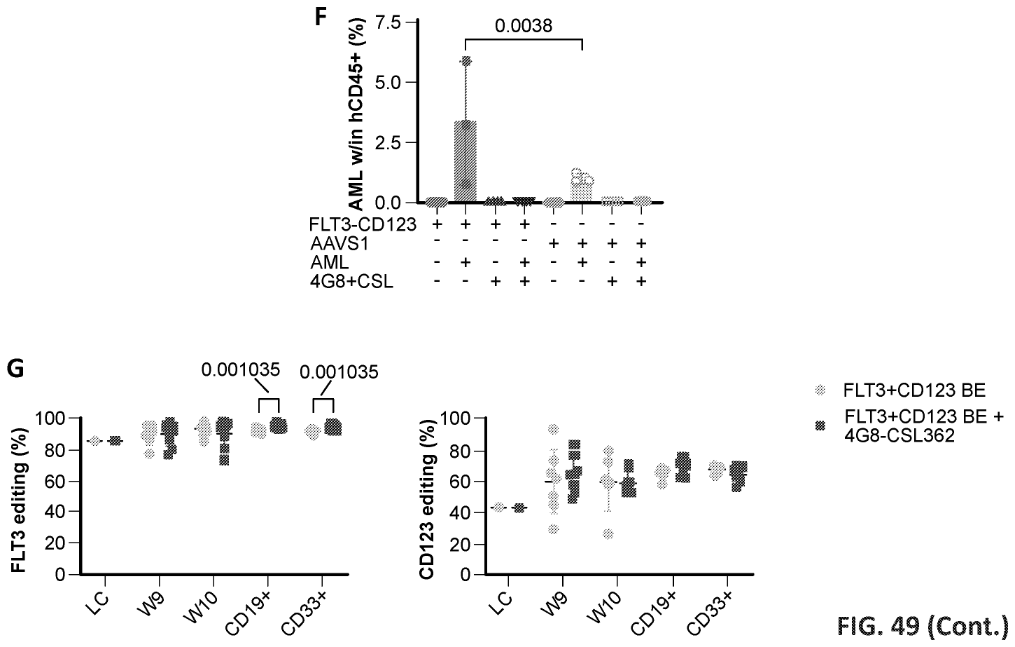

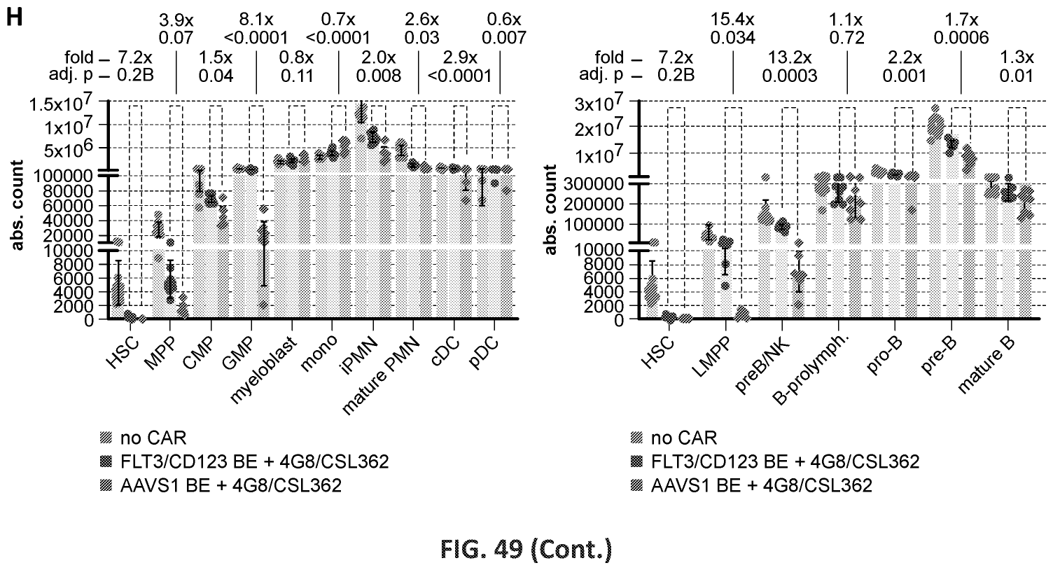

We reasoned that editing of two or more epitopes might enable even more effective immunotherapies by simultaneously attacking multiple AML targets without increasing hematopoietic toxicity. To assess the feasibility of multiple epitope-editing on primary HSPCs, we co-electroporated SpRY-ABE8e mRNA with two gRNAs on CD34+ cells and observed editing efficiencies comparable to those measured on the same targets after single BE, without overt increase in cellular toxicity (FIG. 48A and FIG. 42D,F). To obtain proof-of-concept of protection from multiple CAR-T therapies, we performed double editing of FLT3 and CD123 on a dual reporter K562 cell line (achieving >55% double-edited cells, FIG. 48B) and treated the sorted single and double edited cells with dual-specificity CAR-T cells, obtained by co-transduction with both 4G8 and CSL362 CARs. After 2 days of co-culture, only the double epitope edited cells did not induce T cell activation (CD69), degranulation (CD107a) or proliferation (CellTrace dilution) and displayed complete protection from killing while preserving expression of FLT3 and CD123 (FIG. 48B,C).

To model the increased efficacy conferred by dual-targeting immunotherapies, we identified an AML PDX that is not effectively eradicated by 4G8 CAR-T cell treatment (PDX-2 characterized by MLL-AF10 and mutated TP53, FIG. 43F,G) when xeno-transplanted either alone or in combination with human FLT3^{N399} epitope edited hematopoiesis (FIG. 47B,C). 4G8 CAR showed a trend towards higher anti-leukemia efficacy on PDX-2 when administered to FLT3^{N399} engrafted mice (FIG. 47C), suggesting a detrimental role of off-tumor CAR activation for therapeutic success. Next, we tested whether any combination of CAR-T cells targeting two of our selected antigens could provide more effective elimination of PDX-2 AML cells and found that the combination of 4G8 CAR-T with both Fab79-D and CSL362 CAR-T were the most effective (FIG. 47D), which is consistent with improved killing when two essential AML antigens are simultaneously targeted. To obtain a formal proof that double edited HSPCs are resistant to a combined CAR-T cell therapy, we xeno-transplanted dual FLT3^{N399}/CD123^{S59} epitope edited HSPCs or AAVS1^{BE} controls into NBSGW mice. After confirmation of multilineage engraftment (FIG. 47E) and injection of the PDX-2 AML cells, we treated the mice both with 4G8 and CSL362 CAR-T cells (1:1 ratio co-infusion, FIG. 48D). As suggested by our CAR-combination experiment, dual CAR-T therapy was able to fully eradicate AML cells from BM and SP (FIG. 48E,F). Both FLT3 and CD123 edited cells persisted till the endpoint and equally detected within lymphoid and myeloid cells (FIG. 48G). By analyzing the non-malignant hematopoiesis, we observed widespread reduction of myeloid and lymphoid hematopoietic lineages in AAVS1^{BE} mice treated with double-CAR-T cells, with the strongest depletion of GMP, granulocytes, cDC, LMPP, pre-B/NK, pro-B and pre-B cells and a less pronounced but significant reduction across nearly all the other subpopulations, suggesting a mixed depletion pattern in between what we had observed with 4G8 and CSL362 CARs alone (FIG. 48H).

Overall, these data provide the proof-of-concept that multiplex epitope engineering can be efficiently obtained in HSPCs and enables potent multi-target immunotherapies with reduction of overlapping on-target off-tumor hematopoietic toxicities.

### REFERENCES FOR EXAMPLES 15-21

1. Brentjens, R. J. et al. CD19-targeted T cells rapidly induce molecular remissions in adults with chemotherapy-refractory acute lymphoblastic leukemia. Sci Transl Med 5, 177ra38 (2013).
2. Turtle, C. J. et al. Durable molecular remissions in chronic lymphocytic leukemia treated with CD19-specific chimeric antigen receptor-modified T cells after failure of ibrutinib. J. Clin. Oncol. 35, 3010-3020 (2017).
3. Fry, T. J. et al. CD22-targeted CAR T cells induce remission in B-ALL that is naive or resistant to CD19-targeted CAR immunotherapy. Nat. Med. 24, 20 (2018).
4. Brentjens, R. J. et al. Safety and persistence of adoptively transferred autologous CD19-targeted T cells in patients with relapsed or chemotherapy refractory B-cell leukemias. Blood 118, 4817 (2011).
5. Spiegel, J. Y. et al. CAR T cells with dual targeting of CD19 and CD22 in adult patients with recurrent or refractory B cell malignancies: a phase 1 trial. Nat Med 27, 1419 (2021).
6. Kantarjian, H. et al. Acute myeloid leukemia: current progress and future directions. Blood Cancer J 11, 41 (2021).
7 Longo, D. L., Döhner, H., Weisdorf, D. J. & Bloomfield, C. D. Acute Myeloid Leukemia. n engl j med 12, 1136-52 (2015).
8. Döhner, H. et al. Diagnosis and management of AML in adults: 2017 ELN recommendations from an international expert panel. Blood vol. 129 424-447 Preprint at https://doi.org/10.1182/blood-2016-08-733196 (2017).
9. Isidori, A. et al. Immunotherapy in Acute Myeloid Leukemia: Where We Stand. Article 11, 1 (2021).
10. Pollyea, D. A. & Jordan, C. T. Therapeutic targeting of acute myeloid leukemia stem cells. Blood vol. 129 1627-1635 Preprint at https://doi.org/10.1182/blood-2016-10-696039 (2017).
11. Hofmann, S. et al. Chimeric Antigen Receptor (CAR) T Cell Therapy in Acute Myeloid Leukemia (AML). J Clin Med 8, 200 (2019).
12. Haubner, S. et al. Coexpression profile of leukemic stem cell markers for combinatorial targeted therapy in AML. Leukemia 2018 33:1 33, 64-74 (2018).
13. Kügler, M. et al. A recombinant trispecific single-chain Fv derivative directed against CD123 and CD33 mediates effective elimination of acute myeloid leukaemia cells by dual targeting. Br J Haematol 150, 574-86 (2010).
14. Lambert, J. et al. Gemtuzumab ozogamicin for de novo acute myeloid leukemia: final efficacy and safety updates from the open-label, phase III ALFA-0701 trial. Haematologica 104, 113-119 (2019).
15. Chen, L. et al. Targeting FLT3 by chimeric antigen receptor T cells for the treatment of acute myeloid leukemia. Leukemia vol. 31 1830-1834 Preprint at https://doi.org/10.1038/leu.2017.147 (2017).
16. el Khawanky, N. et al. Demethylating therapy increases anti-CD123 CAR T cell cytotoxicity against acute myeloid leukemia. Nature Communications 2021 12:1 12, 1-20 (2021).
17. Zhang, H. et al. Successful Anti-CLL1 CAR T-Cell Therapy in Secondary Acute Myeloid Leukemia. Front Oncol 10, 685 (2020).
18. Kenderian, S. S. et al. CD33-specific chimeric antigen receptor T cells exhibit potent preclinical activity against human acute myeloid leukemia. Leukemia 29, 1637-47 (2015).
19. Wang, J. et al. CAR-T cells targeting CLL-1 as an approach to treat acute myeloid leukemia. doi: 10.1186/s13045-017-0553-5.
20. Casucci, M. et al. CD44v6-targeted T cells mediate potent antitumor effects against acute myeloid leukemia and multiple myeloma. (2013) doi: 10.1182/blood-2013-04.
21. Myburgh, R. et al. Anti-human CD117 CAR T-cells efficiently eliminate healthy and malignant CD117-expressing hematopoietic cells. Leukemia 34, 2688-2703 (2020).
22. Jetani, H. et al. CAR T-cells targeting FLT3 have potent activity against FLT3−ITD+AML and act synergistically with the FLT3-inhibitor crenolanib. Leukemia 32, 1168-1179 (2018).
23. F, B. et al. Gene-edited stem cells enable CD33-directed immune therapy for myeloid malignancies. Proc Natl Acad Sci USA 116, 11978-87.
24. Kim, M. Y. et al. Genetic Inactivation of CD33 in Hematopoietic Stem Cells to Enable CAR T Cell Immunotherapy for Acute Myeloid Leukemia. Cell 173, 1439-1453.e19 (2018).
25. Ulyanova, T., Blasioli, J., Woodford-Thomas, T. A. & Thomas, M. L. The sialoadhesin CD33 is a myeloid-specific inhibitory receptor. Eur. J. Immunol. 29, 3440-3449 (1999).
26. Paul, S. P., Taylor, L. S., Stansbury, E. K. & McVicar, D. W. Myeloid specific human CD33 is an inhibitory receptor with differential ITIM function in recruiting the phosphatases SHP-1 and SHP-2. Blood 96, 483-490 (2000).
27. Ishida, A. et al. Negative regulation of Toll-like receptor-4 signaling through the binding of glycosylphosphatidylinositol-anchored glycoprotein, CD14, with the sialic acid-binding lectin, CD33. J. Biol. Chem. 289, 25341-25350 (2014).
28. Jacoby, E. et al. CD19 CAR immune pressure induces B-precursor acute lymphoblastic leukaemia lineage switch exposing inherent leukaemic plasticity. Nat Commun 7, (2016).
29. Ruella, M. & Maus, M. v. Catch me if you can: Leukemia Escape after CD19-Directed T Cell Immunotherapies. Comput Struct Biotechnol J 14, 357-362 (2016).
30. Sotillo, E. et al. Convergence of Acquired Mutations and Alternative Splicing of CD19 Enables Resistance to CART-19 Immunotherapy. Cancer Discov 5, 1282-1295 (2015).
31. Vago, L. et al. Loss of mismatched HLA in leukemia after stem-cell transplantation. N Engl J Med 361, 478-488 (2009).
32. Grafone, T., Palmisano, M., Nicci, C. & Storti, S. An overview on the role of FLT3-tyrosine kinase receptor in acute myeloid leukemia: Biology and treatment. Oncology Reviews vol. 6 64-74 Preprint at https://doi.org/10.4081/oncol.2012.e8 (2012).
33 Mekawy, M. A., Eissa, D. S., Hamza, M. T., Hamed, G. M. & Youssef, M. K. FLT3 receptor/CD135 expression by flow cytometry in acute myeloid leukemia: Relation to FLT3 gene mutations and mRNA transcripts. Egyptian Journal of Medical Human Genetics 19, 345-351 (2018).
34. F Kuchenbauer, W. K. C. S. A. K. W. H. T. H. Detailed analysis of FLT3 expression levels in acute myeloid leukemia. Haematologica 90, 1617-25 (2005).
35. Valverde, L. R. et al. C-kit receptor (CD117) expression in acute leukemia. Ann Hematol 72, 11-15 (1996).
36. Gao, X. et al. High Expression of c-kit mRNA Predicts Unfavorable Outcome in Adult Patients with t(8;21) Acute Myeloid Leukemia. PLOS One 10, e0124241 (2015).
37. Schwartz, S. et al. Expression of the c-kit receptor (CD117) is a feature of almost all subtypes of de novo acute myeloblastic leukemia (AML), including cytogenetically good-risk AML, and lacks prognostic significance. Leuk Lymphoma 34, 85-94 (1999).
38. Bras, A. E. et al. CD123 expression levels in 846 acute leukemia patients based on standardized immunophenotyping. Cytometry B Clin Cytom 96, 134 (2019).
39. Ehninger, A. et al. Distribution and levels of cell surface expression of CD33 and CD123 in acute myeloid leukemia. Blood Cancer J 4, (2014).
40. Haubner, S. et al. Coexpression profile of leukemic stem cell markers for combinatorial targeted therapy in AML. Leukemia 2018 33:1 33, 64-74 (2018).
41. Perriello, V. M. et al. CD123 Is Consistently Expressed on NPM1-Mutated AML Cells. Cancers (Basel) 13, 496 (2021).
42. Kandeel, E. Z., Madney, Y., Eldin, D. N. & Shafik, N. F. Overexpression of CD200 and CD123 is a major influential factor in the clinical course of pediatric acute myeloid leukemia. Exp Mol Pathol 118, (2021).
43. Cheng, J., Qu, L., Wang, J., Cheng, L. & Wang, Y. High expression of FLT3 is a risk factor in leukemia. Mol Med Rep 17, 2885 (2018).
44. Hofmann, M. et al. Generation, selection and preclinical characterization of an Fc-optimized FLT3 antibody for the treatment of myeloid leukemia. (2012) doi: 10.1038/leu.2011.372.
45. Reshetnyak, A. v et al. Structural basis for KIT receptor tyrosine kinase inhibition by antibodies targeting the D4 membrane-proximal region. doi: 10.1073/pnas. 1317118110.
46. Herzog, E. et al. Pharmacodynamic Activity and Preclinical Safety of CSL362, a Novel Humanised, Affinity Matured Monoclonal Antibody Against Human Interleukin 3 Receptor. Blood 120, 1524-1524 (2012).
47. Xie, L. H. et al. CD123 target validation and preclinical evaluation of ADCC activity of anti-CD123 antibody CSL362 in combination with NKs from AML patients in remission. Blood Cancer J 7, (2017).
48. Broughton, S. E. et al. Dual Mechanism of Interleukin-3 Receptor Blockade by an Anti-Cancer Antibody. CellReports 8, 410-419 (2014).
49. Li, Y. et al. A novel Flt3-deficient HIS mouse model with selective enhancement of human DC development. Eur J Immunol 46, 1291-1299 (2016).
50 Warmuth, M., Kim, S., Gu, X. J., Xia, G. & Adrián, F. Ba/F3 cells and their use in kinase drug discovery. Curr Opin Oncol 19, 55-60 (2007).
51. el Khawanky, N. et al. Demethylating therapy increases anti-CD123 CAR T cell cytotoxicity against acute myeloid leukemia. Nat Commun 12, (2021).
52. Vavassori, V. et al. Modeling, optimization, and comparable efficacy of T cell and hematopoietic stem cell gene editing for treating hyper-IgM syndrome. EMBO Mol Med 13, (2021).
53. Cieri, N. et al. IL-7 and IL-15 instruct the generation of human memory stem T cells from naive precursors. Blood 121, 573-584 (2013).
54. Genovese, P. et al. Targeted genome editing in human repopulating haematopoietic stem cells. Nature 510, 235-240 (2014).
55. Lennartsson, J. & Rönnstrand, L. Stem cell factor receptor/c-Kit: From basic Science to clinical implications. Physiol Rev 92, 1619-1649 (2012).
56. Basso-Ricci, L. et al. Multiparametric Whole Blood Dissection: A one-shot comprehensive picture of the human hematopoietic system. Cytometry 91, 952 (2017).
57. Dolgin, E. First CD123-targeted drug approved after wowing in rare cancer. Nat Biotechnol 37, 202-203 (2019).
58. Biasco, L. et al. In Vivo Tracking of Human Hematopoiesis Reveals Patterns of Clonal Dynamics during Early and Steady-State Reconstitution Phases. Cell Stem Cell 19, 107-119 (2016).
59. Leibowitz, M. L. et al. Chromothripsis as an on-target consequence of CRISPR-Cas9 genome editing. Nat Genet 53, 895-905 (2021).
60. Schiroli, G. et al. Precise Gene Editing Preserves Hematopoietic Stem Cell Function following Transient p53-Mediated DNA Damage Response. Cell Stem Cell 24, 551-565.e8 (2019).
61. Ferrari, S. et al. Efficient gene editing of human long-term hematopoietic stem cells validated by clonal tracking. Nat Biotechnol 38, 1298 (2020).
62. Gill, S. et al. Preclinical targeting of human acute myeloid leukemia and myeloablation using chimeric antigen receptor-modified T cells. Blood 123, 2343-2354 (2014).
63. Russkamp, N. F., Myburgh, R., Kiefer, J. D., Neri, D. & Manz, M. G. Anti-CD117 immunotherapy to eliminate hematopoietic and leukemia stem cells. Exp Hematol 95, 31-45 (2021).
64. Hand, T. H. et al. Catalytically Enhanced Cas9 through Directed Protein Evolution. CRISPR Journal 4, 223-232 (2021).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A genetically engineered hematopoietic stem cell (HSPC), comprising a genetically engineered FLT3 gene, wherein the genetically engineered FLT3 gene is engineered such that its encoded protein has reduced binding to a therapeutic anti-FLT3 antibody.

2. The genetically engineered HSPC of claim 1, wherein the genetically engineered FLT3 gene comprises at least one mutation in exon 9 of the FLT3 gene.

3. The genetically engineered HSPC of claim 2, wherein at least one mutation in exon 9 of the genetically engineered FLT3 gene results in a polypeptide bearing a mutation at position N399.

4. The genetically engineered HSPC of claim 3, wherein the mutation at position N399 is N399D or N399G.

5. The genetically engineered HSPC of claim 1, wherein the therapeutic anti-FLT3 antibody is anti-FLT3 clone 4G8 antibody.

6. The genetically engineered HSPC of claim 1, wherein the therapeutic anti-FLT3 antibody is an antibody that has the same six CDRs as, or competes with, 4G8 antibody.

7. The genetically engineered HSPC of claim 1, wherein the genetically engineered HSPCs are genetically engineered using a CRISPR system comprising a guide nucleic acid and a nuclease.

8. The genetically engineered HSPC of claim 7, wherein the nuclease is either Streptococcus pyogenes Cas9 (SpCas9), Staphylococcus aureus (SaCas9), Lachnospiraceae bacterium Cas12a (LbCas12a), or Acidaminococcus sp. BV3L6 (AsCas12a).

9. The genetically engineered HSPC of claim 8, wherein the CRISPR system comprises SpCas9.

10. The genetically engineered HSPC of claim 8 or claim 9, wherein the guide nucleic acid is selected from the group consisting of SEQ ID NO: 13, SEQ ID NO: 14, SEQ ID NO: 15, and SEQ ID NO: 16.

11. The genetically engineered HSPC of claim 10, wherein CRISPR system further comprises a template DNA.

12. The genetically engineered HSPC of claim 11, wherein the template DNA is selected from the group consisting of SEQ ID NO: 40, SEQ ID NO: 41, SEQ ID NO: 42, and SEQ ID NO: 43.

13. The genetically engineered HSPC of claim 7, wherein the nuclease is a catalytically impaired SpCas9 linked to a base editor enzyme.

14. The genetically engineered HSPC of claim 13, wherein the base editor enzyme is a nucleotide deaminase.

15. The genetically engineered HSPC of claim 14, wherein the nucleotide deaminase is either a cytosine deaminase or an adenosine deaminase.

16. The genetically engineered HSPC of claim 13, wherein the catalytically impaired SpCas9 is NG-SpCas9 or SpRY-SpCas9.

17. The genetically engineered HSPC of any one of claims 13-16, wherein the catalytically impaired SpCas9 comprises a mutation at position D10A.

18. The genetically engineered HSPC of claim 17, wherein the catalytically impaired SpCas9 further comprises a mutation at position K918N.

19. The genetically engineered HSPC of any one of claims 13-18, wherein the guide RNA is selected from the group consisting of SEQ ID NO: 17, SEQ ID NO: 18, SEQ ID NO: 19, SEQ ID NO: 20, and SEQ ID NO: 23.

20. The genetically engineered HSPC of claim 1, wherein the genetically engineered FLT3 gene encodes a polypeptide which comprises the amino acid sequence of SEQ ID NO: 51 or SEQ ID NO: 52.

21. A population of genetically engineered hematopoietic stem cells (HSPCs), comprising the genetically engineered HSPCs of any one of claims 1-20.

22. A method of treating a hematopoietic malignancy, the method comprising administering to a human subject:
(a) the population of genetically engineered hematopoietic stem cells of claim 21, and
(b) a therapeutically effective amount of at least one agent comprising an anti-FLT3 antibody binding domain or an antibody or antibody fragment comprising the anti-FLT3 binding domain.

23. The method of claim 22, wherein the at least one agent comprises a Chimeric Antigen Receptor-T (CAR-T) cell comprising the anti-FLT3 antibody binding domain.

24. The method of claim 22, wherein the hematopoietic malignancy is B-lymphoblastic leukemia (BLL), acute myeloid leukemia (AML), or T-cell acute lymphoblastic leukemia (T-ALL).

25. The method of claim 22, further comprising obtaining HSPCs from a biological sample from the human subject and genetically engineering the HSPCs from the biological sample from the human subject, thereby forming the population of genetically engineered HSPCs.

26. The method of claim 25, wherein the biological sample is bone marrow cells, blood, cord blood cells, or mobilized peripheral blood-derived CD34+ hematopoietic stem and progenitor cells.

27. A genetically engineered hematopoietic stem cell (HSPC), comprising a genetically engineered CD123 gene, wherein the genetically engineered CD123 gene is engineered such that its encoded protein has reduced binding to a therapeutic anti-CD123 antibody.

28. The genetically engineered HSPC of claim 27, wherein the therapeutic anti-CD123 antibody is clone 7G3 antibody or its humanized counterpart CSL362.

29. The genetically engineered HSPC of claim 28, wherein the genetically engineered CD123 gene comprises at least one mutation in exon 2 of the CD123 gene.

30. The genetically engineered HSPC of claim 29, wherein at least one mutation in exon 2 of the genetically engineered CD123 gene results in a polypeptide bearing a mutation at position S59.

31. The genetically engineered HSPC of claim 30, wherein the mutation at S59 is S59P or S59F.

32. The genetically engineered HSPC of claim 27, wherein the therapeutic anti-CD123 antibody is anti-CD123 clone 6H6 antibody or anti-CD123 clone S18016F antibody.

33. The genetically engineered HSPC of claim 32, wherein the genetically engineered CD123 gene comprises at least one mutation in exon 3 of the CD123 gene.

34. The genetically engineered HSPC of claim 32, wherein at least one mutation in exon 3 of the genetically engineered CD123 gene results in a polypeptide bearing a mutation at position P88.

35. The genetically engineered HSPC of claim 34, wherein the mutation at P88 is P88L or P88S.

36. The genetically engineered HSPC of claim 31, wherein the genetically engineered HSPCs are genetically engineered using a CRISPR system comprising a guide nucleic acid and a nuclease.

37. The genetically engineered HSPC of claim 36, wherein the guide nucleic acid is selected from the group consisting of SEQ ID NO: 24, SEQ ID NO: 27, SEQ ID NO: 30, SEQ ID NO: 32, SEQ ID NO: 33, and SEQ ID NO: 34.

38. The genetically engineered HSPC of claim 35, wherein the nuclease is a catalytically impaired SpCas9 linked to a base editor enzyme.

39. The genetically engineered HSPC of claim 38, wherein the base editor enzyme is a nucleotide deaminase.

40. The genetically engineered HSPC of claim 38, wherein the base editor enzyme is either a cytosine deaminase or an adenosine deaminase.

41. The genetically engineered HSPC of claim 38, wherein the catalytically impaired SpCas9 is NG-SpCas9 or SpRY-SpCas9.

42. The genetically engineered HSPC of any one of claims 37-41, wherein the catalytically impaired SpCas9 comprises a mutation at position D10A.

43. The genetically engineered HSPC of claim 42, wherein the catalytically impaired SpCas9 further comprises a mutation at position K918N.

44. The genetically engineered HSPC of claim 27, wherein the genetically engineered CD123 gene encodes a polypeptide which comprises the amino acid sequence of SEQ ID NO: 54, SEQ ID NO: 55, SEQ ID NO: 56, SEQ ID NO: 57, or SEQ ID NO: 58.

45. A population of genetically engineered hematopoietic stem cells (HSPCs), comprising the genetically engineered HSPCs of any one of claims 27-44.

46. A method of treating a hematopoietic malignancy, the method comprising administering to a human subject:
(a) the population of genetically engineered hematopoietic stem cells of claim 45, and
(b) a therapeutically effective amount of at least one agent comprising an anti-CD123 antibody binding domain or an antibody or antibody fragment comprising the anti-CD123 binding domain.

47. The method of claim 46, wherein the at least one agent comprises a Chimeric Antigen Receptor-T (CAR-T) cell comprising the anti-CD123 antibody binding domain.

48. The method of claim 46, wherein the hematopoietic malignancy is B-lymphoblastic leukemia (BLL), acute myeloid leukemia (AML), T-cell acute lymphoblastic leukemia (T-ALL), or Blastic Plasmacytoid Dendritic Cell Leukemia (BPCDN).

49. The method of claim 46, further comprising obtaining HSPCs from a biological sample from the human subject and genetically engineering the HSPCs from the biological sample from the human subject, thereby forming the population of genetically engineered HSPCs.

50. The method of claim 49, wherein the biological sample is bone marrow cells, blood, cord blood cells, or mobilized peripheral blood-derived CD34+ hematopoietic stem and progenitor cells.

51. A population of genetically engineered hematopoietic stem cells (HSPCs) comprising:
(i) a genetically engineered FLT3 gene, wherein the genetically engineered FLT3 gene encodes a protein that has reduced binding to a therapeutic anti-FLT3 antibody, and
(ii) a genetically engineered CD123 gene, wherein the genetically engineered CD123 gene encodes a protein that has reduced binding to a therapeutic anti-CD123 antibody.

52. The population of HSPCs of claim 51, wherein the genetically engineered FLT3 gene comprises at least one mutation in exon 9 of the FLT3 gene.

53. The population of HSPCs of claim 52, wherein at least one mutation in exon 9 of the genetically engineered FLT3 gene results in a polypeptide bearing a mutation at position N399.

54. The population of HSPCs of claim 51, wherein the genetically engineered CD123 gene comprises at least one mutation in exon 2 of the CD123 gene.

55. The population of HSPCs of claim 54, wherein at least one mutation in exon 2 of the genetically engineered CD123 gene results in a polypeptide bearing a mutation at position S59.

56. The population of HSPCs of claim 51, wherein the therapeutic anti-FLT3 antibody is anti-FLT3 clone 4G8 antibody.

57. The population of HSPCs of claim 51, wherein the therapeutic anti-CD123 antibody is anti-CD123 clone 7G3 antibody or CSL362 antibody.

58. The population of HSPCs of claim 51, wherein the population of HSPCs are genetically engineered using a CRISPR system comprising at least two guide nucleic acids and a nuclease.

59. The population of HSPCs of claim 58, wherein the at least two guide nucleic acids are 1) SEQ ID NO: 18 or SEQ ID NO: 20 and 2) SEQ ID NO: 24 or SEQ ID NO: 27.

60. The population of HSPCs of claim 59, wherein the at least two guide nucleic acids are SEQ ID NO: 20 and SEQ ID NO: 27.

61. The population of HSPCs of claim 58, wherein the nuclease is a catalytically impaired SpCas9 linked to a base editor enzyme.

62. The population of HSPCs of claim 61, wherein the base editor enzyme is a nucleotide deaminase.

63. The population of HSPCs of claim 61, wherein the base editor enzyme is either a cytosine deaminase or an adenosine deaminase.

64. The population of HSPCs of claim 61, wherein the catalytically impaired SpCas9 is NG-SpCas9 or SpRY-SpCas9.

65. The population of HSPCs of any one of claims 61 to 64, wherein the catalytically impaired SpCas9 comprises a mutation at position D10A.

66. The population of HSPCs of claim 65, wherein the SpCas9 further comprises a mutation at position K918N.

67. A method of treating a hematopoietic malignancy, the method comprising administering to a human subject:
(a) the population of HSPCs of any one of claims 51-66, and
(b) a therapeutically effective amount of at least one agent comprising one or both of: (1) an anti-FLT3 antibody binding domain or an antibody or antibody fragment comprising the anti-FLT3 binding domain, and/or (2) an anti-CD123 antibody binding domain or an antibody or antibody fragment comprising the anti-CD123 binding domain.

68. The method of claim 67, wherein the at least one agent comprises a Chimeric Antigen Receptor-T (CAR-T) cell comprising the anti-FLT3 antibody binding domain and/or the anti-CD123 antibody binding domain.

69. The method of claim 67, wherein the hematopoietic malignancy is B-lymphoblastic leukemia (BLL), acute myeloid leukemia (AML), T-cell acute lymphoblastic leukemia (T-ALL), or Blastic Plasmacytoid Dendritic Cell Leukemia (BPCDN).

70. The method of claim 67, further comprising obtaining HSPCs from a biological sample from the human subject and genetically engineering the HSPCs from the biological sample from the human subject, thereby forming the population of genetically engineered HSPCs.

71. The method of claim 70, wherein the biological sample is bone marrow cells, blood, cord blood cells, or mobilized peripheral blood-derived CD34+ hematopoietic stem and progenitor cells.

72. A genetically engineered hematopoietic stem cell (HSPC), comprising a genetically engineered KIT gene, wherein the genetically engineered KIT gene is engineered such that its encoded protein has reduced binding to a therapeutic anti-KIT antibody.

73. The genetically engineered HSPC of claim 72, wherein the genetically engineered KIT gene comprises at least one mutation in exon 7 of the KIT gene.

74. The genetically engineered HSPC of claim 73, wherein at least one mutation in exon 7 of the genetically engineered KIT gene results in a polypeptide bearing a mutation at position H378.

75. The genetically engineered HSPC of claim 74, wherein the mutation at position H378 is H378R.

76. The genetically engineered HSPC of claim 72, wherein the therapeutic anti-KIT antibody is anti-KIT clone Fab79D antibody.

77. The genetically engineered HSPC of claim 72, wherein the genetically engineered HSPCs are genetically engineered using a CRISPR system comprising a guide nucleic acid and a nuclease.

78. The genetically engineered HSPC of claim 72, wherein the guide nucleic acid is selected from the group consisting of SEQ ID NO: 36, SEQ ID NO: 37, SEQ ID NO: 38, and SEQ ID NO: 39.

79. The genetically engineered HSPC of claim 77, wherein the nuclease is a catalytically impaired SpCas9 linked to a base editor enzyme.

80. The genetically engineered HSPC of claim 79, wherein the base editor enzyme is a nucleotide deaminase.

81. The genetically engineered HSPC of claim 79, wherein the base editor enzyme is either a cytosine deaminase or an adenosine deaminase.

82. The genetically engineered HSPC of claim 79, wherein the catalytically impaired SpCas9 is NG-SpCas9 or SpRY-SpCas9.

83. The genetically engineered HSPC of any one of claims 79-82, wherein the catalytically impaired SpCas9 comprises a mutation at position D10A.

84. The genetically engineered HSPC of claim 83, wherein the SpCas9 further comprises a mutation at position K918N.

85. A population of genetically engineered hematopoietic stem cells (HSPCs), comprising the genetically engineered HSPCs of any one of claims 72-84.

86. A method of treating a hematopoietic malignancy, the method comprising administering to a human subject:
(a) the population of genetically engineered hematopoietic stem cells of claim 85, and
(b) a therapeutically effective amount of at least one agent comprising the anti-KIT antibody binding domain or an antibody or antibody fragment comprising the anti-KIT binding domain.

87. The method of claim 86, wherein the at least one agent comprises a Chimeric Antigen Receptor-T (CAR-T) cell comprising the anti-KIT antibody binding domain.

88. The method of claim 86, wherein the hematopoietic malignancy is B-lymphoblastic leukemia (BLL), acute myeloid leukemia (AML), or T acute lymphoblastic leukemia (T-ALL).

89. The method of claim 86, further comprising obtaining HSPCs from a biological sample from the human subject and genetically engineering the HSPCs from the biological sample from the human subject, thereby forming the population of genetically engineered HSPCs.

90. The method of claim 89, wherein the biological sample is bone marrow cells, blood, cord blood cells, or mobilized peripheral blood-derived CD34+ hematopoietic stem and progenitor cells.

91. A population of genetically engineered hematopoietic stem cells (HSPCs) comprising:
(i) a genetically engineered KIT gene, wherein the genetically engineered KIT gene encodes a protein that has reduced binding to a therapeutic anti-KIT antibody, and
(ii) a genetically engineered CD123 gene, wherein the genetically engineered CD123 gene encodes a protein that has reduced binding to a therapeutic anti-CD123 antibody.

92. The population of HSPCs of claim 91, wherein the genetically engineered KIT gene comprises at least one mutation in exon 7 of the KIT gene.

93. The population of HSPCs of claim 92, wherein at least one mutation in exon 7 of the genetically engineered KIT gene results in a polypeptide bearing a mutation at position H378.

94. The population of HSPCs of claim 91, wherein the genetically engineered CD123 gene comprises at least one mutation in exon 2 of the CD123 gene.

95. The population of HSPCs of claim 94, wherein at least one mutation in exon 2 of the genetically engineered CD123 gene results in a polypeptide bearing a mutation at position S59.

96. The population of HSPCs of claim 91, wherein the therapeutic anti-KIT antibody is anti-KIT clone Fab79D antibody.

97. The population of HSPCs of claim 91, wherein the therapeutic anti-CD123 antibody is anti-CD123 clone 7G3 antibody or CSL362 antibody.

98. The population of HSPCs of claim 91, wherein the population of HSPCs are genetically engineered using a CRISPR system comprising at least two guide nucleic acids and a nuclease.

99. The population of HSPCs of claim 98, wherein the at least two guide nucleic acids are 1) SEQ ID NO: 36, SEQ ID NO: 37, SEQ ID NO: 38, or SEQ ID NO: 39 and 2) SEQ ID NO: 24 or SEQ ID NO: 27.

100. The population of HSPCs of claim 99, wherein the at least two guide nucleic acids are SEQ ID NO: 37 and SEQ ID NO: 27.

101. The population of HSPCs of claim 98, wherein the nuclease is a catalytically impaired SpCas9 linked to a base editor enzyme.

102. The population of HSPCs of claim 101, wherein the base editor enzyme is a nucleotide deaminase.

103. The population of HSPCs of claim 101, wherein the base editor enzyme is either a cytosine deaminase or an adenosine deaminase.

104. The population of HSPCs of claim 101, wherein the catalytically impaired SpCas9 is NG-SpCas9 or SpRY-SpCas9.

105. The population of HSPCs of any one of claims 101 to 104, wherein the catalytically impaired SpCas9 comprises a mutation at position D10A.

106. The population of HSPCs of claim 105, wherein the SpCas9 further comprises a mutation at position K918N.

107. A method of treating a hematopoietic malignancy, the method comprising administering to a human subject:
(a) the population of HSPCs of any one of claims 91-106, and
(b) a therapeutically effective amount of at least one agent comprising one or both of: (1) an anti-KIT antibody binding domain or an antibody or antibody fragment comprising the anti-KIT binding domain, and/or (2) an anti-CD123 antibody binding domain or an antibody or antibody fragment comprising the anti-CD123 binding domain.

108. The method of claim 107, wherein the at least one agent comprises a Chimeric Antigen Receptor-T (CAR-T) cell comprising the anti-KIT antibody binding domain and/or the anti-CD123 antibody binding domain.

109. The method of claim 107, wherein the hematopoietic malignancy is B-lymphoblastic leukemia (BLL), acute myeloid leukemia (AML), T-cell acute lymphoblastic leukemia (T-ALL), or Blastic Plasmacytoid Dendritic Cell Leukemia (BPCDN).

110. The method of claim 107, further comprising obtaining HSPCs from a biological sample from the human subject and genetically engineering the HSPCs from the biological sample from the human subject, thereby forming the population of genetically engineered HSPCs.

111. The method of claim 110, wherein the biological sample is bone marrow cells, blood, cord blood cells, or mobilized peripheral blood-derived CD34+ hematopoietic stem and progenitor cells.

112. A population of genetically engineered hematopoietic stem cells (HSPCs) comprising:
(i) a genetically engineered FLT3 gene, wherein the genetically engineered FLT3 gene encodes a protein that has reduced binding to a therapeutic anti-FLT3 antibody, and
(ii) a genetically engineered KIT gene, wherein the genetically engineered KIT gene encodes a protein that has reduced binding to a therapeutic anti-KIT antibody.

113. The population of HSPCs of claim 112, wherein the genetically engineered FLT3 gene comprises at least one mutation in exon 9 of the FLT3 gene.

114. The population of HSPCs of claim 113, wherein at least one mutation in exon 9 of the genetically engineered FLT3 gene results in a polypeptide bearing a mutation at position N399.

115. The population of HSPCs of claim 112, wherein the genetically engineered KIT gene comprises at least one mutation in exon 7 of the KIT gene.

116. The population of HSPCs of claim 115, wherein at least one mutation in exon 7 of the genetically engineered KIT gene results in a polypeptide bearing a mutation at position H378.

117. The population of HSPCs of claim 112, wherein the therapeutic anti-FLT3 antibody is anti-FLT3 clone 4G8 antibody.

118. The population of HSPCs of claim 112, wherein the therapeutic anti-KIT antibody is anti-KIT clone Fab79D antibody.

119. The population of HSPCs of claim 112, wherein the population of HSPCs are genetically engineered using a CRISPR system comprising at least two guide nucleic acids and a nuclease.

120. The population of HSPCs of claim 119, wherein the at least two guide nucleic acids are 1) SEQ ID NO: 18 or SEQ ID NO: 20 and 2) SEQ ID NO: 36, SEQ ID NO: 37, SEQ ID NO: 38, or SEQ ID NO: 39.

121. The population of HSPCs of claim 120, wherein the at least two guide nucleic acids are SEQ ID NO: 20 and SEQ ID NO: 37.

122. The population of HSPCs of claim 119, wherein the nuclease is a catalytically impaired SpCas9 linked to a base editor enzyme.

123. The population of HSPC of claim 122, wherein the base editor enzyme is a nucleotide deaminase.

124. The population of HSPC of claim 122, wherein the base editor enzyme is either a cytosine deaminase or an adenosine deaminase.

125. The population of HSPCs of claim 122, wherein the catalytically impaired SpCas9 is NG-SpCas9 or SpRY-SpCas9.

126. The population of HSPCs of any one of claims 122 to 125, wherein the catalytically impaired SpCas9 comprises a mutation at position D10A.

127. The population of HSPCs of claim 126, wherein the SpCas9 further comprises a mutation at position K918N.

128. A method of treating a hematopoietic malignancy, the method comprising administering to a human subject:
(a) the population of HSPCs of any one of claims 112-127, and
(b) a therapeutically effective amount of at least one agent comprising one or both of: (1) an anti-FLT3 antibody binding domain or an antibody or antibody fragment comprising the anti-FLT3 binding domain, and/or (2) an anti-KIT antibody binding domain or an antibody or antibody fragment comprising the anti-KIT binding domain.

129. The method of claim 128, wherein the at least one agent comprises a Chimeric Antigen Receptor-T (CAR-T) cell comprising the anti-FLT3 antibody binding domain and/or the anti-KIT antibody binding domain.

130. The method of claim 128, wherein the hematopoietic malignancy is B-lymphoblastic leukemia (BLL), acute myeloid leukemia (AML), T-cell acute lymphoblastic leukemia (T-ALL, or Blastic Plasmacytoid Dendritic Cell Leukemia (BPCDN).

131. The method of claim 128, further comprising obtaining HSPCs from a biological sample from the human subject and genetically engineering the HSPCs from the biological sample from the human subject, thereby forming the population of genetically engineered HSPCs.

132. The method of claim 131, wherein the biological sample is bone marrow cells, blood, cord blood cells, or mobilized peripheral blood-derived CD34+ hematopoietic stem and progenitor cells.

133. A chimeric antigen receptor (CAR) comprising a polypeptide comprising:
(a) one or more epitope binding fragments that binds to an epitope of one or more cell-surface lineage-specific proteins,
(b) a hinge domain,
(c) a transmembrane domain,
(d) a co-stimulatory domain, and
(e) a cytoplasmic signaling domain,
wherein the one or more cell-surface lineage-specific proteins are selected from FLT3, CD123, and/or KIT.

134. The CAR of claim 133, wherein the cell-surface lineage-specific protein is FLT3 and the CAR comprises the amino acid sequence of SEQ ID NO: 73.

135. The CAR of claim 133, wherein the cell-surface lineage-specific protein is FLT3 and the one or more epitope binding fragments comprises the one or more epitope binding fragments from SEQ ID NO: 73.

136. The CAR of claim 133, wherein the cell-surface lineage-specific protein is FLT3 and the one or more epitope binding fragments comprise the following CDR sequences: GYTFTSYWMH (SEQ ID NO: 96), EIDPSDSYKDYNQKFK (SEQ ID NO: 97, RAITTTPFDF (SEQ ID NO: 98), RASQSISNNLH (SEQ ID NO: 99), YASQSIS (SEQ ID NO: 100), and QQSNTWPYT (SEQ ID NO: 101).

137. The CAR of claim 133, wherein the cell-surface lineage-specific protein is CD123 and the CAR comprises the amino acid sequence of SEQ ID NO: 75, SEQ ID NO: 86, or SEQ ID NO: 87.

138. The CAR of claim 133, wherein the cell-surface lineage-specific protein is CD123 and the one or more epitope binding fragments comprises the one or more epitope binding fragments from SEQ ID NO: 75, SEQ ID NO: 86, or SEQ ID NO: 87.

139. The CAR of claim 133, wherein the cell-surface lineage-specific protein is CD123 and the one or more epitope binding fragments comprise the following CDR sequences: GYSFTDYYMK (SEQ ID NO: 104), DIIPSNGATFYNQKFKG (SEQ ID NO: 105), ARSHLLRASWFAY (SEQ ID NO: 106), SQSLLNSGNQKNYLT (SEQ ID NO: 107), WASTRES (SEQ ID NO: 108), and QNDYSYPYT (SEQ ID NO: 109).

140. The CAR of claim 133, wherein the cell-surface lineage-specific protein is CD123 and the one or more epitope binding fragments comprise the following CDR sequences: DIIPSNGATFYNQKFKG (SEQ ID NO: 105), SQSLLNSGNQKNYLT (SEQ ID NO: 107), WASTRES (SEQ ID NO: 108), and QNDYSYPYT (SEQ ID NO: 109).

141. The CAR of claim 133, wherein the cell-surface lineage-specific protein is KIT and the CAR comprises the amino acid sequence of SEQ ID NO: 69 or SEQ ID NO: 71.

142. The CAR of claim 133, wherein the cell-surface lineage-specific protein is KIT and the one or more epitope binding fragments comprises the one or more epitope binding fragments from SEQ ID NO: 69 or SEQ ID NO: 71.

143. The CAR of claim of claim 133, wherein the cell-surface lineage-specific protein is KIT and the one or more epitope binding fragments comprise the following CDR sequences: GFNISVYMMH (SEQ ID NO: 88), SIYPYSGYTYYADSVKG (SEQ ID NO: 89), ARYVYHALDY (SEQ ID NO: 90), RASQRGLRNVAVA (SEQ ID NO: 91, SASSLYS (SEQ ID NO: 92), and QQWAVHSLIT (SEQ ID NO: 93).

144. The CAR of claim 133, wherein the one or more cell-surface lineage-specific proteins are FLT3 and CD123 and the CAR comprises the amino acid sequence of SEQ ID NO: 77 or SEQ ID NO: 79.

145. The CAR of claim 133, wherein the one or more cell-surface lineage-specific proteins are FLT3 and CD123 and the one or more epitope binding fragments comprises the one or more epitope binding fragments from SEQ ID NO: 77 or SEQ ID NO: 79.

146. The CAR of claim 133, wherein the one or more cell-surface lineage-specific proteins are FLT3 and CD123 and the one or more epitope binding fragments comprise the following CDR sequences: GYTFTSYWMH (SEQ ID NO: 96), EIDPSDSYKDYNQKFK (SEQ ID NO: 97), RAITTTPFDF (SEQ ID NO: 98), RASQSISNNLH (SEQ ID NO: 99), YASQSIS (SEQ ID NO: 100), QQSNTWPYT (SEQ ID NO: 101), GYSFTDYYMK (SEQ ID NO: 104), DIIPSNGATFYNQKFKG (SEQ ID NO: 105), ARSHLLRASWFAY (SEQ ID NO: 106), SQSLLNSGNQKNYLT (SEQ ID NO: 107), WASTRES (SEQ ID NO: 108), and QNDYSYPYT (SEQ ID NO: 109).

147. The CAR of any one of claims 133 to 146, wherein the hinge domain is a CD28 hinge, an IgG4 hinge, or a CD8α hinge.

148. The CAR of any one of claims 133 to 147, wherein the transmembrane domain is a CD28 TM, a CD8α TM, or a 4-1BB TM.

149. The CAR of any one of claims 133 to 148, wherein the co-stimulatory domain is CD28z, 4-1BB, ICOS, or OX40.

150. The CAR of any one of claims 133 to 149, wherein the cytoplasmic signaling domain is CD3z.

151. A cell expressing the CAR of any one of claims 133-150.

152. The cell of claim 151, wherein the cell is an immune cell.

153. The cell of claim 152, wherein the immune cell is a T-cell.

154. A method of treating a hematopoietic malignancy, the method comprising administering to a human subject:
(a) a population of genetically engineered hematopoietic stem cells, and
(b) the cells of any one of claims 151-153.

155. A polypeptide sequence comprising a polypeptide sequence that is at least 80% identical to the sequence set forth in SEQ ID NO: 51, wherein the polypeptide sequence comprises a mutation at N399D and wherein the polypeptide sequence has reduced binding to a therapeutic anti-FLT3 antibody.

156. A polypeptide sequence comprising a polypeptide sequence that is at least 80% identical to the sequence set forth in SEQ ID NO: 52, wherein the polypeptide sequence comprises a mutation at N399G and wherein the polypeptide sequence has reduced binding to a therapeutic anti-FLT3 antibody.

157. A polypeptide sequence comprising a polypeptide sequence that is at least 80% identical to the sequence set forth in SEQ ID NO: 54, wherein the polypeptide sequence comprises a mutation at S59P and wherein the polypeptide sequence has reduced binding to a therapeutic anti-CD123 antibody.

158. A polypeptide sequence comprising a polypeptide sequence that is at least 80% identical to the sequence set forth in SEQ ID NO: 55, wherein the polypeptide sequence comprises mutations at Y58H and S59P and wherein the polypeptide sequence has reduced binding to a therapeutic anti-CD123 antibody.

159. A polypeptide sequence comprising a polypeptide sequence that is at least 80% identical to the sequence set forth in SEQ ID NO: 56, wherein the polypeptide sequence comprises a mutation at S59F and wherein the polypeptide sequence has reduced binding to a therapeutic anti-CD123 antibody.

160. A polypeptide sequence comprising a polypeptide sequence that is at least 80% identical to the sequence set forth in SEQ ID NO: 57, wherein the polypeptide sequence comprises a mutation at P88S and wherein the polypeptide sequence has reduced binding to a therapeutic anti-CD123 antibody.

161. A polypeptide sequence comprising a polypeptide sequence that is at least 80% identical to the sequence set forth in SEQ ID NO: 58, wherein the polypeptide sequence comprises a mutation at P88L and wherein the polypeptide sequence has reduced binding to a therapeutic anti-CD123 antibody.

162. A polypeptide sequence comprising a polypeptide sequence that is at least 80% identical to the sequence set forth in SEQ ID NO: 67, wherein the polypeptide sequence comprises mutations at F316S, M318V, I319K, V323I, 1334V, E360K, P363V, E366D, E376Q, and H378R and wherein the polypeptide sequence has reduced binding to a therapeutic anti-KIT antibody.

163. A polypeptide sequence comprising a polypeptide sequence that is at least 80% identical to the sequence set forth in SEQ ID NO: 68, wherein the polypeptide sequence comprises a mutation at H378R and wherein the polypeptide sequence has reduced binding to a therapeutic anti-KIT antibody.

164. A nucleic encoding the polypeptide of any one of claims 155-163.
